(19) United States
(12) Patent Application Publication
(10) Pub. No.: US 2022/0089737 A1
(43) Pub. Date: Mar. 24, 2022

(54) MULTI-SPECIFIC IMMUNE TARGETING MOLECULES AND USES THEREOF

(71) Applicant: JANSSEN BIOTECH, INC., Horsham, PA (US)

(72) Inventors: Rajkumar GANESAN, Blue Bell, PA (US); Michael Riis HANSEN, Broomall, PA (US); Iqbal S. GREWAL, Newtown, PA (US); Sanjaya SINGH, Blue Bell, PA (US)

(73) Assignee: JANSSEN BIOTECH, INC., Horsham, PA (US)

(21) Appl. No.: 17/472,237

(22) Filed: Sep. 10, 2021

Related U.S. Application Data
(60) Provisional application No. 63/165,050, filed on Mar. 23, 2021.
(60) Provisional application No. 63/077,458, filed on Sep. 11, 2020.
(60) Provisional application No. 63/077,415, filed on Sep. 11, 2020.
(60) Provisional application No. 63/077,407, filed on Sep. 11, 2020.

(51) Int. Cl. C07K 16/28; C07K 16/30; C07K 16/46; A61P 35/00

(52) U.S. Cl. CPC C07K 16/2818; C07K 16/3069; C07K 16/2878; C07K 2317/31; A61P 35/00; C07K 2317/24; C07K 2317/565; C07K 16/468

(57) ABSTRACT

Provided herein are trispecific antibodies or antigen binding fragments thereof that bind to Vβ17, CD28 and another target (e.g., a cancer antigen, such as BCMA or PSMA) are described. Also described are nucleic acids encoding the antibodies, compositions comprising the antibodies, methods of producing the antibodies, and methods of using the antibodies for treating or preventing diseases.

### CROSS-REFERENCE TO RELATED APPLICATIONS

This application claims the benefit of U.S. Ser. No. 63/077,458 filed Sep. 11, 2020; U.S. Ser. No. 63/077,415 filed Sep. 11, 2020; U.S. Ser. No. 63/077,407 filed Sep. 11, 2020; and U.S. Ser. No. 63/165,050 filed Mar. 23, 2021, the disclosure of each of which is incorporated by reference herein in its entirety.

### FIELD

Provided herein are, among other things, molecules that bind to Vβ17, CD28 and a cancer antigen (e.g., a tumor associated antigen (TAA)), including trispecific antibodies that bind to Vβ17, CD28 and the cancer antigen, as well as antigen binding fragments thereof. Provided herein are, among other things, molecules that bind to Vβ17, CD28 and BCMA, including trispecific antibodies that bind to Vβ17, CD28 and BCMA, and antigen binding fragments thereof. Provided herein are, among other things, molecules that bind to Vβ17, CD28 and PSMA, including trispecific antibodies that bind to Vβ17, CD28 and PSMA, and antigen binding fragments thereof. Also provided are nucleic acids and expression vectors encoding the antibodies, recombinant cells containing the vectors, and compositions comprising the antibodies. Methods of making the antibodies, and methods of using the antibodies to modulate an immune response, are also provided.

### REFERENCE TO SEQUENCE LISTING SUBMITTED ELECTRONICALLY

This application contains a sequence listing, which is submitted electronically via EFS-Web as an ASCII formatted sequence listing with a file “14620-275-999_SL.txt” and a creation date of Sep. 6, 2021 and having a size of 980,276 bytes. The sequence listing submitted via EFS-Web is part of the specification and is herein incorporated by reference in its entirety.

### SUMMARY

In one aspect, provided herein is a trispecific antibody comprising a first binding domain that binds to Vβ17, a second binding domain that binds to a second target, and a third binding domain that binds to CD28. In some embodiments, the second target is a cancer antigen. In some embodiments, the second target is a tumor-specific antigen. In some embodiments, the second target is a tumor associated antigen (TAA). In some embodiments, the second target is a neoantigen. In some embodiments, the second target is BCMA. In some embodiments, the second target is PSMA.

In another aspect, provided is a trispecific antibody comprising: (a) a first binding domain that binds to Vβ17, (b) a second binding domain that binds to cancer antigen, and (c) a third binding domain that binds to CD28. In one embodiment, the cancer antigen is BCMA. In one embodiment, the cancer antigen is PSMA.

In some embodiments of the trispecific antibody, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:9; and (ii) a VL comprising a VL CDR1, a VL CDR2, and a VL CDR3 having an amino acid sequence of a VL CDR1, VL CDR2, and VL CDR3, respectively, of SEQ ID NO:10. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:22. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:23. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:24. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:22. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:23. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:24. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:22. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:23. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:24. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:46; and (ii) a VL comprising a VL CDR1, a VL CDR2, and a VL CDR3 having an amino acid sequence of a VL CDR1, VL CDR2, and VL CDR3, respectively, of SEQ ID NO:49. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:77; and (ii) a VL comprising a VL CDR1, a VL CDR2, and a VL CDR3 having an amino acid sequence of a VL CDR1, VL CDR2, and VL CDR3, respectively, of SEQ ID NO:78. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:79; and (ii) a VL comprising a VL CDR1, a VL CDR2, and a VL CDR3 having an amino acid sequence of a VL CDR1, VL CDR2, and VL CDR3, respectively, of SEQ ID NO:80. In some embodiments, the first binding domain comprises a VH having an amino acid sequence of SEQ ID NO:79. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:81; and (ii) a VL comprising a VL CDR1, a VL CDR2, and a VL CDR3 having an amino acid sequence of a VL CDR1, VL CDR2, and VL CDR3, respectively, of SEQ ID NO:82. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:83; and (ii) a VL comprising a VL CDR1, a VL CDR2, and a VL CDR3 having an amino acid sequence of a VL CDR1, VL CDR2, and VL CDR3, respectively, of SEQ ID NO:84. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:85; and (ii) a VL comprising a VL CDR1, a VL CDR2, and a VL CDR3 having an amino acid sequence of a VL CDR1, VL CDR2, and VL CDR3, respectively, of SEQ ID NO:86. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:87; and (ii) a VL comprising a VL CDR1, a VL CDR2, and a VL CDR3 having an amino acid sequence of a VL CDR1, VL CDR2, and VL CDR3, respectively, of SEQ ID NO:88. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:665. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:1084; and (ii) a VL comprising a VL CDR1, a VL CDR2, and a VL CDR3 having an amino acid sequence of a VL CDR1, VL CDR2, and VL CDR3, respectively, of SEQ ID NO:1085. In some embodiments, the VH CDR1, VH CDR2, VH CDR3, VL CDR1, VL CDR2, and VL CDR3 amino acid sequences of the first binding domain are according to the Kabat numbering system. In some embodiments, the VH CDR1, VH CDR2, VH CDR3, VL CDR1, VL CDR2, and VL CDR3 amino acid sequences of the first binding domain are according to the Chothia numbering system. In some embodiments, the VH CDR1, VH CDR2, VH CDR3, VL CDR1, VL CDR2, and VL CDR3 amino acid sequences of the first binding domain are according to the AbM numbering system. In some embodiments, the VH CDR1, VH CDR2, VH CDR3, VL CDR1, VL CDR2, and VL CDR3 amino acid sequences of the first binding domain are according to the Contact numbering system. In some embodiments, the VH CDR1, VH CDR2, VH CDR3, VL CDR1, VL CDR2, and VL CDR3 amino acid sequences of the first binding domain are according to the IMGT numbering system. In some embodiments, the VH CDR1, VH CDR2, VH CDR3, VL CDR1, VL CDR2, and VL CDR3 amino acid sequences of the first binding domain are according to the Exemplary numbering system. In some embodiments, the first binding domain binds to Vβ17 that is present on the surface of a T cell.

In some embodiments, the cancer antigen is present on the surface of a cell.

In some embodiments, the cancer antigen is BCMA. In some embodiments, the BCMA is present on the surface of a B cell. In some embodiments of the trispecific antibody provided herein, the second binding domain that binds to BCMA comprises: (i) a VH comprising a VH CDR1, a VH CDR2, and a VH CDR3 having an amino acid sequence of a VH CDR1, VH CDR2, and VH CDR3, respectively, of SEQ ID NO:95; and (ii) a VL comprising a VL CDR1, a VL CDR2, and a VL CDR3 having an amino acid sequence of a VL CDR1, VL CDR2, and VL CDR3, respectively, of SEQ ID NO:96. In some embodiments of the trispecific antibody provided herein, the second binding domain that binds to BCMA comprises: (i) a VH comprising a VH CDR1, a VH CDR2, and a VH CDR3 having an amino acid sequence of a VH CDR1, VH CDR2, and VH CDR3, respectively, of SEQ ID NO:1052; and (ii) a VL comprising a VL CDR1, a VL CDR2, and a VL CDR3 having an amino acid sequence of a VL CDR1, VL CDR2, and VL CDR3, respectively, of SEQ ID NO:1053. In some embodiments, the VH CDR1, VH CDR2, VH CDR3, VL CDR1, VL CDR2, and VL CDR3 amino acid sequences of the second binding domain are according to the Kabat numbering system. In some embodiments, the VH CDR1, VH CDR2, VH CDR3, VL CDR1, VL CDR2, and VL CDR3 amino acid sequences of the second binding domain are according to the Chothia numbering system. In some embodiments, the VH CDR1, VH CDR2, VH CDR3, VL CDR1, VL CDR2, and VL CDR3 amino acid sequences of the second binding domain are according to the AbM numbering system. In some embodiments, the VH CDR1, VH CDR2, VH CDR3, VL CDR1, VL CDR2, and VL CDR3 amino acid sequences of the second binding domain are according to the Contact numbering system. In some embodiments, the VH CDR1, VH CDR2, VH CDR3, VL CDR1, VL CDR2, and VL CDR3 amino acid sequences of the second binding domain are according to the IMGT numbering system. In some embodiments, the VH CDR1, VH CDR2, VH CDR3, VL CDR1, VL CDR2, and VL CDR3 amino acid sequences of the second binding domain are according to the Exemplary numbering system.

In some embodiments, the cancer antigen is PSMA. In some embodiments, the PSMA is present on the surface of a prostate cell. In some embodiments of the trispecific antibody provided herein, the second binding domain that binds to PSMA comprises: (i) a VH comprising a VH CDR1, a VH CDR2, and a VH CDR3 having an amino acid sequence of a VH CDR1, VH CDR2, and VH CDR3, respectively, of SEQ ID NO:1046; and (ii) a VL comprising a VL CDR1, a VL CDR2, and a VL CDR3 having an amino acid sequence of a VL CDR1, VL CDR2, and VL CDR3, respectively, of SEQ ID NO:1047. In some embodiments, the VH CDR1, VH CDR2, VH CDR3, VL CDR1, VL CDR2, and VL CDR3 amino acid sequences of the second binding domain are according to the Kabat numbering system. In some embodiments, the VH CDR1, VH CDR2, VH CDR3, VL CDR1, VL CDR2, and VL CDR3 amino acid sequences of the second binding domain are according to the Chothia numbering system. In some embodiments, the VH CDR1, VH CDR2, VH CDR3, VL CDR1, VL CDR2, and VL CDR3 amino acid sequences of the second binding domain are according to the AbM numbering system. In some embodiments, the VH CDR1, VH CDR2, VH CDR3, VL CDR1, VL CDR2, and VL CDR3 amino acid sequences of the second binding domain are according to the Contact numbering system. In some embodiments, the VH CDR1, VH CDR2, VH CDR3, VL CDR1, VL CDR2, and VL CDR3 amino acid sequences of the second binding domain are according to the IMGT numbering system. In some embodiments, the VH CDR1, VH CDR2, VH CDR3, VL CDR1, VL CDR2, and VL CDR3 amino acid sequences of the second binding domain are according to the Exemplary numbering system.

In some embodiments of the trispecific antibody provided herein, the third binding domain binds to CD28 that is present on the surface of a T cell.

In some embodiments of the trispecific antibody provided herein, the third binding domain that binds to CD28 comprises: (i) a VH comprising a VH CDR1, a VH CDR2, and a VH CDR3 having an amino acid sequence of a VH CDR1, VH CDR2, and VH CDR3, respectively, of SEQ ID NO:690; and (ii) a VL comprising a VL CDR1, a VL CDR2, and a VL CDR3 having an amino acid sequence of a VL CDR1, VL CDR2, and VL CDR3, respectively, of SEQ ID NO:696. In some embodiments, the third binding domain that binds to CD28 comprises: (i) a VH comprising a VH CDR1, a VH CDR2, and a VH CDR3 having an amino acid sequence of a VH CDR1, VH CDR2, and VH CDR3, respectively, of SEQ ID NO:691; and (ii) a VL comprising a VL CDR1, a VL CDR2, and a VL CDR3 having an amino acid sequence of a VL CDR1, VL CDR2, and VL CDR3, respectively, of SEQ ID NO:697. In some embodiments, the third binding domain that binds to CD28 comprises: (i) a VH comprising a VH CDR1, a VH CDR2, and a VH CDR3 having an amino acid sequence of a VH CDR1, VH CDR2, and VH CDR3, respectively, of SEQ ID NO:692; and (ii) a VL comprising a VL CDR1, a VL CDR2, and a VL CDR3 having an amino acid sequence of a VL CDR1, VL CDR2, and VL CDR3, respectively, of SEQ ID NO:698. In some embodiments, the third binding domain that binds to CD28 comprises: (i) a VH comprising a VH CDR1, a VH CDR2, and a VH CDR3 having an amino acid sequence of a VH CDR1, VH CDR2, and VH CDR3, respectively, of SEQ ID NO:693; and (ii) a VL comprising a VL CDR1, a VL CDR2, and a VL CDR3 having an amino acid sequence of a VL CDR1, VL CDR2, and VL CDR3, respectively, of SEQ ID NO:699. In some embodiments, the VH CDR1, VH CDR2, VH CDR3, VL CDR1, VL CDR2, and VL CDR3 amino acid sequences of the third binding domain are according to the Kabat numbering system. In some embodiments, the VH CDR1, VH CDR2, VH CDR3, VL CDR1, VL CDR2, and VL CDR3 amino acid sequences of the third binding domain are according to the Chothia numbering system. In some embodiments, the VH CDR1, VH CDR2, VH CDR3, VL CDR1, VL CDR2, and VL CDR3 amino acid sequences of the third binding domain are according to the AbM numbering system. In some embodiments, the VH CDR1, VH CDR2, VH CDR3, VL CDR1, VL CDR2, and VL CDR3 amino acid sequences of the third binding domain are according to the Contact numbering system. In some embodiments, the VH CDR1, VH CDR2, VH CDR3, VL CDR1, VL CDR2, and VL CDR3 amino acid sequences of the third binding domain are according to the IMGT numbering system. In some embodiments, the VH CDR1, VH CDR2, VH CDR3, VL CDR1, VL CDR2, and VL CDR3 amino acid sequences of the third binding domain are according to the Exemplary numbering system.

In some embodiments of the trispecific antibody provided herein, the first binding domain is humanized. In some embodiments, the second binding domain is humanized. In some embodiments, the third binding domain is humanized. In some embodiments, the first binding domain and second binding domain are humanized. In some embodiments, the first binding domain and third binding domain are humanized. In some embodiments, the second binding domain and third binding domain are humanized. In some embodiments, the first binding domain, the second binding domain and the third binding domain are humanized.

In some embodiments of the trispecific antibody provided herein, the trispecific antibody is an IgG antibody. In some embodiments, the IgG antibody is an IgG1, IgG2, IgG3, or IgG4 antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody. In some embodiments, the antibody comprises a kappa light chain. In some embodiments, the antibody comprises a lambda light chain.

In some embodiments of the trispecific antibody provided herein, the first binding domain binds a Vβ17 antigen. In some embodiments, the first binding domain binds a Vβ17 epitope. In some embodiments, the VH CDR1, VH CDR2, VH CDR3, VL CDR1, VL CDR2 and VL CDR3 form a binding site for an antigen of the Vβ17. In some embodiments, the VH CDR1, VH CDR2, VH CDR3, VL CDR1, VL CDR2 and VL CDR3 form a binding site for an epitope of the Vβ17. In some embodiments, the first binding domain specifically binds to Vβ17.

In some embodiments of the trispecific antibody provided herein, the second binding domain binds an antigen of BCMA. In some embodiments, the second binding domain binds an epitope of BCMA. In some embodiments, the VH CDR1, VH CDR2, VH CDR3, VL CDR1, VL CDR2 and VL CDR3 form a binding site for an antigen of BCMA. In some embodiments, the VH CDR1, VH CDR2, VH CDR3, VL CDR1, VL CDR2 and VL CDR3 form a binding site for an epitope of BCMA. In some embodiments, the second binding domain specifically binds to BCMA.

In some embodiments of the trispecific antibody provided herein, second binding domain binds an antigen of PSMA. In some embodiments, the second binding domain binds an epitope of PSMA. In some embodiments, the VH CDR1, VH CDR2, VH CDR3, VL CDR1, VL CDR2 and VL CDR3 form a binding site for an antigen of PSMA. In some embodiments, the VH CDR1, VH CDR2, VH CDR3, VL CDR1, VL CDR2 and VL CDR3 form a binding site for an epitope of PSMA. In some embodiments, the second binding domain specifically binds to PSMA.

In some embodiments of the trispecific antibody provided herein, third binding domain binds an antigen of CD28. In some embodiments, the third binding domain binds an epitope of CD28. In some embodiments, the VH CDR1, VH CDR2, VH CDR3, VL CDR1, VL CDR2 and VL CDR3 form a binding site for an antigen of CD28. In some embodiments, the VH CDR1, VH CDR2, VH CDR3, VL CDR1, VL CDR2 and VL CDR3 form a binding site for an epitope of CD28. In some embodiments, the third binding domain specifically binds to CD28.

In some embodiments of the trispecific antibody provided herein, the trispecific antibody is multivalent.

In another aspect, provided is a trispecific antibody comprising: a first means capable of binding Vβ17 on the surface of a T cell; a second means capable of binding a cancer antigen on the surface of a cancer cell; and a third means capable of binding CD28 on the surface of the T cell. In some embodiments, the cancer antigen is BCMA and the cancer cell is a B cell cancer cell. In some embodiments, the cancer antigen is PSMA and the cancer cell is a prostate cancer cell.

In another aspect, provided is a nucleic acid encoding a trispecific antibody provided herein. In another aspect, provided is a vector comprising a nucleic acid encoding a trispecific antibody provided herein. In another aspect, provided is a host cell comprising a vector comprising a nucleic acid encoding a trispecific antibody provided herein.

In another aspect, provided is a kit comprising a vector comprising a nucleic acid encoding a trispecific antibody provided herein. In another aspect, provided is a kit comprising a trispecific antibody provided herein and packaging for the same.

In one aspect, provided is a pharmaceutical composition comprising a trispecific antibody provided herein, and a pharmaceutically acceptable carrier. In another aspect, provided is a method of producing a pharmaceutical composition comprising a trispecific antibody provided herein, comprising combining the trispecific antibody with a pharmaceutically acceptable carrier to obtain the pharmaceutical composition.

In another aspect, provided is a method of activating a T cell expressing Vβ17, comprising contacting the T cell with the trispecific antibody provided herein.

In another aspect, provided is a process for making an antibody that binds to more than one target molecule, the process comprising: a step for performing a function of obtaining a first binding domain capable of binding to Vβ17 on a T cell; a step for performing a function of obtaining a second binding domain capable of binding to cancer antigen on a cancer cell; a step for performing a function of obtaining a third binding domain capable of binding to CD28 on a T cell; and a step for performing a function of providing an antibody capable of binding to a Vβ17 antigen on the T cell, a cancer antigen on the cancer cell and the CD28 antigen on the T cell. In some embodiments of the process, (i) the step for performing a function of obtaining a second binding domain that binds to the cancer antigen on the cancer cell is repeated n times, and further comprising n steps for performing a function of providing a first binding domain that binds to Vβ17 present on a T cell and n number of target molecules, wherein n is at least 2, or (ii) the step for performing a function of obtaining a third binding domain that binds to the CD28 on the T cell is repeated n times, and further comprising n steps for performing a function of providing a first binding domain that binds to Vβ17 present on a T cell and n number of target molecules, wherein n is at least 2.

In another aspect, provided is a method of directing a T cell expressing Vβ17 and CD28 to a B cell, the method comprising contacting the T cell with a trispecific antibody provided herein, wherein the contacting directs the T cell to the B cell. In another aspect, provides is a method of inhibiting growth or proliferation of B cells expressing BCMA on the cell surface, the method comprising contacting the B cells with a trispecific antibody provided herein, wherein contacting the B cells with the pharmaceutical composition or the antibody or the trispecific antibody inhibits growth or proliferation of the B cells. In some embodiments, the B cells are in the presence of a T cell expressing Vβ17 while in contact with the trispecific antibody. In some embodiments, the B cells are in the presence of a T cell expressing CD28 while in contact with the trispecific antibody.

In another aspect, provided is a method of directing a T cell expressing Vβ17 to a cancer cell, the method comprising contacting the T cell with a trispecific antibody provided herein, wherein the contacting directs the T cell to the cancer cell.

In another aspect, provided is a method of inhibiting the growth of a cancer cell, comprising contacting a trispecific antibody provided herein with the cancer cell having the cancer antigen present on the surface of the cancer cell, wherein the contacting is in the presence of a T cell expressing the Vβ17, and wherein the contacting results in the inhibition of the growth of the cancer cell. In another aspect, provided is a method of inhibiting the proliferation of a cancer cell, comprising contacting a trispecific antibody provided herein with the cancer cell having the cancer antigen present on the surface of the cancer cell, wherein the contacting is in the presence of a T cell expressing the Vβ17, and wherein the contacting results in the inhibition of the proliferation of the cancer cell.

In another aspect, provided is a method of eliminating a cancer cell in a subject, comprising contacting a trispecific antibody provided herein with the cancer cell having the cancer antigen present on the surface of the cancer cell, wherein the contacting is in the presence of a T cell expressing the Vβ17, and wherein the contacting results in the elimination of the cancer cell. In another aspect, provided is a method of treating a disease in a subject, comprising administering an effective amount of a trispecific antibody provided herein to the subject, wherein the disease is caused all or in part by a cancer cell having the cancer antigen present on the surface of the cancer cell. In some embodiments, the subject is a human. In some embodiments, the subject is a subject in need thereof.

In some embodiments, the cancer antigen is present on the surface of a cancer cell.

In some embodiments, the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer antigen is an angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PAP, PDGFRα, PSA, PSA3, PSMA, RANKL, SLAMF7, STEAPI, TARP, TROP2, VEGF, or VEGF-R antigen. In some embodiments, the cancer antigen is a CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, or BRCA1 antigen.

In some embodiments, (i) the adrenal cancer is an adrenocortical carcinoma (ACC), adrenal cortex cancer, pheochromocytoma, or neuroblastoma; (ii) the anal cancer is a squamous cell carcinoma, cloacogenic carcinoma, adenocarcinoma, basal cell carcinoma, or melanoma; (iii) the appendix cancer is a neuroendocrine tumor (NET), mucinous adenocarcinoma, goblet cell carcinoid, intestinal-type adenocarcinoma, or signet-ring cell adenocarcinoma; (iv) the bile duct cancer is an extrahepatic bile duct cancer, adenocarcinomas, hilar bile duct cancer, perihilar bile duct cancer, distal bile duct cancer, or intrahepatic bile duct cancer; (v) the bladder cancer is transitional cell carcinoma (TCC), papillary carcinoma, flat carcinoma, squamous cell carcinoma, adenocarcinoma, small-cell carcinoma, or sarcoma; (vi) the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer; (vii) the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma; (viii) the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer; (ix) the cervical cancer is a squamous cell carcinoma, or adenocarcinoma; (x) the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma; (xi) the esophageal cancer is an adenocarcinoma or squamous cell carcinoma; (xii) the gall bladder cancer is an adenocarcinoma, papillary adenocarcinoma, adenosquamous carcinoma, squamous cell carcinoma, small cell carcinoma, or sarcoma; (xiii) the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT); (xiv) the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer; (xv) the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL); (xvi) the intestinal cancer is a small intestine cancer, small bowel cancer, adenocarcinoma, sarcoma, gastrointestinal stromal tumors, carcinoid tumors, or lymphoma; (xvii) the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma; (xviii) the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS); (xix) the liver cancer is a hepatocellular carcinoma (HCC), fibrolamellar HCC, cholangiocarcinoma, angiosarcoma, or liver metastasis; (xx) the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor; (xxi) the melanoma is a superficial spreading melanoma, nodular melanoma, acral-lentiginous melanoma, lentigo maligna melanoma, amelanotic melanoma, desmoplastic melanoma, ocular melanoma, or metastatic melanoma; (xxii) the mesothelioma is a pleural mesothelioma, peritoneal mesothelioma, pericardial mesothelioma, or testicular mesothelioma; (xxiii) the multiple myeloma is an active myeloma or smoldering myeloma; (xxiv) the neuroendocrine tumor, is a gastrointestinal neuroendocrine tumor, pancreatic neuroendocrine tumor, or lung neuroendocrine tumor; (xxv) the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma; (xxvi) the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer; (xxvii) the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst; (xxviii) the pancreatic cancer is a pancreatic exocrine gland cancer, pancreatic endocrine gland cancer, or pancreatic adenocarcinoma, islet cell tumor, or neuroendocrine tumor; (xxix) the prostate cancer is a prostate adenocarcinoma, prostate sarcoma, transitional cell carcinoma, small cell carcinoma, or neuroendocrine tumor; (xxx) the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer; (xxxi) the skin cancer is a basal cell carcinoma, squamous cell carcinoma, melanoma, Merkel cell carcinoma, Kaposi sarcoma (KS), actinic keratosis, skin lymphoma, or keratoacanthoma; (xxxii) the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma; (xxxiii) the spinal cancer is a spinal metastatic tumor; (xxxiv) the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid; (xxxv) the testicular cancer is a seminoma, non-seminoma, embryonal carcinoma, yolk sac carcinoma, choriocarcinoma, teratoma, gonadal stromal tumor, leydig cell tumor, or sertoli cell tumor; (xxxiv) the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer; (xxxv) the thyroid cancer is a papillary carcinoma, follicular carcinoma, Hürthle cell carcinoma, medullary thyroid carcinoma, or anaplastic carcinoma; (xxxvi) the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma; (xxxvii) the vaginal cancer is a squamous cell carcinoma, adenocarcinoma, melanoma, or sarcoma; or (xxxviii) the vulvar cancer is a squamous cell carcinoma or adenocarcinoma.

In some embodiments, wherein the cancer antigen is BCMA. In some embodiments, the cancer cell is a B cell. In some embodiments, the cancer is a lymphoma. In some embodiments, the cancer is a leukemia.

In some embodiments, the cancer antigen is PSMA. In some embodiments, the cancer cell is a prostate cancer cell. In some embodiments, the cancer is a prostate cancer.

In another aspect, provided herein is a trispecific antibody comprising a first binding domain that binds to Vβ17, a second binding domain that binds to BCMA, and a third binding domain that binds to CD28.

In some embodiments, the first binding domain of the trispecific antibody comprises a VH comprising a VH CDR1 having an amino acid sequence of SEQ ID NO:48, a VH CDR2 having an amino acid sequence of SEQ ID NO:49, and a VH CDR3 having an amino acid sequence of SEQ ID NO:50; and a VL comprising a VL CDR1 having an amino acid sequence of SEQ ID NO:51, a VL CDR2 having an amino acid sequence of SEQ ID NO:52, and a VL CDR3 having an amino acid sequence of SEQ ID NO:53. In some embodiments, the first binding domain of the trispecific antibody comprises a VH having an amino acid sequence of SEQ ID NO:77; a VL having an amino acid sequence of SEQ ID NO:78; or a VH having an amino acid sequence of SEQ ID NO:77, and a VL having an amino acid sequence of SEQ ID NO:78.

In some embodiments, the first binding domain of the trispecific antibody comprises a VH comprising a VH CDR1 having an amino acid sequence of SEQ ID NO:48, a VH CDR2 having an amino acid sequence of SEQ ID NO:54, and a VH CDR3 having an amino acid sequence of SEQ ID NO:55; and a VL comprising a VL CDR1 having an amino acid sequence of SEQ ID NO:56, a VL CDR2 having an amino acid sequence of SEQ ID NO:57, and a VL CDR3 having an amino acid sequence of SEQ ID NO:58. In some embodiments, the first binding domain of the trispecific antibody comprises a VH having an amino acid sequence of SEQ ID NO:79; a VL having an amino acid sequence of SEQ ID NO:80; or a VH having an amino acid sequence of SEQ ID NO:79, and a VL having an amino acid sequence of SEQ ID NO:80.

In some embodiments, the first binding domain of the trispecific antibody comprises a VH comprising a VH CDR1 having an amino acid sequence of SEQ ID NO:59, a VH CDR2 having an amino acid sequence of SEQ ID NO:49, and a VH CDR3 having an amino acid sequence of SEQ ID NO:60; and a VL comprising a VL CDR1 having an amino acid sequence of SEQ ID NO:61, a VL CDR2 having an amino acid sequence of SEQ ID NO:62, and a VL CDR3 having an amino acid sequence of SEQ ID NO:63. In some embodiments, the first binding domain of the trispecific antibody comprises a VH having an amino acid sequence of SEQ ID NO:81; a VL having an amino acid sequence of SEQ ID NO:82; or a VH having an amino acid sequence of SEQ ID NO:81, and a VL having an amino acid sequence of SEQ ID NO:82.

In some embodiments, the first binding domain of the trispecific antibody comprises a VH comprising a VH CDR1 having an amino acid sequence of SEQ ID NO:64, a VH CDR2 having an amino acid sequence of SEQ ID NO:65, and a VH CDR3 having an amino acid sequence of SEQ ID NO:66; and a VL comprising a VL CDR1 having an amino acid sequence of SEQ ID NO:67, a VL CDR2 having an amino acid sequence of SEQ ID NO:68, and a VL CDR3 having an amino acid sequence of SEQ ID NO:69. In some embodiments, the first binding domain of the trispecific antibody comprises a VH having an amino acid sequence of SEQ ID NO:83; a VL having an amino acid sequence of SEQ ID NO:84; or a VH having an amino acid sequence of SEQ ID NO:83, and a VL having an amino acid sequence of SEQ ID NO:84.

In some embodiments the first binding domain of the trispecific antibody comprises a VH comprising a VH CDR1 having an amino acid sequence of SEQ ID NO:70, a VH CDR2 having an amino acid sequence of SEQ ID NO:49, and a VH CDR3 having an amino acid sequence of SEQ ID NO:71; and a VL comprising a VL CDR1 having an amino acid sequence of SEQ ID NO:61, a VL CDR2 having an amino acid sequence of SEQ ID NO:72, and a VL CDR3 having an amino acid sequence of SEQ ID NO:73. In some embodiments, the first binding domain of the trispecific antibody comprises a VH having an amino acid sequence of SEQ ID NO:85; a VL having an amino acid sequence of SEQ ID NO:86; or a VH having an amino acid sequence of SEQ ID NO:85, and a VL having an amino acid sequence of SEQ ID NO:86.

In some embodiments, the first binding domain of the trispecific antibody comprises a VH comprising a VH CDR1 having an amino acid sequence of SEQ ID NO:48, a VH CDR2 having an amino acid sequence of SEQ ID NO:74, and a VH CDR3 having an amino acid sequence of SEQ ID NO:75; and a VL comprising a VL CDR1 having an amino acid sequence of SEQ ID NO:56, a VL CDR2 having an amino acid sequence of SEQ ID NO:57, and a VL CDR3 having an amino acid sequence of SEQ ID NO:76. In some embodiments, the first binding domain of the trispecific antibody comprises a VH having an amino acid sequence of SEQ ID NO:87; a VL having an amino acid sequence of SEQ ID NO:88; or a VH having an amino acid sequence of SEQ ID NO:87, and a VL having an amino acid sequence of SEQ ID NO:88.

In some embodiments, the first binding domain of the trispecific antibody comprises a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and a VL comprising a VL CDR1 having an amino acid sequence of SEQ ID NO:666, a VL CDR2 having an amino acid sequence of SEQ ID NO:5, and a VL CDR3 having an amino acid sequence of SEQ ID NO:6. In some embodiments, the first binding domain of the trispecific antibody comprises a VH having an amino acid sequence of SEQ ID NO:21; a VL having an amino acid sequence of SEQ ID NO:665; or a VH having an amino acid sequence of SEQ ID NO:21, and a VL having an amino acid sequence of SEQ ID NO:665.

In some embodiments of the trispecific antibody provided herein, the Vβ17 is present on the surface of a T cell.

In some embodiments, the cancer antigen is BCMA. In some embodiments, the second binding domain of the trispecific antibody comprises a VH comprising a VH CDR1 having an amino acid sequence of SEQ ID NO:89, a VH CDR2 having an amino acid sequence of SEQ ID NO:90, and a VH CDR3 having an amino acid sequence of SEQ ID NO:91; and a VL comprising a VL CDR1 having an amino acid sequence of SEQ ID NO:92, a VL CDR2 having an amino acid sequence of SEQ ID NO:93, and a VL CDR3 having an amino acid sequence of SEQ ID NO:94. In some embodiments, the second binding domain of the trispecific antibody comprises a VH having an amino acid sequence of SEQ ID NO:95; a VL having an amino acid sequence of SEQ ID NO:96; or a VH having an amino acid sequence of SEQ ID NO:95, and a VL having an amino acid sequence of SEQ ID NO:96. In other embodiments of the trispecific antibody provided herein, the BCMA is present on the surface of a cell. In some embodiments, the cell is a B cell. In some embodiments, the cell is a cancer cell.

In some embodiments, the cancer antigen is PSMA. In some embodiments, the second binding domain of the trispecific antibody comprises a VH comprising a VH CDR1 having an amino acid sequence of SEQ ID NO:1019, a VH CDR2 having an amino acid sequence of SEQ ID NO:1020, and a VH CDR3 having an amino acid sequence of SEQ ID NO:1021; and a VL comprising a VL CDR1 having an amino acid sequence of SEQ ID NO:1034, a VL CDR2 having an amino acid sequence of SEQ ID NO:1035, and a VL CDR3 having an amino acid sequence of SEQ ID NO:1036. In some embodiments, the second binding domain of the trispecific antibody comprises a VH having an amino acid sequence of SEQ ID NO:1046; a VL having an amino acid sequence of SEQ ID NO:1047; or a VH having an amino acid sequence of SEQ ID NO:1046, and a VL having an amino acid sequence of SEQ ID NO:1047. In other embodiments of the trispecific antibody provided herein, the PSMA is present on the surface of a cell. In some embodiments, the cell is a prostate cell. In some embodiments, the cell is a cancer cell.

In some embodiments, the third binding domain of the trispecific antibody comprises a VH comprising a VH CDR1 having an amino acid sequence of SEQ ID NO:702, a VH CDR2 having an amino acid sequence of SEQ ID NO:708, and a VH CDR3 having an amino acid sequence of SEQ ID NO:714; and a VL comprising a VL CDR1 having an amino acid sequence of SEQ ID NO:794, a VL CDR2 having an amino acid sequence of SEQ ID NO:800, and a VL CDR3 having an amino acid sequence of SEQ ID NO:806. In some embodiments, the third binding domain of the trispecific antibody comprises a VH having an amino acid sequence of SEQ ID NO:690; a VL having an amino acid sequence of SEQ ID NO:696; or a VH having an amino acid sequence of SEQ ID NO:690, and a VL having an amino acid sequence of SEQ ID NO:696.

In some embodiments, the third binding domain of the trispecific antibody comprises a VH comprising a VH CDR1 having an amino acid sequence of SEQ ID NO:703, a VH CDR2 having an amino acid sequence of SEQ ID NO:709, and a VH CDR3 having an amino acid sequence of SEQ ID NO:715; and a VL comprising a VL CDR1 having an amino acid sequence of SEQ ID NO:795, a VL CDR2 having an amino acid sequence of SEQ ID NO:801, and a VL CDR3 having an amino acid sequence of SEQ ID NO:807. In some embodiments, the third binding domain of the trispecific antibody comprises a VH having an amino acid sequence of SEQ ID NO:691; a VL having an amino acid sequence of SEQ ID NO:697; or a VH having an amino acid sequence of SEQ ID NO:691, and a VL having an amino acid sequence of SEQ ID NO:697.

In some embodiments, the third binding domain of the trispecific antibody comprises a VH comprising a VH CDR1 having an amino acid sequence of SEQ ID NO:704, a VH CDR2 having an amino acid sequence of SEQ ID NO:710, and a VH CDR3 having an amino acid sequence of SEQ ID NO:716; and a VL comprising a VL CDR1 having an amino acid sequence of SEQ ID NO:796, a VL CDR2 having an amino acid sequence of SEQ ID NO:802, and a VL CDR3 having an amino acid sequence of SEQ ID NO:808. In some embodiments, the third binding domain of the trispecific antibody comprises a VH having an amino acid sequence of SEQ ID NO:692; a VL having an amino acid sequence of SEQ ID NO:698; or a VH having an amino acid sequence of SEQ ID NO:692, and a VL having an amino acid sequence of SEQ ID NO:698.

In some embodiments, the third binding domain of the trispecific antibody comprises a VH comprising a VH CDR1 having an amino acid sequence of SEQ ID NO:705, a VH CDR2 having an amino acid sequence of SEQ ID NO:711, and a VH CDR3 having an amino acid sequence of SEQ ID NO:717; and a VL comprising a VL CDR1 having an amino acid sequence of SEQ ID NO:797, a VL CDR2 having an amino acid sequence of SEQ ID NO:803, and a VL CDR3 having an amino acid sequence of SEQ ID NO:809. In some embodiments, the third binding domain of the trispecific antibody comprises a VH having an amino acid sequence of SEQ ID NO:693; a VL having an amino acid sequence of SEQ ID NO:699; or a VH having an amino acid sequence of SEQ ID NO:693, and a VL having an amino acid sequence of SEQ ID NO:699.

In some embodiments of the trispecific antibody provided herein, CD28 is present on the surface of a T cell. In some embodiments of the trispecific antibody provided herein, CD28 is present on the surface of a B cell.

Any combination of first binding domain, second binding domain, and third binding domain provided herein is contemplated for the trispecific antibodies.

In some embodiments, the trispecific antibody first binding domain is humanized, the second binding domain is humanized, the third binding domain is humanized, the first binding domain and second binding domain are humanized, the first binding domain and third binding domain are humanized, the second binding domain and third binding domain are humanized or all three binding domains are humanized. In some embodiments, the trispecific antibody is an IgG antibody. In some embodiments, the trispecific antibody is an IgG1, IgG2, IgG3, or IgG4 antibody.

In some embodiments of the trispecific antibody provided herein, first binding domain binds a Vβ17 antigen. In some embodiments of the trispecific antibody provided herein, the first binding domain binds a Vβ17 epitope. In some embodiments of the trispecific antibody provided herein, VH CDR1, VH CDR2, VH CDR3, VL CDR1, VL CDR2 and VL CDR3 form a binding site for an antigen of the Vβ17. In some embodiments of the trispecific antibody provided herein, VH CDR1, VH CDR2, VH CDR3, VL CDR1, VL CDR2 and VL CDR3 form a binding site for an epitope of the Vβ17.

In some embodiments of the trispecific antibody provided herein, the second binding domain binds an antigen of BCMA. In some embodiments of the trispecific antibody provided herein, the second binding domain binds an epitope of BCMA. In some embodiments of the trispecific antibody provided herein, the VH CDR1, VH CDR2, VH CDR3, VL CDR1, VL CDR2 and VL CDR3 form a binding site for an antigen of BCMA. In some embodiments of the trispecific antibody provided herein, the VH CDR1, VH CDR2, VH CDR3, VL CDR1, VL CDR2 and VL CDR3 form a binding site for an epitope of BCMA.

In some embodiments of the trispecific antibody provided herein, the second binding domain binds an antigen of PSMA. In some embodiments of the trispecific antibody provided herein, the second binding domain binds an epitope of PSMA. In some embodiments of the trispecific antibody provided herein, the VH CDR1, VH CDR2, VH CDR3, VL CDR1, VL CDR2 and VL CDR3 form a binding site for an antigen of PSMA. In some embodiments of the trispecific antibody provided herein, the VH CDR1, VH CDR2, VH CDR3, VL CDR1, VL CDR2 and VL CDR3 form a binding site for an epitope of PSMA.

In some embodiments of the trispecific antibody provided herein, the third binding domain binds an antigen of CD28. In some embodiments of the trispecific antibody provided herein, the third binding domain binds an epitope of CD28. In some embodiments of the trispecific antibody provided herein, the VH CDR1, VH CDR2, VH CDR3, VL CDR1, VL CDR2 and VL CDR3 form a binding site for an antigen of CD28. In embodiments of the trispecific antibody provided herein, the VH CDR1, VH CDR2, VH CDR3, VL CDR1, VL CDR2 and VL CDR3 form a binding site for an epitope of CD28.

In some embodiments of the trispecific antibody provided herein, the B cell is killed when the trispecific antibody binds to the Vβ17 on the surface of the T cell, CD28 on the surface of the T cell, and BCMA on the surface of the B cell. In some embodiments of the trispecific antibody provided herein, the B cell is killed when the trispecific antibody binds to the Vβ17 on the surface of the T cell, CD28 on the surface of the T cell, and BCMA on the surface of the B cell. In certain embodiments, the B cell is a cancerous B cell.

In some embodiments of the trispecific antibody provided herein, the B cell is killed when the trispecific antibody binds to the Vβ17 on the surface of the T cell, CD28 on the surface of the T cell, and PSMA on the surface of the prostate cell. In some embodiments of the trispecific antibody provided herein, the prostate cell is killed when the trispecific antibody binds to the Vβ17 on the surface of the T cell, CD28 on the surface of the T cell, and PSMA on the surface of the prostate cell. In certain embodiments, the prostate cell is a cancerous prostate cell. Other target cells expressing PSMA are also contemplated.

In some embodiments, the trispecific antibody induces T cell dependent cytotoxicity of the B cell in vitro with an EC₅₀ of less than about 500 pM. In some embodiments, the trispecific antibody induces T cell dependent cytotoxicity of the B cell in vitro with an EC₅₀ of less than about 300 pM. In some embodiments, the trispecific antibody induces T cell dependent cytotoxicity of the B cell in vitro with an EC₅₀ of less than about 160 pM. In some embodiments, the trispecific antibody EC₅₀ is assessed with a mixture of effector T cells and target B cells. In some embodiments of the trispecific antibody provided herein, the effector cell to target cell ratio is about 0.01 to 1 to about 10 to 1. In some embodiments of the trispecific antibody provided herein, the effector cell to target cell ratio is about 0.1 to 1 to about 5 to 1. In some embodiments of the trispecific antibody provided herein, the effector cell to target cell ratio is about 1:1.

In some embodiments of the trispecific antibody provided herein, the T cell releases cytokines when the trispecific antibody binds to CD28 on the surface of the T cell. In some embodiments of the trispecific antibody provided herein, the cytokine is a chemokine, interferon, interleukin, or a protein belonging to the tumour necrosis factor superfamily. In some embodiments of the trispecific antibody provided herein, the chemokine is a CC chemokine, CXC chemokine, C chemokine or a CX3C chemokine. In some embodiments of the trispecific antibody provided herein, the interferon is a Type I interferon, Type 2 interferon or a Type 3 interferon. In some embodiments of the trispecific antibody provided herein, the interleukin is an IL-1, IL-2, IL-3, IL-4, IL-5, IL-6, IL-7, IL-8, IL-9, IL-10, IL-11, IL-12, IL-13, IL-15 or an IL-17. In some embodiments of the trispecific antibody provided herein, the protein belonging to the tumour necrosis factor superfamily is lymphotoxin alpha, tumor necrosis factor, lymphotoxin beta, OX40 ligand, CD40 ligand, Fas ligand, CD27 ligand, CD30 ligand, CD137 ligand, TNF-related apoptosis-inducing ligand, receptor activator of nuclear factor kappa-B ligand, TNF-related weak inducer of apoptosis, proliferation-inducing ligand, B-cell activating factor, LIGHT, vascular endothelial growth inhibitor, TNF superfamily member 18, or ectodysplasin A.

In some embodiments, the trispecific antibody is multivalent. In some embodiments, the trispecific antibody is capable of binding at least five antigens.

In one aspect, provided herein is a trispecific antibody comprising a first means capable of binding Vβ17 on the surface of the T cell; a second means capable of binding BCMA on the surface of the B cell; and a third means capable of binding CD28 on the surface of the T cell. In some embodiments of the trispecific antibody provided herein, the first means capable of binding Vβ17 binds a Vβ17 antigen. In some embodiments of the trispecific antibody provided herein, the first means capable of binding Vβ17 binds a Vβ17 epitope. In some embodiments of the trispecific antibody provided herein, the first means capable of binding BCMA binds a BCMA antigen. In some embodiments of the trispecific antibody provided herein, the first means capable of binding BCMA binds a BCMA epitope. In some embodiments of the trispecific antibody provided herein, the first means capable of binding CD28 binds a CD28 antigen. In some embodiments of the trispecific antibody provided herein, the first means capable of binding CD28 binds a CD28 epitope. In some embodiments of the trispecific antibody provided herein, the second means capable of binding BCMA binds a BCMA antigen. In some embodiments of the trispecific antibody provided herein, the second means capable of binding BCMA binds a BCMA epitope. In some embodiments of the trispecific antibody provided herein, the third means capable of binding CD28 binds a CD28 antigen. In some embodiments of the trispecific antibody provided herein, the third means capable of binding CD28 binds a CD28 epitope.

In one aspect, provided herein is a trispecific antibody comprising a first means capable of binding Vβ17 on the surface of the T cell; a second means capable of binding PSMA on the surface of the prostate cell; and a third means capable of binding CD28 on the surface of the T cell. In some embodiments of the trispecific antibody provided herein, the first means capable of binding Vβ17 binds a Vβ17 antigen. In some embodiments of the trispecific antibody provided herein, the first means capable of binding Vβ17 binds a Vβ17 epitope. In some embodiments of the trispecific antibody provided herein, the first means capable of binding PSMA binds a PSMA antigen. In some embodiments of the trispecific antibody provided herein, the first means capable of binding PSMA binds a PSMA epitope. In some embodiments of the trispecific antibody provided herein, the first means capable of binding CD28 binds a CD28 antigen. In some embodiments of the trispecific antibody provided herein, the first means capable of binding CD28 binds a CD28 epitope. In some embodiments of the trispecific antibody provided herein, the second means capable of binding PSMA binds a PSMA antigen. In some embodiments of the trispecific antibody provided herein, the second means capable of binding PSMA binds a PSMA epitope. In some embodiments of the trispecific antibody provided herein, the third means capable of binding CD28 binds a CD28 antigen. In some embodiments of the trispecific antibody provided herein, the third means capable of binding CD28 binds a CD28 epitope.

In one aspect, provided herein is a nucleic acid encoding the trispecific antibody. In one aspect, provided herein is a vector comprising the nucleic acid encoding the trispecific antibody. In one aspect, provided herein is a host cell comprising the vector comprising the nucleic acid encoding the trispecific antibody. In one aspect, provided herein is a kit comprising the vector comprising the nucleic acid encoding the trispecific antibody and packaging for the same. In one aspect, the packaging comprises a compartment for holding the vector.

In one aspect, provided herein is a pharmaceutical composition comprising the trispecific antibody and a pharmaceutically acceptable carrier. In one aspect, provided herein is a method of producing the pharmaceutical composition comprising combining the antibody with a pharmaceutically acceptable carrier to obtain the pharmaceutical composition.

In one aspect, provided herein is a method of directing a T cell expressing Vβ17 and CD28 to a target cell, the method comprising contacting the T cell with the trispecific antibody, wherein the contacting directs the T cell to the target cell. In one aspect, provided herein is a method of directing a T cell expressing Vβ17 to a target cell, the method comprising contacting the T cell with the trispecific antibody, wherein the contacting directs the T cell to the target cell.

In certain embodiments, the target cell is a cancer cell. In one embodiment, the target cell is a B cell. In one embodiment, the target cell is a cancerous B cell. In certain embodiments, the target cell expresses BCMA. In one embodiment, the target cell is a prostate cell. In one embodiment, the target cell is a prostate cancer cell. In certain embodiments, the target cell expresses PSMA.

In one aspect, provided herein is a method of inhibiting growth or proliferation of target cells expressing a cancer antigen on the cell surface, the method comprising contacting the target cells with the trispecific antibody, wherein contacting the target cells with the pharmaceutical composition or the antibody or the trispecific antibody inhibits growth or proliferation of the target cells. In one aspect, provided herein is a method of inhibiting growth or proliferation of target cells expressing a cancer antigen on the cell surface, the method comprising contacting the target cells with the trispecific antibody, wherein contacting the target cells with the pharmaceutical composition or the antibody or the trispecific antibody inhibits growth or proliferation of the target cells.

In some embodiments of the method provided herein, the target cells are in the presence of a T cell expressing Vβ17 while in contact with the trispecific antibody. In some embodiments of the method provided herein, the target cells are in the presence of a T cell expressing CD28 while in contact with the trispecific antibody.

In one aspect, provided herein is a method for eliminating target cells in a subject, comprising administering an effective amount of the trispecific antibody to the subject. In one aspect, provided herein is a method for eliminating target cells expressing a cancer antigen in a subject, comprising administering an effective amount of the trispecific antibody to the subject.

In one aspect, provided herein is a method treating a disease caused all or in part by target cells expressing a cancer antigen in a subject, comprising administering an effective amount of the trispecific antibody to the subject. In some embodiments of the method, the disease is cancer. In some embodiments, the cancer antigen is BCMA. In some embodiments of the method, the disease is a leukemia. In some embodiments of the method, the disease is a lymphoma. In some embodiments, the cancer antigen is PSMA. In some embodiments of the method, the disease is a prostate cancer. In one aspect, provided herein is a method treating a disease caused all or in part by target cells expressing CD28 in a subject, comprising administering an effective amount of the trispecific antibody provided herein to the subject. In some embodiments of the method, the subject has a cancer. In some embodiments of the method, the subject has a leukemia. In some embodiments of the method, the subject has a lymphoma. In some embodiments of the method, the subject has a prostate cancer. In some embodiments of the method, the subject is a subject in need thereof. In some embodiments of the method, the subject is a human.

In one aspect, provided herein is a trispecific antibody as defined herein for use in therapy.

In one aspect, provided herein is a trispecific antibody as defined herein for use in a method of treating cancer in a subject. In some embodiments, the cancer is a leukemia. In some embodiments of the method, the cancer is a lymphoma. In some embodiments of the method, the cancer is a prostate cancer. In one aspect, the use comprises administering an effective amount of the trispecific antibody to the subject.

In one aspect, provided herein is a process for making an antibody that binds to more than one target molecule, the process comprising a step for performing a function of obtaining a binding domain capable of binding to Vβ17 on an T cell; a step for performing a function of obtaining a binding domain capable of binding to a cancer antigen on a target cell; a step for performing a function of obtaining a binding domain capable of binding to CD28 on a target cell; and a step for performing a function of providing an antibody capable of binding to a Vβ17 (e.g., Vβ17 antigen) on a T cell, a cancer antigen (e.g., tumor associated antigen) on a target cell and a CD28 (e.g., CD28 antigen) on a target cell. In some embodiments of the process, the step for performing a function of obtaining a binding domain capable of binding to the cancer antigen is repeated n times and further comprising n steps for performing a function of providing a binding domain capable of binding to a Vβ17 on a T cell and a CD28 on a target cell and n number of target molecules, wherein n is at least 2. In some embodiments of the process, the step for performing a function of obtaining a binding domain capable of binding to CD28 is repeated n times and further comprising n steps for performing a function of providing a binding domain capable of binding to a Vβ17 on a T cell and a cancer antigen on a target cell and n number of target molecules, wherein n is at least 2. In some embodiments of the process, the binding domain capable of binding to Vβ17 binds a Vβ17 antigen. In some embodiments of the process, the binding domain capable of binding to Vβ17 binds a Vβ17 epitope. In some embodiments of the process, the binding domain capable of binding to a cancer antigen binds an epitope. In some embodiments of the process, the binding domain capable of binding to CD28 binds a CD28 antigen. In some embodiments of the process, the domain capable of binding to CD28 binds a CD28 epitope.

In one aspect, provided herein is a method of directing a T cell expressing Vβ17 and CD28 to a B cell, the method comprising contacting the T cell with the trispecific antibody, wherein the contacting directs the T cell to the B cell. In one aspect, provided herein is a method of directing a T cell expressing Vβ17 to a B cell, the method comprising contacting the T cell with the trispecific antibody, wherein the contacting directs the T cell to the B cell.

In one aspect, provided herein is a method of inhibiting growth or proliferation of B cells expressing BCMA on the cell surface, the method comprising contacting the B cells with the trispecific antibody, wherein contacting the B cells with the pharmaceutical composition or the antibody or the trispecific antibody inhibits growth or proliferation of the B cells. In one aspect, provided herein is a method of inhibiting growth or proliferation of B cells expressing CD28 on the cell surface, the method comprising contacting the B cells with the trispecific antibody, wherein contacting the B cells with the pharmaceutical composition or the antibody or the trispecific antibody inhibits growth or proliferation of the B cells.

In some embodiments of the method provided herein, the B cells are in the presence of a T cell expressing Vβ17 while in contact with the trispecific antibody. In some embodiments of the method provided herein, the B cells are in the presence of a T cell expressing CD28 while in contact with the trispecific antibody.

In one aspect, provided herein is a method for eliminating B cells expressing BCMA in a subject, comprising administering an effective amount of the trispecific antibody to the subject. In one aspect, provided herein is a method for eliminating B cells expressing CD28 in a subject, comprising administering an effective amount of the trispecific antibody to the subject.

In one aspect, provided herein is a method treating a disease caused all or in part by B cells expressing BCMA in a subject, comprising administering an effective amount of the trispecific antibody to the subject. In some embodiments of the method, the disease is cancer. In some embodiments of the method, the disease is a leukemia. In some embodiments of the method, the disease is a lymphoma. In one aspect, provided herein is a method treating a disease caused all or in part by B cells expressing CD28 in a subject, comprising administering an effective amount of the trispecific antibody to the subject. In some embodiments of the method, the disease is cancer. In some embodiments of the method, the disease is a leukemia. In some embodiments of the method, the disease is a lymphoma.

In some embodiments of the method, the subject has a cancer. In some embodiments of the method, the subject has a leukemia. In some embodiments of the method, the subject has a lymphoma. In some embodiments of the method, the subject is a subject in need thereof. In some embodiments of the method, the subject is a human.

In one aspect, provided herein is a trispecific antibody as defined herein for use in therapy.

In one aspect, provided herein is a trispecific antibody as defined herein for use in a method of treating cancer in a subject. In some embodiments, the cancer is a leukemia. In some embodiments of the method, the cancer is a lymphoma. In one aspect, the use comprises administering an effective amount of the trispecific antibody to the subject.

In one aspect, provided herein is a method of activating a T cell expressing Vβ17, comprising contacting the T cell with the trispecific antibody. In one aspect, provided herein is a method of activating a T cell expressing CD28, comprising contacting the T cell with the trispecific antibody. In some embodiments of the method, the contacting results in an increase in cytokine expression, as compared to a control T cell expressing CD28.

In one aspect, provided herein is a process for making an antibody that binds to more than one target molecule, the process comprising a step for performing a function of obtaining a binding domain capable of binding to Vβ17 on an T cell; a step for performing a function of obtaining a binding domain capable of binding to BCMA on a B cell; a step for performing a function of obtaining a binding domain capable of binding to CD28 on a T cell; and a step for performing a function of providing an antibody capable of binding to a Vβ17 (e.g., Vβ17 antigen) on a T cell, a BCMA (e.g., BCMA antigen) on a B cell and a CD28 (e.g., CD28 antigen) on a T cell. In some embodiments of the process, the step for performing a function of obtaining a binding domain capable of binding to BCMA is repeated n times and further comprising n steps for performing a function of providing a binding domain capable of binding to a Vβ17 on a T cell and a CD28 on a T cell and n number of target molecules, wherein n is at least 2. In some embodiments of the process, the step for performing a function of obtaining a binding domain capable of binding to CD28 is repeated n times and further comprising n steps for performing a function of providing a binding domain capable of binding to a Vβ17 on a T cell and a BCMA on a B cell and n number of target molecules, wherein n is at least 2. In some embodiments of the process, the binding domain capable of binding to Vβ17 binds a Vβ17 antigen. In some embodiments of the process, the binding domain capable of binding to Vβ17 binds a Vβ17 epitope. In some embodiments of the process, the binding domain capable of binding to BCMA binds a BCMA antigen. In some embodiments of the process, the binding domain capable of binding to BCMA binds a BCMA epitope. In some embodiments of the process, the binding domain capable of binding to CD28 binds a CD28 antigen. In some embodiments of the process, the domain capable of binding to CD28 binds a CD28 epitope.

In one aspect, provided herein is a process for making an antibody that binds to more than one target molecule, the process comprising a step for performing a function of obtaining a binding domain capable of binding to Vβ17 on an T cell; a step for performing a function of obtaining a binding domain capable of binding to BCMA on a B cell; a step for performing a function of obtaining a binding domain capable of binding to CD28 on a B cell; and a step for performing a function of providing an antibody capable of binding to a Vβ17 (e.g., Vβ17 antigen) on a T cell, a BCMA (e.g., BCMA antigen) on a B cell and a CD28 (e.g., CD28 antigen) on a B cell. In some embodiments of the process, the step for performing a function of obtaining a binding domain capable of binding to BCMA is repeated n times and further comprising n steps for performing a function of providing a binding domain capable of binding to a Vβ17 on a T cell and a CD28 on a B cell and n number of target molecules, wherein n is at least 2. In some embodiments of the process, the step for performing a function of obtaining a binding domain capable of binding to CD28 is repeated n times and further comprising n steps for performing a function of providing a binding domain capable of binding to a Vβ17 on a T cell and a BCMA on a B cell and n number of target molecules, wherein n is at least 2. In some embodiments of the process, the binding domain capable of binding to Vβ17 binds a Vβ17 antigen. In some embodiments of the process, the binding domain capable of binding to Vβ17 binds a Vβ17 epitope. In some embodiments of the process, the binding domain capable of binding to BCMA binds a BCMA antigen. In some embodiments of the process, the binding domain capable of binding to BCMA binds a BCMA epitope. In some embodiments of the process, the binding domain capable of binding to CD28 binds a CD28 antigen. In some embodiments of the process, the domain capable of binding to CD28 binds a CD28 epitope.

### BRIEF DESCRIPTION OF THE DRAWINGS

The foregoing summary, as well as the following detailed description of specific embodiments of the present application, will be better understood when read in conjunction with the appended drawings. It should be understood, however, that the application is not limited to the precise embodiments shown in the drawings.

FIG. 1 shows the binding of an anti-Vβ17/anti-BCMA/anti-CD28 trispecific antibody to recruit T-cells to a cancer cell and to induce cancer cell death.

FIG. 2 shows that anti-Vβ17/anti-BCMA/anti-CD28 trispecific antibodies show potent binding on Pan T cells. Antibodies with C28B19, C28B103 and C28B105 clones showed robust binding to Pan T cells in a dose dependent manner.

FIG. 3 shows that anti-Vβ17/anti-BCMA/anti-CD28 trispecific antibodies show potent binding on H929 cells using BCMA and CD28. Vβ17×CD28×BCMA trispecific antibodies showed potent binding to H929 cells in a CD28 and BCMA dependent manner.

FIGS. 4A-4C show engagement of CD28 potently enhances the activation of vβ17 t cells in plate bound agonism assay. FIG. 4A shows CD25 activation of Vβ17+ T cells at 96 hrs. FIG. 4B shows CD71 activation of Vβ17+ T cells at 96 hrs. FIG. 4C shows proliferation of Vβ17+ T cells at 96 hrs.

FIGS. 5A-5G show engagement of CD28 potently enhances the activation of Vβ17 T cells in the presence of H929 cells. FIGS. 5A-5B show activation of Vβ17 T cells by upregulation of CD25 in the presence of H929 cells at 96 hrs. FIGS. 5C-5D show activation of Vβ17 T cells by upregulation of CD71 in the presence of H929 cells at 96 hrs. FIGS. 5D-5F show activation of Vβ17 T cells by upregulation of CD71 in the presence of H929 cells at 96 hrs.

FIGS. 6A-6C show engagement of CD28 does not induce exhaustion of Vβ17 T cells. FIG. 6A shows LAG3 was induced only on a small fraction of the Vβ17 T cells and no upregulation was seen on the Vβ17− T cells. Overall, only 20% of the Vβ17 T cells were found to express LAG3. FIG. 6B shows PD1 to be upregulated on Vβ17+ T cells in the presence of both the Vβ17×BCMA antibodies and the Vβ17×CD28×BCMA antibodies. FIG. 6C shows TIM3 was induced only on a small fraction of the Vβ17 T cells and no upregulation was seen on the Vβ17− T cells. Overall, only 20% of the Vβ17 T cells were found to express TIM3 cells.

FIGS. 7A-7C show engagement of CD28 potently enhances the cytotoxicity induced by Vβ17 T cells. FIG. 7A shows cytotoxicity mediated by anti-Vβ17/anti-BCMA/anti-CD28 trispecific antibodies. FIG. 7B shows cytotoxicity mediated by anti-Vβ17/anti-BCMA/anti-CD28 trispecific antibodies. FIG. 7C shows cytotoxicity mediated by anti-Vβ17/anti-BCMA/anti-CD28 trispecific antibodies.

FIGS. 8A-8D show engagement of CD28 potently enhances the cytokine secretion. FIGS. 8A-8D show Vβ17×CD28×BCMA trispecific antibodies showed superior cytokine release in comparison to Vβ17×BCMA antibodies and Null×CD28×BCMA antibodies.

FIG. 9 shows expression of co-stimulatory ligands on BCMA expression in H929 cell lines. Both tested multiple myeloma cell lines, MM1.R and H929 were found to express CD28, while no expression of 4IBBL was observed on either of the cell lines.

FIG. 10 shows a glyph illustration of the following Vβ17×CD28×BCMA trispecific antibodies disclosed herein and their descriptions: VB28B35, VB28B36, VB28B38, VB28B39, and VB28B40.

FIG. 11 shows glyph illustration of the following Vβ17×CD28×BCMA trispecific antibodies disclosed herein and their descriptions: VB28B16, VB28B17, VB28B18, VB28B19, VB28B20, VB28B21, VB28B22, VB28B23, VB28B24, VB28B25, VB28B26, and VB28B27.

FIG. 12 shows Glyph illustration of the following Vβ17×CD28×PSMA trispecific antibodies disclosed herein and their descriptions: VB28B28, VB28B29, VB28B31, VB28B32, and VB28B33.

FIG. 13 shows Bcl-xL levels in Vβ17+ T cells upon stimulation with anti-Vβ17/anti-CD28/anti-BCMA trispecific antibodies. Anti-apoptotic protein BcL-xL is induced in Vβ17+ T cells with CD28 activation.

FIG. 14 shows activation profile of Vβ17− T cells with anti-Vβ17/anti-CD28 controls.

FIG. 15 shows cytokine release from anti-Vβ17/anti-CD28 controls. Cytokine secretion is enhanced with CD28 costimulation.

FIG. 16 shows activation profile of Vβ17− T cells with anti-Vβ17/anti-CD28 controls.

FIG. 17 shows cytokine release from anti-Vβ17/anti-CD28 controls. Cytokine secretion is enhanced with CD28 costimulation.

FIG. 18 shows the activation profile of Vβ17+ T cells with Vβ17/null or null/null controls. No activation of Vβ17+ T cells with Null arm controls.

FIG. 19 shows cytotoxicity with anti-Vβ17/anti-CD28/anti-BCMA antibodies and their controls. Depletion of Vβ17+ T cells results in complete loss of cytotoxicity induced by anti-Vβ17/anti-BCMA antibody. No cytotoxicity was observed with anti-CD28/null bispecific.

FIG. 20 shows cytotoxicity with anti-Vβ17/anti-CD28/anti-BCMA antibodies and their controls. Depletion of Vβ17+ T cells results in complete loss of cytotoxicity induced by anti-Vβ17/anti-BCMA antibody. No cytotoxicity was observed with anti-CD28/null bispecific.

FIG. 21 shows the activation profile of Vβ17+ T cells and Vβ17− T cells, with anti-Vβ17/anti-CD28/anti-BCMA antibodies in Donor 1. Combining CD28 costimulation with Vβ17 TCR stimulation potently enhances the activation of Vβ17+ T cells compared to Vβ17 TCR stimulation alone. Anti-Vβ17/anti-CD28/anti-BCMA antibody shows higher activation compared to anti-Vβ17/anti-BCMA antibody, there was no activation with anti-CD28/null bispecific antibody, and activation was observed with anti-CD28/anti-BCMA antibody.

FIG. 22 shows the activation profile of Vβ17+ T cells and Vβ17− T cells, with anti-Vβ17/anti-CD28/anti-BCMA antibodies in Donor 2. Combining CD28 costimulation with Vβ17 TCR stimulation potently enhances the activation of Vβ17+ T cells compared to Vβ17 TCR stimulation alone. Anti-Vβ17/anti-CD28/anti-BCMA antibody shows higher activation compared to anti-Vβ17/anti-BCMA antibody, there was no activation with anti-CD28/null bispecific antibody, and activation was observed with anti-CD28/anti-BCMA antibody.

FIG. 23 shows cytokine release in Pan T cells expressing or depleted of Vβ17 in Donor 1. Cytokine secretion is enhanced with CD28 costimulation in a Vβ17 T cell dependent manner.

FIG. 24 shows cytokine release in Pan T cells expressing or depleted of Vβ17 in Donor 2. Cytokine secretion is enhanced with CD28 costimulation in a Vβ17 T cell dependent manner.

FIG. 25 shows the activation profile of Vβ17+ T cells and Vβ17− T cells with anti-Vβ17/anti-CD28/anti-BCMA antibodies in Donor 1. Combining CD28 costimulation with Vβ17 TCR stimulation potently enhances the activation of Vβ17+ cells compared to Vβ17 TCR stimulation alone. Anti-Vβ17/anti-CD28/anti-BCMA antibody shows higher activation as compared to anti-Vβ17/anti-BCMA antibody. There was no activation observed with anti-CD28/null bispecific antibody but activation was observed with anti-CD28/anti-BCMA antibody.

FIG. 26 shows the activation profile of Vβ17+ T cells and Vβ17− T cells with anti-Vβ17/anti-CD28/anti-BCMA antibodies in Donor 1. Combining CD28 costimulation with Vβ17 TCR stimulation potently enhances the activation of Vβ17+ cells compared to Vβ17 TCR stimulation alone. Anti-Vβ17/anti-CD28/anti-BCMA antibody shows higher activation as compared to anti-Vβ17/anti-BCMA antibody. There was no activation observed with anti-CD28/null bispecific antibody but activation was observed with anti-CD28/anti-BCMA antibody.

FIG. 27 shows cytokine release in Pan T cells with or depleted of Vβ17 in Donor 1. Cytokine secretion is enhanced with CD28 costimulation in a Vβ17 T cell dependent manner.

FIG. 28 shows cytokine release in Pan T cells with or depleted of Vβ17 in Donor 2. Cytokine secretion is enhanced with CD28 costimulation in a Vβ17 T cell dependent manner.

FIG. 29 shows activation profile of Vβ17+ and Vβ17− T cells with anti-Vβ17/anti-CD28/anti-BCMA antibodies in the absence of target cells. Combining CD28 costimulation with Vβ17 TCR stimulation enhanced the activation of Vβ17+ cells compared to Vβ17 TCR stimulation alone or in the absence of target cells. Anti-Vβ17/anti-CD28/anti-CD28 antibodies show similar activation as compared to anti-Vβ17/anti-CD28/anti-BCMA antibodies, activation is observed with anti-CD28/anti-BCMA antibody, but there is no activation with anti-CD28/null or anti-CD28/anti-BCMA bispecific antibodies.

FIG. 30 shows activation profile of Vβ17+ and Vβ17− T cells with anti-Vβ17/anti-CD28/anti-BCMA antibodies in the absence of target cells. Combining CD28 costimulation with Vβ17 TCR stimulation enhanced the activation of Vβ17+ cells compared to Vβ17 TCR stimulation alone. Anti-Vβ17/anti-CD28/anti-CD28 antibodies show higher activation as compared to anti-Vβ17/anti-BCMA antibodies, activation is observed with anti-CD28/anti-BCMA antibody, but there is no activation with anti-CD28/null bispecific antibodies.

FIG. 31 shows anti-Vβ17/anti-CD28/anti-BCMA antibodies do not induce cytotoxicity against non-TAA cell lines.

FIG. 32 shows anti-Vβ17/anti-CD28/anti-BCMA antibodies do not induce cytotoxicity against non-TAA cell lines.

FIG. 33 shows cytotoxicity mediated by anti-Vβ17/anti-CD28/anti-PSMA antibodies. Dose dependent cytotoxicity was observed with only anti-Vβ17/anti-CD28/anti-PSMA antibodies on C4-2B cells. Antibody with PSMB on the CD28 LC N-terminal does not induce cytotoxicity. There was no cytotoxicity induced with anti-Vβ17/null/anti-PSMA antibodies.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., trispecific antibodies and polynucleotides that encode them, Vβ17 polypeptides and Vβ17 polynucleotides that encode them, CD28 polypeptides and CD28 polynucleotides that encode them, BCMA polypeptides and BCMA polynucleotides that encode them, and PSMA polypeptides and PSMA polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected with a nucleic acid molecule disclosed herein. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed trispecific antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

Antibodies

In one aspect, provided herein is a trispecific antibody comprising a first binding domain that binds to Vβ17, a second binding domain that binds to a second target, and a third binding domain that binds to CD28. In some embodiments, the second target is a cancer antigen. In some embodiments, the second target is a tumor-specific antigen. In some embodiments, the second target is a tumor associated antigen (TAA). In some embodiments, the second target is a neoantigen. In some embodiments, the second target is BCMA. In some embodiments, the second target is PSMA.

Also provided herein are multispecific molecules that bind Vβ17, CD28 and BCMA. In some embodiments, the multispecific molecules are trispecific antibodies. In some embodiments, the trispecific antibody comprises a Vβ17 antibody. Exemplary Vβ17 antibodies are provided herein. In some embodiments, the trispecific antibody comprises a CD28 antibody. Exemplary CD28 antibodies are provided herein. In some embodiments, the trispecific antibody comprises a BCMA antibody. Exemplary BCMA antibodies are provided herein. In some embodiments, the trispecific antibody comprises a Vβ17 antibody, a CD28 antibody, and a BCMA antibody. In some embodiments, the trispecific antibody comprises an antigen binding fragment of a Vβ17 antibody. Exemplary antigen binding fragments of Vβ17 antibodies are provided herein. In some embodiments, the trispecific antibody comprises an antigen binding fragment of a CD28 antibody. Exemplary antigen binding fragments of CD28 antibodies are provided herein. In some embodiments, the trispecific antibody comprises an antigen binding fragment of a BCMA antibody. Exemplary antigen binding fragments of BCMA antibodies are provided herein. In some embodiments, the trispecific antibody comprises an antigen binding fragment of a Vβ17 antibody, an antigen binding fragment of a CD28 antibody, and an antigen binding fragment of a BCMA antibody.

In certain embodiments, provided herein are anti-Vβ17/anti-BCMA/anti-CD28 trispecific antibodies or antigen-binding fragments thereof, nucleic acids and expression vectors encoding the trispecific antibodies, recombinant cells containing the vectors, and compositions comprising the trispecific antibodies. Methods of making the antibodies, and methods of using the trispecific antibodies to treat diseases, including cancer, are also provided. The antibodies disclosed herein possess one or more desirable functional properties. In some embodiments, the trispecific antibodies provided herein have high-affinity binding to Vβ17. In some embodiments, the trispecific antibodies provided herein have high-affinity binding to BCMA. In some embodiments, the trispecific antibodies provided herein have high-affinity binding to CD28. In some embodiments, the trispecific antibodies provided herein have high specificity to Vβ17. In some embodiments, the trispecific antibodies provided herein have high specificity to a BCMA. In some embodiments, the trispecific antibodies provided herein have high specificity to a CD28. In some embodiments, the trispecific antibodies provided herein have the ability to treat or prevent a disease or disorder when administered alone. In some embodiments, the trispecific antibodies provided herein have the ability to treat or prevent a disease or disorder when administered in combination with other therapies. In some embodiments, the disease or disorder is a cancer. In some embodiments, the disease or disorder is a leukemia or lymphoma.

Also provided herein are multispecific molecules that bind Vβ17, CD28 and PSMA. In some embodiments, the multispecific molecules are trispecific antibodies. In some embodiments, the trispecific antibody comprises a Vβ17 antibody. Exemplary Vβ17 antibodies are provided herein. In some embodiments, the trispecific antibody comprises a CD28 antibody. Exemplary CD28 antibodies are provided herein. In some embodiments, the trispecific antibody comprises a PSMA antibody. Exemplary PSMA antibodies are provided herein. In some embodiments, the trispecific antibody comprises a Vβ17 antibody, a CD28 antibody, and a PSMA antibody. In some embodiments, the trispecific antibody comprises an antigen binding fragment of a Vβ17 antibody. Exemplary antigen binding fragments of Vβ17 antibodies are provided herein. In some embodiments, the trispecific antibody comprises an antigen binding fragment of a CD28 antibody. Exemplary antigen binding fragments of CD28 antibodies are provided herein. In some embodiments, the trispecific antibody comprises an antigen binding fragment of a PSMA antibody. Exemplary antigen binding fragments of PSMA antibodies are provided herein. In some embodiments, the trispecific antibody comprises an antigen binding fragment of a Vβ17 antibody, an antigen binding fragment of a CD28 antibody, and an antigen binding fragment of a PSMA antibody.

In certain embodiments, provided herein are anti-Vβ17/anti-PSMA/anti-CD28 trispecific antibodies or antigen-binding fragments thereof, nucleic acids and expression vectors encoding the trispecific antibodies, recombinant cells containing the vectors, and compositions comprising the trispecific antibodies. Methods of making the antibodies, and methods of using the trispecific antibodies to treat diseases, including cancer, are also provided. The antibodies disclosed herein possess one or more desirable functional properties. In some embodiments, the trispecific antibodies provided herein have high-affinity binding to Vβ17. In some embodiments, the trispecific antibodies provided herein have high-affinity binding to PSMA. In some embodiments, the trispecific antibodies provided herein have high-affinity binding to CD28. In some embodiments, the trispecific antibodies provided herein have high specificity to Vβ17. In some embodiments, the trispecific antibodies provided herein have high specificity to a PSMA. In some embodiments, the trispecific antibodies provided herein have high specificity to a CD28. In some embodiments, the trispecific antibodies provided herein have the ability to treat or prevent a disease or disorder when administered alone. In some embodiments, the trispecific antibodies provided herein have the ability to treat or prevent a disease or disorder when administered in combination with other therapies. In some embodiments, the disease or disorder is a cancer. In some embodiments, the disease or disorder is a prostate cancer.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies provided herein can be of any of the five major classes or corresponding sub-classes. In specific embodiments, the antibodies provided herein are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies provided herein can contain a kappa or lambda light chain constant domain. According to particular embodiments, the antibodies disclosed herein include heavy and/or light chain constant regions from rat or human antibodies.

In addition to the heavy and light constant domains, antibodies contain an antigen-binding region that is made up of a light chain variable region (VL) and a heavy chain variable region (VH), each of which contains three domains (i.e., complementarity determining regions 1 (CDR1), CDR2 and CDR3. A “CDR” refers to one of three hypervariable regions (HCDR1, HCDR2 or HCDR3) within the non-framework region of the immunoglobulin (Ig or antibody) VH j-sheet framework, or one of three hypervariable regions (LCDR1, LCDR2 or LCDR3) within the non-framework region of the antibody VL j-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. CDR region sequences have also been defined by AbM, Contact and IMGT. Exemplary CDR region sequences are illustrated herein, for example, in the Sequence Listing, and tables provided in the Examples below. The positions of CDRs within a canonical antibody variable region have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra (1997)). Such nomenclature is similarly well known to those skilled in the art.

The light chain variable region CDR1 domain is interchangeably referred to herein as LCDR1 or VL CDR1. The light chain variable region CDR2 domain is interchangeably referred to herein as LCDR2 or VL CDR2. The light chain variable region CDR3 domain is interchangeably referred to herein as LCDR3 or VL CDR3. The heavy chain variable region CDR1 domain is interchangeably referred to herein as HCDR1 or VH CDR1. The heavy chain variable region CDR2 domain is interchangeably referred to herein as HCDR2 or VH CDR2. The heavy chain variable region CDR1 domain is interchangeably referred to herein as HCDR3 or VH CDR3.

The term “hypervariable region”, such as a VH or VL, when used herein refers to the regions of an antibody variable region that are hypervariable in sequence and/or form structurally defined loops. Generally, antibodies comprise six hypervariable regions; three in the VH (HCDR1, HCDR2, HCDR3), and three in the VL (LCDR1, LCDR2, LCDR3). A number of hypervariable region delineations are in use and are encompassed herein. The “Kabat” CDRs are based on sequence variability and are the most commonly used (see, e.g., Kabat et al., Sequences of Proteins of Immunological Interest, 5th Ed. Public Health Service, National Institutes of Health, Bethesda, Md. (1991)). “Chothia” refers instead to the location of the structural loops (see, e.g., Chothia and Lesk, J. Mol. Biol. 196:901-917 (1987)). The end of the Chothia CDR-HCDR1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The “AbM” hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). “Contact” hypervariable regions are based on an analysis of the available complex crystal structures.

Recently, a universal numbering system has been developed and widely adopted, ImMunoGeneTics (IMGT) Information System© (Lafranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). An Exemplary system, shown herein, combines Kabat and Chothia.

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions may comprise “extended hypervariable regions” as follows: 24-36 or 24-34 (LCDR1), 46-56 or 50-56 (LCDR2) and 89-97 or 89-96 (LCDR3) in the VL and 26-35 or 26-35A (HCDR1), 50-65 or 49-65 (HCDR2) and 93-102, 94-102, or 95-102 (HCDR3) in the VH. CDR sequences, reflecting each of the above numbering schemes, are provided herein, including in the Sequence Listing.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable region, which contains the antigen binding site. The constant region may contain the CH1, CH2 and CH3 regions of the heavy chain and the CL region of the light chain.

The term “framework” or “FR” residues are those variable region residues flanking the CDRs. FR residues are present, for example, in chimeric, humanized, human, domain antibodies, diabodies, linear antibodies, and trispecific antibodies. FR residues are those variable domain residues other than the hypervariable region residues or CDR residues.

As used herein, the term an “isolated antibody” refers to an antibody which is substantially free of other antibodies having different antigenic specificities (e.g., an isolated antibody that specifically binds to Vβ17 is substantially free of antibodies that do not bind to Vβ17; an isolated antibody that specifically binds to a second target (e.g., a cancer antigen, such as BCMA or PSMA) is substantially free of antibodies that do not bind to the second target; an isolated antibody that specifically binds to a third target (e.g., CD28) is substantially free of antibodies that do not bind to the second target (e.g., CD28). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies disclosed herein can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′).

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

The term “specificity” refers to selective recognition of an antigen binding protein (such as an antibody) for a particular epitope of an antigen. Natural antibodies, for example, are monospecific. The term “multispecific” as used herein denotes that an antigen binding protein (such as an antibody) has two or more antigen-binding sites of which at least two bind different antigens. “Bispecific” as used herein denotes that an antigen binding protein has two different antigen-binding specificities.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “trispecific antibody” refers to a multispecific antibody that binds no more than three epitopes or three antigens. A trispecific antibody is characterized by a first immunoglobulin variable domain sequence which has binding specificity for a first epitope (e.g., an epitope on a Vβ17 antigen), a second immunoglobulin variable domain sequence that has binding specificity for a second epitope (e.g., an epitope on a BCMA or PSMA antigen) and a third immunoglobulin variable domain sequence that has binding specificity for a third epitope (e.g., an epitope on a CD28 antigen). In an embodiment, the first, second and third epitopes are on different antigens, e.g., the different proteins (or different subunits of a multimeric protein). In an embodiment, a trispecific antibody comprises a heavy chain variable domain sequence and a light chain variable domain sequence which have binding specificity for a first epitope, a heavy chain variable domain sequence and a light chain variable domain sequence which have binding specificity for a second epitope, and a heavy chain variable domain sequence and a light chain variable domain sequence which have binding specificity for a third epitope. In an embodiment, a trispecific antibody comprises a half antibody, or fragment thereof, having binding specificity for a first epitope, a half antibody, or fragment thereof, having binding specificity for a second epitope, and a half antibody, or fragment thereof, having binding specificity for a third epitope. In an embodiment, a trispecific antibody comprises a scFv, or fragment thereof, having binding specificity for a first epitope, a scFv, or fragment thereof, having binding specificity for a second epitope, and a scFv, or fragment thereof, having binding specificity for a third epitope. In an embodiment, the first epitope is located on Vβ17, the second epitope is located on BCMA and the third epitope is located on CD28. In an embodiment, the first epitope is located on Vβ17, the second epitope is located on PSMA and the third epitope is located on CD28.

The term “valent” as used herein denotes the presence of a specified number of binding sites in an antigen binding protein (such as an antibody). A natural antibody for example or a full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein (such as an antibody).

The term “half antibody” as used herein refers to one immunoglobulin heavy chain associated with one immunoglobulin light chain. One skilled in the art will readily appreciate that a half-antibody can encompass a fragment thereof and can also have an antigen binding domain consisting of a single variable domain, e.g., originating from a camelidae.

As used herein, the term “Vβ17” refers to a T cell receptor, which is expressed in response to an immune response on a cytotoxic T cell. Vβ17-expressing CD8+ T cells are commonly produced in response to influenza A virus exposure in a subject. Vβ17-expressing CD8+ T cells provide great recall in response to influenza exposure in the subject. The term “Vβ17” includes any Vβ17 variant, isoform, and species homolog, which is naturally expressed by cells (including T cells) or can be expressed on cells transfected with genes or cDNA encoding the polypeptide. Unless noted, preferably the Vβ17 is a human Vβ17. An exemplary human Vβ17 amino acid sequence is provided by GenBank Accession Number AAB49730.1.

As used herein, the term “BCMA” refers to B-cell maturation antigen, also known as tumor necrosis factor receptor superfamily member 17 (TNFRSF17), is a protein that in humans is encoded by the TNFRSF17 gene. BCMA is a cell surface receptor of the TNF receptor superfamily which recognizes B-cell activating factor. BCMA is preferentially expressed in mature B lymphocytes. The term “BCMA” includes any BCMA variant, isoform, and species homolog, which is naturally expressed by cells (including B cells) or can be expressed on cells transfected with genes or cDNA encoding the polypeptide. Unless noted, preferably the BCMA is a human BCMA. An exemplary human BCMA nucleotide sequence is provided by GenBank Accession Number BC058291. There are four major haplotypes of the BCMA gene in the human genome, and in the present disclosure the term “BCMA” is meant to encompass all four (Kawasaki et al., Genes Immun. 2:276-9, 2001).

As used herein, the term “prostate-specific membrane antigen” or “PSMA” refers to a type II membrane protein expressed on certain cells. The term “PSMA” as used herein includes the protein referred as HGNC: 3788, Entrez Gene: 2346, Ensembl: ENSG00000086205, OMIM: 600934, and UniProtKB: Q04609. The term “PSMA” includes any PSMA variant, isoform, and species homolog, which is naturally expressed by cells (including prostate cells) or can be expressed on cells transfected with genes or cDNA encoding the polypeptide. In specific embodiments, the PSMA is a human PSMA. The term “antibody against PSMA” or “anti-PSMA antibody” as used herein relates to an antibody specifically binding to PSMA.

As used herein, the term “CD28” refers to Cluster of Differentiation 28, which is constitutively expressed on the surface of T cells and some natural killer cells. CD28 is also expressed on some B cells. CD28 is a type I transmembrane glycoprotein and is a member of the Immunoglobulin family by virtue of its single Ig variable-like extracellular domain. The term “CD28” includes any CD28 variant, isoform, and species homolog, which is naturally expressed by cells (including T cells) or can be expressed on cells transfected with genes or cDNA encoding the polypeptide. Unless noted, preferably the CD28 is a human CD28. An exemplary human CD28 amino acid sequence is disclosed in NCBI Accession No. NP_006130.

As used herein, an antibody that “specifically binds to Vβ17” refers to an antibody that binds to a Vβ17, preferably a human Vβ17, with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹⁰ M or less.

As used herein, an antibody that “specifically binds to BCMA” refers to an antibody that binds to a BCMA, preferably a human BCMA, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less.

As used herein, an antibody that “specifically binds to CD28” refers to an antibody that binds to a CD28, preferably a human CD28, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less.

The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system.

The smaller the value of the KD of an antibody, the higher affinity that the antibody binds to a target antigen.

In one aspect, provided is a Vβ17×CD28 trispecific antibody that binds to Vβ17, a second target, and CD28. In some embodiments, the second target is a cancer antigen. In some embodiments, the second target is a tumor-specific antigen. In some embodiments, the second target is a tumor associated antigen (TAA). In some embodiments, the second target is a neoantigen. In some embodiments, the second target is BCMA. In some embodiments, the second target is PSMA. In some embodiments, the antibody is a humanized antibody.

In some embodiments, the Vβ17×CD28 trispecific antibody is a Vβ17×(cancer antigen)×CD28 trispecific antibody. In some embodiments, the Vβ17×CD28 trispecific antibody is a Vβ17×TAA×CD28 trispecific antibody. In some embodiments, the Vβ17×CD28 trispecific antibody is a Vβ17×BCMA×CD28 trispecific antibody. In some embodiments, the Vβ17×CD28 trispecific antibody is a Vβ17×PSMA×CD28 trispecific antibody. Various exemplary Vβ17×CD28 trispecific antibodies are provided herein.

In certain embodiments, provided herein is an Vβ17×CD28 trispecific antibody comprising a VH region, VL region, VH CDR1, VH CDR2, VH CDR3, VL CDR1, VL CDR2, and/or VL CDR3 of any one of the antibodies described herein. In some embodiments, provided herein is a Vβ17×CD28 trispecific antibody comprising a VH region of any one of the antibodies described herein. In some embodiments, provided herein is a Vβ17×CD28 trispecific antibody comprising a VL region of any one of the antibodies described herein. In some embodiments, provided herein is a Vβ17×CD28 trispecific antibody comprising a VH region of any one of the antibodies described herein, and a VL region of any one of the antibodies described herein. In some embodiments, provided herein is a Vβ17×CD28 trispecific antibody comprising a VH CDR1, VH CDR2, and VH CDR3 of any one of the antibodies described herein. In some embodiments, provided herein is a Vβ17×CD28 trispecific antibody comprising a VL CDR1, VL CDR2, and VL CDR3 of any one of the antibodies described herein. In some embodiments, provided herein is a Vβ17×CD28 trispecific antibody comprising a VH CDR1, VH CDR2, and VH CDR3 of any one of the antibodies described herein; and a VL CDR1, VL CDR2, and VL CDR3 of any one of the antibodies described herein. Representative VH and VL amino acid sequences, including VH CDR1, VH CDR2, VH CDR3, VL CDR1, VL CDR2 and VL CDR3 amino acid sequences, of Vβ17×CD28 trispecific antibodies provided herein are provided in the Sequence Listing, as well as the Figures, and Tables in the Examples section.

In certain embodiments, provided is a Vβ17×CD28 trispecific antibody that is an intact antibody. In other embodiments, provided is an antigen binding fragment of the Vβ17×CD28 trispecific antibody. In some embodiments, the antigen binding fragment of the Vβ17×CD28 trispecific antibody is a functional fragment. In some embodiments, the antigen binding fragment is a diabody. In some embodiments, the antigen binding fragment is a Fab. In some embodiments, the antigen binding fragment is a Fab′. In some embodiments, the antigen binding fragment is a F(ab′)2. In some embodiments, the antigen binding fragment is a Fv fragment. In some embodiments, the antigen binding fragment is a disulfide stabilized Fv fragment (dsFv). In some embodiments, the antigen binding fragment is a (dsFv)₂. In some embodiments, the antigen binding fragment is a trispecific dsFv (dsFv-dsFv′). In some embodiments, the antigen binding fragment is a disulfide stabilized diabody (ds diabody). In some embodiments, the antigen binding fragment is a single-chain antibody molecule (scFv). In some embodiments, the antigen binding fragment is a single domain antibody (sdAb). In some embodiments, the antigen binding fragment is an scFv dimer (bivalent diabody). In some embodiments, the antigen binding fragment is a multispecific antibody formed from a portion of an antibody comprising one or more CDRs. In some embodiments, the antigen binding fragment is a camelized single domain antibody. In some embodiments, the antigen binding fragment is a nanobody. In some embodiments, the antigen binding fragment is a domain antibody. In some embodiments, the antigen binding fragment is a bivalent domain antibody. In some embodiments, the antigen binding fragment is an antibody fragment that binds to an antigen but does not comprise a complete antibody structure. In some embodiments, the Vβ17×CD28 trispecific antibody comprises a single chain antibody. In some embodiments, Vβ17×CD28 trispecific antibody comprises a single domain antibody. In certain embodiments, the Vβ17×CD28 trispecific antibody comprises a nanobody. In certain embodiments, the Vβ17×CD28 trispecific antibody comprises a VHH antibody. In certain embodiments, Vβ17×CD28 trispecific antibody comprises a llama antibody. In some embodiments, the Vβ17×CD28 trispecific antibody does not comprise a single chain antibody. In some embodiments, the Vβ17×CD28 trispecific antibody does not comprise a single domain antibody. In certain embodiments, the Vβ17×CD28 trispecific antibody does not comprise a nanobody. In certain embodiments, Vβ17×CD28 trispecific antibody does not comprise a VHH antibody. In certain embodiments, the Vβ17×CD28 trispecific antibody does not comprise a llama antibody. It will be appreciated that any form of trispecific antibody known in the art is contemplated.

In some embodiments, the Vβ17×CD28 trispecific antibody is comprised in a multispecific antibody. In certain embodiments, the Vβ17×CD28 trispecific antibody comprises an antigen binding fragment of an anti-Vβ17 antibody provided herein. In certain embodiments, the Vβ17×CD28 trispecific antibody comprises an antigen binding fragment of an anti-CD28 antibody provided herein. In certain embodiments, the Vβ17×CD28 trispecific antibody comprises an antigen binding fragment of an anti-BCMA antibody provided herein. In certain embodiments, the Vβ17×CD28 trispecific antibody comprises an antigen binding fragment of an anti-PSMA antibody provided herein.

In some embodiments, the Vβ17×CD28 trispecific antibody is an agonistic antibody. In certain embodiments, the Vβ17×CD28 trispecific antibody activates T cells. In other embodiments, the Vβ17×CD28 trispecific antibody is an antagonistic antibody. In certain embodiments, the Vβ17×CD28 trispecific antibody inactivates T cells. In some embodiments, the Vβ17×CD28 trispecific antibody blocks activation of T cells. In some embodiments, the Vβ17×CD28 trispecific antibody modulates the activity of T cells. In some embodiments, the Vβ17×CD28 trispecific antibody neither activates or inactivates the activity of T cells. In specific embodiments, the T cells are human T cells. In specific embodiments, provided is a trispecific antibody comprising a Vβ17×BCMA×CD28 antibody provided herein in a knob-in-hole format. In some embodiments, an Vβ17×CD28 trispecific antibody provided herein may be comprised in a trispecific antibody. In some embodiments, an anti-Vβ17×CD28 trispecific antibody provided herein is comprised in a multispecific antibody.

In certain embodiments, a trispecific antibody provided herein comprises a first binding domain comprising an anti-Vβ17 antibody provided herein that binds to a Vβ17 epitope, a second binding domain comprising an anti-PSMA antibody provided herein that binds to a PSMA epitope, and a third binding domain comprising an anti-CD28 antibody provided herein that binds to a CD28 epitope. In certain embodiments, a trispecific antibody provided herein comprises a first binding domain comprising an anti-Vβ17 antigen binding fragment provided herein that binds to a Vβ17 epitope, a second binding domain comprising an anti-PSMA antigen binding fragment provided herein that binds to a PSMA epitope, and a third binding domain comprising an anti-CD28 antigen binding fragment provided herein that binds to a CD28 epitope.

In one aspect, provided herein is an antibody that binds to Vβ17, BCMA and CD28. In one aspect, provided herein is an antibody that binds to Vβ17, BCMA or CD28. In some embodiments, the anti-Vβ17/anti-BCMA/anti-CD28 antibody is a humanized antibody.

In one aspect, provided herein is an antibody that binds to Vβ17, BCMA and CD28. In one aspect, provided herein is an antibody that binds to Vβ17, BCMA or CD28. In some embodiments, the anti-Vβ17/anti-BCMA/anti-CD28 antibody is a humanized antibody.

In certain embodiments, provided herein is an anti-Vβ17/anti-BCMA/anti-CD28 trispecific antibody comprising a VH region, VL region, VH CDR1, VH CDR2, VH CDR3, VL CDR1, VL CDR2, and/or VL CDR3 of any one of the antibodies described herein. In some embodiments, provided herein is an anti-Vβ17/anti-BCMA/anti-CD28 trispecific antibody comprising a VH region of any one of the antibodies described herein. In some embodiments, provided herein is an anti-Vβ17/anti-BCMA/anti-CD28 trispecific antibody comprising a VL region of any one of the antibodies described herein. In some embodiments, provided herein is an anti-Vβ17/anti-BCMA/anti-CD28 trispecific antibody comprising a VH region of any one of the antibodies described herein, and a VL region of any one of the antibodies described herein. In some embodiments, provided herein is an anti-Vβ17/anti-BCMA/anti-CD28 trispecific antibody comprising a VH CDR1, VH CDR2, and VH CDR3 of any one of the antibodies described herein. In some embodiments, provided herein is an anti-Vβ17/anti-BCMA/anti-CD28 trispecific antibody comprising a VL CDR1, VL CDR2, and VL CDR3 of any one of the antibodies described herein. In some embodiments, provided herein is an anti-Vβ17/anti-BCMA/anti-CD28 trispecific antibody comprising a VH CDR1, VH CDR2, and VH CDR3 of any one of the antibodies described herein; and a VL CDR1, VL CDR2, and VL CDR3 of any one of the antibodies described herein. Representative VH and VL amino acid sequences, including VH CDR1, VH CDR2, VH CDR3, VL CDR1, VL CDR2 and VL CDR3 amino acid sequences, of anti-Vβ17/anti-BCMA/anti-CD28 trispecific antibodies provided herein are provided in the Sequence Listing.

In certain embodiments, provided is an anti-Vβ17/anti-BCMA/anti-CD28 antibody that is an intact antibody. In other embodiments, provided is an anti-Vβ17/anti-BCMA/anti-CD28 antibody is an antigen binding fragment of the anti-Vβ17/anti-BCMA/anti-CD28 antibody. In some embodiments, the antigen binding fragment of the anti-Vβ17/anti-BCMA/anti-CD28 antibody is a functional fragment. In some embodiments, the antigen binding fragment is a diabody. In some embodiments, the antigen binding fragment is a Fab. In some embodiments, the antigen binding fragment is a Fab′. In some embodiments, the antigen binding fragment is a F(ab′)2. In some embodiments, the antigen binding fragment is a Fv fragment. In some embodiments, the antigen binding fragment is a disulfide stabilized Fv fragment (dsFv). In some embodiments, the antigen binding fragment is a (dsFv)₂. In some embodiments, the antigen binding fragment is a trispecific dsFv (dsFv-dsFv′). In some embodiments, the antigen binding fragment is a disulfide stabilized diabody (ds diabody). In some embodiments, the antigen binding fragment is a single-chain antibody molecule (scFv). In some embodiments, the antigen binding fragment is a single domain antibody (sdAb). In some embodiments, the antigen binding fragment is an scFv dimer (bivalent diabody). In some embodiments, the antigen binding fragment is a multispecific antibody formed from a portion of an antibody comprising one or more CDRs. In some embodiments, the antigen binding fragment is a camelized single domain antibody. In some embodiments, the antigen binding fragment is a nanobody. In some embodiments, the antigen binding fragment is a domain antibody. In some embodiments, the antigen binding fragment is a bivalent domain antibody. In some embodiments, the antigen binding fragment is an antibody fragment that binds to an antigen but does not comprise a complete antibody structure. In some embodiments, the anti-Vβ17/anti-BCMA/anti-CD28 trispecific antibody comprises a single chain antibody. In some embodiments, the anti-Vβ17/anti-BCMA/anti-CD28 trispecific antibody comprises a single domain antibody. In certain embodiments, the anti-Vβ17/anti-BCMA/anti-CD28 trispecific antibody comprises a nanobody. In certain embodiments, the anti-Vβ17/anti-BCMA/anti-CD28 trispecific antibody comprises a VHH antibody. In certain embodiments, the anti-Vβ17/anti-BCMA/anti-CD28 trispecific antibody comprises a llama antibody. In some embodiments, the anti-Vβ17/anti-BCMA/anti-CD28 trispecific antibody does not comprise a single chain antibody. In some embodiments, the anti-Vβ17/anti-BCMA/anti-CD28 trispecific antibody does not comprise a single domain antibody. In certain embodiments, the anti-Vβ17/anti-BCMA/anti-CD28 trispecific antibody does not comprise a nanobody. In certain embodiments, the anti-Vβ17/anti-BCMA/anti-CD28 trispecific antibody does not comprise a VHH antibody. In certain embodiments, the anti-Vβ17/anti-BCMA/anti-CD28 trispecific antibody does not comprise a llama antibody. It will be appreciated that any form of trispecific antibody known in the art is contemplated.

In some embodiments, the anti-Vβ17/anti-BCMA/anti-CD28 antibody is a multispecific antibody. In other embodiments, the anti-Vβ17/anti-BCMA/anti-CD28 is a trispecific antibody. In certain embodiments, the multispecific antibody comprises an antigen binding fragment of an anti-Vβ17 antibody, an anti-BCMA antibody, and an anti-CD28 antibody provided herein. In other embodiments, the trispecific antibody comprises an antigen binding fragment of an anti-Vβ17 antibody, an anti-BCMA antibody, and an anti-CD28 antibody provided herein. In some embodiments, the anti-Vβ17/anti-BCMA/anti-CD28 antibody is an agonistic antibody. In certain embodiments, the anti-Vβ17/anti-BCMA/anti-CD28 antibody activates T cells. In other embodiments, the anti-Vβ17/anti-BCMA/anti-CD28 antibody is an antagonistic antibody. In certain embodiments, the anti-Vβ17/anti-BCMA/anti-CD28 antibody inactivates T cells. In some embodiments, the anti-Vβ17/anti-BCMA/anti-CD28 antibody blocks activation of T cells. In some embodiments, the anti-Vβ17/anti-BCMA/anti-CD28 antibody modulates the activity of T cells. In some embodiments, the anti-Vβ17/anti-BCMA/anti-CD28 antibody neither activates or inactivates the activity of T cells. In specific embodiments, the T cells are human T cells. In specific embodiments, provided is a trispecific antibody comprising a Vβ17×BCMA×CD28 antibody provided herein in a knob-in-hole format. In some embodiments, an anti-Vβ17/anti-BCMA/anti-CD28 antibody provided herein may be comprised in a trispecific antibody. In some embodiments, an anti-Vβ17/anti-BCMA/anti-CD28 trispecific antibody provided herein may be comprised in a multispecific antibody.

In certain embodiments, a trispecific antibody provided herein comprises a first binding domain comprising an anti-Vβ17 antibody provided herein that binds to a Vβ17 epitope, a second binding domain comprising an anti-BCMA antibody provided herein that binds to a BCMA epitope, and a third binding domain comprising an anti-CD28 antibody provided herein that binds to a CD28 epitope. In certain embodiments, a trispecific antibody provided herein comprises a first binding domain comprising an anti-Vβ17 antigen binding fragment provided herein that binds to a Vβ17 epitope, a second binding domain comprising an anti-BCMA antigen binding fragment provided herein that binds to a BCMA epitope, and a third binding domain comprising an anti-CD28 antigen binding fragment provided herein that binds to a CD28 epitope.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system. Exemplary sets of 6 CDRs (VH CDR1-3 and VL CDR1-3) of certain antibody embodiments are provided herein. Other sets of CDRs are contemplated and within the scope of the antibody embodiments provided herein.

In one aspect, provided herein is a trispecific antibody comprising: (a) a first binding domain that binds to Vβ17, (b) a second binding domain that binds to second target, and (c) a third binding domain that binds to CD28. In specific embodiments, the second target is a cancer antigen. Thus, in certain aspects, provided herein is a trispecific antibody comprising: (a) a first binding domain that binds to Vβ17, (b) a second binding domain that binds to cancer antigen, and (c) a third binding domain that binds to CD28. In some embodiments, the cancer antigen is a tumor associated antigen (TAA). In some embodiments, the cancer antigen is a tumor specific antigen. In some embodiments, the cancer antigen is a neoantigen. In some embodiments, the cancer antigen is BCMA. In some embodiments, the cancer antigen is PSMA. Thus, in one aspect, provided herein is a trispecific antibody comprising: (a) a first binding domain that binds to Vβ17, (b) a second binding domain that binds to BCMA, and (c) a third binding domain that binds to CD28. In another aspect, provided herein is a trispecific antibody comprising: (a) a first binding domain that binds to Vβ17, (b) a second binding domain that binds to BCMA, and (c) a third binding domain that binds to CD28.

In some embodiments, the first binding domain that binds to Vβ17 comprises VH CDR1, VH CDR2, and VH CDR3 amino acid sequences of a Vβ17 antibody provided herein. In some embodiments, the first binding domain that binds to Vβ17 comprises VL CDR1, VL CDR2 and VL CDR3 amino acid sequences of a Vβ17 antibody provided herein. In some embodiments, the first binding domain that binds to Vβ17 comprises VH CDR1, VH CDR2, VH CDR3, VL CDR1, VL CDR2 and VL CDR3 amino acid sequences of a Vβ17 antibody provided herein. In other embodiments, the first binding domain that binds to Vβ17 comprises VH amino acid sequence of a Vβ17 antibody provided herein. In other embodiments, the first binding domain that binds to Vβ17 comprises a VL amino acid sequence of a Vβ17 antibody provided herein. In other embodiments, the first binding domain that binds to Vβ17 comprises VH and VL amino acid sequences of a Vβ17 antibody provided herein. In other embodiments, the first binding domain that binds to Vβ17 comprises heavy chain amino acid sequence of a Vβ17 antibody provided herein. In other embodiments, the first binding domain that binds to Vβ17 comprises a light chain amino acid sequence of a Vβ17 antibody provided herein. In other embodiments, the first binding domain that binds to Vβ17 comprises heavy chain and light chain amino acid sequences of a Vβ17 antibody provided herein. In some embodiments, the Vβ17 antibody is clone E17.5F. In some embodiments, the Vβ17 antibody is clone B17B1. In some embodiments, the Vβ17 antibody is clone B17H1. In some embodiments, the Vβ17 antibody is clone B17H3. In some embodiments, the Vβ17 antibody is clone B17H4. In some embodiments, the Vβ17 antibody is clone B17H5. In some embodiments, the Vβ17 antibody is clone. In some embodiments, the Vβ17 antibody is clone B17B14. In some embodiments, the Vβ17 antibody is clone B17B15. In some embodiments, the Vβ17 antibody is clone B17B16. In some embodiments, the Vβ17 antibody is clone B17B17. In some embodiments, the Vβ17 antibody is clone B17B18. In some embodiments, the Vβ17 antibody is clone B17B19. In some embodiments, the Vβ17 antibody is clone B17B20. In some embodiments, the Vβ17 antibody is clone B17B21. In some embodiments, the Vβ17 antibody is clone B17B22. In some embodiments, the Vβ17 antibody is clone B17B2. In some embodiments, the Vβ17 antibody is clone Vb17_202B4D1. In some embodiments, the Vβ17 antibody is clone Vb17_210E10A1. In some embodiments, the Vβ17 antibody is clone B17B663. In some embodiments, the Vβ17 antibody is clone B17B694. In some embodiments, the Vβ17 antibody is clone B17B698. In some embodiments, the Vβ17 antibody is clone B17B733. In some embodiments, the Vβ17 antibody is clone Vb17_N33S. Other Vβ17 antibodies, including antigen binding fragments thereof, are also contemplated as the first binding arm that binds to Vβ17 of in the trispecific antibodies provided herein.

In some embodiments, the first binding domain that binds to Vβ17 comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain that binds to Vβ17 comprises: (i) a VH comprising a VH CDR1 having an amino acid sequence of SEQ ID NO:97, a VH CDR2 having an amino acid sequence of SEQ ID NO:98, and a VH CDR3 having an amino acid sequence of SEQ ID NO:99; and (ii) a VL comprising a VL CDR1 having an amino acid sequence of SEQ ID NO:100, a VL CDR2 having an amino acid sequence of SEQ ID NO:101, and a VL CDR3 having an amino acid sequence of SEQ ID NO:102. In some embodiments, the first binding domain that binds to Vβ17 comprises: (i) a VH comprising a VH CDR1 having an amino acid sequence of SEQ ID NO:103, a VH CDR2 having an amino acid sequence of SEQ ID NO:104, and a VH CDR3 having an amino acid sequence of SEQ ID NO:105; and (ii) a VL comprising a VL CDR1 having an amino acid sequence of SEQ ID NO:106, a VL CDR2 having an amino acid sequence of SEQ ID NO:107, and a VL CDR3 having an amino acid sequence of SEQ ID NO:108. In some embodiments, the first binding domain that binds to Vβ17 comprises: (i) a VH comprising a VH CDR1 having an amino acid sequence of SEQ ID NO:109, a VH CDR2 having an amino acid sequence of SEQ ID NO:110, and a VH CDR3 having an amino acid sequence of SEQ ID NO:111; and (ii) a VL comprising a VL CDR1 having an amino acid sequence of SEQ ID NO:112, a VL CDR2 having an amino acid sequence of SEQ ID NO:113, and a VL CDR3 having an amino acid sequence of SEQ ID NO:114. In some embodiments, the first binding domain that binds to Vβ17 comprises: (i) a VH comprising a VH CDR1 having an amino acid sequence of SEQ ID NO:115, a VH CDR2 having an amino acid sequence of SEQ ID NO:116, and a VH CDR3 having an amino acid sequence of SEQ ID NO:117; and (ii) a VL comprising a VL CDR1 having an amino acid sequence of SEQ ID NO:118, a VL CDR2 having an amino acid sequence of SEQ ID NO:119, and a VL CDR3 having an amino acid sequence of SEQ ID NO:120. In some embodiments, the first binding domain that binds to Vβ17 comprises: (i) a VH comprising a VH CDR1 having an amino acid sequence of SEQ ID NO:121, a VH CDR2 having an amino acid sequence of SEQ ID NO:122, and a VH CDR3 having an amino acid sequence of SEQ ID NO:123; and (ii) a VL comprising a VL CDR1 having an amino acid sequence of SEQ ID NO:124, a VL CDR2 having an amino acid sequence of SEQ ID NO:125, and a VL CDR3 having an amino acid sequence of SEQ ID NO:126. In some embodiments, the first binding domain that binds to Vβ17 comprises: (i) a VH comprising a VH CDR1 having an amino acid sequence of SEQ ID NO:127, a VH CDR2 having an amino acid sequence of SEQ ID NO:128, and a VH CDR3 having an amino acid sequence of SEQ ID NO:129; and (ii) a VL comprising a VL CDR1 having an amino acid sequence of SEQ ID NO:130, a VL CDR2 having an amino acid sequence of SEQ ID NO:131, and a VL CDR3 having an amino acid sequence of SEQ ID NO:132. In some embodiments, the first binding domain that binds to Vβ17 comprises: (i) a VH comprising a VH CDR1 having an amino acid sequence of SEQ ID NO:133, a VH CDR2 having an amino acid sequence of SEQ ID NO:134, and a VH CDR3 having an amino acid sequence of SEQ ID NO:135; and (ii) a VL comprising a VL CDR1 having an amino acid sequence of SEQ ID NO:136, a VL CDR2 having an amino acid sequence of SEQ ID NO:137, and a VL CDR3 having an amino acid sequence of SEQ ID NO:138. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:25; and (ii) a VL comprising a VL CDR1, a VL CDR2, and a VL CDR3 having an amino acid sequence of a VL CDR1, VL CDR2, and VL CDR3, respectively, of SEQ ID NO:26. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:7; and (ii) a VL comprising a VL CDR1, a VL CDR2, and a VL CDR3 having an amino acid sequence of a VL CDR1, VL CDR2, and VL CDR3, respectively, of SEQ ID NO:8. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:9; and (ii) a VL comprising a VL CDR1, a VL CDR2, and a VL CDR3 having an amino acid sequence of a VL CDR1, VL CDR2, and VL CDR3, respectively, of SEQ ID NO:10. In some embodiments, the first binding domain comprises a VH having an amino acid sequence of SEQ ID NO:25. In some embodiments, the first binding domain comprises a VL having an amino acid sequence of SEQ ID NO:26. In some embodiments, the first binding domain comprises a VH having an amino acid sequence of SEQ ID NO:25, and a VL having an amino acid sequence of SEQ ID NO:26. In some embodiments, the first binding domain comprises a heavy chain having an amino acid sequence of SEQ ID NO:7. In some embodiments, the first binding domain comprises a light chain having an amino acid sequence of SEQ ID NO:8. In some embodiments, the first binding domain comprises a heavy chain having an amino acid sequence of SEQ ID NO:7, and a light chain having an amino acid sequence of SEQ ID NO:8. In some embodiments, the first binding domain comprises a heavy chain having an amino acid sequence of SEQ ID NO:9. In some embodiments, the first binding domain comprises a light chain having an amino acid sequence of SEQ ID NO:10. In some embodiments, the first binding domain comprises a heavy chain having an amino acid sequence of SEQ ID NO:9, and a light chain having an amino acid sequence of SEQ ID NO:10. In some embodiments, the first binding domain comprises a VH having an amino acid sequence having at least 95% identity to an amino acid sequence of SEQ ID NO:25. In some embodiments, the first binding domain comprises a VL having an amino acid sequence having at least 95% identity to an amino acid sequence of SEQ ID NO:26. In some embodiments, the first binding domain comprises a VH having an amino acid sequence having at least 95% identity to an amino acid sequence of SEQ ID NO:25, and a VL having an amino acid sequence having at least 95% identity to an amino acid sequence of SEQ ID NO:26. In some embodiments, the first binding domain comprises a heavy chain having an amino acid sequence having at least 95% identity to an amino acid sequence of SEQ ID NO:7. In some embodiments, the first binding domain comprises a light chain having an amino acid sequence having at least 95% identity to an amino acid sequence of SEQ ID NO:8. In some embodiments, the first binding domain comprises a heavy chain having an amino acid sequence having at least 95% identity to an amino acid sequence of SEQ ID NO:7, and a light chain having an amino acid sequence having at least 95% identity to an amino acid sequence of SEQ ID NO:8. In some embodiments, the first binding domain comprises a heavy chain having an amino acid sequence having at least 95% identity to an amino acid sequence of SEQ ID NO:9. In some embodiments, the first binding domain comprises a light chain having an amino acid sequence having at least 95% identity to an amino acid sequence of SEQ ID NO:10. In some embodiments, the first binding domain comprises a heavy chain having an amino acid sequence having at least 95% identity to an amino acid sequence of SEQ ID NO:9, and a light chain having an amino acid sequence having at least 95% identity to an amino acid sequence of SEQ ID NO:10.

In some embodiments, the first binding domain that binds to Vβ17 comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain that binds to Vβ17 comprises: (i) a VH comprising a VH CDR1 having an amino acid sequence of SEQ ID NO:139, a VH CDR2 having an amino acid sequence of SEQ ID NO:140, and a VH CDR3 having an amino acid sequence of SEQ ID NO:141; and (ii) a VL comprising a VL CDR1 having an amino acid sequence of SEQ ID NO:142, a VL CDR2 having an amino acid sequence of SEQ ID NO:143, and a VL CDR3 having an amino acid sequence of SEQ ID NO:144. In some embodiments, the first binding domain that binds to Vβ17 comprises: (i) a VH comprising a VH CDR1 having an amino acid sequence of SEQ ID NO:145, a VH CDR2 having an amino acid sequence of SEQ ID NO:146, and a VH CDR3 having an amino acid sequence of SEQ ID NO:147; and (ii) a VL comprising a VL CDR1 having an amino acid sequence of SEQ ID NO:148, a VL CDR2 having an amino acid sequence of SEQ ID NO:149, and a VL CDR3 having an amino acid sequence of SEQ ID NO:150. In some embodiments, the first binding domain that binds to Vβ17 comprises: (i) a VH comprising a VH CDR1 having an amino acid sequence of SEQ ID NO:151, a VH CDR2 having an amino acid sequence of SEQ ID NO:152, and a VH CDR3 having an amino acid sequence of SEQ ID NO:153; and (ii) a VL comprising a VL CDR1 having an amino acid sequence of SEQ ID NO:154, a VL CDR2 having an amino acid sequence of SEQ ID NO:155, and a VL CDR3 having an amino acid sequence of SEQ ID NO:156. In some embodiments, the first binding domain that binds to Vβ17 comprises: (i) a VH comprising a VH CDR1 having an amino acid sequence of SEQ ID NO:157, a VH CDR2 having an amino acid sequence of SEQ ID NO:158, and a VH CDR3 having an amino acid sequence of SEQ ID NO:159; and (ii) a VL comprising a VL CDR1 having an amino acid sequence of SEQ ID NO:160, a VL CDR2 having an amino acid sequence of SEQ ID NO:161, and a VL CDR3 having an amino acid sequence of SEQ ID NO:162. In some embodiments, the first binding domain that binds to Vβ17 comprises: (i) a VH comprising a VH CDR1 having an amino acid sequence of SEQ ID NO:163, a VH CDR2 having an amino acid sequence of SEQ ID NO:164, and a VH CDR3 having an amino acid sequence of SEQ ID NO:165; and (ii) a VL comprising a VL CDR1 having an amino acid sequence of SEQ ID NO:166, a VL CDR2 having an amino acid sequence of SEQ ID NO:167, and a VL CDR3 having an amino acid sequence of SEQ ID NO:168. In some embodiments, the first binding domain that binds to Vβ17 comprises: (i) a VH comprising a VH CDR1 having an amino acid sequence of SEQ ID NO:169, a VH CDR2 having an amino acid sequence of SEQ ID NO:170, and a VH CDR3 having an amino acid sequence of SEQ ID NO:171; and (ii) a VL comprising a VL CDR1 having an amino acid sequence of SEQ ID NO:172, a VL CDR2 having an amino acid sequence of SEQ ID NO:173, and a VL CDR3 having an amino acid sequence of SEQ ID NO:174. In some embodiments, the first binding domain that binds to Vβ17 comprises: (i) a VH comprising a VH CDR1 having an amino acid sequence of SEQ ID NO:175, a VH CDR2 having an amino acid sequence of SEQ ID NO:176, and a VH CDR3 having an amino acid sequence of SEQ ID NO:177; and (ii) a VL comprising a VL CDR1 having an amino acid sequence of SEQ ID NO:178, a VL CDR2 having an amino acid sequence of SEQ ID NO:179, and a VL CDR3 having an amino acid sequence of SEQ ID NO:180. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:22. In some embodiments, the first binding domain comprises a VH having an amino acid sequence of SEQ ID NO:19. In some embodiments, the first binding domain comprises a VL having an amino acid sequence of SEQ ID NO:22. In some embodiments, the first binding domain comprises a VH having an amino acid sequence of SEQ ID NO:19, and a VL having an amino acid sequence of SEQ ID NO:22. In some embodiments, the first binding domain comprises a VH having an amino acid sequence having at least 95% identity to an amino acid sequence of SEQ ID NO:19. In some embodiments, the first binding domain comprises a VL having an amino acid sequence having at least 95% identity to an amino acid sequence of SEQ ID NO:22. In some embodiments, the first binding domain comprises a VH having an amino acid sequence having at least 95% identity to an amino acid sequence of SEQ ID NO:19, and a VL having an amino acid sequence having at least 95% identity to an amino acid sequence of SEQ ID NO:22.

In some embodiments, the first binding domain that binds to Vβ17 comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain that binds to Vβ17 comprises: (i) a VH comprising a VH CDR1 having an amino acid sequence of SEQ ID NO:139, a VH CDR2 having an amino acid sequence of SEQ ID NO:140, and a VH CDR3 having an amino acid sequence of SEQ ID NO:141; and (ii) a VL comprising a VL CDR1 having an amino acid sequence of SEQ ID NO:184, a VL CDR2 having an amino acid sequence of SEQ ID NO:185, and a VL CDR3 having an amino acid sequence of SEQ ID NO:186. In some embodiments, the first binding domain that binds to Vβ17 comprises: (i) a VH comprising a VH CDR1 having an amino acid sequence of SEQ ID NO:145, a VH CDR2 having an amino acid sequence of SEQ ID NO:146, and a VH CDR3 having an amino acid sequence of SEQ ID NO:147; and (ii) a VL comprising a VL CDR1 having an amino acid sequence of SEQ ID NO:190, a VL CDR2 having an amino acid sequence of SEQ ID NO:191, and a VL CDR3 having an amino acid sequence of SEQ ID NO:192. In some embodiments, the first binding domain that binds to Vβ17 comprises: (i) a VH comprising a VH CDR1 having an amino acid sequence of SEQ ID NO:151, a VH CDR2 having an amino acid sequence of SEQ ID NO:152, and a VH CDR3 having an amino acid sequence of SEQ ID NO:153; and (ii) a VL comprising a VL CDR1 having an amino acid sequence of SEQ ID NO:196, a VL CDR2 having an amino acid sequence of SEQ ID NO:197, and a VL CDR3 having an amino acid sequence of SEQ ID NO:198. In some embodiments, the first binding domain that binds to Vβ17 comprises: (i) a VH comprising a VH CDR1 having an amino acid sequence of SEQ ID NO:157, a VH CDR2 having an amino acid sequence of SEQ ID NO:158, and a VH CDR3 having an amino acid sequence of SEQ ID NO:159; and (ii) a VL comprising a VL CDR1 having an amino acid sequence of SEQ ID NO:202, a VL CDR2 having an amino acid sequence of SEQ ID NO:203, and a VL CDR3 having an amino acid sequence of SEQ ID NO:204. In some embodiments, the first binding domain that binds to Vβ17 comprises: (i) a VH comprising a VH CDR1 having an amino acid sequence of SEQ ID NO:163, a VH CDR2 having an amino acid sequence of SEQ ID NO:164, and a VH CDR3 having an amino acid sequence of SEQ ID NO:165; and (ii) a VL comprising a VL CDR1 having an amino acid sequence of SEQ ID NO:208, a VL CDR2 having an amino acid sequence of SEQ ID NO:209, and a VL CDR3 having an amino acid sequence of SEQ ID NO:210. In some embodiments, the first binding domain that binds to Vβ17 comprises: (i) a VH comprising a VH CDR1 having an amino acid sequence of SEQ ID NO:169, a VH CDR2 having an amino acid sequence of SEQ ID NO:170, and a VH CDR3 having an amino acid sequence of SEQ ID NO:171; and (ii) a VL comprising a VL CDR1 having an amino acid sequence of SEQ ID NO:214, a VL CDR2 having an amino acid sequence of SEQ ID NO:215, and a VL CDR3 having an amino acid sequence of SEQ ID NO:216. In some embodiments, the first binding domain that binds to Vβ17 comprises: (i) a VH comprising a VH CDR1 having an amino acid sequence of SEQ ID NO:175, a VH CDR2 having an amino acid sequence of SEQ ID NO:176, and a VH CDR3 having an amino acid sequence of SEQ ID NO:177; and (ii) a VL comprising a VL CDR1 having an amino acid sequence of SEQ ID NO:220, a VL CDR2 having an amino acid sequence of SEQ ID NO:221, and a VL CDR3 having an amino acid sequence of SEQ ID NO:222. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:23. In some embodiments, the first binding domain comprises a VH having an amino acid sequence of SEQ ID NO:19. In some embodiments, the first binding domain comprises a VL having an amino acid sequence of SEQ ID NO:23. In some embodiments, the first binding domain comprises a VH having an amino acid sequence of SEQ ID NO:19, and a VL having an amino acid sequence of SEQ ID NO:23. In some embodiments, the first binding domain comprises a VH having an amino acid sequence having at least 95% identity to an amino acid sequence of SEQ ID NO:19. In some embodiments, the first binding domain comprises a VL having an amino acid sequence having at least 95% identity to an amino acid sequence of SEQ ID NO:23. In some embodiments, the first binding domain comprises a VH having an amino acid sequence having at least 95% identity to an amino acid sequence of SEQ ID NO:19, and a VL having an amino acid sequence having at least 95% identity to an amino acid sequence of SEQ ID NO:23.

In some embodiments, the first binding domain that binds to Vβ17 comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain that binds to Vβ17 comprises: (i) a VH comprising a VH CDR1 having an amino acid sequence of SEQ ID NO:139, a VH CDR2 having an amino acid sequence of SEQ ID NO:140, and a VH CDR3 having an amino acid sequence of SEQ ID NO:141; and (ii) a VL comprising a VL CDR1 having an amino acid sequence of SEQ ID NO:226, a VL CDR2 having an amino acid sequence of SEQ ID NO:227, and a VL CDR3 having an amino acid sequence of SEQ ID NO:228. In some embodiments, the first binding domain that binds to Vβ17 comprises: (i) a VH comprising a VH CDR1 having an amino acid sequence of SEQ ID NO:145, a VH CDR2 having an amino acid sequence of SEQ ID NO:146, and a VH CDR3 having an amino acid sequence of SEQ ID NO:147; and (ii) a VL comprising a VL CDR1 having an amino acid sequence of SEQ ID NO:232, a VL CDR2 having an amino acid sequence of SEQ ID NO:233, and a VL CDR3 having an amino acid sequence of SEQ ID NO:234. In some embodiments, the first binding domain that binds to Vβ17 comprises: (i) a VH comprising a VH CDR1 having an amino acid sequence of SEQ ID NO:151, a VH CDR2 having an amino acid sequence of SEQ ID NO:152, and a VH CDR3 having an amino acid sequence of SEQ ID NO:153; and (ii) a VL comprising a VL CDR1 having an amino acid sequence of SEQ ID NO:238, a VL CDR2 having an amino acid sequence of SEQ ID NO:239, and a VL CDR3 having an amino acid sequence of SEQ ID NO:240. In some embodiments, the first binding domain that binds to Vβ17 comprises: (i) a VH comprising a VH CDR1 having an amino acid sequence of SEQ ID NO:157, a VH CDR2 having an amino acid sequence of SEQ ID NO:158, and a VH CDR3 having an amino acid sequence of SEQ ID NO:159; and (ii) a VL comprising a VL CDR1 having an amino acid sequence of SEQ ID NO:244, a VL CDR2 having an amino acid sequence of SEQ ID NO:245, and a VL CDR3 having an amino acid sequence of SEQ ID NO:246. In some embodiments, the first binding domain that binds to Vβ17 comprises: (i) a VH comprising a VH CDR1 having an amino acid sequence of SEQ ID NO:163, a VH CDR2 having an amino acid sequence of SEQ ID NO:164, and a VH CDR3 having an amino acid sequence of SEQ ID NO:165; and (ii) a VL comprising a VL CDR1 having an amino acid sequence of SEQ ID NO:250, a VL CDR2 having an amino acid sequence of SEQ ID NO:251, and a VL CDR3 having an amino acid sequence of SEQ ID NO:252. In some embodiments, the first binding domain that binds to Vβ17 comprises: (i) a VH comprising a VH CDR1 having an amino acid sequence of SEQ ID NO:169, a VH CDR2 having an amino acid sequence of SEQ ID NO:170, and a VH CDR3 having an amino acid sequence of SEQ ID NO:171; and (ii) a VL comprising a VL CDR1 having an amino acid sequence of SEQ ID NO:256, a VL CDR2 having an amino acid sequence of SEQ ID NO:257, and a VL CDR3 having an amino acid sequence of SEQ ID NO:258. In some embodiments, the first binding domain that binds to Vβ17 comprises: (i) a VH comprising a VH CDR1 having an amino acid sequence of SEQ ID NO:175, a VH CDR2 having an amino acid sequence of SEQ ID NO:176, and a VH CDR3 having an amino acid sequence of SEQ ID NO:177; and (ii) a VL comprising a VL CDR1 having an amino acid sequence of SEQ ID NO:262, a VL CDR2 having an amino acid sequence of SEQ ID NO:263, and a VL CDR3 having an amino acid sequence of SEQ ID NO:264. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:24. In some embodiments, the first binding domain comprises a VH having an amino acid sequence of SEQ ID NO:19. In some embodiments, the first binding domain comprises a VL having an amino acid sequence of SEQ ID NO:24. In some embodiments, the first binding domain comprises a VH having an amino acid sequence of SEQ ID NO:19, and a VL having a n amino acid sequence of SEQ ID NO:24. In some embodiments, the first binding domain comprises a VH having an amino acid sequence having at least 95% identity to an amino acid sequence of SEQ ID NO:19. In some embodiments, the first binding domain comprises a VL having an amino acid sequence having at least 95% identity to an amino acid sequence of SEQ ID NO:24. In some embodiments, the first binding domain comprises a VH having an amino acid sequence having at least 95% identity to an amino acid sequence of SEQ ID NO:19, and a VL having a n amino acid sequence of SEQ ID NO:24.

In some embodiments, the first binding domain that binds to Vβ17 comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain that binds to Vβ17 comprises: (i) a VH comprising a VH CDR1 having an amino acid sequence of SEQ ID NO:181, a VH CDR2 having an amino acid sequence of SEQ ID NO:182, and a VH CDR3 having an amino acid sequence of SEQ ID NO:183; and (ii) a VL comprising a VL CDR1 having an amino acid sequence of SEQ ID NO:142, a VL CDR2 having an amino acid sequence of SEQ ID NO:143, and a VL CDR3 having an amino acid sequence of SEQ ID NO:144. In some embodiments, the first binding domain that binds to Vβ17 comprises: (i) a VH comprising a VH CDR1 having an amino acid sequence of SEQ ID NO:187, a VH CDR2 having an amino acid sequence of SEQ ID NO:188, and a VH CDR3 having an amino acid sequence of SEQ ID NO:189; and (ii) a VL comprising a VL CDR1 having an amino acid sequence of SEQ ID NO:148, a VL CDR2 having an amino acid sequence of SEQ ID NO:149, and a VL CDR3 having an amino acid sequence of SEQ ID NO:150. In some embodiments, the first binding domain that binds to Vβ17 comprises: (i) a VH comprising a VH CDR1 having an amino acid sequence of SEQ ID NO:193, a VH CDR2 having an amino acid sequence of SEQ ID NO:194, and a VH CDR3 having an amino acid sequence of SEQ ID NO:195; and (ii) a VL comprising a VL CDR1 having an amino acid sequence of SEQ ID NO:154, a VL CDR2 having an amino acid sequence of SEQ ID NO:155, and a VL CDR3 having an amino acid sequence of SEQ ID NO:156. In some embodiments, the first binding domain that binds to Vβ17 comprises: (i) a VH comprising a VH CDR1 having an amino acid sequence of SEQ ID NO:199, a VH CDR2 having an amino acid sequence of SEQ ID NO:200, and a VH CDR3 having an amino acid sequence of SEQ ID NO:201; and (ii) a VL comprising a VL CDR1 having an amino acid sequence of SEQ ID NO:160, a VL CDR2 having an amino acid sequence of SEQ ID NO:161, and a VL CDR3 having an amino acid sequence of SEQ ID NO:162. In some embodiments, the first binding domain that binds to Vβ17 comprises: (i) a VH comprising a VH CDR1 having an amino acid sequence of SEQ ID NO:205, a VH CDR2 having an amino acid sequence of SEQ ID NO:206, and a VH CDR3 having an amino acid sequence of SEQ ID NO:207; and (ii) a VL comprising a VL CDR1 having an amino acid sequence of SEQ ID NO:166, a VL CDR2 having an amino acid sequence of SEQ ID NO:167, and a VL CDR3 having an amino acid sequence of SEQ ID NO:168. In some embodiments, the first binding domain that binds to Vβ17 comprises: (i) a VH comprising a VH CDR1 having an amino acid sequence of SEQ ID NO:211, a VH CDR2 having an amino acid sequence of SEQ ID NO:212, and a VH CDR3 having an amino acid sequence of SEQ ID NO:213; and (ii) a VL comprising a VL CDR1 having an amino acid sequence of SEQ ID NO:172, a VL CDR2 having an amino acid sequence of SEQ ID NO:173, and a VL CDR3 having an amino acid sequence of SEQ ID NO:174. In some embodiments, the first binding domain that binds to Vβ17 comprises: (i) a VH comprising a VH CDR1 having an amino acid sequence of SEQ ID NO:217, a VH CDR2 having an amino acid sequence of SEQ ID NO:218, and a VH CDR3 having an amino acid sequence of SEQ ID NO:219 and (ii) a VL comprising a VL CDR1 having an amino acid sequence of SEQ ID NO:178, a VL CDR2 having an amino acid sequence of SEQ ID NO:179, and a VL CDR3 having an amino acid sequence of SEQ ID NO:180. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:22. In some embodiments, the first binding domain comprises a VH having an amino acid sequence of SEQ ID NO:20. In some embodiments, the first binding domain comprises a VL having an amino acid sequence of SEQ ID NO:22. In some embodiments, the first binding domain comprises a VH having an amino acid sequence of SEQ ID NO:20, and a VL having an amino acid sequence of SEQ ID NO:22. In some embodiments, the first binding domain comprises a VH having an amino acid sequence having at least 95% identity to an amino acid sequence of SEQ ID NO:20. In some embodiments, the first binding domain comprises a VL having an amino acid sequence having at least 95% identity to an amino acid sequence of SEQ ID NO:22. In some embodiments, the first binding domain comprises a VH having an amino acid sequence having at least 95% identity to an amino acid sequence of SEQ ID NO:20, and a VL having an amino acid sequence having at least 95% identity to an amino acid sequence of SEQ ID NO:22.

In some embodiments, the first binding domain that binds to Vβ17 comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain that binds to Vβ17 comprises: (i) a VH comprising a VH CDR1 having an amino acid sequence of SEQ ID NO:181, a VH CDR2 having an amino acid sequence of SEQ ID NO:182, and a VH CDR3 having an amino acid sequence of SEQ ID NO:183; and (ii) a VL comprising a VL CDR1 having an amino acid sequence of SEQ ID NO:184, a VL CDR2 having an amino acid sequence of SEQ ID NO:185, and a VL CDR3 having an amino acid sequence of SEQ ID NO:186. In some embodiments, the first binding domain that binds to Vβ17 comprises: (i) a VH comprising a VH CDR1 having an amino acid sequence of SEQ ID NO:187, a VH CDR2 having an amino acid sequence of SEQ ID NO:188, and a VH CDR3 having an amino acid sequence of SEQ ID NO:189; and (ii) a VL comprising a VL CDR1 having an amino acid sequence of SEQ ID NO:190, a VL CDR2 having an amino acid sequence of SEQ ID NO:191, and a VL CDR3 having an amino acid sequence of SEQ ID NO:192. In some embodiments, the first binding domain that binds to Vβ17 comprises: (i) a VH comprising a VH CDR1 having an amino acid sequence of SEQ ID NO:193, a VH CDR2 having an amino acid sequence of SEQ ID NO:194, and a VH CDR3 having an amino acid sequence of SEQ ID NO:195; and (ii) a VL comprising a VL CDR1 having an amino acid sequence of SEQ ID NO:196, a VL CDR2 having an amino acid sequence of SEQ ID NO:197, and a VL CDR3 having an amino acid sequence of SEQ ID NO:198. In some embodiments, the first binding domain that binds to Vβ17 comprises: (i) a VH comprising a VH CDR1 having an amino acid sequence of SEQ ID NO:199, a VH CDR2 having an amino acid sequence of SEQ ID NO:200, and a VH CDR3 having an amino acid sequence of SEQ ID NO:201; and (ii) a VL comprising a VL CDR1 having an amino acid sequence of SEQ ID NO:202, a VL CDR2 having an amino acid sequence of SEQ ID NO:203, and a VL CDR3 having an amino acid sequence of SEQ ID NO:204. In some embodiments, the first binding domain that binds to Vβ17 comprises: (i) a VH comprising a VH CDR1 having an amino acid sequence of SEQ ID NO:205, a VH CDR2 having an amino acid sequence of SEQ ID NO:206, and a VH CDR3 having an amino acid sequence of SEQ ID NO:207; and (ii) a VL comprising a VL CDR1 having an amino acid sequence of SEQ ID NO:208, a VL CDR2 having an amino acid sequence of SEQ ID NO:209, and a VL CDR3 having an amino acid sequence of SEQ ID NO:210. In some embodiments, the first binding domain that binds to Vβ17 comprises: (i) a VH comprising a VH CDR1 having an amino acid sequence of SEQ ID NO:211, a VH CDR2 having an amino acid sequence of SEQ ID NO:212, and a VH CDR3 having an amino acid sequence of SEQ ID NO:213; and (ii) a VL comprising a VL CDR1 having an amino acid sequence of SEQ ID NO:214, a VL CDR2 having an amino acid sequence of SEQ ID NO:215, and a VL CDR3 having an amino acid sequence of SEQ ID NO:216. In some embodiments, the first binding domain that binds to Vβ17 comprises: (i) a VH comprising a VH CDR1 having an amino acid sequence of SEQ ID NO:217, a VH CDR2 having an amino acid sequence of SEQ ID NO:218, and a VH CDR3 having an amino acid sequence of SEQ ID NO:219; and (ii) a VL comprising a VL CDR1 having an amino acid sequence of SEQ ID NO:220, a VL CDR2 having an amino acid sequence of SEQ ID NO:221, and a VL CDR3 having an amino acid sequence of SEQ ID NO:222. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:23. In some embodiments, the first binding domain comprises a VH having an amino acid sequence of SEQ ID NO:20. In some embodiments, the first binding domain comprises a VL having an amino acid sequence of SEQ ID NO:23. In some embodiments, the first binding domain comprises a VH having an amino acid sequence of SEQ ID NO:20, and a VL having an amino acid sequence of SEQ ID NO:23. In some embodiments, the first binding domain comprises a VH having an amino acid sequence having at least 95% identity to an amino acid sequence of SEQ ID NO:20. In some embodiments, the first binding domain comprises a VL having an amino acid sequence having at least 95% identity to an amino acid sequence of SEQ ID NO:23. In some embodiments, the first binding domain comprises a VH having an amino acid sequence having at least 95% identity to an amino acid sequence of SEQ ID NO:20, and a VL having an amino acid sequence having at least 95% identity to an amino acid sequence of SEQ ID NO:23.

In some embodiments, the first binding domain that binds to Vβ17 comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3 and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain that binds to Vβ17 comprises: (i) a VH comprising a VH CDR1 having an amino acid sequence of SEQ ID NO:181, a VH CDR2 having an amino acid sequence of SEQ ID NO:182, and a VH CDR3 having an amino acid sequence of SEQ ID NO:183; and (ii) a VL comprising a VL CDR1 having an amino acid sequence of SEQ ID NO:226, a VL CDR2 having an amino acid sequence of SEQ ID NO:227, and a VL CDR3 having an amino acid sequence of SEQ ID NO:228. In some embodiments, the first binding domain that binds to Vβ17 comprises: (i) a VH comprising a VH CDR1 having an amino acid sequence of SEQ ID NO:187, a VH CDR2 having an amino acid sequence of SEQ ID NO:188, and a VH CDR3 having an amino acid sequence of SEQ ID NO:189; and (ii) a VL comprising a VL CDR1 having an amino acid sequence of SEQ ID NO:232, a VL CDR2 having an amino acid sequence of SEQ ID NO:233, and a VL CDR3 having an amino acid sequence of SEQ ID NO:234. In some embodiments, the first binding domain that binds to Vβ17 comprises: (i) a VH comprising a VH CDR1 having an amino acid sequence of SEQ ID NO:193, a VH CDR2 having an amino acid sequence of SEQ ID NO:194, and a VH CDR3 having an amino acid sequence of SEQ ID NO:195; and (ii) a VL comprising a VL CDR1 having an amino acid sequence of SEQ ID NO:238, a VL CDR2 having an amino acid sequence of SEQ ID NO:239, and a VL CDR3 having an amino acid sequence of SEQ ID NO:240. In some embodiments, the first binding domain that binds to Vβ17 comprises: (i) a VH comprising a VH CDR1 having an amino acid sequence of SEQ ID NO:199, a VH CDR2 having an amino acid sequence of SEQ ID NO:200, and a VH CDR3 having an amino acid sequence of SEQ ID NO:201; and (ii) a VL comprising a VL CDR1 having an amino acid sequence of SEQ ID NO:244, a VL CDR2 having an amino acid sequence of SEQ ID NO:245, and a VL CDR3 having an amino acid sequence of SEQ ID NO:246. In some embodiments, the first binding domain that binds to Vβ17 comprises: (i) a VH comprising a VH CDR1 having an amino acid sequence of SEQ ID NO:205, a VH CDR2 having an amino acid sequence of SEQ ID NO:206, and a VH CDR3 having an amino acid sequence of SEQ ID NO:207; and (ii) a VL comprising a VL CDR1 having an amino acid sequence of SEQ ID NO:250, a VL CDR2 having an amino acid sequence of SEQ ID NO:251, and a VL CDR3 having an amino acid sequence of SEQ ID NO:252. In some embodiments, the first binding domain that binds to Vβ17 comprises: (i) a VH comprising a VH CDR1 having an amino acid sequence of SEQ ID NO:211, a VH CDR2 having an amino acid sequence of SEQ ID NO:212, and a VH CDR3 having an amino acid sequence of SEQ ID NO:213; and (ii) a VL comprising a VL CDR1 having an amino acid sequence of SEQ ID NO:256, a VL CDR2 having an amino acid sequence of SEQ ID NO:257, and a VL CDR3 having an amino acid sequence of SEQ ID NO:258. In some embodiments, the first binding domain that binds to Vβ17 comprises: (i) a VH comprising a VH CDR1 having an amino acid sequence of SEQ ID NO:217, a VH CDR2 having an amino acid sequence of SEQ ID NO:218, and a VH CDR3 having an amino acid sequence of SEQ ID NO:219; and (ii) a VL comprising a VL CDR1 having an amino acid sequence of SEQ ID NO:262, a VL CDR2 having an amino acid sequence of SEQ ID NO:263, and a VL CDR3 having an amino acid sequence of SEQ ID NO:264. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:24. In some embodiments, the first binding domain comprises a VH having an amino acid sequence of SEQ ID NO:20. In some embodiments, the first binding domain comprises a VL having an amino acid sequence of SEQ ID NO:24. In some embodiments, the first binding domain comprises a VH having an amino acid sequence of SEQ ID NO:20, and a VL having an amino acid sequence of SEQ ID NO:24. In some embodiments, the first binding domain comprises a VH having an amino acid sequence having at least 95% identity to an amino acid sequence of SEQ ID NO:20. In some embodiments, the first binding domain comprises a VL having an amino acid sequence having at least 95% identity to an amino acid sequence of SEQ ID NO:24. In some embodiments, the first binding domain comprises a VH having an amino acid sequence having at least 95% identity to an amino acid sequence of SEQ ID NO:20, and a VL having an amino acid sequence having at least 95% identity to an amino acid sequence of SEQ ID NO:24.

In some embodiments, the first binding domain that binds to Vβ17 comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain that binds to Vβ17 comprises: (i) a VH comprising a VH CDR1 having an amino acid sequence of SEQ ID NO:223, a VH CDR2 having an amino acid sequence of SEQ ID NO:224, and a VH CDR3 having an amino acid sequence of SEQ ID NO:225; and (ii) a VL comprising a VL CDR1 having an amino acid sequence of SEQ ID NO:142, a VL CDR2 having an amino acid sequence of SEQ ID NO:143, and a VL CDR3 having an amino acid sequence of SEQ ID NO:144. In some embodiments, the first binding domain that binds to Vβ17 comprises: (i) a VH comprising a VH CDR1 having an amino acid sequence of SEQ ID NO:229, a VH CDR2 having an amino acid sequence of SEQ ID NO:230, and a VH CDR3 having an amino acid sequence of SEQ ID NO:231; and (ii) a VL comprising a VL CDR1 having an amino acid sequence of SEQ ID NO:148, a VL CDR2 having an amino acid sequence of SEQ ID NO:149, and a VL CDR3 having an amino acid sequence of SEQ ID NO:150. In some embodiments, the first binding domain that binds to Vβ17 comprises: (i) a VH comprising a VH CDR1 having an amino acid sequence of SEQ ID NO:235, a VH CDR2 having an amino acid sequence of SEQ ID NO:236, and a VH CDR3 having an amino acid sequence of SEQ ID NO:237; and (ii) a VL comprising a VL CDR1 having an amino acid sequence of SEQ ID NO:154, a VL CDR2 having an amino acid sequence of SEQ ID NO:155, and a VL CDR3 having an amino acid sequence of SEQ ID NO:156. In some embodiments, the first binding domain that binds to Vβ17 comprises: (i) a VH comprising a VH CDR1 having an amino acid sequence of SEQ ID NO:241, a VH CDR2 having an amino acid sequence of SEQ ID NO:242, and a VH CDR3 having an amino acid sequence of SEQ ID NO:243; and (ii) a VL comprising a VL CDR1 having an amino acid sequence of SEQ ID NO:160, a VL CDR2 having an amino acid sequence of SEQ ID NO:161, and a VL CDR3 having an amino acid sequence of SEQ ID NO:162. In some embodiments, the first binding domain that binds to Vβ17 comprises: (i) a VH comprising a VH CDR1 having an amino acid sequence of SEQ ID NO:247, a VH CDR2 having an amino acid sequence of SEQ ID NO:248, and a VH CDR3 having an amino acid sequence of SEQ ID NO:249; and (ii) a VL comprising a VL CDR1 having an amino acid sequence of SEQ ID NO:166, a VL CDR2 having an amino acid sequence of SEQ ID NO:167, and a VL CDR3 having an amino acid sequence of SEQ ID NO:168. In some embodiments, the first binding domain that binds to Vβ17 comprises: (i) a VH comprising a VH CDR1 having an amino acid sequence of SEQ ID NO:253, a VH CDR2 having an amino acid sequence of SEQ ID NO:254, and a VH CDR3 having an amino acid sequence of SEQ ID NO:255; and (ii) a VL comprising a VL CDR1 having an amino acid sequence of SEQ ID NO:172, a VL CDR2 having an amino acid sequence of SEQ ID NO:173, and a VL CDR3 having an amino acid sequence of SEQ ID NO:174. In some embodiments, the first binding domain that binds to Vβ17 comprises: (i) a VH comprising a VH CDR1 having an amino acid sequence of SEQ ID NO:259, a VH CDR2 having an amino acid sequence of SEQ ID NO:260, and a VH CDR3 having an amino acid sequence of SEQ ID NO:261; and (ii) a VL comprising a VL CDR1 having an amino acid sequence of SEQ ID NO:178, a VL CDR2 having an amino acid sequence of SEQ ID NO:179, and a VL CDR3 having an amino acid sequence of SEQ ID NO:180. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:22. In some embodiments, the first binding domain comprises a VH having an amino acid sequence of SEQ ID NO:21. In some embodiments, the first binding domain comprises a VL having an amino acid sequence of SEQ ID NO:22. In some embodiments, the first binding domain comprises a VH having an amino acid sequence of SEQ ID NO:21, and a VL having an amino acid sequence of SEQ ID NO:22. In some embodiments, the first binding domain comprises a VH having an amino acid sequence having at least 95% identity to an amino acid sequence of SEQ ID NO:21. In some embodiments, the first binding domain comprises a VL having an amino acid sequence having at least 95% identity to an amino acid sequence of SEQ ID NO:22. In some embodiments, the first binding domain comprises a VH having an amino acid sequence having at least 95% identity to an amino acid sequence of SEQ ID NO:21, and a VL having an amino acid sequence having at least 95% identity to an amino acid sequence of SEQ ID NO:22.

In some embodiments, the first binding domain that binds to Vβ17 comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain that binds to Vβ17 comprises: (i) a VH comprising a VH CDR1 having an amino acid sequence of SEQ ID NO:223, a VH CDR2 having an amino acid sequence of SEQ ID NO:224, and a VH CDR3 having an amino acid sequence of SEQ ID NO:225; and (ii) a VL comprising a VL CDR1 having an amino acid sequence of SEQ ID NO:184, a VL CDR2 having an amino acid sequence of SEQ ID NO:185, and a VL CDR3 having an amino acid sequence of SEQ ID NO:186. In some embodiments, the first binding domain that binds to Vβ17 comprises: (i) a VH comprising a VH CDR1 having an amino acid sequence of SEQ ID NO:229, a VH CDR2 having an amino acid sequence of SEQ ID NO:230, and a VH CDR3 having an amino acid sequence of SEQ ID NO:231; and (ii) a VL comprising a VL CDR1 having an amino acid sequence of SEQ ID NO:190, a VL CDR2 having an amino acid sequence of SEQ ID NO:191, and a VL CDR3 having an amino acid sequence of SEQ ID NO:192. In some embodiments, the first binding domain that binds to Vβ17 comprises: (i) a VH comprising a VH CDR1 having an amino acid sequence of SEQ ID NO:235, a VH CDR2 having an amino acid sequence of SEQ ID NO:236, and a VH CDR3 having an amino acid sequence of SEQ ID NO:237; and (ii) a VL comprising a VL CDR1 having an amino acid sequence of SEQ ID NO:196, a VL CDR2 having an amino acid sequence of SEQ ID NO:197, and a VL CDR3 having an amino acid sequence of SEQ ID NO:198. In some embodiments, the first binding domain that binds to Vβ17 comprises: (i) a VH comprising a VH CDR1 having an amino acid sequence of SEQ ID NO:241, a VH CDR2 having an amino acid sequence of SEQ ID NO:242, and a VH CDR3 having an amino acid sequence of SEQ ID NO:243; and (ii) a VL comprising a VL CDR1 having an amino acid sequence of SEQ ID NO:202, a VL CDR2 having an amino acid sequence of SEQ ID NO:203, and a VL CDR3 having an amino acid sequence of SEQ ID NO:204. In some embodiments, the first binding domain that binds to Vβ17 comprises: (i) a VH comprising a VH CDR1 having an amino acid sequence of SEQ ID NO:247, a VH CDR2 having an amino acid sequence of SEQ ID NO:248, and a VH CDR3 having an amino acid sequence of SEQ ID NO:249; and (ii) a VL comprising a VL CDR1 having an amino acid sequence of SEQ ID NO:208, a VL CDR2 having an amino acid sequence of SEQ ID NO:209, and a VL CDR3 having an amino acid sequence of SEQ ID NO:210. In some embodiments, the first binding domain that binds to Vβ17 comprises: (i) a VH comprising a VH CDR1 having an amino acid sequence of SEQ ID NO:253, a VH CDR2 having an amino acid sequence of SEQ ID NO:254, and a VH CDR3 having an amino acid sequence of SEQ ID NO:255; and (ii) a VL comprising a VL CDR1 having an amino acid sequence of SEQ ID NO:214, a VL CDR2 having an amino acid sequence of SEQ ID NO:215, and a VL CDR3 having an amino acid sequence of SEQ ID NO:216. In some embodiments, the first binding domain that binds to Vβ17 comprises: (i) a VH comprising a VH CDR1 having an amino acid sequence of SEQ ID NO:259, a VH CDR2 having an amino acid sequence of SEQ ID NO:260, and a VH CDR3 having an amino acid sequence of SEQ ID NO:261; and (ii) a VL comprising a VL CDR1 having an amino acid sequence of SEQ ID NO:220, a VL CDR2 having an amino acid sequence of SEQ ID NO:221, and a VL CDR3 having an amino acid sequence of SEQ ID NO:222. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:23. In some embodiments, the first binding domain comprises a VH having an amino acid sequence of SEQ ID NO:21. In some embodiments, the first binding domain comprises a VL having an amino acid sequence of SEQ ID NO:23. In some embodiments, the first binding domain comprises a VH having an amino acid sequence of SEQ ID NO:21, and a VL having an amino acid sequence of SEQ ID NO:23. In some embodiments, the first binding domain comprises a VH having an amino acid sequence having at least 95% identity to an amino acid sequence of SEQ ID NO:21. In some embodiments, the first binding domain comprises a VL having an amino acid sequence having at least 95% identity to an amino acid sequence of SEQ ID NO:23. In some embodiments, the first binding domain comprises a VH having an amino acid sequence having at least 95% identity to an amino acid sequence of SEQ ID NO:21, and a VL having an amino acid sequence having at least 95% identity to an amino acid sequence of SEQ ID NO:23.

In some embodiments, the first binding domain that binds to Vβ17 comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain that binds to Vβ17 comprises: (i) a VH comprising a VH CDR1 having an amino acid sequence of SEQ ID NO:223, a VH CDR2 having an amino acid sequence of SEQ ID NO:224, and a VH CDR3 having an amino acid sequence of SEQ ID NO:225; and (ii) a VL comprising a VL CDR1 having an amino acid sequence of SEQ ID NO:226, a VL CDR2 having an amino acid sequence of SEQ ID NO:227, and a VL CDR3 having an amino acid sequence of SEQ ID NO:228. In some embodiments, the first binding domain that binds to Vβ17 comprises: (i) a VH comprising a VH CDR1 having an amino acid sequence of SEQ ID NO:229, a VH CDR2 having an amino acid sequence of SEQ ID NO:230, and a VH CDR3 having an amino acid sequence of SEQ ID NO:231; and (ii) a VL comprising a VL CDR1 having an amino acid sequence of SEQ ID NO:232, a VL CDR2 having an amino acid sequence of SEQ ID NO:233, and a VL CDR3 having an amino acid sequence of SEQ ID NO:234. In some embodiments, the first binding domain that binds to Vβ17 comprises: (i) a VH comprising a VH CDR1 having an amino acid sequence of SEQ ID NO:235, a VH CDR2 having an amino acid sequence of SEQ ID NO:236, and a VH CDR3 having an amino acid sequence of SEQ ID NO:237; and (ii) a VL comprising a VL CDR1 having an amino acid sequence of SEQ ID NO:238, a VL CDR2 having an amino acid sequence of SEQ ID NO:239, and a VL CDR3 having an amino acid sequence of SEQ ID NO:240. In some embodiments, the first binding domain that binds to Vβ17 comprises: (i) a VH comprising a VH CDR1 having an amino acid sequence of SEQ ID NO:241, a VH CDR2 having an amino acid sequence of SEQ ID NO:242, and a VH CDR3 having an amino acid sequence of SEQ ID NO:243; and (ii) a VL comprising a VL CDR1 having an amino acid sequence of SEQ ID NO:244, a VL CDR2 having an amino acid sequence of SEQ ID NO:245, and a VL CDR3 having an amino acid sequence of SEQ ID NO:246. In some embodiments, the first binding domain that binds to Vβ17 comprises: (i) a VH comprising a VH CDR1 having an amino acid sequence of SEQ ID NO:247, a VH CDR2 having an amino acid sequence of SEQ ID NO:248, and a VH CDR3 having an amino acid sequence of SEQ ID NO:249; and (ii) a VL comprising a VL CDR1 having an amino acid sequence of SEQ ID NO:250, a VL CDR2 having an amino acid sequence of SEQ ID NO:251, and a VL CDR3 having an amino acid sequence of SEQ ID NO:252. In some embodiments, the first binding domain that binds to Vβ17 comprises: (i) a VH comprising a VH CDR1 having an amino acid sequence of SEQ ID NO:253, a VH CDR2 having an amino acid sequence of SEQ ID NO:254, and a VH CDR3 having an amino acid sequence of SEQ ID NO:255; and (ii) a VL comprising a VL CDR1 having an amino acid sequence of SEQ ID NO:256, a VL CDR2 having an amino acid sequence of SEQ ID NO:257, and a VL CDR3 having an amino acid sequence of SEQ ID NO:258. In some embodiments, the first binding domain that binds to Vβ17 comprises: (i) a VH comprising a VH CDR1 having an amino acid sequence of SEQ ID NO:259, a VH CDR2 having an amino acid sequence of SEQ ID NO:260, and a VH CDR3 having an amino acid sequence of SEQ ID NO:261; and (ii) a VL comprising a VL CDR1 having an amino acid sequence of SEQ ID NO:262, a VL CDR2 having an amino acid sequence of SEQ ID NO:263, and a VL CDR3 having an amino acid sequence of SEQ ID NO:264. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:24. In some embodiments, the first binding domain comprises a VH having an amino acid sequence of SEQ ID NO:21. In some embodiments, the first binding domain comprises a VL having an amino acid sequence of SEQ ID NO:24. In some embodiments, the first binding domain comprises a VH having an amino acid sequence of SEQ ID NO:21, and a VL having an amino acid sequence of SEQ ID NO:24. In some embodiments, the first binding domain comprises a VH having an amino acid sequence having at least 95% identity to an amino acid sequence of SEQ ID NO:21. In some embodiments, the first binding domain comprises a VL having an amino acid sequence having at least 95% identity to an amino acid sequence of SEQ ID NO:24. In some embodiments, the first binding domain comprises a VH having an amino acid sequence having at least 95% identity to an amino acid sequence of SEQ ID NO:21, and a VL having an amino acid sequence having at least 95% identity to an amino acid sequence of SEQ ID NO:24.

In some embodiments, the first binding domain that binds to Vβ17 comprises: (i) a VH comprising a VH CDR1 having an amino acid sequence of SEQ ID NO:45, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain that binds to Vβ17 comprises: (i) a VH comprising a VH CDR1 having an amino acid sequence of SEQ ID NO:265, a VH CDR2 having an amino acid sequence of SEQ ID NO:266, and a VH CDR3 having an amino acid sequence of SEQ ID NO:267; and (ii) a VL comprising a VL CDR1 having an amino acid sequence of SEQ ID NO:268, a VL CDR2 having an amino acid sequence of SEQ ID NO:269, and a VL CDR3 having an amino acid sequence of SEQ ID NO:270. In some embodiments, the first binding domain that binds to Vβ17 comprises: (i) a VH comprising a VH CDR1 having an amino acid sequence of SEQ ID NO:271, a VH CDR2 having an amino acid sequence of SEQ ID NO:272, and a VH CDR3 having an amino acid sequence of SEQ ID NO:273; and (ii) a VL comprising a VL CDR1 having an amino acid sequence of SEQ ID NO:274, a VL CDR2 having an amino acid sequence of SEQ ID NO:275, and a VL CDR3 having an amino acid sequence of SEQ ID NO:276. In some embodiments, the first binding domain that binds to Vβ17 comprises: (i) a VH comprising a VH CDR1 having an amino acid sequence of SEQ ID NO:277, a VH CDR2 having an amino acid sequence of SEQ ID NO:278, and a VH CDR3 having an amino acid sequence of SEQ ID NO:279; and (ii) a VL comprising a VL CDR1 having an amino acid sequence of SEQ ID NO:280, a VL CDR2 having an amino acid sequence of SEQ ID NO:281, and a VL CDR3 having an amino acid sequence of SEQ ID NO:282. In some embodiments, the first binding domain that binds to Vβ17 comprises: (i) a VH comprising a VH CDR1 having an amino acid sequence of SEQ ID NO:283, a VH CDR2 having an amino acid sequence of SEQ ID NO:284, and a VH CDR3 having an amino acid sequence of SEQ ID NO:285; and (ii) a VL comprising a VL CDR1 having an amino acid sequence of SEQ ID NO:286, a VL CDR2 having an amino acid sequence of SEQ ID NO:287, and a VL CDR3 having an amino acid sequence of SEQ ID NO:288. In some embodiments, the first binding domain that binds to Vβ17 comprises: (i) a VH comprising a VH CDR1 having an amino acid sequence of SEQ ID NO:289, a VH CDR2 having an amino acid sequence of SEQ ID NO:290, and a VH CDR3 having an amino acid sequence of SEQ ID NO:291; and (ii) a VL comprising a VL CDR1 having an amino acid sequence of SEQ ID NO:292, a VL CDR2 having an amino acid sequence of SEQ ID NO:293, and a VL CDR3 having an amino acid sequence of SEQ ID NO:294. In some embodiments, the first binding domain that binds to Vβ17 comprises: (i) a VH comprising a VH CDR1 having an amino acid sequence of SEQ ID NO:295, a VH CDR2 having an amino acid sequence of SEQ ID NO:296, and a VH CDR3 having an amino acid sequence of SEQ ID NO:297; and (ii) a VL comprising a VL CDR1 having an amino acid sequence of SEQ ID NO:298, a VL CDR2 having an amino acid sequence of SEQ ID NO:299, and a VL CDR3 having an amino acid sequence of SEQ ID NO:300. In some embodiments, the first binding domain that binds to Vβ17 comprises: (i) a VH comprising a VH CDR1 having an amino acid sequence of SEQ ID NO:301, a VH CDR2 having an amino acid sequence of SEQ ID NO:302, and a VH CDR3 having an amino acid sequence of SEQ ID NO:303; and (ii) a VL comprising a VL CDR1 having an amino acid sequence of SEQ ID NO:304, a VL CDR2 having an amino acid sequence of SEQ ID NO:305, and a VL CDR3 having an amino acid sequence of SEQ ID NO:306. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:46; and (ii) a VL comprising a VL CDR1, a VL CDR2, and a VL CDR3 having an amino acid sequence of a VL CDR1, VL CDR2, and VL CDR3, respectively, of SEQ ID NO:49. In some embodiments, the first binding domain comprises a VH having an amino acid sequence of SEQ ID NO:46. In some embodiments, the first binding domain comprises a VL having an amino acid sequence of SEQ ID NO:47. In some embodiments, the first binding domain comprises a VH having an amino acid sequence of SEQ ID NO:46, and a VL having an amino acid sequence of SEQ ID NO:47. In some embodiments, the first binding domain comprises a heavy chain having an amino acid sequence of SEQ ID NO:11. In some embodiments, the first binding domain comprises a light chain having an amino acid sequence of SEQ ID NO:12. In some embodiments, the first binding domain comprises a heavy chain having an amino acid sequence having at least 95% identity to an amino acid sequence of SEQ ID NO:11, and a light chain having an amino acid sequence having at least 95% identity to an amino acid sequence of SEQ ID NO:12. In some embodiments, the first binding domain comprises a VH having an amino acid sequence having at least 95% identity to an amino acid sequence of SEQ ID NO:46. In some embodiments, the first binding domain comprises a VL having an amino acid sequence having at least 95% identity to an amino acid sequence of SEQ ID NO:47. In some embodiments, the first binding domain comprises a VH having an amino acid sequence having at least 95% identity to an amino acid sequence of SEQ ID NO:46, and a VL having an amino acid sequence having at least 95% identity to an amino acid sequence of SEQ ID NO:47. In some embodiments, the first binding domain comprises a heavy chain having an amino acid sequence having at least 95% identity to an amino acid sequence of SEQ ID NO:11. In some embodiments, the first binding domain comprises a light chain having an amino acid sequence having at least 95% identity to an amino acid sequence of SEQ ID NO:12. In some embodiments, the first binding domain comprises a heavy chain having an amino acid sequence having at least 95% identity to an amino acid sequence of SEQ ID NO:11, and a light chain having an amino acid sequence having at least 95% identity to an amino acid sequence of SEQ ID NO:12.

In some embodiments, the first binding domain that binds to Vβ17 comprises: (i) a VH comprising a VH CDR1 having an amino acid sequence of SEQ ID NO:48, a VH CDR2 having an amino acid sequence of SEQ ID NO:49, and a VH CDR3 having an amino acid sequence of SEQ ID NO:50; and (ii) a VL comprising a VL CDR1 having an amino acid sequence of SEQ ID NO:51, a VL CDR2 having an amino acid sequence of SEQ ID NO:52, and a VL CDR3 having an amino acid sequence of SEQ ID NO:53. In some embodiments, the first binding domain that binds to Vβ17 comprises: (i) a VH comprising a VH CDR1 having an amino acid sequence of SEQ ID NO:307, a VH CDR2 having an amino acid sequence of SEQ ID NO:308, and a VH CDR3 having an amino acid sequence of SEQ ID NO:309; and (ii) a VL comprising a VL CDR1 having an amino acid sequence of SEQ ID NO:310, a VL CDR2 having an amino acid sequence of SEQ ID NO:311, and a VL CDR3 having an amino acid sequence of SEQ ID NO:312. In some embodiments, the first binding domain that binds to Vβ17 comprises: (i) a VH comprising a VH CDR1 having an amino acid sequence of SEQ ID NO:313, a VH CDR2 having an amino acid sequence of SEQ ID NO:314, and a VH CDR3 having an amino acid sequence of SEQ ID NO:315; and (ii) a VL comprising a VL CDR1 having an amino acid sequence of SEQ ID NO:316, a VL CDR2 having an amino acid sequence of SEQ ID NO:317, and a VL CDR3 having an amino acid sequence of SEQ ID NO:318. In some embodiments, the first binding domain that binds to Vβ17 comprises: (i) a VH comprising a VH CDR1 having an amino acid sequence of SEQ ID NO:319, a VH CDR2 having an amino acid sequence of SEQ ID NO:320, and a VH CDR3 having an amino acid sequence of SEQ ID NO:321; and (ii) a VL comprising a VL CDR1 having an amino acid sequence of SEQ ID NO:322, a VL CDR2 having an amino acid sequence of SEQ ID NO:323, and a VL CDR3 having an amino acid sequence of SEQ ID NO:324. In some embodiments, the first binding domain that binds to Vβ17 comprises: (i) a VH comprising a VH CDR1 having an amino acid sequence of SEQ ID NO:325, a VH CDR2 having an amino acid sequence of SEQ ID NO:326, and a VH CDR3 having an amino acid sequence of SEQ ID NO:327; and (ii) a VL comprising a VL CDR1 having an amino acid sequence of SEQ ID NO:328, a VL CDR2 having an amino acid sequence of SEQ ID NO:329, and a VL CDR3 having an amino acid sequence of SEQ ID NO:330. In some embodiments, the first binding domain that binds to Vβ17 comprises: (i) a VH comprising a VH CDR1 having an amino acid sequence of SEQ ID NO:331, a VH CDR2 having an amino acid sequence of SEQ ID NO:332, and a VH CDR3 having an amino acid sequence of SEQ ID NO:333; and (ii) a VL comprising a VL CDR1 having an amino acid sequence of SEQ ID NO:334, a VL CDR2 having an amino acid sequence of SEQ ID NO:335, and a VL CDR3 having an amino acid sequence of SEQ ID NO:336. In some embodiments, the first binding domain that binds to Vβ17 comprises: (i) a VH comprising a VH CDR1 having an amino acid sequence of SEQ ID NO:337, a VH CDR2 having an amino acid sequence of SEQ ID NO:338, and a VH CDR3 having an amino acid sequence of SEQ ID NO:339; and (ii) a VL comprising a VL CDR1 having an amino acid sequence of SEQ ID NO:340, a VL CDR2 having an amino acid sequence of SEQ ID NO:341, and a VL CDR3 having an amino acid sequence of SEQ ID NO:342. In some embodiments, the first binding domain that binds to Vβ17 comprises: (i) a VH comprising a VH CDR1 having an amino acid sequence of SEQ ID NO:343, a VH CDR2 having an amino acid sequence of SEQ ID NO:344, and a VH CDR3 having an amino acid sequence of SEQ ID NO:345; and (ii) a VL comprising a VL CDR1 having an amino acid sequence of SEQ ID NO:346, a VL CDR2 having an amino acid sequence of SEQ ID NO:347, and a VL CDR3 having an amino acid sequence of SEQ ID NO:348. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:77; and (ii) a VL comprising a VL CDR1, a VL CDR2, and a VL CDR3 having an amino acid sequence of a VL CDR1, VL CDR2, and VL CDR3, respectively, of SEQ ID NO:78. In some embodiments, the first binding domain comprises a VH having an amino acid sequence of SEQ ID NO:77. In some embodiments, the first binding domain comprises a VL having an amino acid sequence of SEQ ID NO:78. In some embodiments, the first binding domain comprises a VH having an amino acid sequence of SEQ ID NO:77, and a VL having an amino acid sequence of SEQ ID NO:78. In some embodiments, the first binding domain comprises a heavy chain having an amino acid sequence of SEQ ID NO:664. In some embodiments, the first binding domain comprises a light chain having an amino acid sequence of SEQ ID NO:665. In some embodiments, the first binding domain comprises a heavy chain having an amino acid sequence of SEQ ID NO:664, and a light chain having an amino acid sequence of SEQ ID NO:665. In some embodiments, the first binding domain comprises a VH having an amino acid sequence having at least 95% identity to an amino acid sequence of SEQ ID NO:77. In some embodiments, the first binding domain comprises a VL having an amino acid sequence having at least 95% identity to an amino acid sequence of SEQ ID NO:78. In some embodiments, the first binding domain comprises a VH having an amino acid sequence having at least 95% identity to an amino acid sequence of SEQ ID NO:77, and a VL having an amino acid sequence having at least 95% identity to an amino acid sequence of SEQ ID NO:78. In some embodiments, the first binding domain comprises a heavy chain having an amino acid sequence having at least 95% identity to an amino acid sequence of SEQ ID NO:664. In some embodiments, the first binding domain comprises a light chain having an amino acid sequence having at least 95% identity to an amino acid sequence of SEQ ID NO:665. In some embodiments, the first binding domain comprises a heavy chain having an amino acid sequence having at least 95% identity to an amino acid sequence of SEQ ID NO:664, and a light chain having an amino acid sequence having at least 95% identity to an amino acid sequence of SEQ ID NO:665.

In some embodiments, the first binding domain that binds to Vβ17 comprises: (i) a VH comprising a VH CDR1 having an amino acid sequence of SEQ ID NO:48, a VH CDR2 having an amino acid sequence of SEQ ID NO:54, and a VH CDR3 having an amino acid sequence of SEQ ID NO:55; and (ii) a VL comprising a VL CDR1 having an amino acid sequence of SEQ ID NO:56, a VL CDR2 having an amino acid sequence of SEQ ID NO:57, and a VL CDR3 having an amino acid sequence of SEQ ID NO:58. In some embodiments, the first binding domain that binds to Vβ17 comprises: (i) a VH comprising a VH CDR1 having an amino acid sequence of SEQ ID NO:349, a VH CDR2 having an amino acid sequence of SEQ ID NO:350, and a VH CDR3 having an amino acid sequence of SEQ ID NO:351; and (ii) a VL comprising a VL CDR1 having an amino acid sequence of SEQ ID NO:352, a VL CDR2 having an amino acid sequence of SEQ ID NO:353, and a VL CDR3 having an amino acid sequence of SEQ ID NO:354. In some embodiments, the first binding domain that binds to Vβ17 comprises: (i) a VH comprising a VH CDR1 having an amino acid sequence of SEQ ID NO:355, a VH CDR2 having an amino acid sequence of SEQ ID NO:356, and a VH CDR3 having an amino acid sequence of SEQ ID NO:357; and (ii) a VL comprising a VL CDR1 having an amino acid sequence of SEQ ID NO:358, a VL CDR2 having an amino acid sequence of SEQ ID NO:359, and a VL CDR3 having an amino acid sequence of SEQ ID NO:360. In some embodiments, the first binding domain that binds to Vβ17 comprises: (i) a VH comprising a VH CDR1 having an amino acid sequence of SEQ ID NO:361, a VH CDR2 having an amino acid sequence of SEQ ID NO:362, and a VH CDR3 having an amino acid sequence of SEQ ID NO:363; and (ii) a VL comprising a VL CDR1 having an amino acid sequence of SEQ ID NO:364, a VL CDR2 having an amino acid sequence of SEQ ID NO:365, and a VL CDR3 having an amino acid sequence of SEQ ID NO:366. In some embodiments, the first binding domain that binds to Vβ17 comprises: (i) a VH comprising a VH CDR1 having an amino acid sequence of SEQ ID NO:367, a VH CDR2 having an amino acid sequence of SEQ ID NO:368, and a VH CDR3 having an amino acid sequence of SEQ ID NO:369; and (ii) a VL comprising a VL CDR1 having an amino acid sequence of SEQ ID NO:370, a VL CDR2 having an amino acid sequence of SEQ ID NO:371, and a VL CDR3 having an amino acid sequence of SEQ ID NO:372. In some embodiments, the first binding domain that binds to Vβ17 comprises: (i) a VH comprising a VH CDR1 having an amino acid sequence of SEQ ID NO:373, a VH CDR2 having an amino acid sequence of SEQ ID NO:374, and a VH CDR3 having an amino acid sequence of SEQ ID NO:375; and (ii) a VL comprising a VL CDR1 having an amino acid sequence of SEQ ID NO:376, a VL CDR2 having an amino acid sequence of SEQ ID NO:377, and a VL CDR3 having an amino acid sequence of SEQ ID NO:378. In some embodiments, the first binding domain that binds to Vβ17 comprises: (i) a VH comprising a VH CDR1 having an amino acid sequence of SEQ ID NO:379, a VH CDR2 having an amino acid sequence of SEQ ID NO:380, and a VH CDR3 having an amino acid sequence of SEQ ID NO:381; and (ii) a VL comprising a VL CDR1 having an amino acid sequence of SEQ ID NO:382, a VL CDR2 having an amino acid sequence of SEQ ID NO:383, and a VL CDR3 having an amino acid sequence of SEQ ID NO:384. In some embodiments, the first binding domain that binds to Vβ17 comprises: (i) a VH comprising a VH CDR1 having an amino acid sequence of SEQ ID NO:385, a VH CDR2 having an amino acid sequence of SEQ ID NO:386, and a VH CDR3 having an amino acid sequence of SEQ ID NO:387; and (ii) a VL comprising a VL CDR1 having an amino acid sequence of SEQ ID NO:388, a VL CDR2 having an amino acid sequence of SEQ ID NO:389, and a VL CDR3 having an amino acid sequence of SEQ ID NO:390 In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:79; and (ii) a VL comprising a VL CDR1, a VL CDR2, and a VL CDR3 having an amino acid sequence of a VL CDR1, VL CDR2, and VL CDR3, respectively, of SEQ ID NO:80. In some embodiments, the first binding domain comprises a VH having an amino acid sequence of SEQ ID NO:79. In some embodiments, the first binding domain comprises a VL having an amino acid sequence of SEQ ID NO:80. In some embodiments, the first binding domain comprises a VH having an amino acid sequence of SEQ ID NO:79, and a VL having an amino acid sequence of SEQ ID NO:80. In some embodiments, the first binding domain comprises a heavy chain having an amino acid sequence of SEQ ID NO:666. In some embodiments, the first binding domain comprises a light chain having an amino acid sequence of SEQ ID NO:667. In some embodiments, the first binding domain comprises a heavy chain having an amino acid sequence of SEQ ID NO:666, and a light chain having an amino acid sequence of SEQ ID NO:667. In some embodiments, the first binding domain comprises a VH having an amino acid sequence having at least 95% identity to an amino acid sequence of SEQ ID NO:79. In some embodiments, the first binding domain comprises a VL having an amino acid sequence having at least 95% identity to an amino acid sequence of SEQ ID NO:80. In some embodiments, the first binding domain comprises a VH having an amino acid sequence having at least 95% identity to an amino acid sequence of SEQ ID NO:79, and a VL having an amino acid sequence having at least 95% identity to an amino acid sequence of SEQ ID NO:80. In some embodiments, the first binding domain comprises a heavy chain having an amino acid sequence having at least 95% identity to an amino acid sequence of SEQ ID NO:666. In some embodiments, the first binding domain comprises a light chain having an amino acid sequence having at least 95% identity to an amino acid sequence of SEQ ID NO:667. In some embodiments, the first binding domain comprises a heavy chain having an amino acid sequence having at least 95% identity to an amino acid sequence of SEQ ID NO:666, and a light chain having an amino acid sequence having at least 95% identity to an amino acid sequence of SEQ ID NO:667.

In some embodiments, the first binding domain that binds to Vβ17 comprises: (i) a VH comprising a VH CDR1 having an amino acid sequence of SEQ ID NO:59, a VH CDR2 having an amino acid sequence of SEQ ID NO:49, and a VH CDR3 having an amino acid sequence of SEQ ID NO:60; and (ii) a VL comprising a VL CDR1 having an amino acid sequence of SEQ ID NO:61, a VL CDR2 having an amino acid sequence of SEQ ID NO:62, and a VL CDR3 having an amino acid sequence of SEQ ID NO:63. In some embodiments, the first binding domain that binds to Vβ17 comprises: (i) a VH comprising a VH CDR1 having an amino acid sequence of SEQ ID NO:391, a VH CDR2 having an amino acid sequence of SEQ ID NO:392, and a VH CDR3 having an amino acid sequence of SEQ ID NO:393; and (ii) a VL comprising a VL CDR1 having an amino acid sequence of SEQ ID NO:394, a VL CDR2 having an amino acid sequence of SEQ ID NO:395, and a VL CDR3 having an amino acid sequence of SEQ ID NO:396. In some embodiments, the first binding domain that binds to Vβ17 comprises: (i) a VH comprising a VH CDR1 having an amino acid sequence of SEQ ID NO:397, a VH CDR2 having an amino acid sequence of SEQ ID NO:398, and a VH CDR3 having an amino acid sequence of SEQ ID NO:399; and (ii) a VL comprising a VL CDR1 having an amino acid sequence of SEQ ID NO:400, a VL CDR2 having an amino acid sequence of SEQ ID NO:401, and a VL CDR3 having an amino acid sequence of SEQ ID NO:402. In some embodiments, the first binding domain that binds to Vβ17 comprises: (i) a VH comprising a VH CDR1 having an amino acid sequence of SEQ ID NO:403, a VH CDR2 having an amino acid sequence of SEQ ID NO:404, and a VH CDR3 having an amino acid sequence of SEQ ID NO:405; and (ii) a VL comprising a VL CDR1 having an amino acid sequence of SEQ ID NO:406, a VL CDR2 having an amino acid sequence of SEQ ID NO:407, and a VL CDR3 having an amino acid sequence of SEQ ID NO:408. In some embodiments, the first binding domain that binds to Vβ17 comprises: (i) a VH comprising a VH CDR1 having an amino acid sequence of SEQ ID NO:409, a VH CDR2 having an amino acid sequence of SEQ ID NO:410, and a VH CDR3 having an amino acid sequence of SEQ ID NO:411; and (ii) a VL comprising a VL CDR1 having an amino acid sequence of SEQ ID NO:412, a VL CDR2 having an amino acid sequence of SEQ ID NO:413, and a VL CDR3 having an amino acid sequence of SEQ ID NO:414. In some embodiments, the first binding domain that binds to Vβ17 comprises: (i) a VH comprising a VH CDR1 having an amino acid sequence of SEQ ID NO:415, a VH CDR2 having an amino acid sequence of SEQ ID NO:416, and a VH CDR3 having an amino acid sequence of SEQ ID NO:417; and (ii) a VL comprising a VL CDR1 having an amino acid sequence of SEQ ID NO:418, a VL CDR2 having an amino acid sequence of SEQ ID NO:419, and a VL CDR3 having an amino acid sequence of SEQ ID NO:420. In some embodiments, the first binding domain that binds to Vβ17 comprises: (i) a VH comprising a VH CDR1 having an amino acid sequence of SEQ ID NO:421, a VH CDR2 having an amino acid sequence of SEQ ID NO:422, and a VH CDR3 having an amino acid sequence of SEQ ID NO:423; and (ii) a VL comprising a VL CDR1 having an amino acid sequence of SEQ ID NO:424, a VL CDR2 having an amino acid sequence of SEQ ID NO:425, and a VL CDR3 having an amino acid sequence of SEQ ID NO:426. In some embodiments, the first binding domain that binds to Vβ17 comprises: (i) a VH comprising a VH CDR1 having an amino acid sequence of SEQ ID NO:427, a VH CDR2 having an amino acid sequence of SEQ ID NO:428, and a VH CDR3 having an amino acid sequence of SEQ ID NO:429; and (ii) a VL comprising a VL CDR1 having an amino acid sequence of SEQ ID NO:430, a VL CDR2 having an amino acid sequence of SEQ ID NO:431, and a VL CDR3 having an amino acid sequence of SEQ ID NO:432. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:81; and (ii) a VL comprising a VL CDR1, a VL CDR2, and a VL CDR3 having an amino acid sequence of a VL CDR1, VL CDR2, and VL CDR3, respectively, of SEQ ID NO:82. In some embodiments, the first binding domain comprises a VH having an amino acid sequence of SEQ ID NO:81. In some embodiments, the first binding domain comprises a VL having an amino acid sequence of SEQ ID NO:82. In some embodiments, the first binding domain comprises a VH having an amino acid sequence of SEQ ID NO:81, and a VL having an amino acid sequence of SEQ ID NO:82. In some embodiments, the first binding domain comprises a heavy chain having an amino acid sequence of SEQ ID NO:668. In some embodiments, the first binding domain comprises a light chain having an amino acid sequence of SEQ ID NO:669. In some embodiments, the first binding domain comprises a heavy chain having an amino acid sequence of SEQ ID NO:668, and a light chain having an amino acid sequence of SEQ ID NO:669. In some embodiments, the first binding domain comprises a VH having an amino acid sequence having at least 95% identity to an amino acid sequence of SEQ ID NO:81. In some embodiments, the first binding domain comprises a VL having an amino acid sequence having at least 95% identity to an amino acid sequence of SEQ ID NO:82. In some embodiments, the first binding domain comprises a VH having an amino acid sequence having at least 95% identity to an amino acid sequence of SEQ ID NO:81, and a VL having an amino acid sequence having at least 95% identity to an amino acid sequence of SEQ ID NO:82. In some embodiments, the first binding domain comprises a heavy chain having an amino acid sequence having at least 95% identity to an amino acid sequence of SEQ ID NO:668. In some embodiments, the first binding domain comprises a light chain having an amino acid sequence having at least 95% identity to an amino acid sequence of SEQ ID NO:669. In some embodiments, the first binding domain comprises a heavy chain having an amino acid sequence having at least 95% identity to an amino acid sequence of SEQ ID NO:668, and a light chain having an amino acid sequence having at least 95% identity to an amino acid sequence of SEQ ID NO:669.

In some embodiments, the first binding domain that binds to Vβ17 comprises: (i) a VH comprising a VH CDR1 having an amino acid sequence of SEQ ID NO:64, a VH CDR2 having an amino acid sequence of SEQ ID NO:65, and a VH CDR3 having an amino acid sequence of SEQ ID NO:66; and (ii) a VL comprising a VL CDR1 having an amino acid sequence of SEQ ID NO:67, a VL CDR2 having an amino acid sequence of SEQ ID NO:68, and a VL CDR3 having an amino acid sequence of SEQ ID NO:69. In some embodiments, the first binding domain that binds to Vβ17 comprises: (i) a VH comprising a VH CDR1 having an amino acid sequence of SEQ ID NO:433, a VH CDR2 having an amino acid sequence of SEQ ID NO:434, and a VH CDR3 having an amino acid sequence of SEQ ID NO:435; and (ii) a VL comprising a VL CDR1 having an amino acid sequence of SEQ ID NO:436, a VL CDR2 having an amino acid sequence of SEQ ID NO:437, and a VL CDR3 having an amino acid sequence of SEQ ID NO:438. In some embodiments, the first binding domain that binds to Vβ17 comprises: (i) a VH comprising a VH CDR1 having an amino acid sequence of SEQ ID NO:439, a VH CDR2 having an amino acid sequence of SEQ ID NO:440, and a VH CDR3 having an amino acid sequence of SEQ ID NO:441; and (ii) a VL comprising a VL CDR1 having an amino acid sequence of SEQ ID NO:442, a VL CDR2 having an amino acid sequence of SEQ ID NO:443, and a VL CDR3 having an amino acid sequence of SEQ ID NO:444. In some embodiments, the first binding domain that binds to Vβ17 comprises: (i) a VH comprising a VH CDR1 having an amino acid sequence of SEQ ID NO:445, a VH CDR2 having an amino acid sequence of SEQ ID NO:446, and a VH CDR3 having an amino acid sequence of SEQ ID NO:447; and (ii) a VL comprising a VL CDR1 having an amino acid sequence of SEQ ID NO:448, a VL CDR2 having an amino acid sequence of SEQ ID NO:449, and a VL CDR3 having an amino acid sequence of SEQ ID NO:450. In some embodiments, the first binding domain that binds to Vβ17 comprises: (i) a VH comprising a VH CDR1 having an amino acid sequence of SEQ ID NO:451, a VH CDR2 having an amino acid sequence of SEQ ID NO:452, and a VH CDR3 having an amino acid sequence of SEQ ID NO:453; and (ii) a VL comprising a VL CDR1 having an amino acid sequence of SEQ ID NO:454, a VL CDR2 having an amino acid sequence of SEQ ID NO:455, and a VL CDR3 having an amino acid sequence of SEQ ID NO:456. In some embodiments, the first binding domain that binds to Vβ17 comprises: (i) a VH comprising a VH CDR1 having an amino acid sequence of SEQ ID NO:457, a VH CDR2 having an amino acid sequence of SEQ ID NO:458, and a VH CDR3 having an amino acid sequence of SEQ ID NO:459; and (ii) a VL comprising a VL CDR1 having an amino acid sequence of SEQ ID NO:460, a VL CDR2 having an amino acid sequence of SEQ ID NO:461, and a VL CDR3 having an amino acid sequence of SEQ ID NO:462. In some embodiments, the first binding domain that binds to Vβ17 comprises: (i) a VH comprising a VH CDR1 having an amino acid sequence of SEQ ID NO:463, a VH CDR2 having an amino acid sequence of SEQ ID NO:464, and a VH CDR3 having an amino acid sequence of SEQ ID NO:465; and (ii) a VL comprising a VL CDR1 having an amino acid sequence of SEQ ID NO:466, a VL CDR2 having an amino acid sequence of SEQ ID NO:467, and a VL CDR3 having an amino acid sequence of SEQ ID NO:468. In some embodiments, the first binding domain that binds to Vβ17 comprises: (i) a VH comprising a VH CDR1 having an amino acid sequence of SEQ ID NO:469, a VH CDR2 having an amino acid sequence of SEQ ID NO:470, and a VH CDR3 having an amino acid sequence of SEQ ID NO:471; and (ii) a VL comprising a VL CDR1 having an amino acid sequence of SEQ ID NO:472, a VL CDR2 having an amino acid sequence of SEQ ID NO:473, and a VL CDR3 having an amino acid sequence of SEQ ID NO:474. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:83; and (ii) a VL comprising a VL CDR1, a VL CDR2, and a VL CDR3 having an amino acid sequence of a VL CDR1, VL CDR2, and VL CDR3, respectively, of SEQ ID NO:84. In some embodiments, the first binding domain comprises a VH having an amino acid sequence of SEQ ID NO:83. In some embodiments, the first binding domain comprises a VL having an amino acid sequence of SEQ ID NO:84. In some embodiments, the first binding domain comprises a VH having an amino acid sequence of SEQ ID NO:83, and a VL having an amino acid sequence of SEQ ID NO:84. In some embodiments, the first binding domain comprises a heavy chain having an amino acid sequence of SEQ ID NO:670. In some embodiments, the first binding domain comprises a light chain having an amino acid sequence of SEQ ID NO:671. In some embodiments, the first binding domain comprises a heavy chain having an amino acid sequence of SEQ ID NO:670, and a light chain having an amino acid sequence of SEQ ID NO:671. In some embodiments, the first binding domain comprises a VH having an amino acid sequence having at least 95% identity to an amino acid sequence of SEQ ID NO:83. In some embodiments, the first binding domain comprises a VL having an amino acid sequence having at least 95% identity to an amino acid sequence of SEQ ID NO:84. In some embodiments, the first binding domain comprises a VH having an amino acid sequence having at least 95% identity to an amino acid sequence of SEQ ID NO:83, and a VL having an amino acid sequence having at least 95% identity to an amino acid sequence of SEQ ID NO:84. In some embodiments, the first binding domain comprises a heavy chain having an amino acid sequence having at least 95% identity to an amino acid sequence of SEQ ID NO:670. In some embodiments, the first binding domain comprises a light chain having an amino acid sequence having at least 95% identity to an amino acid sequence of SEQ ID NO:671. In some embodiments, the first binding domain comprises a heavy chain having an amino acid sequence having at least 95% identity to an amino acid sequence of SEQ ID NO:670, and a light chain having an amino acid sequence having at least 95% identity to an amino acid sequence of SEQ ID NO:671.

In some embodiments, the first binding domain that binds to Vβ17 comprises: (i) a VH comprising a VH CDR1 having an amino acid sequence of SEQ ID NO:70, a VH CDR2 having an amino acid sequence of SEQ ID NO:49, and a VH CDR3 having an amino acid sequence of SEQ ID NO:71; and (ii) a VL comprising a VL CDR1 having an amino acid sequence of SEQ ID NO:61, a VL CDR2 having an amino acid sequence of SEQ ID NO:72, and a VL CDR3 having an amino acid sequence of SEQ ID NO:73. In some embodiments, the first binding domain that binds to Vβ17 comprises: (i) a VH comprising a VH CDR1 having an amino acid sequence of SEQ ID NO:475, a VH CDR2 having an amino acid sequence of SEQ ID NO:476, and a VH CDR3 having an amino acid sequence of SEQ ID NO:477; and (ii) a VL comprising a VL CDR1 having an amino acid sequence of SEQ ID NO:478, a VL CDR2 having an amino acid sequence of SEQ ID NO:479, and a VL CDR3 having an amino acid sequence of SEQ ID NO:480. In some embodiments, the first binding domain that binds to Vβ17 comprises: (i) a VH comprising a VH CDR1 having an amino acid sequence of SEQ ID NO:481, a VH CDR2 having an amino acid sequence of SEQ ID NO:482, and a VH CDR3 having an amino acid sequence of SEQ ID NO:483; and (ii) a VL comprising a VL CDR1 having an amino acid sequence of SEQ ID NO:484, a VL CDR2 having an amino acid sequence of SEQ ID NO:485, and a VL CDR3 having an amino acid sequence of SEQ ID NO:486. In some embodiments, the first binding domain that binds to Vβ17 comprises: (i) a VH comprising a VH CDR1 having an amino acid sequence of SEQ ID NO:487, a VH CDR2 having an amino acid sequence of SEQ ID NO:488, and a VH CDR3 having an amino acid sequence of SEQ ID NO:489; and (ii) a VL comprising a VL CDR1 having an amino acid sequence of SEQ ID NO:490, a VL CDR2 having an amino acid sequence of SEQ ID NO:491, and a VL CDR3 having an amino acid sequence of SEQ ID NO:492. In some embodiments, the first binding domain that binds to Vβ17 comprises: (i) a VH comprising a VH CDR1 having an amino acid sequence of SEQ ID NO:493, a VH CDR2 having an amino acid sequence of SEQ ID NO:494, and a VH CDR3 having an amino acid sequence of SEQ ID NO:495; and (ii) a VL comprising a VL CDR1 having an amino acid sequence of SEQ ID NO:496, a VL CDR2 having an amino acid sequence of SEQ ID NO:497, and a VL CDR3 having an amino acid sequence of SEQ ID NO:498. In some embodiments, the first binding domain that binds to Vβ17 comprises: (i) a VH comprising a VH CDR1 having an amino acid sequence of SEQ ID NO:499, a VH CDR2 having an amino acid sequence of SEQ ID NO:500, and a VH CDR3 having an amino acid sequence of SEQ ID NO:501; and (ii) a VL comprising a VL CDR1 having an amino acid sequence of SEQ ID NO:502, a VL CDR2 having an amino acid sequence of SEQ ID NO:503, and a VL CDR3 having an amino acid sequence of SEQ ID NO:504. In some embodiments, the first binding domain that binds to Vβ17 comprises: (i) a VH comprising a VH CDR1 having an amino acid sequence of SEQ ID NO:505, a VH CDR2 having an amino acid sequence of SEQ ID NO:506, and a VH CDR3 having an amino acid sequence of SEQ ID NO:507; and (ii) a VL comprising a VL CDR1 having an amino acid sequence of SEQ ID NO:508, a VL CDR2 having an amino acid sequence of SEQ ID NO:509, and a VL CDR3 having an amino acid sequence of SEQ ID NO:510. In some embodiments, the first binding domain that binds to Vβ17 comprises: (i) a VH comprising a VH CDR1 having an amino acid sequence of SEQ ID NO:511, a VH CDR2 having an amino acid sequence of SEQ ID NO:512, and a VH CDR3 having an amino acid sequence of SEQ ID NO:513; and (ii) a VL comprising a VL CDR1 having an amino acid sequence of SEQ ID NO:514, a VL CDR2 having an amino acid sequence of SEQ ID NO:515, and a VL CDR3 having an amino acid sequence of SEQ ID NO:516 In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:85; and (ii) a VL comprising a VL CDR1, a VL CDR2, and a VL CDR3 having an amino acid sequence of a VL CDR1, VL CDR2, and VL CDR3, respectively, of SEQ ID NO:86. In some embodiments, the first binding domain comprises a VH having an amino acid sequence of SEQ ID NO:85. In some embodiments, the first binding domain comprises a VL having an amino acid sequence of SEQ ID NO:86. In some embodiments, the first binding domain comprises a VH having an amino acid sequence of SEQ ID NO:85, and a VL having an amino acid sequence of SEQ ID NO:86. In some embodiments, the first binding domain comprises a heavy chain having an amino acid sequence of SEQ ID NO:672. In some embodiments, the first binding domain comprises a light chain having an amino acid sequence of SEQ ID NO:673. In some embodiments, the first binding domain comprises a heavy chain having an amino acid sequence of SEQ ID NO:672, and a light chain having an amino acid sequence of SEQ ID NO:673. In some embodiments, the first binding domain comprises a VH having an amino acid sequence having at least 95% identity to an amino acid sequence of SEQ ID NO:85. In some embodiments, the first binding domain comprises a VL having an amino acid sequence having at least 95% identity to an amino acid sequence of SEQ ID NO:86. In some embodiments, the first binding domain comprises a VH having an amino acid sequence having at least 95% identity to an amino acid sequence of SEQ ID NO:85, and a VL having an amino acid sequence having at least 95% identity to an amino acid sequence of SEQ ID NO:86. In some embodiments, the first binding domain comprises a heavy chain having an amino acid sequence having at least 95% identity to an amino acid sequence of SEQ ID NO:672. In some embodiments, the first binding domain comprises a light chain having an amino acid sequence having at least 95% identity to an amino acid sequence of SEQ ID NO:673. In some embodiments, the first binding domain comprises a heavy chain having an amino acid sequence having at least 95% identity to an amino acid sequence of SEQ ID NO:672, and a light chain having an amino acid sequence having at least 95% identity to an amino acid sequence of SEQ ID NO:673.

In some embodiments, the first binding domain that binds to Vβ17 comprises: (i) a VH comprising a VH CDR1 having an amino acid sequence of SEQ ID NO:48, a VH CDR2 having an amino acid sequence of SEQ ID NO:74, and a VH CDR3 having an amino acid sequence of SEQ ID NO:75; and (ii) a VL comprising a VL CDR1 having an amino acid sequence of SEQ ID NO:56, a VL CDR2 having an amino acid sequence of SEQ ID NO:57, and a VL CDR3 having an amino acid sequence of SEQ ID NO:76. In some embodiments, the first binding domain that binds to Vβ17 comprises: (i) a VH comprising a VH CDR1 having an amino acid sequence of SEQ ID NO:517, a VH CDR2 having an amino acid sequence of SEQ ID NO:518, and a VH CDR3 having an amino acid sequence of SEQ ID NO:519; and (ii) a VL comprising a VL CDR1 having an amino acid sequence of SEQ ID NO:520, a VL CDR2 having an amino acid sequence of SEQ ID NO:521, and a VL CDR3 having an amino acid sequence of SEQ ID NO:522. In some embodiments, the first binding domain that binds to Vβ17 comprises: (i) a VH comprising a VH CDR1 having an amino acid sequence of SEQ ID NO:523, a VH CDR2 having an amino acid sequence of SEQ ID NO:524, and a VH CDR3 having an amino acid sequence of SEQ ID NO:525; and (ii) a VL comprising a VL CDR1 having an amino acid sequence of SEQ ID NO:526, a VL CDR2 having an amino acid sequence of SEQ ID NO:527, and a VL CDR3 having an amino acid sequence of SEQ ID NO:528. In some embodiments, the first binding domain that binds to Vβ17 comprises: (i) a VH comprising a VH CDR1 having an amino acid sequence of SEQ ID NO:529, a VH CDR2 having an amino acid sequence of SEQ ID NO:530, and a VH CDR3 having an amino acid sequence of SEQ ID NO:531; and (ii) a VL comprising a VL CDR1 having an amino acid sequence of SEQ ID NO:532, a VL CDR2 having an amino acid sequence of SEQ ID NO:533, and a VL CDR3 having an amino acid sequence of SEQ ID NO:534. In some embodiments, the first binding domain that binds to Vβ17 comprises: (i) a VH comprising a VH CDR1 having an amino acid sequence of SEQ ID NO:535, a VH CDR2 having an amino acid sequence of SEQ ID NO:536, and a VH CDR3 having an amino acid sequence of SEQ ID NO:537; and (ii) a VL comprising a VL CDR1 having an amino acid sequence of SEQ ID NO:538, a VL CDR2 having an amino acid sequence of SEQ ID NO:539, and a VL CDR3 having an amino acid sequence of SEQ ID NO:540. In some embodiments, the first binding domain that binds to Vβ17 comprises: (i) a VH comprising a VH CDR1 having an amino acid sequence of SEQ ID NO:541, a VH CDR2 having an amino acid sequence of SEQ ID NO:542, and a VH CDR3 having an amino acid sequence of SEQ ID NO:543; and (ii) a VL comprising a VL CDR1 having an amino acid sequence of SEQ ID NO:544, a VL CDR2 having an amino acid sequence of SEQ ID NO:545, and a VL CDR3 having an amino acid sequence of SEQ ID NO:546. In some embodiments, the first binding domain that binds to Vβ17 comprises: (i) a VH comprising a VH CDR1 having an amino acid sequence of SEQ ID NO:547, a VH CDR2 having an amino acid sequence of SEQ ID NO:548, and a VH CDR3 having an amino acid sequence of SEQ ID NO:549; and (ii) a VL comprising a VL CDR1 having an amino acid sequence of SEQ ID NO:550, a VL CDR2 having an amino acid sequence of SEQ ID NO:551, and a VL CDR3 having an amino acid sequence of SEQ ID NO:552. In some embodiments, the first binding domain that binds to Vβ17 comprises: i) a VH comprising a VH CDR1 having an amino acid sequence of SEQ ID NO:553, a VH CDR2 having an amino acid sequence of SEQ ID NO:554, and a VH CDR3 having an amino acid sequence of SEQ ID NO:555; and (ii) a VL comprising a VL CDR1 having an amino acid sequence of SEQ ID NO:556, a VL CDR2 having an amino acid sequence of SEQ ID NO:557, and a VL CDR3 having an amino acid sequence of SEQ ID NO:558. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:87; and (ii) a VL comprising a VL CDR1, a VL CDR2, and a VL CDR3 having an amino acid sequence of a VL CDR1, VL CDR2, and VL CDR3, respectively, of SEQ ID NO:88. In some embodiments, the first binding domain comprises a VH having an amino acid sequence of SEQ ID NO:87. In some embodiments, the first binding domain comprises a VL having an amino acid sequence of SEQ ID NO:88. In some embodiments, the first binding domain comprises a VH having an amino acid sequence of SEQ ID NO:87, and a VL having an amino acid sequence of SEQ ID NO:88. In some embodiments, the first binding domain comprises a heavy chain having an amino acid sequence of SEQ ID NO:674. In some embodiments, the first binding domain comprises a light chain having an amino acid sequence of SEQ ID NO:675. In some embodiments, the first binding domain comprises a heavy chain having an amino acid sequence of SEQ ID NO:674, and a light chain having an amino acid sequence of SEQ ID NO:675. In some embodiments, the first binding domain comprises a VH having an amino acid sequence having at least 95% identity to an amino acid sequence of SEQ ID NO:87. In some embodiments, the first binding domain comprises a VL having an amino acid sequence having at least 95% identity to an amino acid sequence of SEQ ID NO:88. In some embodiments, the first binding domain comprises a VH having an amino acid sequence having at least 95% identity to an amino acid sequence of SEQ ID NO:87, and a VL having an amino acid sequence having at least 95% identity to an amino acid sequence of SEQ ID NO:88. In some embodiments, the first binding domain comprises a heavy chain having an amino acid sequence having at least 95% identity to an amino acid sequence of SEQ ID NO:674. In some embodiments, the first binding domain comprises a light chain having an amino acid sequence having at least 95% identity to an amino acid sequence of SEQ ID NO:675. In some embodiments, the first binding domain comprises a heavy chain having an amino acid sequence having at least 95% identity to an amino acid sequence of SEQ ID NO:674, and a light chain having an amino acid sequence having at least 95% identity to an amino acid sequence of SEQ ID NO:675.

In some embodiments, the first binding domain that binds to Vβ17 comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:664, a VL CDR2 having an amino acid sequence of SEQ ID NO:5, and a VL CDR3 having an amino acid sequence of SEQ ID NO:6. In some embodiments, the first binding domain that binds to Vβ17 comprises: (i) a VH comprising a VH CDR1 having an amino acid sequence of SEQ ID NO:223, a VH CDR2 having an amino acid sequence of SEQ ID NO:224, and a VH CDR3 having an amino acid sequence of SEQ ID NO:225; and (ii) a VL comprising a VL CDR1 having an amino acid sequence of SEQ ID NO:678, a VL CDR2 having an amino acid sequence of SEQ ID NO:227, and a VL CDR3 having an amino acid sequence of SEQ ID NO:228. In some embodiments, the first binding domain that binds to Vβ17 comprises: (i) a VH comprising a VH CDR1 having an amino acid sequence of SEQ ID NO:229, a VH CDR2 having an amino acid sequence of SEQ ID NO:230, and a VH CDR3 having an amino acid sequence of SEQ ID NO:231; and (ii) a VL comprising a VL CDR1 having an amino acid sequence of SEQ ID NO:679, a VL CDR2 having an amino acid sequence of SEQ ID NO:233, and a VL CDR3 having an amino acid sequence of SEQ ID NO:234. In some embodiments, the first binding domain that binds to Vβ17 comprises: (i) a VH comprising a VH CDR1 having an amino acid sequence of SEQ ID NO:235, a VH CDR2 having an amino acid sequence of SEQ ID NO:236, and a VH CDR3 having an amino acid sequence of SEQ ID NO:237; and (ii) a VL comprising a VL CDR1 having an amino acid sequence of SEQ ID NO:680, a VL CDR2 having an amino acid sequence of SEQ ID NO:239, and a VL CDR3 having an amino acid sequence of SEQ ID NO:240. In some embodiments, the first binding domain that binds to Vβ17 comprises: (i) a VH comprising a VH CDR1 having an amino acid sequence of SEQ ID NO:241, a VH CDR2 having an amino acid sequence of SEQ ID NO:242, and a VH CDR3 having an amino acid sequence of SEQ ID NO:243; and (ii) a VL comprising a VL CDR1 having an amino acid sequence of SEQ ID NO:681, a VL CDR2 having an amino acid sequence of SEQ ID NO:245, and a VL CDR3 having an amino acid sequence of SEQ ID NO:246. In some embodiments, the first binding domain that binds to Vβ17 comprises: (i) a VH comprising a VH CDR1 having an amino acid sequence of SEQ ID NO:241, a VH CDR2 having an amino acid sequence of SEQ ID NO:682, and a VH CDR3 having an amino acid sequence of SEQ ID NO:683; and (ii) a VL comprising a VL CDR1 having an amino acid sequence of SEQ ID NO:684, a VL CDR2 having an amino acid sequence of SEQ ID NO:245, and a VL CDR3 having an amino acid sequence of SEQ ID NO:246. In some embodiments, the first binding domain that binds to Vβ17 comprises: (i) a VH comprising a VH CDR1 having an amino acid sequence of SEQ ID NO:241, a VH CDR2 having an amino acid sequence of SEQ ID NO:687, and a VH CDR3 having an amino acid sequence of SEQ ID NO:683; and (ii) a VL comprising a VL CDR1 having an amino acid sequence of SEQ ID NO:684, a VL CDR2 having an amino acid sequence of SEQ ID NO:245, and a VL CDR3 having an amino acid sequence of SEQ ID NO:246. In some embodiments, the first binding domain that binds to Vβ17 comprises: (i) a VH comprising a VH CDR1 having an amino acid sequence of SEQ ID NO:253, a VH CDR2 having an amino acid sequence of SEQ ID NO:254, and a VH CDR3 having an amino acid sequence of SEQ ID NO:255; and (ii) a VL comprising a VL CDR1 having an amino acid sequence of SEQ ID NO:685, a VL CDR2 having an amino acid sequence of SEQ ID NO:257, and a VL CDR3 having an amino acid sequence of SEQ ID NO:258. In some embodiments, the first binding domain that binds to Vβ17 comprises: i) a VH comprising a VH CDR1 having an amino acid sequence of SEQ ID NO:259, a VH CDR2 having an amino acid sequence of SEQ ID NO:260, and a VH CDR3 having an amino acid sequence of SEQ ID NO:261; and (ii) a VL comprising a VL CDR1 having an amino acid sequence of SEQ ID NO:686, a VL CDR2 having an amino acid sequence of SEQ ID NO:263, and a VL CDR3 having an amino acid sequence of SEQ ID NO:264. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:665. In some embodiments, the first binding domain comprises a VH having an amino acid sequence of SEQ ID NO:21. In some embodiments, the first binding domain comprises a VL having an amino acid sequence of SEQ ID NO:665. In some embodiments, the first binding domain comprises a VH having an amino acid sequence of SEQ ID NO:21, and a VL having an amino acid sequence of SEQ ID NO:665. In some embodiments, the first binding domain comprises a VH having an amino acid sequence having at least 95% identity to an amino acid sequence of SEQ ID NO:21. In some embodiments, the first binding domain comprises a VL having an amino acid sequence having at least 95% identity to an amino acid sequence of SEQ ID NO:665. In some embodiments, the first binding domain comprises a VH having an amino acid sequence having at least 95% identity to an amino acid sequence of SEQ ID NO:21, and a VL having an amino acid sequence having at least 95% identity to an amino acid sequence of SEQ ID NO:665.

In some embodiments, the first binding domain that binds to Vβ17 comprises: (i) a VH comprising a VH CDR1 having an amino acid sequence of SEQ ID NO:1057, a VH CDR2 having an amino acid sequence of SEQ ID NO:1058, and a VH CDR3 having an amino acid sequence of SEQ ID NO:1059; and (ii) a VL comprising a VL CDR1 having an amino acid sequence of SEQ ID NO:1072, a VL CDR2 having an amino acid sequence of SEQ ID NO:1073, and a VL CDR3 having an amino acid sequence of SEQ ID NO:1074. In some embodiments, the first binding domain that binds to Vβ17 comprises: (i) a VH comprising a VH CDR1 having an amino acid sequence of SEQ ID NO:1060, a VH CDR2 having an amino acid sequence of SEQ ID NO:1061, and a VH CDR3 having an amino acid sequence of SEQ ID NO:1062; and (ii) a VL comprising a VL CDR1 having an amino acid sequence of SEQ ID NO:1075, a VL CDR2 having an amino acid sequence of SEQ ID NO:1076, and a VL CDR3 having an amino acid sequence of SEQ ID NO:1077. In some embodiments, the first binding domain that binds to Vβ17 comprises: (i) a VH comprising a VH CDR1 having an amino acid sequence of SEQ ID NO:1054, a VH CDR2 having an amino acid sequence of SEQ ID NO:1055, and a VH CDR3 having an amino acid sequence of SEQ ID NO:1056; and (ii) a VL comprising a VL CDR1 having an amino acid sequence of SEQ ID NO:1069, a VL CDR2 having an amino acid sequence of SEQ ID NO:1070, and a VL CDR3 having an amino acid sequence of SEQ ID NO:1071. In some embodiments, the first binding domain that binds to Vβ17 comprises: (i) a VH comprising a VH CDR1 having an amino acid sequence of SEQ ID NO:1066, a VH CDR2 having an amino acid sequence of SEQ ID NO:1067, and a VH CDR3 having an amino acid sequence of SEQ ID NO:1068; and (ii) a VL comprising a VL CDR1 having an amino acid sequence of SEQ ID NO:1081, a VL CDR2 having an amino acid sequence of SEQ ID NO:1082, and a VL CDR3 having an amino acid sequence of SEQ ID NO:1083. In some embodiments, the first binding domain that binds to Vβ17 comprises: (i) a VH comprising a VH CDR1 having an amino acid sequence of SEQ ID NO:1063, a VH CDR2 having an amino acid sequence of SEQ ID NO:1064, and a VH CDR3 having an amino acid sequence of SEQ ID NO:1065; and (ii) a VL comprising a VL CDR1 having an amino acid sequence of SEQ ID NO:1078, a VL CDR2 having an amino acid sequence of SEQ ID NO:1079, and a VL CDR3 having an amino acid sequence of SEQ ID NO:1080. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:1084; and (ii) a VL comprising a VL CDR1, a VL CDR2, and a VL CDR3 having an amino acid sequence of a VL CDR1, VL CDR2, and VL CDR3, respectively, of SEQ ID NO:1085. In some embodiments, the first binding domain comprises a VH having an amino acid sequence of SEQ ID NO:1084. In some embodiments, the first binding domain comprises a VL having an amino acid sequence of SEQ ID NO:1085. In some embodiments, the first binding domain comprises a VH having an amino acid sequence of SEQ ID NO:1084, and a VL having an amino acid sequence of SEQ ID NO:1085. In some embodiments, the first binding domain comprises a VH having an amino acid sequence having at least 95% identity to an amino acid sequence of SEQ ID NO:1084. In some embodiments, the first binding domain comprises a VL having an amino acid sequence having at least 95% identity to an amino acid sequence of SEQ ID NO:1085. In some embodiments, the first binding domain comprises a VH having an amino acid sequence having at least 95% identity to an amino acid sequence of SEQ ID NO:1084, and a VL having an amino acid sequence having at least 95% identity to an amino acid sequence of SEQ ID NO:1085.

In some embodiments, the second binding domain binds to BCMA. In some embodiments, the second binding domain that binds to BCMA comprises VH CDR1, VH CDR2, and VH CDR3 amino acid sequences of a BCMA antibody provided herein. In some embodiments, the second binding domain that binds to BCMA comprises VL CDR1, VL CDR2 and VL CDR3 amino acid sequences of a BCMA antibody provided herein. In some embodiments, the second binding domain that binds to BCMA comprises VH CDR1, VH CDR2, VH CDR3, VL CDR1, VL CDR2 and VL CDR3 amino acid sequences of a BCMA antibody provided herein. In other embodiments, the second binding domain that binds to BCMA comprises VH amino acid sequence of a BCMA antibody provided herein. In other embodiments, the second binding domain that binds to BCMA comprises a VL amino acid sequence of a BCMA antibody provided herein. In other embodiments, the second binding domain that binds to BCMA comprises VH and VL amino acid sequences of a BCMA antibody provided herein. In other embodiments, the second binding domain that binds to BCMA comprises heavy chain amino acid sequence of a BCMA antibody provided herein. In other embodiments, the second binding domain that binds to BCMA comprises a light chain amino acid sequence of a BCMA antibody provided herein. In other embodiments, the second binding domain that binds to BCMA comprises heavy chain and light chain amino acid sequences of a BCMA antibody provided herein. In some embodiments, the BCMA antibody is clone BCMB519. Other BCMA antibodies, including antigen binding fragments thereof, are also contemplated as the second binding arm that binds to BCMA in the trispecific antibodies provided herein.

In some embodiments, the second binding domain that binds to BCMA comprises: (i) a VH comprising a VH CDR1 having an amino acid sequence of SEQ ID NO:89, a VH CDR2 having an amino acid sequence of SEQ ID NO:90, and a VH CDR3 having an amino acid sequence of SEQ ID NO:91; and (ii) a VL comprising a VL CDR1 having an amino acid sequence of SEQ ID NO:93, a VL CDR2 having an amino acid sequence of SEQ ID NO:94, and a VL CDR3 having an amino acid sequence of SEQ ID NO:94. In some embodiments, the second binding domain that binds to BCMA comprises: (i) a VH comprising a VH CDR1 having an amino acid sequence of SEQ ID NO:622, a VH CDR2 having an amino acid sequence of SEQ ID NO:623, and a VH CDR3 having an amino acid sequence of SEQ ID NO:624; and (ii) a VL comprising a VL CDR1 having an amino acid sequence of SEQ ID NO:625, a VL CDR2 having an amino acid sequence of SEQ ID NO:626, and a VL CDR3 having an amino acid sequence of SEQ ID NO:627. In some embodiments, the second binding domain that binds to BCMA comprises: (i) a VH comprising a VH CDR1 having an amino acid sequence of SEQ ID NO:628, a VH CDR2 having an amino acid sequence of SEQ ID NO:629, and a VH CDR3 having an amino acid sequence of SEQ ID NO:630; and (ii) a VL comprising a VL CDR1 having an amino acid sequence of SEQ ID NO:631, a VL CDR2 having an amino acid sequence of SEQ ID NO:632, and a VL CDR3 having an amino acid sequence of SEQ ID NO:633. In some embodiments, the second binding domain that binds to BCMA comprises: (i) a VH comprising a VH CDR1 having an amino acid sequence of SEQ ID NO:634, a VH CDR2 having an amino acid sequence of SEQ ID NO:635, and a VH CDR3 having an amino acid sequence of SEQ ID NO:636; and (ii) a VL comprising a VL CDR1 having an amino acid sequence of SEQ ID NO:637, a VL CDR2 having an amino acid sequence of SEQ ID NO:638, and a VL CDR3 having an amino acid sequence of SEQ ID NO:639. In some embodiments, the second binding domain that binds to BCMA comprises: (i) a VH comprising a VH CDR1 having an amino acid sequence of SEQ ID NO:640, a VH CDR2 having an amino acid sequence of SEQ ID NO:641, and a VH CDR3 having an amino acid sequence of SEQ ID NO:642; and (ii) a VL comprising a VL CDR1 having an amino acid sequence of SEQ ID NO:643, a VL CDR2 having an amino acid sequence of SEQ ID NO:644, and a VL CDR3 having an amino acid sequence of SEQ ID NO:645. In some embodiments, the second binding domain that binds to BCMA comprises: (i) a VH comprising a VH CDR1 having an amino acid sequence of SEQ ID NO:646, a VH CDR2 having an amino acid sequence of SEQ ID NO:647, and a VH CDR3 having an amino acid sequence of SEQ ID NO:648; and (ii) a VL comprising a VL CDR1 having an amino acid sequence of SEQ ID NO:649, a VL CDR2 having an amino acid sequence of SEQ ID NO:650, and a VL CDR3 having an amino acid sequence of SEQ ID NO:651. In some embodiments, the second binding domain that binds to BCMA comprises: (i) a VH comprising a VH CDR1 having an amino acid sequence of SEQ ID NO:652, a VH CDR2 having an amino acid sequence of SEQ ID NO:653, and a VH CDR3 having an amino acid sequence of SEQ ID NO:654; and (ii) a VL comprising a VL CDR1 having an amino acid sequence of SEQ ID NO:655, a VL CDR2 having an amino acid sequence of SEQ ID NO:656, and a VL CDR3 having an amino acid sequence of SEQ ID NO:657. In some embodiments, the second binding domain that binds to BCMA comprises: (i) a VH comprising a VH CDR1 having an amino acid sequence of SEQ ID NO:658, a VH CDR2 having an amino acid sequence of SEQ ID NO:659, and a VH CDR3 having an amino acid sequence of SEQ ID NO:660; and (ii) a VL comprising a VL CDR1 having an amino acid sequence of SEQ ID NO:661, a VL CDR2 having an amino acid sequence of SEQ ID NO:662, and a VL CDR3 having an amino acid sequence of SEQ ID NO:663. In some embodiments, the second binding domain that binds to BCMA comprises: (i) a VH comprising a VH CDR1, a VH CDR2, and a VH CDR3 having an amino acid sequence of a VH CDR1, VH CDR2, and VH CDR3, respectively, of SEQ ID NO:95; and (ii) a VL comprising a VL CDR1, a VL CDR2, and a VL CDR3 having an amino acid sequence of a VL CDR1, VL CDR2, and VL CDR3, respectively, of SEQ ID NO:96. In some embodiments, the second binding domain that binds to BCMA comprises a VH having an amino acid sequence of SEQ ID NO:95. In some embodiments, the second binding domain that binds to BCMA comprises a VL having an amino acid sequence of SEQ ID NO:96. In some embodiments, the second binding domain that binds to BCMA comprises a VH having an amino acid sequence of SEQ ID NO:95, and a VL having an amino acid sequence of SEQ ID NO:96. In some embodiments, the second binding domain that binds to BCMA comprises a VH having an amino acid sequence having at least 95% identity to an amino acid sequence of SEQ ID NO:95. In some embodiments, the second binding domain that binds to BCMA comprises a VL having an amino acid sequence having at least 95% identity to an amino acid sequence of SEQ ID NO:96. In some embodiments, the second binding domain that binds to BCMA comprises a VH having an amino acid sequence having at least 95% identity to an amino acid sequence of SEQ ID NO:95, and a VL having an amino acid sequence having at least 95% identity to an amino acid sequence of SEQ ID NO:96. In some embodiments, the second binding domain that binds to BCMA comprises: (i) a VH comprising a VH CDR1, a VH CDR2, and a VH CDR3 having an amino acid sequence of a VH CDR1, VH CDR2, and VH CDR3, respectively, of SEQ ID NO:1052; and (ii) a VL comprising a VL CDR1, a VL CDR2, and a VL CDR3 having an amino acid sequence of a VL CDR1, VL CDR2, and VL CDR3, respectively, of SEQ ID NO:1053. In some embodiments, the second binding domain that binds to BCMA comprises a VH having an amino acid sequence of SEQ ID NO:1052. In some embodiments, the second binding domain that binds to BCMA comprises a VL having an amino acid sequence of SEQ ID NO:1053. In some embodiments, the second binding domain that binds to BCMA comprises a VH having an amino acid sequence of SEQ ID NO:1052, and a VL having an amino acid sequence of SEQ ID NO:1053. In some embodiments, the second binding domain that binds to BCMA comprises a VH having an amino acid sequence having at least 95% identity to an amino acid sequence of SEQ ID NO:1052. In some embodiments, the second binding domain that binds to BCMA comprises a VL having an amino acid sequence having at least 95% identity to an amino acid sequence of SEQ ID NO:1053. In some embodiments, the second binding domain that binds to BCMA comprises a VH having an amino acid sequence having at least 95% identity to an amino acid sequence of SEQ ID NO:1052, and a VL having an amino acid sequence having at least 95% identity to an amino acid sequence of SEQ ID NO:1053.

In some embodiments, the second binding domain binds to PSMA. In some embodiments, the second binding domain that binds to PSMA comprises VH CDR1, VH CDR2, and VH CDR3 amino acid sequences of a PSMA antibody provided herein. In some embodiments, the second binding domain that binds to PSMA comprises VL CDR1, VL CDR2 and VL CDR3 amino acid sequences of a PSMA antibody provided herein. In some embodiments, the second binding domain that binds to PSMA comprises VH CDR1, VH CDR2, VH CDR3, VL CDR1, VL CDR2 and VL CDR3 amino acid sequences of a PSMA antibody provided herein. In other embodiments, the second binding domain that binds to PSMA comprises VH amino acid sequence of a PSMA antibody provided herein. In other embodiments, the second binding domain that binds to PSMA comprises a VL amino acid sequence of a PSMA antibody provided herein. In other embodiments, the second binding domain that binds to PSMA comprises VH and VL amino acid sequences of a PSMA antibody provided herein. In other embodiments, the second binding domain that binds to PSMA comprises heavy chain amino acid sequence of a PSMA antibody provided herein. In other embodiments, the second binding domain that binds to PSMA comprises a light chain amino acid sequence of a PSMA antibody provided herein. In other embodiments, the second binding domain that binds to PSMA comprises heavy chain and light chain amino acid sequences of a PSMA antibody provided herein. In some embodiments, the PSMA antibody is clone PSMB410. Other PSMA antibodies, including antigen binding fragments thereof, are also contemplated as the second binding arm that binds to PSMA in the trispecific antibodies provided herein.

In some embodiments, the second binding domain binds to PSMA. In some embodiments, the second binding domain that binds to PSMA comprises: (i) a VH comprising a VH CDR1 having an amino acid sequence of SEQ ID NO:1019, a VH CDR2 having an amino acid sequence of SEQ ID NO:1020, and a VH CDR3 having an amino acid sequence of SEQ ID NO:1021; and (ii) a VL comprising a VL CDR1 having an amino acid sequence of SEQ ID NO:1034, a VL CDR2 having an amino acid sequence of SEQ ID NO:1035, and a VL CDR3 having an amino acid sequence of SEQ ID NO:1036. In some embodiments, the second binding domain that binds to PSMA comprises: (i) a VH comprising a VH CDR1 having an amino acid sequence of SEQ ID NO:1022, a VH CDR2 having an amino acid sequence of SEQ ID NO:1023, and a VH CDR3 having an amino acid sequence of SEQ ID NO:1024; and (ii) a VL comprising a VL CDR1 having an amino acid sequence of SEQ ID NO:1037, a VL CDR2 having an amino acid sequence of SEQ ID NO:1038, and a VL CDR3 having an amino acid sequence of SEQ ID NO:1039. In some embodiments, the second binding domain that binds to PSMA comprises: (i) a VH comprising a VH CDR1 having an amino acid sequence of SEQ ID NO:1016, a VH CDR2 having an amino acid sequence of SEQ ID NO:1017, and a VH CDR3 having an amino acid sequence of SEQ ID NO:1018; and (ii) a VL comprising a VL CDR1 having an amino acid sequence of SEQ ID NO:1031, a VL CDR2 having an amino acid sequence of SEQ ID NO:1032, and a VL CDR3 having an amino acid sequence of SEQ ID NO:1033. In some embodiments, the second binding domain that binds to PSMA comprises: (i) a VH comprising a VH CDR1 having an amino acid sequence of SEQ ID NO:1028, a VH CDR2 having an amino acid sequence of SEQ ID NO:1029, and a VH CDR3 having an amino acid sequence of SEQ ID NO:1030; and (ii) a VL comprising a VL CDR1 having an amino acid sequence of SEQ ID NO:1043, a VL CDR2 having an amino acid sequence of SEQ ID NO:1044, and a VL CDR3 having an amino acid sequence of SEQ ID NO:1045. In some embodiments, the second binding domain that binds to PSMA comprises: (i) a VH comprising a VH CDR1 having an amino acid sequence of SEQ ID NO:1025, a VH CDR2 having an amino acid sequence of SEQ ID NO:1026, and a VH CDR3 having an amino acid sequence of SEQ ID NO:1027; and (ii) a VL comprising a VL CDR1 having an amino acid sequence of SEQ ID NO:1040, a VL CDR2 having an amino acid sequence of SEQ ID NO:1041, and a VL CDR3 having an amino acid sequence of SEQ ID NO:1042. In some embodiments, the second binding domain that binds to PSMA comprises: a VH comprising a VH CDR1, a VH CDR2, and a VH CDR3 having an amino acid sequence of a VH CDR1, VH CDR2, and VH CDR3, respectively, of SEQ ID NO:1046. In some embodiments, the second binding domain that binds to PSMA comprises: a VL comprising a VL CDR1, a VL CDR2, and a VL CDR3 having an amino acid sequence of a VL CDR1, VL CDR2, and VL CDR3, respectively, of SEQ ID NO:1047. In some embodiments, the second binding domain that binds to PSMA comprises: (i) a VH comprising a VH CDR1, a VH CDR2, and a VH CDR3 having an amino acid sequence of a VH CDR1, VH CDR2, and VH CDR3, respectively, of SEQ ID NO:1046; and (ii) a VL comprising a VL CDR1, a VL CDR2, and a VL CDR3 having an amino acid sequence of a VL CDR1, VL CDR2, and VL CDR3, respectively, of SEQ ID NO:1047. In some embodiments, the second binding domain that binds to PSMA comprises a VH having an amino acid sequence of SEQ ID NO:1046. In some embodiments, the second binding domain that binds to PSMA comprises a VL having an amino acid sequence of SEQ ID NO:1047. In some embodiments, the second binding domain that binds to PSMA comprises a VH having an amino acid sequence of SEQ ID NO:1046, and a VL having an amino acid sequence of SEQ ID NO:1047. In some embodiments, the second binding domain that binds to PSMA comprises a VH having an amino acid sequence having at least 95% identity to an amino acid sequence of SEQ ID NO:1046. In some embodiments, the second binding domain that binds to PSMA comprises a VL having an amino acid sequence having at least 95% identity to an amino acid sequence of SEQ ID NO:1047. In some embodiments, the second binding domain that binds to PSMA comprises a VH having an amino acid sequence having at least 95% identity to an amino acid sequence of SEQ ID NO:1046, and a VL having an amino acid sequence having at least 95% identity to an amino acid sequence of SEQ ID NO:1047.

In some embodiments, the third binding domain that binds to CD28 comprises VH CDR1, VH CDR2, and VH CDR3 amino acid sequences of a CD28 antibody provided herein. In some embodiments, the third binding domain that binds to CD28 comprises VL CDR1, VL CDR2 and VL CDR3 amino acid sequences of a CD28 antibody provided herein. In some embodiments, the third binding domain that binds to CD28 comprises VH CDR1, VH CDR2, VH CDR3, VL CDR1, VL CDR2 and VL CDR3 amino acid sequences of a CD28 antibody provided herein. In other embodiments, the third binding domain that binds to CD28 comprises VH amino acid sequence of a CD28 antibody provided herein. In other embodiments, the third binding domain that binds to CD28 comprises a VL amino acid sequence of a CD28 antibody provided herein. In other embodiments, the third binding domain that binds to CD28 comprises VH and VL amino acid sequences of a CD28 antibody provided herein. In other embodiments, the third binding domain that binds to CD28 comprises heavy chain amino acid sequence of a CD28 antibody provided herein. In other embodiments, the third binding domain that binds to CD28 comprises a light chain amino acid sequence of a CD28 antibody provided herein. In other embodiments, the third binding domain that binds to CD28 comprises heavy chain and light chain amino acid sequences of a CD28 antibody provided herein. In some embodiments, the CD28 antibody is clone C28B11. In some embodiments, the CD28 antibody is clone C28B19. In some embodiments, the CD28 antibody is clone C28B103. In some embodiments, the CD28 antibody is clone C28B105. Other CD28 antibodies, including antigen binding fragments thereof, are also contemplated as the third binding arm that binds to CD28 in the trispecific antibodies provided herein.

In some embodiments, the third binding domain that binds to CD28 comprises: (i) a VH comprising a VH CDR1 having an amino acid sequence of SEQ ID NO:702, a VH CDR2 having an amino acid sequence of SEQ ID NO:708, and a VH CDR3 having an amino acid sequence of SEQ ID NO:714; and (ii) a VL comprising a VL CDR1 having an amino acid sequence of SEQ ID NO:794, a VL CDR2 having an amino acid sequence of SEQ ID NO:800, and a VL CDR3 having an amino acid sequence of SEQ ID NO:806. In some embodiments, the third binding domain that binds to CD28 comprises: (i) a VH comprising a VH CDR1 having an amino acid sequence of SEQ ID NO:721, a VH CDR2 having an amino acid sequence of SEQ ID NO:727, and a VH CDR3 having an amino acid sequence of SEQ ID NO:733; and (ii) a VL comprising a VL CDR1 having an amino acid sequence of SEQ ID NO:812, a VL CDR2 having an amino acid sequence of SEQ ID NO:818, and a VL CDR3 having an amino acid sequence of SEQ ID NO:824. In some embodiments, the third binding domain that binds to CD28 comprises: (i) a VH comprising a VH CDR1 having an amino acid sequence of SEQ ID NO:739, a VH CDR2 having an amino acid sequence of SEQ ID NO:746, and a VH CDR3 having an amino acid sequence of SEQ ID NO:752; and (ii) a VL comprising a VL CDR1 having an amino acid sequence of SEQ ID NO:830, a VL CDR2 having an amino acid sequence of SEQ ID NO:836, and a VL CDR3 having an amino acid sequence of SEQ ID NO:842. In some embodiments, the third binding domain that binds to CD28 comprises: (i) a VH comprising a VH CDR1 having an amino acid sequence of SEQ ID NO:758, a VH CDR2 having an amino acid sequence of SEQ ID NO:764, and a VH CDR3 having an amino acid sequence of SEQ ID NO:770; and (ii) a VL comprising a VL CDR1 having an amino acid sequence of SEQ ID NO:848, a VL CDR2 having an amino acid sequence of SEQ ID NO:854, and a VL CDR3 having an amino acid sequence of SEQ ID NO:860. In some embodiments, the third binding domain that binds to CD28 comprises: (i) a VH comprising a VH CDR1 having an amino acid sequence of SEQ ID NO:776, a VH CDR2 having an amino acid sequence of SEQ ID NO:782, and a VH CDR3 having an amino acid sequence of SEQ ID NO:788; and (ii) a VL comprising a VL CDR1 having an amino acid sequence of SEQ ID NO:866, a VL CDR2 having an amino acid sequence of SEQ ID NO:872, and a VL CDR3 having an amino acid sequence of SEQ ID NO:878. In some embodiments, the third binding domain that binds to CD28 comprises: (i) a VH comprising a VH CDR1, a VH CDR2, and a VH CDR3 having an amino acid sequence of a VH CDR1, VH CDR2, and VH CDR3, respectively, of SEQ ID NO:690; and (ii) a VL comprising a VL CDR1, a VL CDR2, and a VL CDR3 having an amino acid sequence of a VL CDR1, VL CDR2, and VL CDR3, respectively, of SEQ ID NO:696. In some embodiments, the third binding domain that binds to CD28 comprises a VH having an amino acid sequence of SEQ ID NO:690. In some embodiments, the third binding domain that binds to CD28 comprises a VL having an amino acid sequence of SEQ ID NO:696. In some embodiments, the third binding domain that binds to CD28 comprises a VH having an amino acid sequence of SEQ ID NO:690, and a VL having an amino acid sequence of SEQ ID NO:696. In some embodiments, the third binding domain that binds to CD28 comprises a VH having an amino acid sequence having at least 95% identity to an amino acid sequence of SEQ ID NO:690. In some embodiments, the third binding domain that binds to CD28 comprises a VL having an amino acid sequence having at least 95% identity to an amino acid sequence of SEQ ID NO:696. In some embodiments, the third binding domain that binds to CD28 comprises a VH having an amino acid sequence having at least 95% identity to an amino acid sequence of SEQ ID NO:690, and a VL having an amino acid sequence having at least 95% identity to an amino acid sequence of SEQ ID NO:696.

In some embodiments, the third binding domain that binds to CD28 comprises: (i) a VH comprising a VH CDR1 having an amino acid sequence of SEQ ID NO:703, a VH CDR2 having an amino acid sequence of SEQ ID NO:709, and a VH CDR3 having an amino acid sequence of SEQ ID NO:715; and (ii) a VL comprising a VL CDR1 having an amino acid sequence of SEQ ID NO:795, a VL CDR2 having an amino acid sequence of SEQ ID NO:801, and a VL CDR3 having an amino acid sequence of SEQ ID NO:807. In some embodiments, the third binding domain that binds to CD28 comprises: (i) a VH comprising a VH CDR1 having an amino acid sequence of SEQ ID NO:722, a VH CDR2 having an amino acid sequence of SEQ ID NO:728, and a VH CDR3 having an amino acid sequence of SEQ ID NO:734; and (ii) a VL comprising a VL CDR1 having an amino acid sequence of SEQ ID NO:813, a VL CDR2 having an amino acid sequence of SEQ ID NO:819, and a VL CDR3 having an amino acid sequence of SEQ ID NO:825. In some embodiments, the third binding domain that binds to CD28 comprises: (i) a VH comprising a VH CDR1 having an amino acid sequence of SEQ ID NO:740, a VH CDR2 having an amino acid sequence of SEQ ID NO:747, and a VH CDR3 having an amino acid sequence of SEQ ID NO:753; and (ii) a VL comprising a VL CDR1 having an amino acid sequence of SEQ ID NO:831, a VL CDR2 having an amino acid sequence of SEQ ID NO:837, and a VL CDR3 having an amino acid sequence of SEQ ID NO:843. In some embodiments, the third binding domain that binds to CD28 comprises: (i) a VH comprising a VH CDR1 having an amino acid sequence of SEQ ID NO:759, a VH CDR2 having an amino acid sequence of SEQ ID NO:765, and a VH CDR3 having an amino acid sequence of SEQ ID NO:771; and (ii) a VL comprising a VL CDR1 having an amino acid sequence of SEQ ID NO:849, a VL CDR2 having an amino acid sequence of SEQ ID NO:855, and a VL CDR3 having an amino acid sequence of SEQ ID NO:861. In some embodiments, the third binding domain that binds to CD28 comprises: (i) a VH comprising a VH CDR1 having an amino acid sequence of SEQ ID NO:777, a VH CDR2 having an amino acid sequence of SEQ ID NO:783, and a VH CDR3 having an amino acid sequence of SEQ ID NO:789; and (ii) a VL comprising a VL CDR1 having an amino acid sequence of SEQ ID NO:867, a VL CDR2 having an amino acid sequence of SEQ ID NO:873, and a VL CDR3 having an amino acid sequence of SEQ ID NO:879. In some embodiments, the third binding domain that binds to CD28 comprises: (i) a VH comprising a VH CDR1, a VH CDR2, and a VH CDR3 having an amino acid sequence of a VH CDR1, VH CDR2, and VH CDR3, respectively, of SEQ ID NO:691; and (ii) a VL comprising a VL CDR1, a VL CDR2, and a VL CDR3 having an amino acid sequence of a VL CDR1, VL CDR2, and VL CDR3, respectively, of SEQ ID NO:697. In some embodiments, the third binding domain that binds to CD28 comprises a VH having an amino acid sequence of SEQ ID NO:691. In some embodiments, the third binding domain that binds to CD28 comprises a VL having an amino acid sequence of SEQ ID NO:697. In some embodiments, the third binding domain that binds to CD28 comprises a VH having an amino acid sequence of SEQ ID NO:691, and a VL having an amino acid sequence of SEQ ID NO:697. In some embodiments, the third binding domain that binds to CD28 comprises a VH having an amino acid sequence having at least 95% identity to an amino acid sequence of SEQ ID NO:691. In some embodiments, the third binding domain that binds to CD28 comprises a VL having an amino acid sequence having at least 95% identity to an amino acid sequence of SEQ ID NO:697. In some embodiments, the third binding domain that binds to CD28 comprises a VH having an amino acid sequence having at least 95% identity to an amino acid sequence of SEQ ID NO:691, and a VL having an amino acid sequence having at least 95% identity to an amino acid sequence of SEQ ID NO:697.

In some embodiments, the third binding domain that binds to CD28 comprises: (i) a VH comprising a VH CDR1 having an amino acid sequence of SEQ ID NO:704, a VH CDR2 having an amino acid sequence of SEQ ID NO:710, and a VH CDR3 having an amino acid sequence of SEQ ID NO:716; and (ii) a VL comprising a VL CDR1 having an amino acid sequence of SEQ ID NO:796, a VL CDR2 having an amino acid sequence of SEQ ID NO:802, and a VL CDR3 having an amino acid sequence of SEQ ID NO:808. In some embodiments, the third binding domain that binds to CD28 comprises: (i) a VH comprising a VH CDR1 having an amino acid sequence of SEQ ID NO:723, a VH CDR2 having an amino acid sequence of SEQ ID NO:729, and a VH CDR3 having an amino acid sequence of SEQ ID NO:735; and (ii) a VL comprising a VL CDR1 having an amino acid sequence of SEQ ID NO:814, a VL CDR2 having an amino acid sequence of SEQ ID NO:820, and a VL CDR3 having an amino acid sequence of SEQ ID NO:826. In some embodiments, the third binding domain that binds to CD28 comprises: (i) a VH comprising a VH CDR1 having an amino acid sequence of SEQ ID NO:741, a VH CDR2 having an amino acid sequence of SEQ ID NO:748, and a VH CDR3 having an amino acid sequence of SEQ ID NO:754; and (ii) a VL comprising a VL CDR1 having an amino acid sequence of SEQ ID NO:832, a VL CDR2 having an amino acid sequence of SEQ ID NO:838, and a VL CDR3 having an amino acid sequence of SEQ ID NO:844. In some embodiments, the third binding domain that binds to CD28 comprises: (i) a VH comprising a VH CDR1 having an amino acid sequence of SEQ ID NO:760, a VH CDR2 having an amino acid sequence of SEQ ID NO:766, and a VH CDR3 having an amino acid sequence of SEQ ID NO:772; and (ii) a VL comprising a VL CDR1 having an amino acid sequence of SEQ ID NO:850, a VL CDR2 having an amino acid sequence of SEQ ID NO:856, and a VL CDR3 having an amino acid sequence of SEQ ID NO:862. In some embodiments, the third binding domain that binds to CD28 comprises: (i) a VH comprising a VH CDR1 having an amino acid sequence of SEQ ID NO:778, a VH CDR2 having an amino acid sequence of SEQ ID NO:784, and a VH CDR3 having an amino acid sequence of SEQ ID NO:790; and (ii) a VL comprising a VL CDR1 having an amino acid sequence of SEQ ID NO:868, a VL CDR2 having an amino acid sequence of SEQ ID NO:874, and a VL CDR3 having an amino acid sequence of SEQ ID NO:880. In some embodiments, the third binding domain that binds to CD28 comprises: (i) a VH comprising a VH CDR1, a VH CDR2, and a VH CDR3 having an amino acid sequence of a VH CDR1, VH CDR2, and VH CDR3, respectively, of SEQ ID NO:692; and (ii) a VL comprising a VL CDR1, a VL CDR2, and a VL CDR3 having an amino acid sequence of a VL CDR1, VL CDR2, and VL CDR3, respectively, of SEQ ID NO:698. In some embodiments, the third binding domain that binds to CD28 comprises a VH having an amino acid sequence of SEQ ID NO:692. In some embodiments, the third binding domain that binds to CD28 comprises a VL having an amino acid sequence of SEQ ID NO:698. In some embodiments, the third binding domain that binds to CD28 comprises a VH having an amino acid sequence of SEQ ID NO:692, and a VL having an amino acid sequence of SEQ ID NO:698. In some embodiments, the third binding domain that binds to CD28 comprises a VH having an amino acid sequence having at least 95% identity to an amino acid sequence of SEQ ID NO:692. In some embodiments, the third binding domain that binds to CD28 comprises a VL having an amino acid sequence having at least 95% identity to an amino acid sequence of SEQ ID NO:698. In some embodiments, the third binding domain that binds to CD28 comprises a VH having an amino acid sequence having at least 95% identity to an amino acid sequence of SEQ ID NO:692, and a VL having an amino acid sequence having at least 95% identity to an amino acid sequence of SEQ ID NO:698.

In some embodiments, the third binding domain that binds to CD28 comprises: (i) a VH comprising a VH CDR1 having an amino acid sequence of SEQ ID NO:705, a VH CDR2 having an amino acid sequence of SEQ ID NO:711, and a VH CDR3 having an amino acid sequence of SEQ ID NO:717; and (ii) a VL comprising a VL CDR1 having an amino acid sequence of SEQ ID NO:797, a VL CDR2 having an amino acid sequence of SEQ ID NO:803, and a VL CDR3 having an amino acid sequence of SEQ ID NO:809. In some embodiments, the third binding domain that binds to CD28 comprises: (i) a VH comprising a VH CDR1 having an amino acid sequence of SEQ ID NO:724, a VH CDR2 having an amino acid sequence of SEQ ID NO:730, and a VH CDR3 having an amino acid sequence of SEQ ID NO:736; and (ii) a VL comprising a VL CDR1 having an amino acid sequence of SEQ ID NO:815, a VL CDR2 having an amino acid sequence of SEQ ID NO:821, and a VL CDR3 having an amino acid sequence of SEQ ID NO:827. In some embodiments, the third binding domain that binds to CD28 comprises: (i) a VH comprising a VH CDR1 having an amino acid sequence of SEQ ID NO:742, a VH CDR2 having an amino acid sequence of SEQ ID NO:749, and a VH CDR3 having an amino acid sequence of SEQ ID NO:755; and (ii) a VL comprising a VL CDR1 having an amino acid sequence of SEQ ID NO:833, a VL CDR2 having an amino acid sequence of SEQ ID NO:839, and a VL CDR3 having an amino acid sequence of SEQ ID NO:845. In some embodiments, the third binding domain that binds to CD28 comprises: (i) a VH comprising a VH CDR1 having an amino acid sequence of SEQ ID NO:761, a VH CDR2 having an amino acid sequence of SEQ ID NO:767, and a VH CDR3 having an amino acid sequence of SEQ ID NO:773; and (ii) a VL comprising a VL CDR1 having an amino acid sequence of SEQ ID NO:851, a VL CDR2 having an amino acid sequence of SEQ ID NO:857, and a VL CDR3 having an amino acid sequence of SEQ ID NO:863. In some embodiments, the third binding domain that binds to CD28 comprises: (i) a VH comprising a VH CDR1 having an amino acid sequence of SEQ ID NO:779, a VH CDR2 having an amino acid sequence of SEQ ID NO:785, and a VH CDR3 having an amino acid sequence of SEQ ID NO:791; and (ii) a VL comprising a VL CDR1 having an amino acid sequence of SEQ ID NO:869, a VL CDR2 having an amino acid sequence of SEQ ID NO:875, and a VL CDR3 having an amino acid sequence of SEQ ID NO: 881. In some embodiments, the third binding domain that binds to CD28 comprises: (i) a VH comprising a VH CDR1, a VH CDR2, and a VH CDR3 having an amino acid sequence of a VH CDR1, VH CDR2, and VH CDR3, respectively, of SEQ ID NO:693; and (ii) a VL comprising a VL CDR1, a VL CDR2, and a VL CDR3 having an amino acid sequence of a VL CDR1, VL CDR2, and VL CDR3, respectively, of SEQ ID NO:699. In some embodiments, the third binding domain that binds to CD28 comprises a VH having an amino acid sequence of SEQ ID NO:693. In some embodiments, the third binding domain that binds to CD28 comprises a VL having an amino acid sequence of SEQ ID NO:699. In some embodiments, the third binding domain that binds to CD28 comprises a VH having an amino acid sequence of SEQ ID NO:693, and a VL having an amino acid sequence of SEQ ID NO:699. In some embodiments, the third binding domain that binds to CD28 comprises a VH having an amino acid sequence having at least 95% identity to an amino acid sequence of SEQ ID NO:693. In some embodiments, the third binding domain that binds to CD28 comprises a VL having an amino acid sequence having at least 95% identity to an amino acid sequence of SEQ ID NO:699. In some embodiments, the third binding domain that binds to CD28 comprises a VH having an amino acid sequence having at least 95% identity to an amino acid sequence of SEQ ID NO:693, and a VL having an amino acid sequence having at least 95% identity to an amino acid sequence of SEQ ID NO:699.

In some embodiments, the trispecific antibodies provided herein have a heavy chain having an amino acid sequence of SEQ ID NO:882. In some embodiments, the trispecific antibodies provided herein have a heavy chain having an amino acid sequence of SEQ ID NO:883. In some embodiments, the trispecific antibodies provided herein have a heavy chain having an amino acid sequence of SEQ ID NO:884. In some embodiments, the trispecific antibodies provided herein have a heavy chain having an amino acid sequence of SEQ ID NO:885. In some embodiments, the trispecific antibodies provided herein have a heavy chain having an amino acid sequence of SEQ ID NO:886. In some embodiments, the trispecific antibodies provided herein have a heavy chain having an amino acid sequence of SEQ ID NO:887. In some embodiments, the trispecific antibodies provided herein have a heavy chain having an amino acid sequence of SEQ ID NO:888. In some embodiments, the trispecific antibodies provided herein have a heavy chain having an amino acid sequence of SEQ ID NO:889. In some embodiments, the trispecific antibodies provided herein have a heavy chain having an amino acid sequence of SEQ ID NO:890. In some embodiments, the trispecific antibodies provided herein have a heavy chain having an amino acid sequence of SEQ ID NO:891.

In some embodiments, the trispecific antibodies provided herein have a heavy chain having an amino acid sequence of SEQ ID NO:892. In some embodiments, the trispecific antibodies provided herein have a heavy chain having an amino acid sequence of SEQ ID NO:893. In some embodiments, the trispecific antibodies provided herein have a heavy chain having an amino acid sequence of SEQ ID NO:894. In some embodiments, the trispecific antibodies provided herein have a heavy chain having an amino acid sequence of SEQ ID NO:895. In some embodiments, the trispecific antibodies provided herein have a heavy chain having an amino acid sequence of SEQ ID NO:896. In some embodiments, the trispecific antibodies provided herein have a heavy chain having an amino acid sequence of SEQ ID NO:897. In some embodiments, the trispecific antibodies provided herein have a heavy chain having an amino acid sequence of SEQ ID NO:898. In some embodiments, the trispecific antibodies provided herein have a heavy chain having an amino acid sequence of SEQ ID NO:899. In some embodiments, the trispecific antibodies provided herein have a heavy chain having an amino acid sequence of SEQ ID NO:900. In some embodiments, the trispecific antibodies provided herein have a heavy chain having an amino acid sequence of SEQ ID NO:901.

In some embodiments, the trispecific antibodies provided herein have a light chain having an amino acid sequence of SEQ ID NO:902. In some embodiments, the trispecific antibodies provided herein have a light chain having an amino acid sequence of SEQ ID NO:903. In some embodiments, the trispecific antibodies provided herein have a light chain having an amino acid sequence of SEQ ID NO:904. In some embodiments, the trispecific antibodies provided herein have a light chain having an amino acid sequence of SEQ ID NO:905. In some embodiments, the trispecific antibodies provided herein have a light chain having an amino acid sequence of SEQ ID NO:906. In some embodiments, the trispecific antibodies provided herein have a light chain having an amino acid sequence of SEQ ID NO:907. In some embodiments, the trispecific antibodies provided herein have a light chain having an amino acid sequence of SEQ ID NO:908. In some embodiments, the trispecific antibodies provided herein have a light chain having an amino acid sequence of SEQ ID NO:909. In some embodiments, the trispecific antibodies provided herein have a light chain having an amino acid sequence of SEQ ID NO:910. In some embodiments, the trispecific antibodies provided herein have a light chain having an amino acid sequence of SEQ ID NO:911.

In some embodiments, the trispecific antibodies provided herein have a light chain having an amino acid sequence of SEQ ID NO:912. In some embodiments, the trispecific antibodies provided herein have a light chain having an amino acid sequence of SEQ ID NO:913. In some embodiments, the trispecific antibodies provided herein have a light chain having an amino acid sequence of SEQ ID NO:914. In some embodiments, the trispecific antibodies provided herein have a light chain having an amino acid sequence of SEQ ID NO:915. In some embodiments, the trispecific antibodies provided herein have a light chain having an amino acid sequence of SEQ ID NO:916. In some embodiments, the trispecific antibodies provided herein have a light chain having an amino acid sequence of SEQ ID NO:917. In some embodiments, the trispecific antibodies provided herein have a light chain having an amino acid sequence of SEQ ID NO:918. In some embodiments, the trispecific antibodies provided herein have a light chain having an amino acid sequence of SEQ ID NO:919. In some embodiments, the trispecific antibodies provided herein have a light chain having an amino acid sequence of SEQ ID NO:920. In some embodiments, the trispecific antibodies provided herein have a light chain having an amino acid sequence of SEQ ID NO:921.

In some embodiments, the trispecific antibodies provided herein have a heavy chain having an amino acid sequence of SEQ ID NO:942. In some embodiments, the trispecific antibodies provided herein have a light chain having an amino acid sequence of SEQ ID NO:965. In some embodiments, the trispecific antibodies provided herein have a heavy chain having an amino acid sequence of SEQ ID NO:1049.

In some embodiments, the trispecific antibodies provided herein have a heavy chain having an amino acid sequence of SEQ ID NO:943. In some embodiments, the trispecific antibodies provided herein have a light chain having an amino acid sequence of SEQ ID NO:966.

In some embodiments, the trispecific antibodies provided herein have a heavy chain having an amino acid sequence of SEQ ID NO:944. In some embodiments, the trispecific antibodies provided herein have a light chain having an amino acid sequence of SEQ ID NO:967. In some embodiments, the trispecific antibodies provided herein have a heavy chain having an amino acid sequence of SEQ ID NO:980. In some embodiments, the trispecific antibodies provided herein have a light chain having an amino acid sequence of SEQ ID NO:1001.

In some embodiments, the trispecific antibodies provided herein have a heavy chain having an amino acid sequence of SEQ ID NO:945. In some embodiments, the trispecific antibodies provided herein have a light chain having an amino acid sequence of SEQ ID NO:968. In some embodiments, the trispecific antibodies provided herein have a heavy chain having an amino acid sequence of SEQ ID NO:981. In some embodiments, the trispecific antibodies provided herein have a light chain having an amino acid sequence of SEQ ID NO:1002.

In some embodiments, the trispecific antibodies provided herein have a heavy chain having an amino acid sequence of SEQ ID NO:946. In some embodiments, the trispecific antibodies provided herein have a light chain having an amino acid sequence of SEQ ID NO:969. In some embodiments, the trispecific antibodies provided herein have a heavy chain having an amino acid sequence of SEQ ID NO:982. In some embodiments, the trispecific antibodies provided herein have a light chain having an amino acid sequence of SEQ ID NO:1003.

In some embodiments, the trispecific antibodies provided herein have a heavy chain having an amino acid sequence of SEQ ID NO:947. In some embodiments, the trispecific antibodies provided herein have a light chain having an amino acid sequence of SEQ ID NO:970. In some embodiments, the trispecific antibodies provided herein have a heavy chain having an amino acid sequence of SEQ ID NO:983. In some embodiments, the trispecific antibodies provided herein have a light chain having an amino acid sequence of SEQ ID NO:1004.

In some embodiments, the trispecific antibodies provided herein have a heavy chain having an amino acid sequence of SEQ ID NO:948. In some embodiments, the trispecific antibodies provided herein have a light chain having an amino acid sequence of SEQ ID NO:971. In some embodiments, the trispecific antibodies provided herein have a heavy chain having an amino acid sequence of SEQ ID NO:984. In some embodiments, the trispecific antibodies provided herein have a light chain having an amino acid sequence of SEQ ID NO:1005.

In some embodiments, the trispecific antibodies provided herein have a heavy chain having an amino acid sequence of SEQ ID NO:949. In some embodiments, the trispecific antibodies provided herein have a light chain having an amino acid sequence of SEQ ID NO:972. In some embodiments, the trispecific antibodies provided herein have a heavy chain having an amino acid sequence of SEQ ID NO:985. In some embodiments, the trispecific antibodies provided herein have a light chain having an amino acid sequence of SEQ ID NO:1006.

In some embodiments, the trispecific antibodies provided herein have a heavy chain having an amino acid sequence of SEQ ID NO:950. In some embodiments, the trispecific antibodies provided herein have a light chain having an amino acid sequence of SEQ ID NO:973. In some embodiments, the trispecific antibodies provided herein have a heavy chain having an amino acid sequence of SEQ ID NO:986.

In some embodiments, the trispecific antibodies provided herein have a heavy chain having an amino acid sequence of SEQ ID NO:951. In some embodiments, the trispecific antibodies provided herein have a light chain having an amino acid sequence of SEQ ID NO:974. In some embodiments, the trispecific antibodies provided herein have a heavy chain having an amino acid sequence of SEQ ID NO:987.

In some embodiments, the trispecific antibodies provided herein have a heavy chain having an amino acid sequence of SEQ ID NO:952. In some embodiments, the trispecific antibodies provided herein have a light chain having an amino acid sequence of SEQ ID NO:975. In some embodiments, the trispecific antibodies provided herein have a heavy chain having an amino acid sequence of SEQ ID NO:988.

In some embodiments, the trispecific antibodies provided herein have a heavy chain having an amino acid sequence of SEQ ID NO:953. In some embodiments, the trispecific antibodies provided herein have a light chain having an amino acid sequence of SEQ ID NO:976. In some embodiments, the trispecific antibodies provided herein have a heavy chain having an amino acid sequence of SEQ ID NO:989.

In some embodiments, the trispecific antibodies provided herein have a heavy chain having an amino acid sequence of SEQ ID NO:954. In some embodiments, the trispecific antibodies provided herein have a light chain having an amino acid sequence of SEQ ID NO:977. In some embodiments, the trispecific antibodies provided herein have a heavy chain having an amino acid sequence of SEQ ID NO:990.

In some embodiments, the trispecific antibodies provided herein have a heavy chain having an amino acid sequence of SEQ ID NO:955. In some embodiments, the trispecific antibodies provided herein have a light chain having an amino acid sequence of SEQ ID NO:978. In some embodiments, the trispecific antibodies provided herein have a heavy chain having an amino acid sequence of SEQ ID NO:991.

In some embodiments, the trispecific antibodies provided herein have a heavy chain having an amino acid sequence of SEQ ID NO:1048. In some embodiments, the trispecific antibodies provided herein have a heavy chain having an amino acid sequence of SEQ ID NO:1050. In some embodiments, the trispecific antibodies provided herein have a light chain having an amino acid sequence of SEQ ID NO:1051.

In some embodiments, the trispecific antibodies provided herein have a heavy chain having an amino acid sequence of SEQ ID NO:956. In some embodiments, the trispecific antibodies provided herein have a heavy chain having an amino acid sequence of SEQ ID NO:992. In some embodiments, the trispecific antibodies provided herein have a light chain having an amino acid sequence of SEQ ID NO:1007.

In some embodiments, the trispecific antibodies provided herein have a heavy chain having an amino acid sequence of SEQ ID NO:957. In some embodiments, the trispecific antibodies provided herein have a heavy chain having an amino acid sequence of SEQ ID NO:993. In some embodiments, the trispecific antibodies provided herein have a light chain having an amino acid sequence of SEQ ID NO:1008.

In some embodiments, the trispecific antibodies provided herein have a heavy chain having an amino acid sequence of SEQ ID NO:958. In some embodiments, the trispecific antibodies provided herein have a heavy chain having an amino acid sequence of SEQ ID NO:994. In some embodiments, the trispecific antibodies provided herein have a light chain having an amino acid sequence of SEQ ID NO:1009.

In some embodiments, the trispecific antibodies provided herein have a heavy chain having an amino acid sequence of SEQ ID NO:959. In some embodiments, the trispecific antibodies provided herein have a heavy chain having an amino acid sequence of SEQ ID NO:995. In some embodiments, the trispecific antibodies provided herein have a light chain having an amino acid sequence of SEQ ID NO:1010.

In some embodiments, the trispecific antibodies provided herein have a heavy chain having an amino acid sequence of SEQ ID NO:960. In some embodiments, the trispecific antibodies provided herein have a heavy chain having an amino acid sequence of SEQ ID NO:996. In some embodiments, the trispecific antibodies provided herein have a light chain having an amino acid sequence of SEQ ID NO:1011.

In some embodiments, the trispecific antibodies provided herein have a heavy chain having an amino acid sequence of SEQ ID NO:961. In some embodiments, the trispecific antibodies provided herein have a heavy chain having an amino acid sequence of SEQ ID NO:997. In some embodiments, the trispecific antibodies provided herein have a light chain having an amino acid sequence of SEQ ID NO:1012.

In some embodiments, the trispecific antibodies provided herein have a heavy chain having an amino acid sequence of SEQ ID NO:962. In some embodiments, the trispecific antibodies provided herein have a heavy chain having an amino acid sequence of SEQ ID NO:998. In some embodiments, the trispecific antibodies provided herein have a light chain having an amino acid sequence of SEQ ID NO:1013.

In some embodiments, the trispecific antibodies provided herein have a heavy chain having an amino acid sequence of SEQ ID NO:963. In some embodiments, the trispecific antibodies provided herein have a heavy chain having an amino acid sequence of SEQ ID NO:999. In some embodiments, the trispecific antibodies provided herein have a light chain having an amino acid sequence of SEQ ID NO:1014.

In some embodiments, the trispecific antibodies provided herein have a heavy chain having an amino acid sequence of SEQ ID NO:964. In some embodiments, the trispecific antibodies provided herein have a heavy chain having an amino acid sequence of SEQ ID NO:1000. In some embodiments, the trispecific antibodies provided herein have a light chain having an amino acid sequence of SEQ ID NO:1015.

In one aspect, provided herein is a trispecific antibody comprising: (a) a first binding domain that binds to Vβ17, (b) a second binding domain that binds to a second target, and (c) a third binding domain that binds to CD28.

In some embodiments, the first binding domain that binds to Vβ17 comprises a VH CDR1, VH CDR2, and VH CDR3 of a Vβ17 antibody provided herein. In some embodiments, the first binding domain that binds to Vβ17 comprises a VL CDR1, VL CDR2, and VL CDR3 of a Vβ17 antibody provided herein. In some embodiments, the first binding domain that binds to Vβ17 comprises a VH CDR1, VH CDR2, VH CDR3, VL CDR1, VL CDR2, and VL CDR3 of a Vβ17 antibody provided herein.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:25. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:26. In some embodiments, the first binding domain that binds to Vβ17 comprises a VH having an amino acid sequence of SEQ ID NO:25. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:25; and (ii) a VL comprising a VL CDR1, a VL CDR2, and a VL CDR3 having an amino acid sequence of a VL CDR1, VL CDR2, and VL CDR3, respectively, of SEQ ID NO:26. In some embodiments, the first binding domain that binds to Vβ17 comprises a VH having an amino acid sequence of SEQ ID NO:25. In some embodiments, the first binding domain that binds to Vβ17 comprises a VL having an amino acid sequence of SEQ ID NO:26. In some embodiments, the first binding domain that binds to Vβ17 comprises a VH having an amino acid sequence of SEQ ID NO:25, and a VL having an amino acid sequence of SEQ ID NO:26. In some embodiments, the first binding domain that binds to Vβ17 comprises a VH having an amino acid sequence having at least 95% identity to an amino acid sequence of SEQ ID NO:25. In some embodiments, the first binding domain that binds to Vβ17 comprises a VL having an amino acid sequence having at least 95% identity to an amino acid sequence of SEQ ID NO:26. In some embodiments, the first binding domain that binds to Vβ17 comprises a VH having an amino acid sequence having at least 95% identity to an amino acid sequence of SEQ ID NO:25, and a VL having an amino acid sequence having at least 95% identity to an amino acid sequence of SEQ ID NO:26.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:19. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:22. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:22. In some embodiments, the first binding domain that binds to Vβ17 comprises a VH having an amino acid sequence of SEQ ID NO:19. In some embodiments, the first binding domain that binds to Vβ17 comprises a VL having an amino acid sequence of SEQ ID NO:22. In some embodiments, the first binding domain that binds to Vβ17 comprises a VH having an amino acid sequence of SEQ ID NO:19, and a VL having an amino acid sequence of SEQ ID NO:22. In some embodiments, the first binding domain that binds to Vβ17 comprises a VH having an amino acid sequence having at least 95% identity to an amino acid sequence of SEQ ID NO:19. In some embodiments, the first binding domain that binds to Vβ17 comprises a VL having an amino acid sequence having at least 95% identity to an amino acid sequence of SEQ ID NO:22. In some embodiments, the first binding domain that binds to Vβ17 comprises a VH having an amino acid sequence having at least 95% identity to an amino acid sequence of SEQ ID NO:19, and a VL having an amino acid sequence having at least 95% identity to an amino acid sequence of SEQ ID NO:22.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:20. n some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:23. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:23. In some embodiments, the first binding domain that binds to Vβ17 comprises a VH having an amino acid sequence of SEQ ID NO:20. In some embodiments, the first binding domain that binds to Vβ17 comprises a VL having an amino acid sequence of SEQ ID NO:23. In some embodiments, the first binding domain that binds to Vβ17 comprises a VH having an amino acid sequence of SEQ ID NO:20, and a VL having an amino acid sequence of SEQ ID NO:23. In some embodiments, the first binding domain that binds to Vβ17 comprises a VH having an amino acid sequence having at least 95% identity to an amino acid sequence of SEQ ID NO:20. In some embodiments, the first binding domain that binds to Vβ17 comprises a VL having an amino acid sequence having at least 95% identity to an amino acid sequence of SEQ ID NO:23. In some embodiments, the first binding domain that binds to Vβ17 comprises a VH having an amino acid sequence having at least 95% identity to an amino acid sequence of SEQ ID NO:20, and a VL having an amino acid sequence having at least 95% identity to an amino acid sequence of SEQ ID NO:23.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:21. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:24. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:24. In some embodiments, the first binding domain that binds to Vβ17 comprises a VH having an amino acid sequence of SEQ ID NO:21. In some embodiments, the first binding domain that binds to Vβ17 comprises a VL having an amino acid sequence of SEQ ID NO:24. In some embodiments, the first binding domain that binds to Vβ17 comprises a VH having an amino acid sequence of SEQ ID NO:21, and a VL having an amino acid sequence of SEQ ID NO:24. In some embodiments, the first binding domain that binds to Vβ17 comprises a VH having an amino acid sequence having at least 95% identity to an amino acid sequence of SEQ ID NO:21. In some embodiments, the first binding domain that binds to Vβ17 comprises a VL having an amino acid sequence having at least 95% identity to an amino acid sequence of SEQ ID NO:24. In some embodiments, the first binding domain that binds to Vβ17 comprises a VH having an amino acid sequence having at least 95% identity to an amino acid sequence of SEQ ID NO:21, and a VL having an amino acid sequence having at least 95% identity to an amino acid sequence of SEQ ID NO:24.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:19. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:23. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:23. In some embodiments, the first binding domain that binds to Vβ17 comprises a VH having an amino acid sequence of SEQ ID NO:19. In some embodiments, the first binding domain that binds to Vβ17 comprises a VL having an amino acid sequence of SEQ ID NO:23. In some embodiments, the first binding domain that binds to Vβ17 comprises a VH having an amino acid sequence of SEQ ID NO:19, and a VL having an amino acid sequence of SEQ ID NO:23. In some embodiments, the first binding domain that binds to Vβ17 comprises a VH having an amino acid sequence having at least 95% identity to an amino acid sequence of SEQ ID NO:19. In some embodiments, the first binding domain that binds to Vβ17 comprises a VL having an amino acid sequence having at least 95% identity to an amino acid sequence of SEQ ID NO:23. In some embodiments, the first binding domain that binds to Vβ17 comprises a VH having an amino acid sequence having at least 95% identity to an amino acid sequence of SEQ ID NO:19, and a VL having an amino acid sequence having at least 95% identity to an amino acid sequence of SEQ ID NO:23.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:19. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:24. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:24. In some embodiments, the first binding domain that binds to Vβ17 comprises a VH having an amino acid sequence of SEQ ID NO:19. In some embodiments, the first binding domain that binds to Vβ17 comprises a VL having an amino acid sequence of SEQ ID NO:24. In some embodiments, the first binding domain that binds to Vβ17 comprises a VH having an amino acid sequence of SEQ ID NO:19, and a VL having an amino acid sequence of SEQ ID NO:24. In some embodiments, the first binding domain that binds to Vβ17 comprises a VH having an amino acid sequence having at least 95% identity to an amino acid sequence of SEQ ID NO:19. In some embodiments, the first binding domain that binds to Vβ17 comprises a VL having an amino acid sequence having at least 95% identity to an amino acid sequence of SEQ ID NO:24. In some embodiments, the first binding domain that binds to Vβ17 comprises a VH having an amino acid sequence having at least 95% identity to an amino acid sequence of SEQ ID NO:19, and a VL having an amino acid sequence having at least 95% identity to an amino acid sequence of SEQ ID NO:24.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:20. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:22. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:22. In some embodiments, the first binding domain that binds to Vβ17 comprises a VH having an amino acid sequence of SEQ ID NO:20. In some embodiments, the first binding domain that binds to Vβ17 comprises a VL having an amino acid sequence of SEQ ID NO:22. In some embodiments, the first binding domain that binds to Vβ17 comprises a VH having an amino acid sequence of SEQ ID NO:20, and a VL having an amino acid sequence of SEQ ID NO:22. In some embodiments, the first binding domain that binds to Vβ17 comprises a VH having an amino acid sequence having at least 95% identity to an amino acid sequence of SEQ ID NO:20. In some embodiments, the first binding domain that binds to Vβ17 comprises a VL having an amino acid sequence having at least 95% identity to an amino acid sequence of SEQ ID NO:22. In some embodiments, the first binding domain that binds to Vβ17 comprises a VH having an amino acid sequence having at least 95% identity to an amino acid sequence of SEQ ID NO:20, and a VL having an amino acid sequence having at least 95% identity to an amino acid sequence of SEQ ID NO:22.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:20. n some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:24. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:24. In some embodiments, the first binding domain that binds to Vβ17 comprises a VH having an amino acid sequence of SEQ ID NO:20. In some embodiments, the first binding domain that binds to Vβ17 comprises a VL having an amino acid sequence of SEQ ID NO:24. In some embodiments, the first binding domain that binds to Vβ17 comprises a VH having an amino acid sequence of SEQ ID NO:20, and a VL having an amino acid sequence of SEQ ID NO:24. In some embodiments, the first binding domain that binds to Vβ17 comprises a VH having an amino acid sequence having at least 95% identity to an amino acid sequence of SEQ ID NO:20. In some embodiments, the first binding domain that binds to Vβ17 comprises a VL having an amino acid sequence having at least 95% identity to an amino acid sequence of SEQ ID NO:24. In some embodiments, the first binding domain that binds to Vβ17 comprises a VH having an amino acid sequence having at least 95% identity to an amino acid sequence of SEQ ID NO:20, and a VL having an amino acid sequence having at least 95% identity to an amino acid sequence of SEQ ID NO:24.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:21. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:22. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:22. In some embodiments, the first binding domain that binds to Vβ17 comprises a VH having an amino acid sequence of SEQ ID NO:21. In some embodiments, the first binding domain that binds to Vβ17 comprises a VL having an amino acid sequence of SEQ ID NO:22. In some embodiments, the first binding domain that binds to Vβ17 comprises a VH having an amino acid sequence of SEQ ID NO:21, and a VL having an amino acid sequence of SEQ ID NO:22. In some embodiments, the first binding domain that binds to Vβ17 comprises a VH having an amino acid sequence having at least 95% identity to an amino acid sequence of SEQ ID NO:21. In some embodiments, the first binding domain that binds to Vβ17 comprises a VL having an amino acid sequence having at least 95% identity to an amino acid sequence of SEQ ID NO:22. In some embodiments, the first binding domain that binds to Vβ17 comprises a VH having an amino acid sequence having at least 95% identity to an amino acid sequence of SEQ ID NO:21, and a VL having an amino acid sequence having at least 95% identity to an amino acid sequence of SEQ ID NO:22.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:21. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:23. In some embodiments, the first binding domain that binds to Vβ17 comprises a VH having an amino acid sequence of SEQ ID NO:21. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:23. In some embodiments, the first binding domain that binds to Vβ17 comprises a VH having an amino acid sequence of SEQ ID NO:21. In some embodiments, the first binding domain that binds to Vβ17 comprises a VL having an amino acid sequence of SEQ ID NO:23. In some embodiments, the first binding domain that binds to Vβ17 comprises a VH having an amino acid sequence of SEQ ID NO:21, and a VL having an amino acid sequence of SEQ ID NO:23. In some embodiments, the first binding domain that binds to Vβ17 comprises a VH having an amino acid sequence having at least 95% identity to an amino acid sequence of SEQ ID NO:21. In some embodiments, the first binding domain that binds to Vβ17 comprises a VL having an amino acid sequence having at least 95% identity to an amino acid sequence of SEQ ID NO:23. In some embodiments, the first binding domain that binds to Vβ17 comprises a VH having an amino acid sequence having at least 95% identity to an amino acid sequence of SEQ ID NO:21, and a VL having an amino acid sequence having at least 95% identity to an amino acid sequence of SEQ ID NO:23.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:1084. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:1085. In some embodiments, the first binding domain that binds to Vβ17 comprises a VH having an amino acid sequence of SEQ ID NO:1084. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:1084; and (ii) a VL comprising a VL CDR1, a VL CDR2, and a VL CDR3 having an amino acid sequence of a VL CDR1, VL CDR2, and VL CDR3, respectively, of SEQ ID NO:1085. In some embodiments, the first binding domain that binds to Vβ17 comprises a VH having an amino acid sequence of SEQ ID NO:1084. In some embodiments, the first binding domain that binds to Vβ17 comprises a VL having an amino acid sequence of SEQ ID NO:1085. In some embodiments, the first binding domain that binds to Vβ17 comprises a VH having an amino acid sequence of SEQ ID NO:1084, and a VL having an amino acid sequence of SEQ ID NO:1085. In some embodiments, the first binding domain that binds to Vβ17 comprises a VH having an amino acid sequence having at least 95% identity to an amino acid sequence of SEQ ID NO:1084. In some embodiments, the first binding domain that binds to Vβ17 comprises a VL having an amino acid sequence having at least 95% identity to an amino acid sequence of SEQ ID NO:1085. In some embodiments, the first binding domain that binds to Vβ17 comprises a VH having an amino acid sequence having at least 95% identity to an amino acid sequence of SEQ ID NO:1084, and a VL having an amino acid sequence having at least 95% identity to an amino acid sequence of SEQ ID NO:1085.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:46. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:47. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:46; and (ii) a VL comprising a VL CDR1, a VL CDR2, and a VL CDR3 having an amino acid sequence of a VL CDR1, VL CDR2, and VL CDR3, respectively, of SEQ ID NO:47. In some embodiments, the first binding domain that binds to Vβ17 comprises a VH having an amino acid sequence of SEQ ID NO:46. In some embodiments, the first binding domain that binds to Vβ17 comprises a VL having an amino acid sequence of SEQ ID NO:47. In some embodiments, the first binding domain that binds to Vβ17 comprises a VH having an amino acid sequence of SEQ ID NO:46, and a VL having an amino acid sequence of SEQ ID NO:47. In some embodiments, the first binding domain that binds to Vβ17 comprises a VH having an amino acid sequence having at least 95% identity to an amino acid sequence of SEQ ID NO:46. In some embodiments, the first binding domain that binds to Vβ17 comprises a VL having an amino acid sequence having at least 95% identity to an amino acid sequence of SEQ ID NO:47. In some embodiments, the first binding domain that binds to Vβ17 comprises a VH having an amino acid sequence having at least 95% identity to an amino acid sequence of SEQ ID NO:46, and a VL having an amino acid sequence having at least 95% identity to an amino acid sequence of SEQ ID NO:47.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:77. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:78. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:77; and (ii) a VL comprising a VL CDR1, a VL CDR2, and a VL CDR3 having an amino acid sequence of a VL CDR1, VL CDR2, and VL CDR3, respectively, of SEQ ID NO:78. In some embodiments, the first binding domain that binds to Vβ17 comprises a VH having an amino acid sequence of SEQ ID NO:77. In some embodiments, the first binding domain that binds to Vβ17 comprises a VL having an amino acid sequence of SEQ ID NO:78. In some embodiments, the first binding domain that binds to Vβ17 comprises a VH having an amino acid sequence of SEQ ID NO:77, and a VL having an amino acid sequence of SEQ ID NO:78. In some embodiments, the first binding domain that binds to Vβ17 comprises a VH having an amino acid sequence having at least 95% identity to an amino acid sequence of SEQ ID NO:77. In some embodiments, the first binding domain that binds to Vβ17 comprises a VL having an amino acid sequence having at least 95% identity to an amino acid sequence of SEQ ID NO:78. In some embodiments, the first binding domain that binds to Vβ17 comprises a VH having an amino acid sequence having at least 95% identity to an amino acid sequence of SEQ ID NO:77, and a VL having an amino acid sequence having at least 95% identity to an amino acid sequence of SEQ ID NO:78.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:79. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:80. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:79; and (ii) a VL comprising a VL CDR1, a VL CDR2, and a VL CDR3 having an amino acid sequence of a VL CDR1, VL CDR2, and VL CDR3, respectively, of SEQ ID NO:80. In some embodiments, the first binding domain that binds to Vβ17 comprises a VH having an amino acid sequence of SEQ ID NO:79. In some embodiments, the first binding domain that binds to Vβ17 comprises a VL having an amino acid sequence of SEQ ID NO:80. In some embodiments, the first binding domain that binds to Vβ17 comprises a VH having an amino acid sequence of SEQ ID NO:79, and a VL having an amino acid sequence of SEQ ID NO:80. In some embodiments, the first binding domain that binds to Vβ17 comprises a VH having an amino acid sequence having at least 95% identity to an amino acid sequence of SEQ ID NO:79. In some embodiments, the first binding domain that binds to Vβ17 comprises a VL having an amino acid sequence having at least 95% identity to an amino acid sequence of SEQ ID NO:80. In some embodiments, the first binding domain that binds to Vβ17 comprises a VH having an amino acid sequence having at least 95% identity to an amino acid sequence of SEQ ID NO:79, and a VL having an amino acid sequence having at least 95% identity to an amino acid sequence of SEQ ID NO:80.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:81. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:82. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:81; and (ii) a VL comprising a VL CDR1, a VL CDR2, and a VL CDR3 having an amino acid sequence of a VL CDR1, VL CDR2, and VL CDR3, respectively, of SEQ ID NO:82. In some embodiments, the first binding domain that binds to Vβ17 comprises a VH having an amino acid sequence of SEQ ID NO:81. In some embodiments, the first binding domain that binds to Vβ17 comprises a VL having an amino acid sequence of SEQ ID NO:82. In some embodiments, the first binding domain that binds to Vβ17 comprises a VH having an amino acid sequence of SEQ ID NO:81, and a VL having an amino acid sequence of SEQ ID NO:82. In some embodiments, the first binding domain that binds to Vβ17 comprises a VH having an amino acid sequence having at least 95% identity to an amino acid sequence of SEQ ID NO:81. In some embodiments, the first binding domain that binds to Vβ17 comprises a VL having an amino acid sequence having at least 95% identity to an amino acid sequence of SEQ ID NO:82. In some embodiments, the first binding domain that binds to Vβ17 comprises a VH having an amino acid sequence having at least 95% identity to an amino acid sequence of SEQ ID NO:81, and a VL having an amino acid sequence having at least 95% identity to an amino acid sequence of SEQ ID NO:82.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:83. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:84. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:83; and (ii) a VL comprising a VL CDR1, a VL CDR2, and a VL CDR3 having an amino acid sequence of a VL CDR1, VL CDR2, and VL CDR3, respectively, of SEQ ID NO:84. In some embodiments, the first binding domain that binds to Vβ17 comprises a VH having an amino acid sequence of SEQ ID NO:83. In some embodiments, the first binding domain that binds to Vβ17 comprises a VL having an amino acid sequence of SEQ ID NO:84. In some embodiments, the first binding domain that binds to Vβ17 comprises a VH having an amino acid sequence of SEQ ID NO:83, and a VL having an amino acid sequence of SEQ ID NO:84. In some embodiments, the first binding domain that binds to Vβ17 comprises a VH having an amino acid sequence having at least 95% identity to an amino acid sequence of SEQ ID NO:83. In some embodiments, the first binding domain that binds to Vβ17 comprises a VL having an amino acid sequence having at least 95% identity to an amino acid sequence of SEQ ID NO:84. In some embodiments, the first binding domain that binds to Vβ17 comprises a VH having an amino acid sequence having at least 95% identity to an amino acid sequence of SEQ ID NO:83, and a VL having an amino acid sequence having at least 95% identity to an amino acid sequence of SEQ ID NO:84.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:85. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:86. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:85; and (ii) a VL comprising a VL CDR1, a VL CDR2, and a VL CDR3 having an amino acid sequence of a VL CDR1, VL CDR2, and VL CDR3, respectively, of SEQ ID NO:86. In some embodiments, the first binding domain that binds to Vβ17 comprises a VH having an amino acid sequence of SEQ ID NO:85. In some embodiments, the first binding domain that binds to Vβ17 comprises a VL having an amino acid sequence of SEQ ID NO:86. In some embodiments, the first binding domain that binds to Vβ17 comprises a VH having an amino acid sequence of SEQ ID NO:85, and a VL having an amino acid sequence of SEQ ID NO:86. In some embodiments, the first binding domain that binds to Vβ17 comprises a VH having an amino acid sequence having at least 95% identity to an amino acid sequence of SEQ ID NO:85. In some embodiments, the first binding domain that binds to Vβ17 comprises a VL having an amino acid sequence having at least 95% identity to an amino acid sequence of SEQ ID NO:86. In some embodiments, the first binding domain that binds to Vβ17 comprises a VH having an amino acid sequence having at least 95% identity to an amino acid sequence of SEQ ID NO:85, and a VL having an amino acid sequence having at least 95% identity to an amino acid sequence of SEQ ID NO:86.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:87. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:88. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:87; and (ii) a VL comprising a VL CDR1, a VL CDR2, and a VL CDR3 having an amino acid sequence of a VL CDR1, VL CDR2, and VL CDR3, respectively, of SEQ ID NO:88. In some embodiments, the first binding domain that binds to Vβ17 comprises a VH having an amino acid sequence of SEQ ID NO:87. In some embodiments, the first binding domain that binds to Vβ17 comprises a VL having an amino acid sequence of SEQ ID NO:88. In some embodiments, the first binding domain that binds to Vβ17 comprises a VH having an amino acid sequence of SEQ ID NO:87, and a VL having an amino acid sequence of SEQ ID NO:88. In some embodiments, the first binding domain that binds to Vβ17 comprises a VH having an amino acid sequence having at least 95% identity to an amino acid sequence of SEQ ID NO:87. In some embodiments, the first binding domain that binds to Vβ17 comprises a VL having an amino acid sequence having at least 95% identity to an amino acid sequence of SEQ ID NO:88. In some embodiments, the first binding domain that binds to Vβ17 comprises a VH having an amino acid sequence having at least 95% identity to an amino acid sequence of SEQ ID NO:87, and a VL having an amino acid sequence having at least 95% identity to an amino acid sequence of SEQ ID NO:88.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:21. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:665. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:665. In some embodiments, the first binding domain that binds to Vβ17 comprises a VH having an amino acid sequence of SEQ ID NO:21. In some embodiments, the first binding domain that binds to Vβ17 comprises a VL having an amino acid sequence of SEQ ID NO:665. In some embodiments, the first binding domain that binds to Vβ17 comprises a VH having an amino acid sequence of SEQ ID NO:21, and a VL having an amino acid sequence of SEQ ID NO:665. In some embodiments, the first binding domain that binds to Vβ17 comprises a VH having an amino acid sequence having at least 95% identity to an amino acid sequence of SEQ ID NO:21. In some embodiments, the first binding domain that binds to Vβ17 comprises a VL having an amino acid sequence having at least 95% identity to an amino acid sequence of SEQ ID NO:665. In some embodiments, the first binding domain that binds to Vβ17 comprises a VH having an amino acid sequence having at least 95% identity to an amino acid sequence of SEQ ID NO:21, and a VL having an amino acid sequence having at least 95% identity to an amino acid sequence of SEQ ID NO:665.

In some embodiments, the first binding domain that binds to Vβ17 comprises: a VH comprising a VH CDR1, a VH CDR2, and a VH CDR3 having an amino acid sequence of a VH CDR1, VH CDR2, and VH CDR3, respectively, of SEQ ID NO:1084. In some embodiments, the first binding domain that binds to Vβ17 comprises: a VL comprising a VL CDR1, a VL CDR2, and a VL CDR3 having an amino acid sequence of a VL CDR1, VL CDR2, and VL CDR3, respectively, of SEQ ID NO:1085. In some embodiments, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:1084; and (ii) a VL comprising a VL CDR1, a VL CDR2, and a VL CDR3 having an amino acid sequence of a VL CDR1, VL CDR2, and VL CDR3, respectively, of SEQ ID NO:1085. In some embodiments, the first binding domain that binds to Vβ17 comprises a VH having an amino acid sequence of SEQ ID NO:1084. In some embodiments, the first binding domain that binds to Vβ17 comprises a VL having an amino acid sequence of SEQ ID NO:1085. In some embodiments, the first binding domain that binds to Vβ17 comprises a VH having an amino acid sequence of SEQ ID NO:1084, and a VL having an amino acid sequence of SEQ ID NO:1085. In some embodiments, the first binding domain that binds to Vβ17 comprises a VH having an amino acid sequence having at least 95% identity to an amino acid sequence of SEQ ID NO:1084. In some embodiments, the first binding domain that binds to Vβ17 comprises a VL having an amino acid sequence having at least 95% identity to an amino acid sequence of SEQ ID NO:1085. In some embodiments, the first binding domain that binds to Vβ17 comprises a VH having an amino acid sequence having at least 95% identity to an amino acid sequence of SEQ ID NO:1084, and a VL having an amino acid sequence having at least 95% identity to an amino acid sequence of SEQ ID NO:1085.

In some embodiments, the second target is a cancer antigen. Exemplary cancer antigens are provided infra.

In some embodiments, the second target is a tumor-specific antigen.

In some embodiments, the second target is a tumor associated antigen (TAA).

In some embodiments, the second target is a neoantigen.

In some embodiments, the second target is BCMA. Thus, in some embodiments, provided is a trispecific antibody comprising: (a) a first binding domain that binds to Vβ17, (b) a second binding domain that binds to BCMA, and (c) a third binding domain that binds to CD28. In some embodiments, the first binding domain that binds to Vβ17 comprises a VH CDR1, VH CDR2, and VH CDR3 of a Vβ17 antibody provided herein. In some embodiments, the first binding domain that binds to Vβ17 comprises a VL CDR1, VL CDR2, and VL CDR3 amino acid sequence of a Vβ17 antibody provided herein. In some embodiments, the first binding domain that binds to Vβ17 comprises a VH CDR1, VH CDR2, VH CDR3, VL CDR1, VL CDR2, and VL CDR3 amino acid sequence of a Vβ17 antibody provided herein. In some embodiments, the second binding domain that binds to BCMA comprises a VH CDR1, VH CDR2, and VH CDR3 amino acid sequence of a BCMA antibody provided herein. In some embodiments, the second binding domain that binds to BCMA comprises a VL CDR1, VL CDR2, and VL CDR3 amino acid sequence of a BCMA antibody provided herein. In some embodiments, the second binding domain that binds to BCMA comprises a VH CDR1, VH CDR2, VH CDR3, VL CDR1, VL CDR2, and VL CDR3 amino acid sequence of a BCMA antibody provided herein. In some embodiments, the third binding domain that binds to CD28 comprises a VH CDR1, VH CDR2, and VH CDR3 amino acid sequence of a CD28 antibody provided herein. In some embodiments, the third binding domain that binds to CD28 comprises a VL CDR1, VL CDR2, and VL CDR3 amino acid sequence of a CD28 antibody provided herein. In some embodiments, the third binding domain that binds to CD28 comprises a VH CDR1, VH CDR2, VH CDR3, VL CDR1, VL CDR2, and VL CDR3 amino acid sequence of a CD28 antibody provided herein.

In some embodiments, the second binding domain that binds to BCMA comprises: a VH comprising a VH CDR1, a VH CDR2, and a VH CDR3 having an amino acid sequence of a VH CDR1, VH CDR2, and VH CDR3, respectively, of SEQ ID NO:95. In some embodiments, the second binding domain that binds to BCMA comprises: a VL comprising a VL CDR1, a VL CDR2, and a VL CDR3 having an amino acid sequence of a VL CDR1, VL CDR2, and VL CDR3, respectively, of SEQ ID NO:96. In some embodiments, the second binding domain that binds to BCMA comprises: (i) a VH comprising a VH CDR1, a VH CDR2, and a VH CDR3 having an amino acid sequence of a VH CDR1, VH CDR2, and VH CDR3, respectively, of SEQ ID NO:95; and (ii) a VL comprising a VL CDR1, a VL CDR2, and a VL CDR3 having an amino acid sequence of a VL CDR1, VL CDR2, and VL CDR3, respectively, of SEQ ID NO:96. In some embodiments, the second binding domain that binds to BCMA comprises: (i) a VH comprising a VH CDR1 having an amino acid sequence of SEQ ID NO:89, a VH CDR2 having an amino acid sequence of SEQ ID NO:90, and a VH CDR3 having an amino acid sequence of SEQ ID NO:91; and (ii) a VL comprising a VL CDR1 having an amino acid sequence of SEQ ID NO:93, a VL CDR2 having an amino acid sequence of SEQ ID NO:94, and a VL CDR3 having an amino acid sequence of SEQ ID NO:94. In some embodiments, the second binding domain that binds to BCMA comprises: (i) a VH comprising a VH CDR1 having an amino acid sequence of SEQ ID NO:622, a VH CDR2 having an amino acid sequence of SEQ ID NO:623, and a VH CDR3 having an amino acid sequence of SEQ ID NO:624; and (ii) a VL comprising a VL CDR1 having an amino acid sequence of SEQ ID NO:625, a VL CDR2 having an amino acid sequence of SEQ ID NO:626, and a VL CDR3 having an amino acid sequence of SEQ ID NO:627. In some embodiments, the second binding domain that binds to BCMA comprises: (i) a VH comprising a VH CDR1 having an amino acid sequence of SEQ ID NO:628, a VH CDR2 having an amino acid sequence of SEQ ID NO:629, and a VH CDR3 having an amino acid sequence of SEQ ID NO:630; and (ii) a VL comprising a VL CDR1 having an amino acid sequence of SEQ ID NO:631, a VL CDR2 having an amino acid sequence of SEQ ID NO:632, and a VL CDR3 having an amino acid sequence of SEQ ID NO:633. In some embodiments, the second binding domain that binds to BCMA comprises: (i) a VH comprising a VH CDR1 having an amino acid sequence of SEQ ID NO:634, a VH CDR2 having an amino acid sequence of SEQ ID NO:635, and a VH CDR3 having an amino acid sequence of SEQ ID NO:636; and (ii) a VL comprising a VL CDR1 having an amino acid sequence of SEQ ID NO:637, a VL CDR2 having an amino acid sequence of SEQ ID NO:638, and a VL CDR3 having an amino acid sequence of SEQ ID NO:639. In some embodiments, the second binding domain that binds to BCMA comprises: (i) a VH comprising a VH CDR1 having an amino acid sequence of SEQ ID NO:640, a VH CDR2 having an amino acid sequence of SEQ ID NO:641, and a VH CDR3 having an amino acid sequence of SEQ ID NO:642; and (ii) a VL comprising a VL CDR1 having an amino acid sequence of SEQ ID NO:643, a VL CDR2 having an amino acid sequence of SEQ ID NO:644, and a VL CDR3 having an amino acid sequence of SEQ ID NO:645. In some embodiments, the second binding domain that binds to BCMA comprises: (i) a VH comprising a VH CDR1 having an amino acid sequence of SEQ ID NO:646, a VH CDR2 having an amino acid sequence of SEQ ID NO:647, and a VH CDR3 having an amino acid sequence of SEQ ID NO:648; and (ii) a VL comprising a VL CDR1 having an amino acid sequence of SEQ ID NO:649, a VL CDR2 having an amino acid sequence of SEQ ID NO:650, and a VL CDR3 having an amino acid sequence of SEQ ID NO:651. In some embodiments, the second binding domain that binds to BCMA comprises: (i) a VH comprising a VH CDR1 having an amino acid sequence of SEQ ID NO:652, a VH CDR2 having an amino acid sequence of SEQ ID NO:653, and a VH CDR3 having an amino acid sequence of SEQ ID NO:654; and (ii) a VL comprising a VL CDR1 having an amino acid sequence of SEQ ID NO:655, a VL CDR2 having an amino acid sequence of SEQ ID NO:656, and a VL CDR3 having an amino acid sequence of SEQ ID NO:657. In some embodiments, the second binding domain that binds to BCMA comprises: (i) a VH comprising a VH CDR1 having an amino acid sequence of SEQ ID NO:658, a VH CDR2 having an amino acid sequence of SEQ ID NO:659, and a VH CDR3 having an amino acid sequence of SEQ ID NO:660; and (ii) a VL comprising a VL CDR1 having an amino acid sequence of SEQ ID NO:661, a VL CDR2 having an amino acid sequence of SEQ ID NO:662, and a VL CDR3 having an amino acid sequence of SEQ ID NO:663. In some embodiments, the second binding domain that binds to BCMA comprises a VH having an amino acid sequence of SEQ ID NO:95. In some embodiments, the second binding domain that binds to BCMA comprises a VL having an amino acid sequence of SEQ ID NO:96. In some embodiments, the second binding domain that binds to BCMA comprises a VH having an amino acid sequence of SEQ ID NO:95, and a VL having an amino acid sequence of SEQ ID NO:96. In some embodiments, the second binding domain that binds to BCMA comprises a VH having an amino acid sequence having at least 95% identity to an amino acid sequence of SEQ ID NO:95. In some embodiments, the second binding domain that binds to BCMA comprises a VL having an amino acid sequence having at least 95% identity to an amino acid sequence of SEQ ID NO:96. In some embodiments, the second binding domain that binds to BCMA comprises a VH having an amino acid sequence having at least 95% identity to an amino acid sequence of SEQ ID NO:95, and a VL having an amino acid sequence having at least 95% identity to an amino acid sequence of SEQ ID NO:96. In some embodiments, the second binding domain that binds to BCMA comprises a VH having an amino acid sequence of SEQ ID NO:1052. In some embodiments, the second binding domain that binds to BCMA comprises a VL having an amino acid sequence of SEQ ID NO:1053. In some embodiments, the second binding domain that binds to BCMA comprises a VH having an amino acid sequence of SEQ ID NO:1052, and a VL having an amino acid sequence of SEQ ID NO:1053. In some embodiments, the second binding domain that binds to BCMA comprises a VH having an amino acid sequence having at least 95% identity to an amino acid sequence of SEQ ID NO:1052. In some embodiments, the second binding domain that binds to BCMA comprises a VL having an amino acid sequence having at least 95% identity to an amino acid sequence of SEQ ID NO:1053. In some embodiments, the second binding domain that binds to BCMA comprises a VH having an amino acid sequence having at least 95% identity to an amino acid sequence of SEQ ID NO:1052, and a VL having an amino acid sequence having at least 95% identity to an amino acid sequence of SEQ ID NO:1053.

In some embodiments, the second target is PSMA. Thus, in some embodiments, provided is a trispecific antibody comprising: (a) a first binding domain that binds to Vβ17, (b) a second binding domain that binds to PSMA, and (c) a third binding domain that binds to CD28. In some embodiments, the first binding domain that binds to Vβ17 comprises a VH CDR1, VH CDR2, and VH CDR3 of a Vβ17 antibody provided herein. In some embodiments, the first binding domain that binds to Vβ17 comprises a VL CDR1, VL CDR2, and VL CDR3 of a Vβ17 antibody provided herein. In some embodiments, the first binding domain that binds to Vβ17 comprises a VH CDR1, VH CDR2, VH CDR3, VL CDR1, VL CDR2, and VL CDR3 of a Vβ17 antibody provided herein. In some embodiments, the second binding domain that binds to PSMA comprises a VH CDR1, VH CDR2, and VH CDR3 of a PSMA antibody provided herein. In some embodiments, the second binding domain that binds to PSMA comprises a VL CDR1, VL CDR2, and VL CDR3 of a PSMA antibody provided herein. In some embodiments, the second binding domain that binds to PSMA comprises a VH CDR1, VH CDR2, VH CDR3, VL CDR1, VL CDR2, and VL CDR3 of a PSMA antibody provided herein. In some embodiments, the third binding domain that binds to CD28 comprises a VH CDR1, VH CDR2, and VH CDR3 of a CD28 antibody provided herein. In some embodiments, the third binding domain that binds to CD28 comprises a VL CDR1, VL CDR2, and VL CDR3 of a CD28 antibody provided herein. In some embodiments, the third binding domain that binds to CD28 comprises a VH CDR1, VH CDR2, VH CDR3, VL CDR1, VL CDR2, and VL CDR3 of a CD28 antibody provided herein.

In some embodiments, the second binding domain that binds to PSMA comprises: a VH comprising a VH CDR1, a VH CDR2, and a VH CDR3 having an amino acid sequence of a VH CDR1, VH CDR2, and VH CDR3, respectively, of SEQ ID NO:1046. In some embodiments, the second binding domain that binds to PSMA comprises: a VL comprising a VL CDR1, a VL CDR2, and a VL CDR3 having an amino acid sequence of a VL CDR1, VL CDR2, and VL CDR3, respectively, of SEQ ID NO:1047. In some embodiments, the second binding domain that binds to PSMA comprises: (i) a VH comprising a VH CDR1, a VH CDR2, and a VH CDR3 having an amino acid sequence of a VH CDR1, VH CDR2, and VH CDR3, respectively, of SEQ ID NO:1046; and (ii) a VL comprising a VL CDR1, a VL CDR2, and a VL CDR3 having an amino acid sequence of a VL CDR1, VL CDR2, and VL CDR3, respectively, of SEQ ID NO:1047. In some embodiments, the second binding domain that binds to PSMA comprises a VH having an amino acid sequence of SEQ ID NO:1046. In some embodiments, the second binding domain that binds to PSMA comprises a VL having an amino acid sequence of SEQ ID NO:1047. In some embodiments, the second binding domain that binds to PSMA comprises a VH having an amino acid sequence of SEQ ID NO:1046, and a VL having an amino acid sequence of SEQ ID NO:1047. In some embodiments, the second binding domain that binds to PSMA comprises a VH having an amino acid sequence having at least 95% identity to an amino acid sequence of SEQ ID NO:1046. In some embodiments, the second binding domain that binds to PSMA comprises a VL having an amino acid sequence having at least 95% identity to an amino acid sequence of SEQ ID NO:1047. In some embodiments, the second binding domain that binds to PSMA comprises a VH having an amino acid sequence having at least 95% identity to an amino acid sequence of SEQ ID NO:1046, and a VL having an amino acid sequence having at least 95% identity to an amino acid sequence of SEQ ID NO:1047. In some embodiments, the second binding domain that binds to PSMA comprises: (i) a VH comprising a VH CDR1 having an amino acid sequence of SEQ ID NO:1019, a VH CDR2 having an amino acid sequence of SEQ ID NO:1020, and a VH CDR3 having an amino acid sequence of SEQ ID NO:1021; and (ii) a VL comprising a VL CDR1 having an amino acid sequence of SEQ ID NO:1034, a VL CDR2 having an amino acid sequence of SEQ ID NO:1035, and a VL CDR3 having an amino acid sequence of SEQ ID NO:1036. In some embodiments, the second binding domain that binds to PSMA comprises: (i) a VH comprising a VH CDR1 having an amino acid sequence of SEQ ID NO:1022, a VH CDR2 having an amino acid sequence of SEQ ID NO:1023, and a VH CDR3 having an amino acid sequence of SEQ ID NO:1024; and (ii) a VL comprising a VL CDR1 having an amino acid sequence of SEQ ID NO:1037, a VL CDR2 having an amino acid sequence of SEQ ID NO:1038, and a VL CDR3 having an amino acid sequence of SEQ ID NO:1039. In some embodiments, the second binding domain that binds to PSMA comprises: (i) a VH comprising a VH CDR1 having an amino acid sequence of SEQ ID NO:1016, a VH CDR2 having an amino acid sequence of SEQ ID NO:1017, and a VH CDR3 having an amino acid sequence of SEQ ID NO:1018; and (ii) a VL comprising a VL CDR1 having an amino acid sequence of SEQ ID NO:1031, a VL CDR2 having an amino acid sequence of SEQ ID NO:1032, and a VL CDR3 having an amino acid sequence of SEQ ID NO:1033. In some embodiments, the second binding domain that binds to PSMA comprises: (i) a VH comprising a VH CDR1 having an amino acid sequence of SEQ ID NO:1028, a VH CDR2 having an amino acid sequence of SEQ ID NO:1029, and a VH CDR3 having an amino acid sequence of SEQ ID NO:1030; and (ii) a VL comprising a VL CDR1 having an amino acid sequence of SEQ ID NO:1043, a VL CDR2 having an amino acid sequence of SEQ ID NO:1044, and a VL CDR3 having an amino acid sequence of SEQ ID NO:1045. In some embodiments, the second binding domain that binds to PSMA comprises: (i) a VH comprising a VH CDR1 having an amino acid sequence of SEQ ID NO:1025, a VH CDR2 having an amino acid sequence of SEQ ID NO:1026, and a VH CDR3 having an amino acid sequence of SEQ ID NO:1027; and (ii) a VL comprising a VL CDR1 having an amino acid sequence of SEQ ID NO:1040, a VL CDR2 having an amino acid sequence of SEQ ID NO:1041, and a VL CDR3 having an amino acid sequence of SEQ ID NO:1042.

In some embodiments, the third binding domain that binds to CD28 comprises: a VH comprising a VH CDR1, a VH CDR2, and a VH CDR3 having an amino acid sequence of a VH CDR1, VH CDR2, and VH CDR3, respectively, of SEQ ID NO:690. In some embodiments, the third binding domain that binds to CD28 comprises: a VL comprising a VL CDR1, a VL CDR2, and a VL CDR3 having an amino acid sequence of a VL CDR1, VL CDR2, and VL CDR3, respectively, of SEQ ID NO:696. In some embodiments, the third binding domain that binds to CD28 comprises: (i) a VH comprising a VH CDR1, a VH CDR2, and a VH CDR3 having an amino acid sequence of a VH CDR1, VH CDR2, and VH CDR3, respectively, of SEQ ID NO:690; and (ii) a VL comprising a VL CDR1, a VL CDR2, and a VL CDR3 having an amino acid sequence of a VL CDR1, VL CDR2, and VL CDR3, respectively, of SEQ ID NO:696. In some embodiments, the third binding domain that binds to CD28 comprises a VH having an amino acid sequence of SEQ ID NO:690. In some embodiments, the third binding domain that binds to CD28 comprises a VL having an amino acid sequence of SEQ ID NO:696. In some embodiments, the third binding domain that binds to CD28 comprises a VH having an amino acid sequence of SEQ ID NO:690, and a VL having an amino acid sequence of SEQ ID NO:696. In some embodiments, the third binding domain that binds to CD28 comprises a VH having an amino acid sequence having at least 95% identity to an amino acid sequence of SEQ ID NO:690. In some embodiments, the third binding domain that binds to CD28 comprises a VL having an amino acid sequence having at least 95% identity to an amino acid sequence of SEQ ID NO:696. In some embodiments, the third binding domain that binds to CD28 comprises a VH having an amino acid sequence having at least 95% identity to an amino acid sequence of SEQ ID NO:690, and a VL having an amino acid sequence having at least 95% identity to an amino acid sequence of SEQ ID NO:696.

In some embodiments, the third binding domain that binds to CD28 comprises: a VH comprising a VH CDR1, a VH CDR2, and a VH CDR3 having an amino acid sequence of a VH CDR1, VH CDR2, and VH CDR3, respectively, of SEQ ID NO:691. In some embodiments, the third binding domain that binds to CD28 comprises: a VL comprising a VL CDR1, a VL CDR2, and a VL CDR3 having an amino acid sequence of a VL CDR1, VL CDR2, and VL CDR3, respectively, of SEQ ID NO:697. In some embodiments, the third binding domain that binds to CD28 comprises: (i) a VH comprising a VH CDR1, a VH CDR2, and a VH CDR3 having an amino acid sequence of a VH CDR1, VH CDR2, and VH CDR3, respectively, of SEQ ID NO:691; and (ii) a VL comprising a VL CDR1, a VL CDR2, and a VL CDR3 having an amino acid sequence of a VL CDR1, VL CDR2, and VL CDR3, respectively, of SEQ ID NO:697. In some embodiments, the third binding domain that binds to CD28 comprises a VH having an amino acid sequence of SEQ ID NO:691. In some embodiments, the third binding domain that binds to CD28 comprises a VL having an amino acid sequence of SEQ ID NO:697. In some embodiments, the third binding domain that binds to CD28 comprises a VH having an amino acid sequence of SEQ ID NO:691, and a VL having an amino acid sequence of SEQ ID NO:697. In some embodiments, the third binding domain that binds to CD28 comprises a VH having an amino acid sequence having at least 95% identity to an amino acid sequence of SEQ ID NO:691. In some embodiments, the third binding domain that binds to CD28 comprises a VL having an amino acid sequence having at least 95% identity to an amino acid sequence of SEQ ID NO:697. In some embodiments, the third binding domain that binds to CD28 comprises a VH having an amino acid sequence having at least 95% identity to an amino acid sequence of SEQ ID NO:691, and a VL having an amino acid sequence having at least 95% identity to an amino acid sequence of SEQ ID NO:697.

In some embodiments, the third binding domain that binds to CD28 comprises: a VH comprising a VH CDR1, a VH CDR2, and a VH CDR3 having an amino acid sequence of a VH CDR1, VH CDR2, and VH CDR3, respectively, of SEQ ID NO:692. In some embodiments, the third binding domain that binds to CD28 comprises: a VL comprising a VL CDR1, a VL CDR2, and a VL CDR3 having an amino acid sequence of a VL CDR1, VL CDR2, and VL CDR3, respectively, of SEQ ID NO:698. In some embodiments, the third binding domain that binds to CD28 comprises: (i) a VH comprising a VH CDR1, a VH CDR2, and a VH CDR3 having an amino acid sequence of a VH CDR1, VH CDR2, and VH CDR3, respectively, of SEQ ID NO:692; and (ii) a VL comprising a VL CDR1, a VL CDR2, and a VL CDR3 having an amino acid sequence of a VL CDR1, VL CDR2, and VL CDR3, respectively, of SEQ ID NO:698. In some embodiments, the third binding domain that binds to CD28 comprises a VH having an amino acid sequence of SEQ ID NO:692. In some embodiments, the third binding domain that binds to CD28 comprises a VL having an amino acid sequence of SEQ ID NO:698. In some embodiments, the third binding domain that binds to CD28 comprises a VH having an amino acid sequence of SEQ ID NO:692, and a VL having an amino acid sequence of SEQ ID NO:698. In some embodiments, the third binding domain that binds to CD28 comprises a VH having an amino acid sequence having at least 95% identity to an amino acid sequence of SEQ ID NO:692. In some embodiments, the third binding domain that binds to CD28 comprises a VL having an amino acid sequence having at least 95% identity to an amino acid sequence of SEQ ID NO:698. In some embodiments, the third binding domain that binds to CD28 comprises a VH having an amino acid sequence having at least 95% identity to an amino acid sequence of SEQ ID NO:692, and a VL having an amino acid sequence having at least 95% identity to an amino acid sequence of SEQ ID NO:698.

In some embodiments, the third binding domain that binds to CD28 comprises: a VH comprising a VH CDR1, a VH CDR2, and a VH CDR3 having an amino acid sequence of a VH CDR1, VH CDR2, and VH CDR3, respectively, of SEQ ID NO:693. In some embodiments, the third binding domain that binds to CD28 comprises: a VL comprising a VL CDR1, a VL CDR2, and a VL CDR3 having an amino acid sequence of a VL CDR1, VL CDR2, and VL CDR3, respectively, of SEQ ID NO:699. In some embodiments, the third binding domain that binds to CD28 comprises: (i) a VH comprising a VH CDR1, a VH CDR2, and a VH CDR3 having an amino acid sequence of a VH CDR1, VH CDR2, and VH CDR3, respectively, of SEQ ID NO:693; and (ii) a VL comprising a VL CDR1, a VL CDR2, and a VL CDR3 having an amino acid sequence of a VL CDR1, VL CDR2, and VL CDR3, respectively, of SEQ ID NO:699. In some embodiments, the third binding domain that binds to CD28 comprises a VH having an amino acid sequence of SEQ ID NO:693. In some embodiments, the third binding domain that binds to CD28 comprises a VL having an amino acid sequence of SEQ ID NO:699. In some embodiments, the third binding domain that binds to CD28 comprises a VH having an amino acid sequence of SEQ ID NO:693, and a VL having an amino acid sequence of SEQ ID NO:699. In some embodiments, the third binding domain that binds to CD28 comprises a VH having an amino acid sequence having at least 95% identity to an amino acid sequence of SEQ ID NO:693. In some embodiments, the third binding domain that binds to CD28 comprises a VL having an amino acid sequence having at least 95% identity to an amino acid sequence of SEQ ID NO:699. In some embodiments, the third binding domain that binds to CD28 comprises a VH having an amino acid sequence having at least 95% identity to an amino acid sequence of SEQ ID NO:693, and a VL having an amino acid sequence having at least 95% identity to an amino acid sequence of SEQ ID NO:699.

In some embodiments, the trispecific antibodies include IgG-like molecules with complementary CH3 domains that promote heterodimerization; recombinant IgG-like dual targeting molecules, wherein the three sides of the molecule each contain the Fab fragment or part of the Fab fragment of at least three different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof, Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody) (EMD Serono), the Biclonic (Merus) and the DuoBody (Genmab A/S).

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (InnClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)₂-Fab (National Research Center for Antibody Medicine-China).

In some embodiments, Fab fusion trispecific antibodies include F(ab)₂ (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Trispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Publ. No. WO2006/028936) can be used to generate full length bispecific and trispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T3661_K392M_T394W/F405A_Y407V, T366L_K392M_T394W/F405A_Y407V, L351Y_Y407A/T366A_K409F, L351Y_Y407A/T366V K409F Y407A/T366A_K409F, or T350V_L351Y_F405A Y407V/T350V_T366L_K392L_T394W as described in U.S. Pat. Publ. No. US2012/0149876 or U.S. Pat. Publ. No. US2013/0195849.

According to another particular aspect, the invention relates to an isolated Vβ17×CD28 trispecific antibody or antigen-binding fragment thereof that induces antibody-dependent cell-mediated cytotoxicity (ADCC). The trispecific antibody or antigen-binding fragment thereof can, for example, induce ADCC in vitro. The trispecific antibody or antigen-binding fragment thereof can induce ADCC with an EC₅₀ of less than about 1 pM. In certain embodiments, the Vβ17×CD28 trispecific antibody or antigen-binding fragment thereof comprises an IgG1, IgG2, IgG3, or IgG4 backbone. In one such embodiment, the Vβ17×CD28 trispecific antibody or antigen-binding fragment thereof has an antibody backbone of the IgG4 isotype. In one embodiment, the Vβ17×CD28 trispecific antibody is an anti-Vβ17/anti-BCMA/anti-CD28 trispecific antibody. In one embodiment, the Vβ17×CD28 trispecific antibody is an anti-Vβ17/anti-PSMA/anti-CD28 trispecific antibody.

In some embodiments described herein, ADCC elicited by the Vβ17×CD28 trispecific antibodies can also be enhanced by certain substitutions in the antibody Fc. Exemplary substitutions include, for example, substitutions at amino acid positions 256, 290, 298, 312, 356, 330, 333, 334, 360, 378 or 430 (residue numbering according to the EU index) as described in U.S. Pat. No. 6,737,056.

According to another particular aspect, the invention relates to an isolated Vβ17×CD28 trispecific antibody or antigen-binding fragment thereof capable of inducing T-cell dependent cytotoxicity in Vβ17-expressing cells, CD28-expressing cells, and/or target-expressing cells. In some embodiments, the target is BCMA. In some embodiment, the target is PSMA. The trispecific antibody or antigen-binding fragment thereof can, for example, induce T-cell dependent cytotoxicity in Vβ17-expressing cells, CD28-expressing cells and/or target-expressing cells in vitro with an EC₅₀ value of less than about 2 nM. In certain embodiments, the EC₅₀ is less than about 2.0 nM, less than about 1.9 nM, less than about 1.8 nM, less than about 1.7 nM, less than about 1.6 nM, less than about 1.5 nM, less than about 1.4 nM, less than about 1.3 nM, less than about 1.2 nM, less than about 1.1 nM, less than about 1.0 nM, less than about 0.9 nM, less than about 0.8 nM, less than about 0.7 nM, less than about 0.6 nM, less than about 0.5 nM, less than about 0.4 nM, less than about 0.3 nM, less than about 0.2 nM, and less than about 0.1 nM.

In some embodiments, the antibody described herein is a multispecific antibody.

In some embodiments, the trispecific antibody provided herein does not comprise a single chain antibody. In some embodiments, the trispecific antibody provided herein does not comprise a single domain antibody. In certain embodiments, the trispecific antibody provided herein does not comprise a nanobody. In certain embodiments, the trispecific antibody provided herein does not comprise a VHH antibody. In certain embodiments, the trispecific antibody provided herein does not comprise a llama antibody.

In certain embodiments, the trispecific antibody or antigen-binding fragment thereof induces T cell dependent cytotoxicity of a B cell in vitro with an EC₅₀ of less than about 160 pM, when assessed in vitro at an effector to target cell ratio of 1:1.

In some embodiments, Vβ17 is present on the surface of a T cell. In some embodiments, CD28 is present on the surface of a T cell. In some embodiments, CD28 is present on the surface of a T cell.

In some embodiments, the cancer antigen is present on the surface of a cancer cell. In some embodiments, BCMA is present on the surface of a target cell. In some embodiments, the Vβ17 is present on the surface of a T cell, BCMA is on the surface of a target cell and CD28 is on the surface of a T cell. In some embodiments, the Vβ17 is present on the surface of a T cell, BCMA is on the surface of a target cell and CD28 is on the surface of a target cell. In some embodiments, the target cell is killed when the trispecific antibody binds to the Vβ17 on the surface of the T cell, the CD28 on the surface of the T cell and the BCMA on the surface of the target cell. In some embodiments, the target cell is killed when the trispecific antibody binds to the Vβ17 on the surface of the T cell, the CD28 on the surface of the T cell and the BCMA on the surface of the target cell. In certain embodiments, the target cell is a B cell. In certain embodiments, the target is a B cell cancer cell.

In some embodiments, PSMA is present on the surface of a target cell. In some embodiments, the Vβ17 is present on the surface of a T cell, PSMA is on the surface of a target cell and CD28 is on the surface of a T cell. In some embodiments, the Vβ17 is present on the surface of a T cell, PSMA is on the surface of a target cell and CD28 is on the surface of a target cell. In some embodiments, the target cell is killed when the trispecific antibody binds to the Vβ17 on the surface of the T cell, the CD28 on the surface of the T cell and the PSMA on the surface of the target cell. In some embodiments, the target cell is killed when the trispecific antibody binds to the Vβ17 on the surface of the T cell, the CD28 on the surface of the T cell and the PSMA on the surface of the target cell. In certain embodiments, the target cell is a prostate cell. In certain embodiments, the target is a prostate cancer cell.

In some embodiments, the trispecific antibody induces T cell dependent cytotoxicity of the target cell in vitro with an EC₅₀ of less than about 500 pM. In some embodiments, the trispecific antibody induces T cell dependent cytotoxicity of the target cell in vitro with an EC₅₀ of less than about 300 pM. In some embodiments, the trispecific antibody induces T cell dependent cytotoxicity of the target cell in vitro with an EC₅₀ of less than about 160 pM. In some embodiments, the EC₅₀ is assessed with a mixture of effector T cells and target B cells. In some embodiments, the T cells are αβ T cells. In some embodiments, the effector cell to target cell ratio is about 0.01 to 1 to about 5 to 1. In some embodiments, the effector cell to target cell ratio is about 0.1 to 1 to about 2 to 1. In some embodiments, the effector cell to target cell ratio is about 1:1.

In certain embodiments, the EC₅₀ is less than about 1 pM, less than about 0.9 pM, less than about 0.8 pM, less than about 0.7 pM, less than about 0.6 pM, less than about 0.5 pM, less than about 0.4 pM, less than about 0.300 pM, less than about 0.2 pM, less than about 0.19 pM, less than about 0.18 pM, less than about 0.17 pM, less than about 0.16 pM, less than about 0.15 pM, less than about 0.14 pM, less than about 0.13 pM, less than about 0.12 pM, less than about 0.11 pM, less than about 0.1 pM, less than about 0.09 pM, less than about 0.08 pM, less than about 0.07 pM, less than about 0.06 pM, less than about 0.05 pM, less than about 0.04 pM, less than about 0.03 pM, less than about 0.02 pM, or less than about 0.01 pM. In certain embodiments, the EC₅₀ is less than about 1000 pM, less than about 900 pM, less than about 800 pM, less than about 700 pM, less than about 600 pM, less than about 500 pM, less than about 400 pM, less than about 300 pM, less than about 200 pM, less than about 190 pM, less than about 180 pM, less than about 170 pM, less than about 160 pM, less than about 150 pM, less than about 140 pM, less than about 130 pM, less than about 120 pM, less than about 110 pM, less than about 100 pM, less than about 90 pM, less than about 80 pM, less than about 70 pM, less than about 60 pM, less than about 50 pM, less than about 40 pM, less than about 30 pM, less than about 20 pM, or less than about 10 pM.

In certain embodiments, the effector to target cell ratio can, for example, be 0.01:1, 0.02:1, 0.03:1, 0.04:1, 0.05:1, 0.06:1, 0.07:1, 0.08:1, 0.09:1, 1:1, 2:1, 3:1, 4:1, 5:1, 6:1, 7:1, 8:1, 9:1, or 10:1.

In some embodiments of the trispecific antibody provided herein, the T cell releases cytokines when the trispecific antibody binds to CD28 on the surface of the T cell. In other embodiments provided herein is a method of activating a T cell comprising contacting the T cell with a trispecific antibody provided herein, wherein the T cell releases cytokines upon activation by the trispecific antibody. In some embodiments, the cytokine is a chemokine. In some embodiments, the cytokine is an interferon. In some embodiments, the cytokine is an interleukin. In some embodiments, the cytokine is a protein belonging to the tumour necrosis factor superfamily. In some embodiments, the chemokine is a CC chemokine. In some embodiments, the chemokine is a CXC chemokine. In some embodiments, the chemokine is a C chemokine or a CX3C chemokine. In some embodiments. In some embodiments, the interferon is a Type I interferon. In some embodiments, the interferon is a Type 2 interferon. In some embodiments, the interferon is a Type 3 interferon. In some embodiments, the interleukin is an IL-1. In some embodiments, the interleukin is an IL-2. In some embodiments, the interleukin is an IL-3. In some embodiments, the interleukin is an IL-4. In some embodiments, the interleukin is an IL-5. In some embodiments, the interleukin is an IL-6. In some embodiments, the interleukin is an IL-7. In some embodiments, the interleukin is an IL-8. In some embodiments, the interleukin is an IL-9. In some embodiments, the interleukin is an IL-10. In some embodiments, the interleukin is an IL-11. In some embodiments, the interleukin is an IL-12. In some embodiments, the interleukin is an IL-13. In some embodiments, the interleukin is an IL-15 or an IL-17. In some embodiments, the protein belonging to the tumour necrosis factor superfamily is a lymphotoxin alpha. In some embodiments, the protein belonging to the tumour necrosis factor superfamily is a tumor necrosis factor. In some embodiments, the protein belonging to the tumour necrosis factor superfamily is a lymphotoxin beta. In some embodiments, the protein belonging to the tumour necrosis factor superfamily is an OX40 ligand. In some embodiments, the protein belonging to the tumour necrosis factor superfamily is a CD40 ligand. In some embodiments, the protein belonging to the tumour necrosis factor superfamily is a Fas ligand. In some embodiments, the protein belonging to the tumour necrosis factor superfamily is a CD27 ligand. In some embodiments, the protein belonging to the tumour necrosis factor superfamily is a CD30 ligand. In some embodiments, the protein belonging to the tumour necrosis factor superfamily is a CD137 ligand. In some embodiments, the protein belonging to the tumour necrosis factor superfamily is a TNF-related apoptosis-inducing ligand. In some embodiments, the protein belonging to the tumour necrosis factor superfamily is a receptor activator of nuclear factor kappa-B ligand. In some embodiments, the protein belonging to the tumour necrosis factor superfamily is a TNF-related weak inducer of apoptosis. In some embodiments, the protein belonging to the tumour necrosis factor superfamily is a proliferation-inducing ligand. In some embodiments, the protein belonging to the tumour necrosis factor superfamily is a B-cell activating factor. In some embodiments, the protein belonging to the tumour necrosis factor superfamily is a LIGHT. In some embodiments, the protein belonging to the tumour necrosis factor superfamily is a vascular endothelial growth inhibitor. In some embodiments, the protein belonging to the tumour necrosis factor superfamily is a TNF superfamily member 18. In some embodiments, the protein belonging to the tumour necrosis factor superfamily is a or ectodysplasin A.

In certain embodiments, the concentration of the trispecific antibody or antigen-binding fragment thereof is about 0.000005 ng/mL, about 0.00005 ng/mL, about 0.0005, about 0.005 ng/mL, about 0.01 ng/mL, about 0.02 ng/mL, about 0.03 ng/mL, about 0.04 ng/mL, about 0.05 ng/mL, about 0.06 ng/mL, about 0.07 ng/mL, about 0.08 ng/mL, about 0.09 ng/mL, about 0.1 ng/mL, about 0.5 ng/mL, about 1.0 ng/mL, about 10 ng/mL, about 20 ng/mL about, about 30 ng/mL about 40 ng/mL, about 50 ng/mL, about 60 ng/mL, about 70 ng/mL, about 80 ng/mL, about 90 ng/mL, about 100 ng/mL, or about 1000 ng/mL.

In another aspect, provided herein is an antibody that competes for binding to Vβ17 with any of the Vβ17 antibodies described herein. In another aspect, provided herein is an antibody that binds to the same epitope as any of the Vβ17 antibodies described herein. In another aspect, provided is a Vβ17 antibody that binds an epitope on Vβ17 that overlaps with the epitope on Vβ17 bound by a Vβ17 antibody described herein. In some embodiments, the Vβ17 antibody comprises a VH CDR1, VH CDR2, and VH CDR3 of a Vβ17 antibody provided herein. In some embodiments, the Vβ17 antibody comprises a VL CDR1, VL CDR2, and VL CDR3 of a Vβ17 antibody provided herein. In some embodiments, the Vβ17 antibody comprises a VH CDR1, VH CDR2, VH CDR3, a VL CDR1, VL CDR2, and VL CDR3 of a Vβ17 antibody provided herein. In some embodiments, the Vβ17 antibody comprises a VH of a Vβ17 antibody provided herein. In some embodiments, the Vβ17 antibody comprises a VL of a Vβ17 antibody provided herein. In some embodiments, the Vβ17 antibody comprises a VH and a VL of a Vβ17 antibody provided herein. In some embodiments, the Vβ17 antibody comprises a VH CDR1, VH CDR2, VH CDR3, a VL CDR1, VL CDR2, and VL CDR3 of a Vβ17 antibody provided herein. In some embodiments, the VH CDR1, VH CDR2, VH CDR3, VL CDR1, VL CDR2, and VL CDR3 amino acid sequences of the Vβ17 antibody are according to the Kabat numbering system. In some embodiments, the VH CDR1, VH CDR2, VH CDR3, VL CDR1, VL CDR2, and VL CDR3 amino acid sequences of the Vβ17 antibody are according to the Chothia numbering system. In some embodiments, the VH CDR1, VH CDR2, VH CDR3, VL CDR1, VL CDR2, and VL CDR3 amino acid sequences of the Vβ17 antibody are according to the AbM numbering system. In some embodiments, the VH CDR1, VH CDR2, VH CDR3, VL CDR1, VL CDR2, and VL CDR3 amino acid sequences of the Vβ17 antibody are according to the Contact numbering system. In some embodiments, the VH CDR1, VH CDR2, VH CDR3, VL CDR1, VL CDR2, and VL CDR3 amino acid sequences of the Vβ17 antibody are according to the IMGT numbering system. In certain embodiments, the Vβ17 antibody is a multispecific antibody. In some embodiments, the Vβ17 antibody is a bispecific antibody. In some embodiments, the Vβ17 antibody is a trispecific antibody.

In another aspect, provided is an antibody that competes for binding to Vβ17 with a Vβ17 reference antibody. In another aspect, provided is a Vβ17 antibody that binds to the same Vβ17 epitope as a Vβ17 reference antibody. In another aspect, provided is a Vβ17 antibody that binds an epitope on Vβ17 that overlaps with the epitope on Vβ17 bound by a Vβ17 reference antibody. In some embodiments, the Vβ17 reference antibody comprises a VH CDR1, VH CDR2, and VH CDR3 of a Vβ17 reference antibody provided herein. In some embodiments, the Vβ17 reference antibody comprises a VL CDR1, VL CDR2, and VL CDR3 of a Vβ17 reference antibody provided herein. In some embodiments, the Vβ17 reference antibody comprises a VH CDR1, VH CDR2, VH CDR3, a VL CDR1, VL CDR2, and VL CDR3 of a Vβ17 reference antibody provided herein. In some embodiments, the Vβ17 reference antibody comprises a VH of a Vβ17 reference antibody provided herein. In some embodiments, the Vβ17 reference antibody comprises a VL of a Vβ17 reference antibody provided herein. In some embodiments, the Vβ17 reference antibody comprises a VH and a VL of a Vβ17 reference antibody provided herein. In some embodiments, the Vβ17 reference antibody comprises a VH CDR1, VH CDR2, VH CDR3, a VL CDR1, VL CDR2, and VL CDR3 of a Vβ17 reference antibody provided herein. In some embodiments, the VH CDR1, VH CDR2, VH CDR3, VL CDR1, VL CDR2, and VL CDR3 amino acid sequences of the Vβ17 reference antibody are according to the Kabat numbering system. In some embodiments, the VH CDR1, VH CDR2, VH CDR3, VL CDR1, VL CDR2, and VL CDR3 amino acid sequences of the Vβ17 reference antibody are according to the Chothia numbering system. In some embodiments, the VH CDR1, VH CDR2, VH CDR3, VL CDR1, VL CDR2, and VL CDR3 amino acid sequences of the Vβ17 reference antibody are according to the AbM numbering system. In some embodiments, the VH CDR1, VH CDR2, VH CDR3, VL CDR1, VL CDR2, and VL CDR3 amino acid sequences of the Vβ17 reference antibody are according to the Contact numbering system. In some embodiments, the VH CDR1, VH CDR2, VH CDR3, VL CDR1, VL CDR2, and VL CDR3 amino acid sequences of the Vβ17 reference antibody are according to the IMGT numbering system. In certain embodiments, the antibody is a multispecific antibody. In some embodiments, the antibody is a bispecific antibody. In certain embodiments, the Vβ17 reference antibody is a multispecific antibody. In some embodiments, the Vβ17 reference antibody is a bispecific antibody. In some embodiments, the Vβ17 reference antibody is a trispecific antibody.

In another aspect, provided herein is an antibody that competes for binding to CD28 with any of the CD28 antibodies described herein. In another aspect, provided herein is an antibody that binds to the same epitope as any of the CD28 antibodies described herein. In another aspect, provided is a CD28 antibody that binds an epitope on CD28 that overlaps with the epitope on CD28 bound by a CD28 antibody described herein. In some embodiments, the CD28 antibody comprises a VH CDR1, VH CDR2, and VH CDR3 of a CD28 antibody provided herein. In some embodiments, the CD28 antibody comprises a VL CDR1, VL CDR2, and VL CDR3 of a CD28 antibody provided herein. In some embodiments, the CD28 antibody comprises a VH CDR1, VH CDR2, VH CDR3, a VL CDR1, VL CDR2, and VL CDR3 of a CD28 antibody provided herein. In some embodiments, the CD28 antibody comprises a VH of a CD28 antibody provided herein. In some embodiments, the CD28 antibody comprises a VL of a CD28 antibody provided herein. In some embodiments, the CD28 antibody comprises a VH and a VL of a CD28 antibody provided herein. In some embodiments, the CD28 antibody comprises a VH CDR1, VH CDR2, VH CDR3, a VL CDR1, VL CDR2, and VL CDR3 of a CD28 antibody provided herein. In some embodiments, the VH CDR1, VH CDR2, VH CDR3, VL CDR1, VL CDR2, and VL CDR3 amino acid sequences of the CD28 antibody are according to the Kabat numbering system. In some embodiments, the VH CDR1, VH CDR2, VH CDR3, VL CDR1, VL CDR2, and VL CDR3 amino acid sequences of the CD28 antibody are according to the Chothia numbering system. In some embodiments, the VH CDR1, VH CDR2, VH CDR3, VL CDR1, VL CDR2, and VL CDR3 amino acid sequences of the CD28 antibody are according to the AbM numbering system. In some embodiments, the VH CDR1, VH CDR2, VH CDR3, VL CDR1, VL CDR2, and VL CDR3 amino acid sequences of the CD28 antibody are according to the Contact numbering system. In some embodiments, the VH CDR1, VH CDR2, VH CDR3, VL CDR1, VL CDR2, and VL CDR3 amino acid sequences of the CD28 antibody are according to the IMGT numbering system. In certain embodiments, the CD28 antibody is a multispecific antibody. In some embodiments, the CD28 antibody is a bispecific antibody. In some embodiments, the CD28 antibody is a trispecific antibody.

In another aspect, provided is an antibody that competes for binding to CD28 with a CD28 reference antibody. In another aspect, provided is a CD28 antibody that binds to the same CD28 epitope as a CD28 reference antibody. In another aspect, provided is a CD28 antibody that binds an epitope on CD28 that overlaps with the epitope on CD28 bound by a CD28 reference antibody. In some embodiments, the CD28 reference antibody comprises a VH CDR1, VH CDR2, and VH CDR3 of a CD28 reference antibody provided herein. In some embodiments, the CD28 reference antibody comprises a VL CDR1, VL CDR2, and VL CDR3 of a CD28 reference antibody provided herein. In some embodiments, the CD28 reference antibody comprises a VH CDR1, VH CDR2, VH CDR3, a VL CDR1, VL CDR2, and VL CDR3 of a CD28 reference antibody provided herein. In some embodiments, the CD28 reference antibody comprises a VH of a CD28 reference antibody provided herein. In some embodiments, the CD28 reference antibody comprises a VL of a CD28 reference antibody provided herein. In some embodiments, the CD28 reference antibody comprises a VH and a VL of a CD28 reference antibody provided herein. In some embodiments, the CD28 reference antibody comprises a VH CDR1, VH CDR2, VH CDR3, a VL CDR1, VL CDR2, and VL CDR3 of a CD28 reference antibody provided herein. In some embodiments, the VH CDR1, VH CDR2, VH CDR3, VL CDR1, VL CDR2, and VL CDR3 amino acid sequences of the CD28 reference antibody are according to the Kabat numbering system. In some embodiments, the VH CDR1, VH CDR2, VH CDR3, VL CDR1, VL CDR2, and VL CDR3 amino acid sequences of the CD28 reference antibody are according to the Chothia numbering system. In some embodiments, the VH CDR1, VH CDR2, VH CDR3, VL CDR1, VL CDR2, and VL CDR3 amino acid sequences of the CD28 reference antibody are according to the AbM numbering system. In some embodiments, the VH CDR1, VH CDR2, VH CDR3, VL CDR1, VL CDR2, and VL CDR3 amino acid sequences of the CD28 reference antibody are according to the Contact numbering system. In some embodiments, the VH CDR1, VH CDR2, VH CDR3, VL CDR1, VL CDR2, and VL CDR3 amino acid sequences of the CD28 reference antibody are according to the IMGT numbering system. In certain embodiments, the antibody is a multispecific antibody. In some embodiments, the antibody is a bispecific antibody. In certain embodiments, the CD28 reference antibody is a multispecific antibody. In some embodiments, the CD28 reference antibody is a bispecific antibody. In some embodiments, the CD28 reference antibody is a trispecific antibody.

In some embodiments described herein, immune effector properties of the trispecific antibodies provided herein can be enhanced or silenced through Fc modifications by techniques known to those skilled in the art. For example, Fc effector functions such as Clq binding, complement dependent cytotoxicity (CDC), antibody-dependent cell-mediated cytotoxicity (ADCC), antibody-dependent cell-mediated phagocytosis (ADCP), down regulation of cell surface receptors (e.g., B cell receptor; BCR), etc. can be provided and/or controlled by modifying residues in the Fc responsible for these activities.

“Antibody-dependent cell-mediated cytotoxicity” or “ADCC” refers to a cell-mediated reaction in which non-specific cytotoxic cells that express Fc receptors (FcRs) (e.g. Natural Killer (NK) cells, neutrophils, and macrophages) recognize bound antibody on a target cell and subsequently cause lysis of the target cell.

The ability of antibodies to induce ADCC can be enhanced by engineering their oligosaccharide component. Human IgG1 or IgG3 are N-glycosylated at Asn297 with the majority of the glycans in the well-known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved FcγRIIIa binding without altering antigen binding or CDC activity. Such Abs can be achieved using different methods reported to lead to the successful expression of relatively high defucosylated antibodies bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the α-1,6-fucosyltrasferase (FUT8) gene (Mori et al., Biotechnol Bioeng 88:901-908, 2004), or coexpression of 0-1,4-N-acetylglucosaminyltransferase III and golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

According to another particular aspect, the invention relates to an isolated Vβ17×CD28 trispecific antibody or antigen-binding fragment thereof, wherein the Vβ17×CD28 trispecific antibody or antigen-binding fragment thereof is chimeric.

According to another particular aspect, the invention relates to an isolated Vβ17×CD28 trispecific antibody or antigen-binding fragment thereof, wherein the Vβ17×CD28 trispecific antibody or antigen-binding fragment thereof is human or humanized. In some embodiments, the Vβ17×CD28 trispecific antibody is an anti-Vβ17/anti-TAA/anti-CD28 trispecific antibody. In some embodiments, the Vβ17×CD28 trispecific antibody is an anti-Vβ17/anti-BCMA/anti-CD28 trispecific antibody. In some embodiments, the Vβ17×CD28 trispecific antibody is an anti-Vβ17/anti-PSMA/anti-CD28 trispecific antibody.

In another general aspect, the invention relates to an isolated humanized Vβ17×CD28 trispecific antibody or antigen-binding fragment thereof.

In certain embodiments, the isolated humanized anti-Vβ17/anti-BCMA/anti-CD28 trispecific antibody or antigen-binding fragment thereof comprises an amino acid sequence with at least 85%, preferably 90%, more preferably 95% or more, such as 95%, 96%, 97%, 98%, or 99% identity to the amino acid sequence of SEQ ID NO:28. In certain embodiments, the humanized Vβ17 monoclonal antibody or antigen-binding fragment thereof comprises the amino acid sequence of SEQ ID NO:28.

In some embodiments, the first binding domain is human. In some embodiments, the second binding domain is human. In some embodiments, the third binding domain is human. In other embodiments, the first binding domain and the second binding domain are human. In other embodiments, the first binding domain and the third binding domain are human. In other embodiments, the second binding domain and the third binding domain are human. In other embodiments, the first binding domain the second binding domain and the third binding domain are human. In some embodiments, the first binding domain is humanized. In some embodiments, the second binding domain is humanized. In some embodiments, the third binding domain is humanized. In other embodiments, the first binding domain and the second binding domain are humanized. In other embodiments, the first binding domain and the third binding domain are humanized. In other embodiments, the second binding domain and the third binding domain are humanized. In other embodiments, the first binding domain the second binding domain and the third binding domain are humanized. In other embodiments, the first binding domain is human and the second binding domain and third binding domain are humanized. In other embodiments, the second binding domain is human and the first binding domain and third binding domain are humanized. In other embodiments, the third binding domain is human and the second binding domain and first binding domain are humanized. In other embodiments, the first binding domain is humanized and the second binding domain and third binding domain are human. In other embodiments, the second binding domain is humanized and the first binding domain and third binding domain are human. In other embodiments, the third binding domain is humanized and the second binding domain and first binding domain are human.

In some embodiments, the trispecific antibody is an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody.

In some embodiments, the trispecific antibody is multivalent. In some embodiments, the trispecific antibody is capable of binding at least five antigens.

In certain embodiments, the trispecific antibodies provided herein are part of a multispecific antibody. In some embodiments, the multispecific antibody comprises a first binding domain that binds to a Vβ17 antigen. In some embodiments, the multispecific antibody comprises a first binding domain that binds to a Vβ17 antigen and comprises a second binding domain that binds to a cancer antigen, as provided herein. In some embodiments, the multispecific antibody comprises a first binding domain that binds to a Vβ17 antigen, a second binding domain that binds to a cancer antigen, and a third binding domain that binds to a CD28 antigen, as provided herein. In some embodiments, the multispecific antibody comprises a first binding domain that binds to a Vβ17 antigen and comprises a second binding domain that binds to a TAA, as provided herein. In some embodiments, the multispecific antibody comprises a first binding domain that binds to a Vβ17 antigen, a second binding domain that binds to a TAA, and a third binding domain that binds to a CD28 antigen, as provided herein. In some embodiments, the multispecific antibody comprises a first binding domain that binds to a Vβ17 antigen and comprises a second binding domain that binds to a BCMA antigen, as provided herein. In some embodiments, the multispecific antibody comprises a first binding domain that binds to a Vβ17 antigen, a second binding domain that binds to a BCMA antigen, and a third binding domain that binds to a CD28 antigen, as provided herein. In some embodiments, the multispecific antibody comprises a first binding domain that binds to a Vβ17 antigen and comprises a second binding domain that binds to a PSMA antigen, as provided herein. In some embodiments, the multispecific antibody comprises a first binding domain that binds to a Vβ17 antigen, a second binding domain that binds to a PSMA antigen, and a third binding domain that binds to a CD28 antigen, as provided herein. In some embodiments of the various antibodies provided herein, the antibody binds to an epitope of a given antigen.

Also provided are isolated nucleic acids encoding the trispecific antibodies or antigen-binding fragments thereof disclosed herein. Also provided are vectors comprising the isolated nucleic acids encoding the trispecific antibodies or antigen-binding fragments thereof disclosed herein. Also provided are host cells comprising the vectors comprising the isolated nucleic acids disclosed herein.

In certain aspects, provided is a nucleic acid encoding an antibody that binds to Vβ17 provided herein. Also provided is a vector comprising a nucleic acid encoding an antibody that binds to Vβ17 provided herein. Also provided is a host cell comprising a vector comprising a nucleic acid encoding an antibody that binds to Vβ17 provided herein. Also provided is a kit comprising the vector comprising a nucleic acid encoding an antibody that binds to Vβ17 provided herein, and packaging for the same. In certain aspects, provided is a nucleic acid encoding an antibody that binds to BCMA provided herein. Also provided is a vector comprising a nucleic acid encoding an antibody that binds to BCMA provided herein. Also provided is a host cell comprising a vector comprising a nucleic acid encoding an antibody that binds to BCMA provided herein. Also provided is a kit comprising the vector comprising a nucleic acid encoding an antibody that binds to BCMA provided herein, and packaging for the same. In certain aspects, provided is a nucleic acid encoding an antibody that binds to PSMA provided herein. Also provided is a vector comprising a nucleic acid encoding an antibody that binds to PSMA provided herein. Also provided is a host cell comprising a vector comprising a nucleic acid encoding an antibody that binds to PSMA provided herein. Also provided is a kit comprising the vector comprising a nucleic acid encoding an antibody that binds to PSMA provided herein, and packaging for the same. In certain aspects, provided is a nucleic acid encoding an antibody that binds to CD28 provided herein. Also provided is a vector comprising a nucleic acid encoding an antibody that binds to CD28 provided herein. Also provided is a host cell comprising a vector comprising a nucleic acid encoding an antibody that binds to CD28 provided herein. Also provided is a kit comprising the vector comprising a nucleic acid encoding an antibody that binds to CD28 provided herein, and packaging for the same.

Also provided is a nucleic acid encoding a trispecific antibody comprising: (a) a first binding domain that binds to Vβ17, (b) a second binding domain that binds to a cancer antigen, and (c) a third binding domain that binds to CD28, as provided herein. Also provided is a vector comprising a nucleic acid encoding a trispecific antibody that binds to Vβ17, a cancer antigen and CD28, provided herein. Also provided is a nucleic acid encoding a trispecific antibody comprising: (a) a first binding domain that binds to Vβ17, (b) a second binding domain that binds to BCMA, and (c) a third binding domain that binds to CD28, as provided herein. Also provided is a vector comprising a nucleic acid encoding a trispecific antibody that binds to Vβ17, BCMA and CD28, provided herein. Also provided is a nucleic acid encoding a trispecific antibody comprising: (a) a first binding domain that binds to Vβ17, (b) a second binding domain that binds to PSMA, and (c) a third binding domain that binds to CD28, as provided herein. Also provided is a vector comprising a nucleic acid encoding a trispecific antibody that binds to Vβ17, PSMA and CD28, provided herein. Also provided is a host cell comprising a vector comprising a nucleic acid encoding the provided trispecific antibody. Also provided is a kit comprising the vector comprising a nucleic acid encoding the provided trispecific antibody, and packaging for the same.

In another general aspect, the invention relates to an isolated nucleic acid encoding a trispecific antibody or antigen-binding fragment thereof disclosed herein. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding trispecific antibodies provided herein can be altered without changing the amino acid sequences of the proteins.

In another general aspect, the invention relates to a vector comprising an isolated nucleic acid encoding a trispecific antibody or antigen-binding fragment thereof disclosed herein. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments provided herein. Such techniques are well known to those skilled in the art in view of the present disclosure.

In another general aspect, the invention relates to a host cell comprising an isolated nucleic acid encoding a trispecific antibody or an antigen-binding fragment thereof provided herein. Any host cell known to those skilled in the art in view of the present disclosure can be used for recombinant expression of antibodies or antigen-binding fragments thereof provided herein. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, provided is a method of producing a trispecific antibody or antigen-binding fragment thereof disclosed herein. The methods comprise culturing a cell comprising a nucleic acid encoding the trispecific antibody or antigen-binding fragment thereof under conditions to produce a trispecific antibody or antigen-binding fragment thereof disclosed herein and recovering the antibody or antigen-binding fragment thereof from the cell or cell culture (e.g., from the supernatant). Expressed antibodies or antigen-binding fragments thereof can be harvested from the cells and purified according to conventional techniques known in the art and as described herein.

Also provided are methods of producing the trispecific antibodies or antigen-binding fragments thereof disclosed herein. The methods can comprise culturing a cell comprising a nucleic acid encoding two heavy chains and two light chains of the trispecific antibody under conditions to produce the heavy and light chains or an antigen-binding fragment thereof, and recovering the heavy and light chains of the trispecific antibody or an antigen-binding fragment thereof from the cell or culture. Following collection of heavy and light chains of the trispecific antibody, the heavy and light chain pairs are mixed in conditions suitable to allow for self-assembly, after which the self-assembled trispecific antibodies are collected.

### Pharmaceutical Compositions

In another general aspect, the invention relates to a pharmaceutical composition comprising the trispecific antibody or antigen-binding fragment thereof provided herein and a pharmaceutically acceptable carrier. Also provided is a pharmaceutical composition comprising an antibody that binds to Vβ17 provided herein, and a pharmaceutically acceptable carrier. Also provided is a pharmaceutical composition comprising an antibody that binds to a cancer antigen provided herein, and a pharmaceutically acceptable carrier. Also provided is a pharmaceutical composition comprising an antibody that binds to BCMA provided herein, and a pharmaceutically acceptable carrier. Also provided is a pharmaceutical composition comprising an antibody that binds to PSMA provided herein, and a pharmaceutically acceptable carrier. Also provided is a pharmaceutical composition comprising an antibody that binds to CD28 provided herein, and a pharmaceutically acceptable carrier. Also provided is a method of producing the pharmaceutical composition, comprising combining the antibody with a pharmaceutically acceptable carrier to obtain the pharmaceutical composition. In another aspect, provided herein is a pharmaceutical composition comprising: (a) a first binding domain that binds to Vβ17, (b) a second binding domain that binds to a cancer antigen, and (c) a third binding domain that binds to CD28, and a pharmaceutically acceptable carrier. In another aspect, provided herein is a pharmaceutical composition comprising: (a) a first binding domain that binds to Vβ17, (b) a second binding domain that binds to BCMA, and (c) a third binding domain that binds to CD28, and a pharmaceutically acceptable carrier In another aspect, provided herein is a pharmaceutical composition comprising: (a) a first binding domain that binds to Vβ17, (b) a second binding domain that binds to PSMA, and (c) a third binding domain that binds to CD28, and a pharmaceutically acceptable carrier. Any of the trispecific antibodies provided herein are contemplated in the pharmaceutical compositions. The term “pharmaceutical composition” as used herein means a product comprising an antibody provided herein together with a pharmaceutically acceptable carrier. Antibodies provided herein and compositions comprising them are also useful in the manufacture of a medicament for therapeutic applications mentioned herein.

Also provided are methods of producing compositions comprising the trispecific antibodies or antigen-binding fragments disclosed herein, such as buffered compositions or purified compositions and the like. For example, the methods may comprise combining the trispecific antibody or antigen-binding fragment thereof with a buffer acceptable that is acceptable for storage and use of the trispecific antibody.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition provided herein. According to particular embodiments, in view of the present disclosure, any pharmaceutically acceptable carrier suitable for use in an antibody pharmaceutical composition can be used herein.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carriers can be used in formulating the pharmaceutical compositions provided herein.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment provided herein, the pH of the formulation is from about 7.0 to about 9.5. In another embodiment provided herein, the pH of the formulation is from about 3.0 to about 7.0.

In another embodiment provided herein, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments provided herein.

In another embodiment provided herein, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments provided herein.

In another embodiment provided herein, the pharmaceutical composition comprises an isotonic agent. Non-limiting examples of isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g. PEG400), and mixtures thereof. Another example of an isotonic agent includes a sugar. Non-limiting examples of sugars can include mono-, di-, or polysaccharides, or water-soluble glycans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethyl-cellulose. Another example of an isotonic agent is a sugar alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative provided herein.

In another embodiment provided herein, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments of the invention.

In another embodiment provided herein, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment provided herein, the pharmaceutical composition comprises a stabilizer, wherein said stabilizer is carboxy-/hydroxycellulose and derivates thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments provided herein.

In further embodiments provided herein, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments provided herein.

In a further embodiment provided herein, the pharmaceutical composition comprises one or more protease inhibitors, such as, e.g., EDTA, and/or benzamidine hydrochloric acid (HCl). The protease inhibitor can be present individually or in the aggregate, in a concentration from about 0.1 mg/ml to about 20 mg/ml. Pharmaceutical compositions comprising each one of these specific protease inhibitors constitute alternative embodiments provided herein.

In another general aspect, the invention relates to a method of producing a pharmaceutical composition comprising a trispecific antibody or antigen-binding fragment thereof disclosed herein, comprising combining a trispecific antibody or antigen-binding fragment thereof with a pharmaceutically acceptable carrier to obtain the pharmaceutical composition.

### Methods of Use

In another aspect, provided herein is a method of activating a T cell expressing Vβ17, comprising contacting the T cell with the trispecific antibody, as provided herein. In another aspect, provided herein is a method of activating a T cell expressing CD28, comprising contacting the T cell with the trispecific antibody, as provided herein. In some embodiments, the contacting results in an increase in cytokine expression, as compared to a control T cell expressing CD28.

In another aspect, provided herein is a method of inactivating a T cell expressing Vβ17, comprising contacting the T cell with the trispecific antibody, as provided herein. In another aspect, provided herein is a method of inactivating a T cell expressing CD28, comprising contacting the T cell with the trispecific antibody, as provided herein.

In another aspect, provided herein is a method of blocking activation of a T cell expressing Vβ17, comprising contacting the T cell with the trispecific antibody, as provided herein. In another aspect, provided herein is a method of blocking activation of a T cell expressing CD28, comprising contacting the T cell with the trispecific antibody, as provided herein.

In another aspect, provided herein is a method of modulating the activation of a T cell expressing Vβ17, comprising contacting the T cell with the trispecific antibody, as provided herein. In another aspect, provided herein is a method of modulating the activation of a T cell expressing CD28, comprising contacting the T cell with the trispecific antibody, as provided herein.

In another aspect, provided herein is a method of directing a T cell expressing Vβ17 to a target cell, the method comprising contacting the T cell with a trispecific antibody provided herein. In some embodiments, the contacting directs the T cell to the target cell. In another aspect, provided herein is a method of directing a T cell expressing CD28 to a target cell, the method comprising contacting the T cell with a trispecific antibody provided herein. In some embodiments, the contacting directs the T cell to the target cell. In another aspect, provided herein is a method of directing a T cell expressing Vβ17 and CD28 to a target cell, the method comprising contacting the T cell with a trispecific antibody provided herein. In some embodiments, the contacting directs the T cell to the target cell.

Also provided is a method of targeting a cancer antigen on the surface of a target cell, the method comprising exposing the target cell to a Vβ17×CD28 trispecific antibody or antigen binding fragment thereof provided herein. Also provided is a method of targeting a cancer antigen on the surface of a target cell, the method comprising exposing the target cell to a pharmaceutical composition comprising a Vβ17×CD28 trispecific antibody or antigen binding fragment thereof provided herein.

In one embodiment, the Vβ17×CD28 trispecific antibody further binds a cancer antigen. In certain embodiments, the cancer antigen is a TAA. In some embodiments, the cancer antigen is BCMA. In some embodiments, the cancer antigen is PSMA. In some embodiments, the target cell is a cancer cell. In some embodiments, the target cell is a B cell. In some embodiments, the target cell is a B cell cancer cell. In some embodiments, the target cell is a prostate cell. In some embodiments, the target cell is a prostate cancer cell.

The functional activity of trispecific antibodies and antigen-binding fragments thereof that bind Vβ17, a cancer antigen and/or CD28 can be characterized by methods known in the art and as described herein. Methods for characterizing antibodies and antigen-binding fragments thereof that bind Vβ17, a cancer antigen and/or CD28 include, but are not limited to, affinity and specificity assays including Biacore, ELISA, and OctetRed analysis; binding assays to detect the binding of antibodies to target cells by FACS; binding assays to detect the binding of antibodies to Vβ17 on T cells, a cancer antigen on target cells, CD28 on target cells and/or CD28 on T cells. According to particular embodiments, the methods for characterizing antibodies and antigen-binding fragments thereof that bind Vβ17, a cancer antigen, and/or CD28 include those described below.

Also provided is a method of directing Vβ17-expressing T cells to a target cell. Also provided is a method of directing CD28-expressing T cells to a target cell. Also provided is a method of directing Vβ17-expressing and CD28-expressing T cells to a target cell. The methods can comprise contacting the Vβ17-expressing and/or CD28-expressing T cell with a Vβ17×CD28 trispecific antibody or antigen binding fragment thereof provided herein, wherein the Vβ17×CD28 trispecific antibody or antigen binding fragment thereof directs the T cell to the target cell.

Also provided is a method for inhibiting growth or proliferation of target cells. The methods can comprise contacting the Vβ17-expressing T cells with a Vβ17×CD28 trispecific antibody or antigen binding fragment thereof provided herein, wherein contacting the target cells with the Vβ17×CD28 trispecific antibody or antigen binding fragment thereof composition inhibits the growth or proliferation of the target cells. The methods can comprise contacting the CD28-expressing T cells with a Vβ17×CD28 trispecific antibody or antigen binding fragment thereof provided herein, wherein contacting the target cells with the Vβ17×CD28 trispecific antibody or antigen binding fragment thereof composition inhibits the growth or proliferation of the target cells. The methods can comprise contacting the Vβ17-expressing and/or CD28-expressing T cells with a Vβ17×CD28 trispecific antibody or antigen binding fragment thereof provided herein, wherein contacting the target cells with the Vβ17×CD28 trispecific antibody or antigen binding fragment thereof composition inhibits the growth or proliferation of the target cells.

In another aspect, provided herein is a method for eliminating target cells in a subject, comprising administering an effective amount of a trispecific antibody, as provided herein, to the subject.

In another general aspect, the invention relates to a method of treating a disease or disorder in a subject in need thereof, comprising administering to the subject a trispecific antibody or antigen binding fragment thereof, or a pharmaceutical composition disclosed herein. In some embodiments, provided is a method for eliminating target cells expressing a cancer antigen or treating a disease caused all or in part by target cells expressing a cancer antigen in a subject, comprising administering an effective amount of a trispecific antibody provided herein to the subject. In some embodiments, the subject is a subject in need thereof. In some embodiments, the subject is a human. In specific embodiments, the trispecific antibody binds Vβ17, CD28 and a cancer antigen.

According to embodiments provided herein, the pharmaceutical composition comprises an effective amount of Vβ17×CD28 trispecific antibody or antigen-binding fragment thereof provided herein.

In one embodiment, the Vβ17×CD28 trispecific antibody further binds a cancer antigen. In certain embodiments, the cancer antigen is a TAA. In some embodiments, the cancer antigen is BCMA. In some embodiments, the cancer antigen is PSMA. In some embodiments, the target cell is a cancer cell. In some embodiments, the target cell is a B cell. In some embodiments, the target cell is a B cell cancer cell. In some embodiments, the target cell is a prostate cell. In some embodiments, the target cell is a prostate cancer cell.

In some embodiments, the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is a adrenal cancer. In some embodiments, the cancer is a anal cancer. In some embodiments, the cancer is an appendix cancer. In some embodiments, the cancer is a bile duct cancer. In some embodiments, the cancer is a bladder cancer. In some embodiments, the cancer is a bone cancer. In some embodiments, the cancer is a brain cancer. In some embodiments, the cancer is a breast cancer. In some embodiments, the cancer is a cervical cancer. In some embodiments, the cancer is a colorectal cancer. In some embodiments, the cancer is a esophageal cancer. In some embodiments, the cancer is a gallbladder cancer. In some embodiments, the cancer is a gestational trophoblastic. In some embodiments, the cancer is a head and neck cancer. In some embodiments, the cancer is a Hodgkin lymphoma. In some embodiments, the cancer is an intestinal cancer. In some embodiments, the cancer is a kidney cancer. In some embodiments, the cancer is a leukemia. In some embodiments, the cancer is a liver cancer. In some embodiments, the cancer is a lung cancer. In some embodiments, the cancer is a melanoma. In some embodiments, the cancer is a mesothelioma. In some embodiments, the cancer is a multiple myeloma. In some embodiments, the cancer is a neuroendocrine tumor. In some embodiments, the cancer is a non-Hodgkin lymphoma. In some embodiments, the cancer is an oral cancer. In some embodiments, the cancer is a ovarian cancer. In some embodiments, the cancer is a pancreatic cancer. In some embodiments, the cancer is a prostate cancer. In some embodiments, the cancer is a sinus cancer. In some embodiments, the cancer is a skin cancer. In some embodiments, the cancer is a soft tissue sarcoma spinal cancer. In some embodiments, the cancer is a stomach cancer. In some embodiments, the cancer is a testicular cancer. In some embodiments, the cancer is a throat cancer. In some embodiments, the cancer is a thyroid cancer. In some embodiments, the cancer is a uterine cancer endometrial cancer. In some embodiments, the cancer is a vaginal cancer. In some embodiments, the cancer is a vulvar cancer.

In some embodiments, the adrenal cancer is an adrenocortical carcinoma (ACC), adrenal cortex cancer, pheochromocytoma, or neuroblastoma. In some embodiments, the anal cancer is a squamous cell carcinoma, cloacogenic carcinoma, adenocarcinoma, basal cell carcinoma, or melanoma. In some embodiments, the appendix cancer is a neuroendocrine tumor (NET), mucinous adenocarcinoma, goblet cell carcinoid, intestinal-type adenocarcinoma, or signet-ring cell adenocarcinoma. In some embodiments, the bile duct cancer is an extrahepatic bile duct cancer, adenocarcinomas, hilar bile duct cancer, perihilar bile duct cancer, distal bile duct cancer, or intrahepatic bile duct cancer. In some embodiments, the bladder cancer is transitional cell carcinoma (TCC), papillary carcinoma, flat carcinoma, squamous cell carcinoma, adenocarcinoma, small-cell carcinoma, or sarcoma. In some embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer. In some embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma. In some embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer. In some embodiments, the cervical cancer is a squamous cell carcinoma, or adenocarcinoma. In some embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma. In some embodiments, the esophageal cancer is an adenocarcinoma or squamous cell carcinoma. In some embodiments, the gall bladder cancer is an adenocarcinoma, papillary adenocarcinoma, adenosquamous carcinoma, squamous cell carcinoma, small cell carcinoma, or sarcoma. In some embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT). In some embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer. In some embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL). In some embodiments, the intestinal cancer is a small intestine cancer, small bowel cancer, adenocarcinoma, sarcoma, gastrointestinal stromal tumors, carcinoid tumors, or lymphoma. In some embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma. In some embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML. In some embodiments, the liver cancer is a hepatocellular carcinoma (HCC), fibrolamellar HCC, cholangiocarcinoma, angiosarcoma, or liver metastasis. In some embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor. In some embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma. In some embodiments, the mesothelioma is a pleural mesothelioma, peritoneal mesothelioma, pericardial mesothelioma, or testicular mesothelioma. In some embodiments, the multiple myeloma is an active myeloma or smoldering myeloma. In some embodiments, the neuroendocrine tumor, is a gastrointestinal neuroendocrine tumor, pancreatic neuroendocrine tumor, or lung neuroendocrine tumor. In some embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma. In some embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer. In some embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst. In some embodiments, the pancreatic cancer is a pancreatic exocrine gland cancer, pancreatic endocrine gland cancer, or pancreatic adenocarcinoma, islet cell tumor, or neuroendocrine tumor. In some embodiments, the prostate cancer is a prostate adenocarcinoma, prostate sarcoma, transitional cell carcinoma, small cell carcinoma, or neuroendocrine tumor. In some embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer. In some embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma. In some embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma. In some embodiments, the spinal cancer is a spinal metastatic tumor. In some embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid. In some embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor. In some embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer. In some embodiments, the thyroid cancer is a papillary carcinoma, follicular carcinoma, Hürthle cell carcinoma, medullary thyroid carcinoma, or anaplastic carcinoma. In some embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma. In some embodiments, the vaginal cancer is a squamous cell carcinoma, adenocarcinoma, melanoma, or sarcoma. In some embodiments, the vulvar cancer is a squamous cell carcinoma or adenocarcinoma.

In some embodiments, the cancer antigen is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PDGFRα, RANKL, SLAMF7, TROP2, VEGF, or VEGF-R. In some embodiments, the cancer antigen is angiopoietin. In some embodiments, the cancer antigen is BCMA. In some embodiments, the cancer antigen is CD19. In some embodiments, the cancer antigen is CD20. In some embodiments, the cancer antigen is CD22. In some embodiments, the cancer antigen is CD25 (IL2-R). In some embodiments, the cancer antigen is CD30. In some embodiments, the cancer antigen is CD33. In some embodiments, the cancer antigen is CD37. In some embodiments, the cancer antigen is CD38. In some embodiments, the cancer antigen is CD52. In some embodiments, the cancer antigen is CD56. In some embodiments, the cancer antigen is CD123 (IL-3R). In some embodiments, the cancer antigen is cMET. In some embodiments, the cancer antigen is DLL/Notch. In some embodiments, the cancer antigen is EGFR. In some embodiments, the cancer antigen is EpCAM. In some embodiments, the cancer antigen is FGF. In some embodiments, the cancer antigen is FGF-R. In some embodiments, the cancer antigen is GD2. In some embodiments, the cancer antigen is HER2. In some embodiments, the cancer antigen is Mesothelin. In some embodiments, the cancer antigen is Nectin-4. In some embodiments, the cancer antigen is PDGFRα. In some embodiments, the cancer antigen is RANKL. In some embodiments, the cancer antigen is SLAMF7. In some embodiments, the cancer antigen is TROP2. In some embodiments, the cancer antigen is VEGF. In some embodiments, the cancer antigen is VEGF-R. In some embodiments, the cancer antigen is PSMA. In some embodiments, the cancer antigen is KLK2.

In some embodiments, the cancer antigen is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, BRCA1, BRCA2, CDK4, CML66, fibronectin, MART-2, p53, Ras, TGF-βRII, or MUC1. In some embodiments, the cancer antigen is CEA. In some embodiments, the cancer antigen is immature laminin receptor. In some embodiments, the cancer antigen is TAG-72. In some embodiments, the cancer antigen is HPV E6. In some embodiments, the cancer antigen is HPV E7. In some embodiments, the cancer antigen is BING-4. In some embodiments, the cancer antigen is calcium-activated chloride channel 2. In some embodiments, the cancer antigen is cyclin-B1. In some embodiments, the cancer antigen is 9D7. In some embodiments, the cancer antigen is EpCAM. In some embodiments, the cancer antigen is EphA3. In some embodiments, the cancer antigen is Her2/neu. In some embodiments, the cancer antigen is telomerase. In some embodiments, the cancer antigen is mesothelin. In some embodiments, the cancer antigen is SAP-1. In some embodiments, the cancer antigen is surviving. In some embodiments, the cancer antigen is a BAGE family antigen. In some embodiments, the cancer antigen is CAGE family antigen. In some embodiments, the cancer antigen is GAGE family antigen. In some embodiments, the cancer antigen is MAGE family antigen. In some embodiments, the cancer antigen is SAGE family antigen. In some embodiments, the cancer antigen is XAGE family antigen. In some embodiments, the cancer antigen is NY-ESO-1/LAGE-1. In some embodiments, the cancer antigen is PRAME. In some embodiments, the cancer antigen is SSX-2. In some embodiments, the cancer antigen is Melan-A. In some embodiments, the cancer antigen is MART-1. In some embodiments, the cancer antigen is Gp100. In some embodiments, the cancer antigen is pmel17. In some embodiments, the cancer antigen is tyrosinase. In some embodiments, the cancer antigen is TRP-1. In some embodiments, the cancer antigen is TRP-2. In some embodiments, the cancer antigen is P. polypeptide. In some embodiments, the cancer antigen is MC1R. In some embodiments, the cancer antigen is prostate-specific antigen. In some embodiments, the cancer antigen is β-catenin. In some embodiments, the cancer antigen is BRCA1. In some embodiments, the cancer antigen is BRCA2. In some embodiments, the cancer antigen is CDK4. In some embodiments, the cancer antigen is CML66. In some embodiments, the cancer antigen is fibronectin. In some embodiments, the cancer antigen is MART-2. In some embodiments, the cancer antigen is p53. In some embodiments, the cancer antigen is Ras. In some embodiments, the cancer antigen is TGF-βRII. In some embodiments, the cancer antigen is MUC1.

Also provided is a trispecific antibody provided herein for use in therapy. Also provided is a trispecific antibody provided herein for use in a method of treating a cancer in a subject. In some embodiments, the cancer is a leukemia. In some embodiments, the cancer is a lymphoma. In certain embodiments, the subject is a subject in need thereof. In a specific embodiment, the subject is a human.

According to embodiments of the invention, the described Vβ17×CD28 trispecific antibody or antigen-binding fragment thereof can be provided in a buffered composition for storage or use. Suitable buffers for the storage of the described Vβ17×CD28 trispecific antibody or antigen-binding fragment thereof would serve to maintain the stability of the antibody or antibody fragment by minimizing deterioration while stored, not promoting aggregation of the antibody or antibody fragment, or minimizing adhesion to the storage vessel.

In another aspect, provided herein is a method of directing a T cell expressing Vβ17 to a cancer cell, the method comprising contacting the T cell with a trispecific antibody provided herein. In some embodiments, the contacting directs the T cell to the cancer cell. In another aspect, provided herein is a method of directing a T cell expressing CD28 to a cancer cell, the method comprising contacting the T cell with a trispecific antibody provided herein. In some embodiments, the contacting directs the T cell to the cancer cell. In another aspect, provided herein is a method of directing a T cell expressing Vβ17 and CD28 to a cancer cell, the method comprising contacting the T cell with a trispecific antibody provided herein. In some embodiments, the contacting directs the T cell to the cancer cell. In specific embodiments, the cancer cell is a cell of a cancer provided herein.

Also provided is a method of targeting cancer antigen on the surface of a cancer cell, the method comprising exposing the cancer cell to an anti-Vβ17/anti-cancer antigen/anti-CD28 trispecific antibody or antigen binding fragment thereof provided herein. Also provided is a method of targeting an antigen on the surface of a cancer cell, the method comprising exposing the cancer cell to a pharmaceutical composition comprising an anti-Vβ17/anti-cancer antigen/anti-CD28 trispecific antibody or antigen binding fragment thereof provided herein.

The functional activity of trispecific antibodies and antigen-binding fragments thereof that bind Vβ17, cancer antigen and/or CD28 can be characterized by methods known in the art and as described herein. Methods for characterizing antibodies and antigen-binding fragments thereof that bind Vβ17, a cancer antigen and/or CD28 include, but are not limited to, affinity and specificity assays including Biacore, ELISA, and OctetRed analysis; binding assays to detect the binding of antibodies to target cells by FACS; binding assays to detect the binding of antibodies to Vβ17 on T cells, cancer antigen on cancer cells, CD28 on cancer cells and/or CD28 on T cells. According to particular embodiments, the methods for characterizing antibodies and antigen-binding fragments thereof that bind Vβ17, an antigen, and/or CD28 include those described below.

Also provided is a method of directing Vβ17-expressing T cells to a cancer cell. Also provided is a method of directing CD28-expressing T cells to a cancer cell. Also provided is a method of directing Vβ17-expressing and CD28-expressing T cells to a cancer cell. The methods can comprise contacting the Vβ17-expressing and/or CD28-expressing T cell with an anti-Vβ17/anti-cancer antigen/anti-CD28 trispecific antibody or antigen binding fragment thereof provided herein, wherein the anti-Vβ17/anti-cancer antigen/anti-CD28 trispecific antibody or antigen binding fragment thereof directs the T cell to the cancer cell.

Also provided is a method for inhibiting growth or proliferation of cancer cells. The methods can comprise contacting the Vβ17-expressing T cells with an anti-Vβ17/anti-cancer antigen/anti-CD28 trispecific antibody or antigen binding fragment thereof provided herein, wherein contacting the cancer cells with the anti-Vβ17/anti-cancer antigen/anti-CD28 trispecific antibody or antigen binding fragment thereof composition inhibits the growth or proliferation of the cancer cells. The methods can comprise contacting the CD28-expressing T cells with an anti-Vβ17/anti-cancer antigen/anti-CD28 trispecific antibody or antigen binding fragment thereof provided herein, wherein contacting the cancer cells with the anti-Vβ17/anti-cancer antigen/anti-CD28 trispecific antibody or antigen binding fragment thereof composition inhibits the growth or proliferation of the cancer cells. The methods can comprise contacting the Vβ17-expressing and/or CD28-expressing T cells with an anti-Vβ17/anti-cancer antigen/anti-CD28 trispecific antibody or antigen binding fragment thereof provided herein, wherein contacting the cancer cells with the anti-Vβ17/anti-cancer antigen/anti-CD28 trispecific antibody or antigen binding fragment thereof composition inhibits the growth or proliferation of the cancer cells.

In another aspect, provided herein is a method for eliminating cancer cells in a subject, comprising administering an effective amount of a trispecific antibody, as provided herein, to the subject.

In another general aspect, the invention relates to a method of treating a disease or disorder in a subject in need thereof, comprising administering to the subject an isolated trispecific antibody or antigen binding fragment thereof, or a pharmaceutical composition disclosed herein. In some embodiments, provided is a method for eliminating cancer cells expressing a cancer antigen or treating a disease caused all or in part by cancer cells expressing a cancer antigen in a subject, comprising administering an effective amount of a trispecific antibody provided herein to the subject. In some embodiments, the subject is a subject in need thereof. In some embodiments, the subject is a human. In specific embodiments, the trispecific antibody binds Vβ17, CD28 and a cancer antigen.

According to embodiments provided herein, the pharmaceutical composition comprises an effective amount of anti-Vβ17/anti-cancer antigen/anti-CD28 trispecific antibody or antigen-binding fragment thereof provided herein.

According to embodiments of the invention, the described anti-Vβ17/anti-cancer antigen/anti-CD28 trispecific antibody or antigen-binding fragment thereof can be provided in a buffered composition for storage or use. Suitable buffers for the storage of the described anti-Vβ17/anti-cancer antigen/anti-CD28 trispecific antibody or antigen-binding fragment thereof would serve to maintain the stability of the antibody or antibody fragment by minimizing deterioration while stored, not promoting aggregation of the antibody or antibody fragment, or minimizing adhesion to the storage vessel.

In certain embodiments, the cancer antigen is a tumor associate antigen. In certain embodiments, the cancer antigen is a tumor specific antigen. In certain embodiments, the cancer antigen is a neoantigen. In certain embodiments, the cancer antigen is a B cell cancer antigen. In certain embodiments, the cancer antigen is a BCMA. In certain embodiments, the cancer antigen is a prostate cancer antigen. In certain embodiments, the cancer antigen is PSMA.

In another aspect, provided herein is a method of directing a T cell expressing Vβ17 to a B cell, the method comprising contacting the T cell with a trispecific antibody provided herein. In some embodiments, the contacting directs the T cell to the B cell. In another aspect, provided herein is a method of directing a T cell expressing CD28 to a B cell, the method comprising contacting the T cell with a trispecific antibody provided herein. In some embodiments, the contacting directs the T cell to the B cell. In another aspect, provided herein is a method of directing a T cell expressing Vβ17 and CD28 to a B cell, the method comprising contacting the T cell with a trispecific antibody provided herein. In some embodiments, the contacting directs the T cell to the B cell.

Also provided is a method of targeting BCMA on the surface of a B cell, the method comprising exposing the B cell to an anti-Vβ17/anti-BCMA/anti-CD28 trispecific antibody or antigen binding fragment thereof provided herein. Also provided is a method of targeting an antigen on the surface of a B cell, the method comprising exposing the B cell to a pharmaceutical composition comprising an anti-Vβ17/anti-BCMA/anti-CD28 trispecific antibody or antigen binding fragment thereof provided herein.

Also provided is a method of targeting CD28 on the surface of a B cell, the method comprising exposing the B cell to an anti-Vβ17/anti-BCMA/anti-CD28 trispecific antibody or antigen binding fragment thereof provided herein. Also provided is a method of targeting an antigen on the surface of a B cell, the method comprising exposing the B cell to a pharmaceutical composition comprising an anti-Vβ17/anti-BCMA/anti-CD28 trispecific antibody or antigen binding fragment thereof provided herein.

The functional activity of trispecific antibodies and antigen-binding fragments thereof that bind Vβ17, BCMA and/or CD28 can be characterized by methods known in the art and as described herein. Methods for characterizing antibodies and antigen-binding fragments thereof that bind Vβ17, BCMA and/or CD28 include, but are not limited to, affinity and specificity assays including Biacore, ELISA, and OctetRed analysis; binding assays to detect the binding of antibodies to target cells by FACS; binding assays to detect the binding of antibodies to Vβ17 on T cells, BCMA on B cells, CD28 on B cells and/or CD28 on T cells. According to particular embodiments, the methods for characterizing antibodies and antigen-binding fragments thereof that bind Vβ17, BCMA, and/or CD28 include those described below.

Also provided is a method of directing Vβ17-expressing T cells to a B cell. Also provided is a method of directing CD28-expressing T cells to a B cell. Also provided is a method of directing Vβ17-expressing and CD28-expressing T cells to a B cell. The methods can comprise contacting the Vβ17-expressing and/or CD28-expressing T cell with an anti-Vβ17/anti-BCMA/anti-CD28 trispecific antibody or antigen binding fragment thereof provided herein, wherein the anti-Vβ17/anti-BCMA/anti-CD28 trispecific antibody or antigen binding fragment thereof directs the T cell to the B cell.

Also provided is a method for inhibiting growth or proliferation of B cells. The methods can comprise contacting the Vβ17-expressing T cells with an anti-Vβ17/anti-BCMA/anti-CD28 trispecific antibody or antigen binding fragment thereof provided herein, wherein contacting the B cells with the anti-Vβ17/anti-BCMA/anti-CD28 trispecific antibody or antigen binding fragment thereof composition inhibits the growth or proliferation of the B cells. The methods can comprise contacting the CD28-expressing T cells with an anti-Vβ17/anti-BCMA/anti-CD28 trispecific antibody or antigen binding fragment thereof provided herein, wherein contacting the B cells with the anti-Vβ17/anti-BCMA/anti-CD28 trispecific antibody or antigen binding fragment thereof composition inhibits the growth or proliferation of the B cells. The methods can comprise contacting the Vβ17-expressing and/or CD28-expressing T cells with an anti-Vβ17/anti-BCMA/anti-CD28 trispecific antibody or antigen binding fragment thereof provided herein, wherein contacting the B cells with the anti-Vβ17/anti-BCMA/anti-CD28 trispecific antibody or antigen binding fragment thereof composition inhibits the growth or proliferation of the B cells.

In another aspect, provided herein is a method for eliminating B cells in a subject, comprising administering an effective amount of a trispecific antibody, as provided herein, to the subject.

In another general aspect, the invention relates to a method of treating a disease or disorder in a subject in need thereof, comprising administering to the subject an isolated trispecific antibody or antigen binding fragment thereof, or a pharmaceutical composition disclosed herein. In some embodiments, provided is a method for eliminating B cells expressing BCMA or treating a disease caused all or in part by B cells expressing BCMA in a subject, comprising administering an effective amount of a trispecific antibody provided herein to the subject. In some embodiments, provided is a method for eliminating B cells expressing CD28 or treating a disease caused all or in part by B cells expressing CD28 in a subject, comprising administering an effective amount of a trispecific antibody provided herein to the subject. In some embodiments, the subject is a subject in need thereof. In some embodiments, the subject is a human. In specific embodiments, the trispecific antibody binds Vβ17, CD28 and BCMA.

According to embodiments provided herein, the pharmaceutical composition comprises an effective amount of anti-Vβ17/anti-BCMA/anti-CD28 trispecific antibody or antigen-binding fragment thereof provided herein.

According to embodiments of the invention, the described anti-Vβ17/anti-BCMA/anti-CD28 trispecific antibody or antigen-binding fragment thereof can be provided in a buffered composition for storage or use. Suitable buffers for the storage of the described anti-Vβ17/anti-BCMA/anti-CD28 trispecific antibody or antigen-binding fragment thereof would serve to maintain the stability of the antibody or antibody fragment by minimizing deterioration while stored, not promoting aggregation of the antibody or antibody fragment, or minimizing adhesion to the storage vessel.

In another aspect, provided herein is a method of directing a T cell expressing Vβ17 to a prostate cell, the method comprising contacting the T cell with a trispecific antibody provided herein. In some embodiments, the contacting directs the T cell to the prostate cell. In another aspect, provided herein is a method of directing a T cell expressing CD28 to a prostate cell, the method comprising contacting the T cell with a trispecific antibody provided herein. In some embodiments, the contacting directs the T cell to the prostate cell. In another aspect, provided herein is a method of directing a T cell expressing Vβ17 and CD28 to a prostate cell, the method comprising contacting the T cell with a trispecific antibody provided herein. In some embodiments, the contacting directs the T cell to the prostate cell. In specific embodiments, the prostate cell is a prostate cancer cell. other PSMA-expressing cells are also contemplated.

Also provided is a method of targeting PSMA on the surface of a prostate cell, the method comprising exposing the prostate cell to an anti-Vβ17/anti-PSMA/anti-CD28 trispecific antibody or antigen binding fragment thereof provided herein. Also provided is a method of targeting an antigen on the surface of a prostate cell, the method comprising exposing the prostate cell to a pharmaceutical composition comprising an anti-Vβ17/anti-PSMA/anti-CD28 trispecific antibody or antigen binding fragment thereof provided herein.

The functional activity of trispecific antibodies and antigen-binding fragments thereof that bind Vβ17, PSMA and/or CD28 can be characterized by methods known in the art and as described herein. Methods for characterizing antibodies and antigen-binding fragments thereof that bind Vβ17, PSMA and/or CD28 include, but are not limited to, affinity and specificity assays including Biacore, ELISA, and OctetRed analysis; binding assays to detect the binding of antibodies to target cells by FACS; binding assays to detect the binding of antibodies to Vβ17 on T cells, PSMA on prostate cells, CD28 on prostate cells and/or CD28 on T cells. According to particular embodiments, the methods for characterizing antibodies and antigen-binding fragments thereof that bind Vβ17, PSMA, and/or CD28 include those described below.

Also provided is a method of directing Vβ17-expressing T cells to a prostate cell. Also provided is a method of directing CD28-expressing T cells to a prostate cell. Also provided is a method of directing Vβ17-expressing and CD28-expressing T cells to a prostate cell. The methods can comprise contacting the Vβ17-expressing and/or CD28-expressing T cell with an anti-Vβ17/anti-PSMA/anti-CD28 trispecific antibody or antigen binding fragment thereof provided herein, wherein the anti-Vβ17/anti-PSMA/anti-CD28 trispecific antibody or antigen binding fragment thereof directs the T cell to the prostate cell.

Also provided is a method for inhibiting growth or proliferation of prostate cells. The methods can comprise contacting the Vβ17-expressing T cells with an anti-Vβ17/anti-PSMA/anti-CD28 trispecific antibody or antigen binding fragment thereof provided herein, wherein contacting the prostate cells with the anti-Vβ17/anti-PSMA/anti-CD28 trispecific antibody or antigen binding fragment thereof composition inhibits the growth or proliferation of the prostate cells. The methods can comprise contacting the CD28-expressing T cells with an anti-Vβ17/anti-PSMA/anti-CD28 trispecific antibody or antigen binding fragment thereof provided herein, wherein contacting the prostate cells with the anti-Vβ17/anti-PSMA/anti-CD28 trispecific antibody or antigen binding fragment thereof composition inhibits the growth or proliferation of the prostate cells. The methods can comprise contacting the Vβ17-expressing and/or CD28-expressing T cells with an anti-Vβ17/anti-PSMA/anti-CD28 trispecific antibody or antigen binding fragment thereof provided herein, wherein contacting the prostate cells with the anti-Vβ17/anti-PSMA/anti-CD28 trispecific antibody or antigen binding fragment thereof composition inhibits the growth or proliferation of the prostate cells.

In another aspect, provided herein is a method for eliminating prostate cells in a subject, comprising administering an effective amount of a trispecific antibody, as provided herein, to the subject.

In another general aspect, the invention relates to a method of treating a disease or disorder in a subject in need thereof, comprising administering to the subject an isolated trispecific antibody or antigen binding fragment thereof, or a pharmaceutical composition disclosed herein. In some embodiments, provided is a method for eliminating prostate cells expressing PSMA or treating a disease caused all or in part by prostate cells expressing PSMA in a subject, comprising administering an effective amount of a trispecific antibody provided herein to the subject. In some embodiments, the subject is a subject in need thereof. In some embodiments, the subject is a human. In specific embodiments, the trispecific antibody binds Vβ17, CD28 and PSMA.

According to embodiments provided herein, the pharmaceutical composition comprises an effective amount of anti-Vβ17/anti-PSMA/anti-CD28 trispecific antibody or antigen-binding fragment thereof provided herein.

According to embodiments of the invention, the described anti-Vβ17/anti-PSMA/anti-CD28 trispecific antibody or antigen-binding fragment thereof can be provided in a buffered composition for storage or use. Suitable buffers for the storage of the described anti-Vβ17/anti-PSMA/anti-CD28 trispecific antibody or antigen-binding fragment thereof would serve to maintain the stability of the antibody or antibody fragment by minimizing deterioration while stored, not promoting aggregation of the antibody or antibody fragment, or minimizing adhesion to the storage vessel.

As used herein, the term “effective amount” refers to an amount of an active ingredient or component that elicits the desired biological or medicinal response in a subject.

According to particular embodiments, an effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

In some embodiments, a Vβ17×CD28 trispecific antibody provided herein is used in combination with a supplemental therapy. In some embodiments, the anti-Vβ17/anti-BCMA/anti-CD28 trispecific antibody provided herein is used in combination with a supplemental therapy. In some embodiments, the anti-Vβ17/anti-PSMA/anti-CD28 trispecific antibody provided herein is used in combination with a supplemental therapy.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

Anti-Vβ17/anti-BCMA/anti-CD28 antibodies provided herein may also be used as agents to detect Vβ17-expressing cells. Thus, in another methods, provided is a method of detecting a cell expressing Vβ17, comprising contacting a cell with a Vβ17 antibody provided herein. Anti-Vβ17/anti-BCMA/anti-CD28 antibodies provided herein may also be used as agents to detect BCMA-expressing cells. Thus, in another methods, provided is a method of detecting a cell expressing BCMA, comprising contacting a cell with a BCMA antibody provided herein. Anti-Vβ17/anti-BCMA/anti-CD28 antibodies provided herein may also be used as agents to detect CD28-expressing cells. Thus, in another methods, provided is a method of detecting a cell expressing CD28, comprising contacting a cell with a CD28 antibody provided herein. In certain embodiments, the detecting is by ELISA. In some embodiments, the detecting is by FACS analysis. Also provided are kits comprising anti-Vβ17/anti-BCMA/anti-CD28 antibody provided herein, and instructions for use.

Anti-Vβ17/anti-PSMA/anti-CD28 antibodies provided herein may also be used as agents to detect Vβ17-expressing cells. Thus, in another methods, provided is a method of detecting a cell expressing Vβ17, comprising contacting a cell with a Vβ17 antibody provided herein. Anti-Vβ17/anti-PSMA/anti-CD28 antibodies provided herein may also be used as agents to detect PSMA-expressing cells. Thus, in another methods, provided is a method of detecting a cell expressing PSMA, comprising contacting a cell with a PSMA antibody provided herein. Anti-Vβ17/anti-PSMA/anti-CD28 antibodies provided herein may also be used as agents to detect CD28-expressing cells. Thus, in another methods, provided is a method of detecting a cell expressing CD28, comprising contacting a cell with a CD28 antibody provided herein. In certain embodiments, the detecting is by ELISA. In some embodiments, the detecting is by FACS analysis. Also provided are kits comprising anti-Vβ17/anti-PSMA/anti-CD28 antibody provided herein, and instructions for use.

Exemplary antibodies are provided herein, as well as the Examples, Tables, Figures and Sequence Listing.

### Embodiments

This invention provides the following non-limiting embodiments.

In a first set of embodiments, provided are:
A1. A trispecific antibody comprising: (a) a first binding domain that binds to Vβ17, (b) a second binding domain that binds to BCMA, and (c) a third binding domain that binds to CD28.
A2. The trispecific antibody of embodiment A1, wherein the first binding domain comprises (i) a VH comprising a VH CDR1 having an amino acid sequence of SEQ ID NO:48, a VH CDR2 having an amino acid sequence of SEQ ID NO:49, and a VH CDR3 having an amino acid sequence of SEQ ID NO:50; and (ii) a VL comprising a VL CDR1 having an amino acid sequence of SEQ ID NO:51, a VL CDR2 having an amino acid sequence of SEQ ID NO:52, and a VL CDR3 having an amino acid sequence of SEQ ID NO:53.
A3. The trispecific antibody of embodiment A2, wherein the antibody comprises:
   a VH having an amino acid sequence of SEQ ID NO:77;
   a VL having an amino acid sequence of SEQ ID NO:78; or
   a VH having an amino acid sequence of SEQ ID NO:77, and a VL having an amino acid sequence of SEQ ID NO:78.
A4. The trispecific antibody of embodiment A1, wherein the first binding domain comprises: (i) a VH comprising a VH CDR1 having an amino acid sequence of SEQ ID NO:48, a VH CDR2 having an amino acid sequence of SEQ ID NO:54, and a VH CDR3 having an amino acid sequence of SEQ ID NO:55; and (ii) a VL comprising a VL CDR1 having an amino acid sequence of SEQ ID NO:56, a VL CDR2 having an amino acid sequence of SEQ ID NO:57, and a VL CDR3 having an amino acid sequence of SEQ ID NO:58.
A5. The trispecific antibody of embodiment A4, wherein the antibody comprises:
   a VH having an amino acid sequence of SEQ ID NO:79;
   a VL having an amino acid sequence of SEQ ID NO:80; or
   a VH having an amino acid sequence of SEQ ID NO:79, and a VL having an amino acid sequence of SEQ ID NO:80.
A6. The trispecific antibody of embodiment A1, wherein the first binding domain comprises (i) a VH comprising a VH CDR1 having an amino acid sequence of SEQ ID NO:59, a VH CDR2 having an amino acid sequence of SEQ ID NO:49, and a VH CDR3 having an amino acid sequence of SEQ ID NO:60; and (ii) a VL comprising a VL CDR1 having an amino acid sequence of SEQ ID NO:61, a VL CDR2 having an amino acid sequence of SEQ ID NO:62, and a VL CDR3 having an amino acid sequence of SEQ ID NO:63.
A7. The trispecific antibody of embodiment A6, wherein the antibody comprises:
   a VH having an amino acid sequence of SEQ ID NO:81;
   a VL having an amino acid sequence of SEQ ID NO: 82; or
   a VH having an amino acid sequence of SEQ ID NO:81, and a VL having an amino acid sequence of SEQ ID NO:82.
A8. The trispecific antibody of embodiment A1, wherein the first binding domain comprises: (i) a VH comprising a VH CDR1 having an amino acid sequence of SEQ ID NO:64, a VH CDR2 having an amino acid sequence of SEQ ID NO:65, and a VH CDR3 having an amino acid sequence of SEQ ID NO:66; and (ii) a VL comprising a VL CDR1 having an amino acid sequence of SEQ ID NO:67, a VL CDR2 having an amino acid sequence of SEQ ID NO:68, and a VL CDR3 having an amino acid sequence of SEQ ID NO:69.
A9. The trispecific antibody of embodiment A8, wherein the antibody comprises:
   a VH having an amino acid sequence of SEQ ID NO:83;
   a VL having an amino acid sequence of SEQ ID NO: 84; or
   a VH having an amino acid sequence of SEQ ID NO: 83, and a VL having an amino acid sequence of SEQ ID NO:84.
A10. The trispecific antibody of embodiment A1, wherein the first binding domain comprises: (i) a VH comprising a VH CDR1 having an amino acid sequence of SEQ ID NO:70, a VH CDR2 having an amino acid sequence of SEQ ID NO:49, and a VH CDR3 having an amino acid sequence of SEQ ID NO:71; and (ii) a VL comprising a VL CDR1 having an amino acid sequence of SEQ ID NO:61, a VL CDR2 having an amino acid sequence of SEQ ID NO:72, and a VL CDR3 having an amino acid sequence of SEQ ID NO:73.
A11. The trispecific antibody of embodiment A10, wherein the antibody comprises:
   a VH having an amino acid sequence of SEQ ID NO:85;
   a VL having an amino acid sequence of SEQ ID NO: 86; or
   a VH having an amino acid sequence of SEQ ID NO: 85, and a VL having an amino acid sequence of SEQ ID NO:86.
A12. The trispecific antibody of embodiment A1, wherein the first binding domain comprises: (i) a VH comprising a VH CDR1 having an amino acid sequence of SEQ ID NO:48, a VH CDR2 having an amino acid sequence of SEQ ID NO:74, and a VH CDR3 having an amino acid sequence of SEQ ID NO:75; and (ii) a VL comprising a VL CDR1 having an amino acid sequence of SEQ ID NO:56, a VL CDR2 having an amino acid sequence of SEQ ID NO:57, and a VL CDR3 having an amino acid sequence of SEQ ID NO:76.
A13. The trispecific antibody of embodiment A12, wherein the antibody comprises:
   a VH having an amino acid sequence of SEQ ID NO:87;
   a VL having an amino acid sequence of SEQ ID NO:88; or
   a VH having an amino acid sequence of SEQ ID NO: 87, and a VL having an amino acid sequence of SEQ ID NO:88.
A14. The trispecific antibody of embodiment A1, wherein the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:666, a VL CDR2 having an amino acid sequence of SEQ ID NO:5, and a VL CDR3 having an amino acid sequence of SEQ ID NO:6.
A15. The trispecific antibody of embodiment A14, wherein the antibody comprises:
   a VH having an amino acid sequence of SEQ ID NO:21;
   a VL having an amino acid sequence of SEQ ID NO:665; or
   a VH having an amino acid sequence of SEQ ID NO:21, and a VL having an amino acid sequence of SEQ ID NO:665.
A16. The trispecific antibody of any one of embodiments A1 to A15, wherein the Vβ17 is present on the surface of a T cell.
A17. The trispecific antibody of any one of embodiments A1 to A16, wherein the second binding domain comprises: (i) a VH comprising a VH CDR1 having an amino acid sequence of SEQ ID NO:89, a VH CDR2 having an amino acid sequence of SEQ ID NO:90, and a VH CDR3 having an amino acid sequence of SEQ ID NO:91; and (ii) a VL comprising a VL CDR1 having an amino acid sequence of SEQ ID NO:92, a VL CDR2 having an amino acid sequence of SEQ ID NO:93, and a VL CDR3 having an amino acid sequence of SEQ ID NO:94.
A18. The trispecific antibody of embodiment A17, wherein the antibody comprises:
   a VH having an amino acid sequence of SEQ ID NO:95;
   a VL having an amino acid sequence of SEQ ID NO:1053; or
   a VH having an amino acid sequence of SEQ ID NO:95, and a VL having an amino acid sequence of SEQ ID NO:1053.
A19. The trispecific antibody of any one of embodiments A1 to A18, wherein the BCMA is present on the surface of a B cell.
A20. The trispecific antibody of any one of embodiments A1 to A19, wherein the second binding domain comprises: (i) a VH comprising a VH CDR1 having an amino acid sequence of SEQ ID NO:702, a VH CDR2 having an amino acid sequence of SEQ ID NO:708, and a VH CDR3 having an amino acid sequence of SEQ ID NO:714; and (ii) a VL comprising a VL CDR1 having an amino acid sequence of SEQ ID NO:794, a VL CDR2 having an amino acid sequence of SEQ ID NO:800, and a VL CDR3 having an amino acid sequence of SEQ ID NO:806.
A21. The trispecific antibody of embodiment A20, wherein the antibody comprises:
   a VH having an amino acid sequence of SEQ ID NO:690;
   a VL having an amino acid sequence of SEQ ID NO:696; or
   a VH having an amino acid sequence of SEQ ID NO:690, and a VL having an amino acid sequence of SEQ ID NO:696.
A22. The trispecific antibody of any one of embodiments A1 to A19, wherein the second binding domain comprises: (i) a VH comprising a VH CDR1 having an amino acid sequence of SEQ ID NO:703, a VH CDR2 having an amino acid sequence of SEQ ID NO:709, and a VH CDR3 having an amino acid sequence of SEQ ID NO:715; and (ii) a VL comprising a VL CDR1 having an amino acid sequence of SEQ ID NO:795, a VL CDR2 having an amino acid sequence of SEQ ID NO:801, and a VL CDR3 having an amino acid sequence of SEQ ID NO:807.
A23. The trispecific antibody of embodiment A22, wherein the antibody comprises:
   a VH having an amino acid sequence of SEQ ID NO:691;
   a VL having an amino acid sequence of SEQ ID NO:697; or
   a VH having an amino acid sequence of SEQ ID NO:691, and a VL having an amino acid sequence of SEQ ID NO:697.
A24. The trispecific antibody of any one of embodiments A1 to A19, wherein the second binding domain comprises: (i) a VH comprising a VH CDR1 having an amino acid sequence of SEQ ID NO:704, a VH CDR2 having an amino acid sequence of SEQ ID NO:710, and a VH CDR3 having an amino acid sequence of SEQ ID NO:716; and (ii) a VL comprising a VL CDR1 having an amino acid sequence of SEQ ID NO:796, a VL CDR2 having an amino acid sequence of SEQ ID NO:802, and a VL CDR3 having an amino acid sequence of SEQ ID NO:808.
A25. The trispecific antibody of embodiment A24, wherein the antibody comprises:
   a VH having an amino acid sequence of SEQ ID NO:692;
   a VL having an amino acid sequence of SEQ ID NO:698; or
   a VH having an amino acid sequence of SEQ ID NO:692, and a VL having an amino acid sequence of SEQ ID NO:698.
A26. The trispecific antibody of any one of embodiments A1 to A19, wherein the second binding domain comprises: (i) a VH comprising a VH CDR1 having an amino acid sequence of SEQ ID NO:705, a VH CDR2 having an amino acid sequence of SEQ ID NO:711, and a VH CDR3 having an amino acid sequence of SEQ ID NO:717; and (ii) a VL comprising a VL CDR1 having an amino acid sequence of SEQ ID NO:797, a VL CDR2 having an amino acid sequence of SEQ ID NO:803, and a VL CDR3 having an amino acid sequence of SEQ ID NO:809.
A27. The trispecific antibody of embodiment A26, wherein the antibody comprises:
   a VH having an amino acid sequence of SEQ ID NO:693;
   a VL having an amino acid sequence of SEQ ID NO:699; or
   a VH having an amino acid sequence of SEQ ID NO:693, and a VL having an amino acid sequence of SEQ ID NO:699.
A28. The trispecific antibody of any one of embodiments A1 to A27, wherein the CD28 is present on the surface of a T cell.
A29. The trispecific antibody of any one of embodiments A1 to A28, wherein the CD28 is present on the surface of a B cell.
A30. The trispecific antibody of any one of embodiments A1 to A29, wherein the first binding domain is humanized, the second binding domain is humanized, the third binding domain is humanized, the first binding domain and second binding domain are humanized, the first binding domain and third binding domain are humanized, the second binding domain and third binding domain are humanized or all three binding domains are humanized.
A31. The trispecific antibody of any one of embodiments A1 to 30, wherein the trispecific antibody is an IgG antibody.
A32. The trispecific antibody of embodiment A31, wherein the IgG antibody is an IgG1, IgG2, IgG3, or IgG4 antibody.
A33. The trispecific antibody of any one of embodiments A1 to A32, wherein the first binding domain binds a Vβ17 antigen.
A34. The trispecific antibody of any one of embodiments A1 to A32, wherein the first binding domain binds a Vβ17 epitope.
A35. The trispecific antibody of any one of embodiments A1 to A32, wherein the VH CDR1, VH CDR2, VH CDR3, VL CDR1, VL CDR2 and VL CDR3 form a binding site for an antigen of the Vβ17.
A36. The trispecific antibody of any one of embodiments A1 to A32, wherein the VH CDR1, VH CDR2, VH CDR3, VL CDR1, VL CDR2 and VL CDR3 form a binding site for an epitope of the Vβ17.
A37. The trispecific antibody of any one of embodiments A1 to A36, wherein the second binding domain binds an antigen of BCMA.
A38. The trispecific antibody of any one of embodiments A1 to A36, wherein the second binding domain binds an epitope of BCMA.
A39. The trispecific antibody of any one of embodiments A1 to A36, wherein the VH CDR1, VH CDR2, VH CDR3, VL CDR1, VL CDR2 and VL CDR3 form a binding site for an antigen of BCMA.
A40. The trispecific antibody of any one of embodiments A1 to A36, wherein the VH CDR1, VH CDR2, VH CDR3, VL CDR1, VL CDR2 and VL CDR3 form a binding site for an epitope of BCMA.
A41. The trispecific antibody of any one of embodiments A1 to A40, wherein the third binding domain binds an antigen of CD28.
A42. The trispecific antibody of any one of embodiments A1 to A40, wherein the third binding domain binds an epitope of CD28.
A43. The trispecific antibody of any one of embodiments A1 to A40, wherein the VH CDR1, VH CDR2, VH CDR3, VL CDR1, VL CDR2 and VL CDR3 form a binding site for an antigen of CD28.
A44. The trispecific antibody of any one of embodiments A1 to A40, wherein the VH CDR1, VH CDR2, VH CDR3, VL CDR1, VL CDR2 and VL CDR3 form a binding site for an epitope of CD28.
A45. The trispecific antibody of any one of embodiments A28 to A44, wherein the B cell is killed when the trispecific antibody binds to the Vβ17 on the surface of the T cell, CD28 on the surface of the T cell, and BCMA on the surface of the B cell.
A46. The trispecific antibody of any one of embodiments A29 to A44, wherein the B cell is killed when the trispecific antibody binds to the Vβ17 on the surface of the T cell, CD28 on the surface of the B cell, and BCMA on the surface of the B cell.
A47. The trispecific antibody of any one of embodiments A45 and A46, wherein the trispecific antibody induces T cell dependent cytotoxicity of the B cell in vitro with an EC₅₀ of less than about 500 pM.
A48. The trispecific antibody of any one of embodiments A45 and A46, wherein the trispecific antibody induces T cell dependent cytotoxicity of the B cell in vitro with an EC₅₀ of less than about 300 pM.
A49. The trispecific antibody of any one of embodiments A45 and A46, wherein the trispecific antibody induces T cell dependent cytotoxicity of the B cell in vitro with an EC₅₀ of less than about 160 pM.
A50. The trispecific antibody of any one of embodiments A47 to A49, wherein the EC₅₀ is assessed with a mixture of effector T cells and target B cells.
A51. The trispecific antibody of embodiment A50, wherein the effector cell to target cell ratio is about 0.01 to 1 to about 10 to 1.
A52. The trispecific antibody of embodiment A50, wherein the effector cell to target cell ratio is about 0.1 to 1 to about 5 to 1.
A53. The trispecific antibody of embodiment A50, wherein the effector cell to target cell ratio is about 1:1.
A54. The trispecific antibody of any one of embodiments A28 to 53, wherein the T cell releases cytokines when the trispecific antibody binds to CD28 on the surface of the T cell.
A55. The trispecific antibody of embodiment A54, wherein the cytokine is a chemokine, interferon, interleukin, or a protein belonging to the tumour necrosis factor superfamily.
A56. The trispecific antibody of embodiment A55, wherein the chemokine is a CC chemokine, CXC chemokine, C chemokine or a CX3C chemokine.
A57. The trispecific antibody of embodiment A55, wherein the interferon is a Type I interferon, Type 2 interferon or a Type 3 interferon.
A58. The trispecific antibody of embodiment A55, wherein the interleukin is an IL-1, IL-2, IL-3, IL-4, IL-5, IL-6, IL-7, IL-8, IL-9, IL-10, IL-11, IL-12, IL-13, IL-15 or an IL-17.
A59. The trispecific antibody of embodiment A55, wherein the protein belonging to the tumour necrosis factor superfamily is lymphotoxin alpha, tumor necrosis factor, lymphotoxin beta, OX40 ligand, CD40 ligand, Fas ligand, CD27 ligand, CD30 ligand, CD137 ligand, TNF-related apoptosis-inducing ligand, receptor activator of nuclear factor kappa-B ligand, TNF-related weak inducer of apoptosis, proliferation-inducing ligand, B-cell activating factor, LIGHT, vascular endothelial growth inhibitor, TNF superfamily member 18, or ectodysplasin A.
A60. The trispecific antibody of any one of embodiments A1 to A59, wherein the trispecific antibody is multivalent.
A61. The trispecific antibody of embodiment A60, wherein the trispecific antibody is capable of binding at least five antigens.
A62. A trispecific antibody comprising: a first means capable of binding Vβ17 on the surface of the T cell; a second means capable of binding BCMA on the surface of the B cell; and a third means capable of binding CD28 on the surface of the T cell.
A63. A trispecific antibody comprising: a first means capable of binding Vβ17 on the surface of the T cell; a second means capable of binding BCMA on the surface of the B cell; and a third means capable of binding CD28 on the surface of the B cell.
A64. The trispecific antibody of any one of embodiments A62 and A63, wherein the first means capable of binding Vβ17 binds a Vβ17 antigen.
A65. The trispecific antibody of any one of embodiments A62 and A63, wherein the first means capable of binding Vβ17 binds a Vβ17 epitope.
A66. The trispecific antibody of any one of embodiments A62 to A65, wherein the second means capable of binding BCMA binds a BCMA antigen.
A67. The trispecific antibody of any one of embodiments A62 to A65, wherein the second means capable of binding BCMA binds a BCMA epitope.
A68. The trispecific antibody of any one of embodiments A62 to A67, wherein the third means capable of binding CD28 binds a CD28 antigen.
A69. The trispecific antibody of any one of embodiments A62 to A67, wherein the third means capable of binding CD28 binds a CD28 epitope.
A70. A nucleic acid encoding the trispecific antibody of any one of embodiments A1 to A69.
A71. A vector comprising the nucleic acid of embodiment A70.
A72. A host cell comprising the vector of embodiment A71.
A73. A kit comprising the vector of embodiment A71 and packaging for the same.
A74. A pharmaceutical composition comprising the trispecific antibody of any one of embodiments A1 to A69, and a pharmaceutically acceptable carrier.
A75. A method of producing the pharmaceutical composition of embodiment A74, comprising combining the antibody with a pharmaceutically acceptable carrier to obtain the pharmaceutical composition.
A76. A method of directing a T cell expressing Vβ17 and CD28 to a B cell, the method comprising contacting the T cell with the trispecific antibody of any one of embodiments A1 to A69, wherein the contacting directs the T cell to the B cell.
A77. A method of inhibiting growth or proliferation of B cells expressing BCMA on the cell surface, the method comprising contacting the B cells with the trispecific antibody of any one of embodiments A1 to A69, wherein contacting the B cells with the pharmaceutical composition or the antibody or the trispecific antibody inhibits growth or proliferation of the B cells.
A78. The method of embodiment A77, wherein the B cells are in the presence of a T cell expressing Vβ17 while in contact with the trispecific antibody.
A79. The method of embodiment A77, wherein the B cells are in the presence of a T cell expressing CD28 while in contact with the trispecific antibody.
A80. A method of directing a T cell expressing Vβ17 to a B cell, the method comprising contacting the T cell with the trispecific antibody of any one of embodiments A1 to A69, wherein the contacting directs the T cell to the B cell.
A81. A method of inhibiting growth or proliferation of B cells expressing CD28 on the cell surface, the method comprising contacting the B cells with the trispecific antibody of any one of embodiments A1 to A69, wherein contacting the B cells with the pharmaceutical composition or the antibody or the trispecific antibody inhibits growth or proliferation of the B cells.
A82. The method of embodiment A81, wherein the B cells are in the presence of a T cell expressing Vβ17 while in contact with the trispecific antibody.
A83. A method for eliminating B cells expressing BCMA in a subject, comprising administering an effective amount of the trispecific antibody of any one of embodiments A1 to A69 to the subject.
A84. A method for eliminating B cells expressing CD28 in a subject, comprising administering an effective amount of the trispecific antibody of any one of embodiments A1 to A69 to the subject.
A85. The method of any one of embodiments A83 and 84, wherein the subject has a cancer.
A86. The method of embodiment A85, wherein the subject has a leukemia.
A87. The method of embodiment A85, wherein the subject has a lymphoma.
A88. A method treating a disease caused all or in part by B cells expressing BCMA in a subject, comprising administering an effective amount of the trispecific antibody of any one of embodiments A1 to A69 to the subject.
A89. A method treating a disease caused all or in part by B cells expressing CD28 in a subject, comprising administering an effective amount of the trispecific antibody of any one of embodiments A1 to A69 to the subject.
A90. The method of any one of embodiments A88 and 89, wherein the disease is cancer.
A91. The method of embodiment A90, wherein the disease is a leukemia.
A92. The method of embodiment A90, wherein the disease is a lymphoma.
A93. The method of any one of embodiments A83 to A92, wherein the subject is a subject in need thereof.
A94. The method of any one of embodiments A83 to A93, wherein the subject is a human.
A95. The trispecific antibody of any one of embodiments A1 to A69 for use in therapy.
A96. The trispecific antibody of any one of embodiments A1 to A69 for use in a method of treating cancer in a subject.
A97. The trispecific antibody for use according to embodiment A96, wherein the cancer is a leukemia.
A98. The trispecific antibody for use according to embodiment A96, wherein the cancer is a lymphoma.
A99. The trispecific antibody for use according to any one of embodiments A96 to A98 wherein the subject is a subject in need thereof.
A100. The trispecific antibody for use according to any one of embodiments A96 to A99, wherein the subject is a human.
A101. A method of activating a T cell expressing Vβ17, comprising contacting the T cell with the trispecific antibody of any one of embodiments A1 to A69.
A102. A method of activating a T cell expressing CD28, comprising contacting the T cell with the trispecific antibody of any one of embodiments A1 to A69.
A103. The method of embodiment A102, wherein the contacting results in an increase in cytokine expression, as compared to a control T cell expressing CD28.
A104. A process for making an antibody that binds to more than one target molecule, the process comprising:
   a step for performing a function of obtaining a binding domain capable of binding to Vβ17 on a T cell;
   a step for performing a function of obtaining a binding domain capable of binding to BCMA on a B cell;
   a step for performing a function of obtaining a binding domain capable of binding to CD28 on a T cell; and
   a step for performing a function of providing an antibody capable of binding to a Vβ17 antigen on the T cell, a BCMA antigen on the B cell and the CD28 antigen on the T cell.
A105. The process of embodiment A104, wherein the step for performing the function of obtaining the binding domain capable of binding to BCMA is repeated n times, wherein the process comprises n steps for performing the function of obtaining the binding domain capable of binding to Vβ17 on the T cell, and wherein the process further comprises n steps for performing the function of obtaining the binding domain capable of binding to CD28 on the T cell, and n number of target molecules, wherein n is at least 2.
A106. The process of embodiment A104, wherein the step for performing the function of obtaining the binding domain capable of binding to CD28 is repeated n times, wherein the process comprises n steps for performing the function of providing the binding domain capable of binding to Vβ17 on the T cell, and wherein the process further comprises n steps for performing the function of obtaining the binding domain capable of binding to BCMA on the B cell, and n number of target molecules, wherein n is at least 2.
A107. A process for making an antibody that binds to more than one target molecule, the process comprising:
   a step for performing a function of obtaining a binding domain capable of binding to Vβ17 on a T cell;
   a step for performing a function of obtaining a binding domain capable of binding to BCMA on a B cell;
   a step for performing a function of obtaining a binding domain capable of binding to CD28 on a B cell; and
   a step for performing a function of providing an antibody capable of binding to the Vβ17 antigen on the T cell, the BCMA antigen on the B cell and the CD28 antigen on the B cell.
A108. The process of embodiment A107, wherein the step for performing the function of obtaining the binding domain capable of binding to BCMA is repeated n times, wherein the process comprises n steps for performing the function of providing the binding domain capable of binding to Vβ17 on the T cell, and wherein the process further comprises n steps for performing the function of obtaining the binding domain capable of binding to CD28 on the B cell, and n number of target molecules, wherein n is at least 2.
A109. The process of embodiment A107, wherein the step for performing the function of obtaining the binding domain capable of binding to CD28 is repeated n times, and wherein the process comprises n steps for performing the function of providing the binding domain capable of binding to Vβ17 on the T cell, and wherein the process further comprises n steps for performing the function of obtaining the binding domain capable of binding to BCMA on the B cell, and n number of target molecules, wherein n is at least 2.
A110. The process of any one of embodiments A104 to A109, wherein the binding domain capable of binding to Vβ17 binds a Vβ17 antigen.
A111. The process of any one of embodiments A104 to A109, wherein the binding domain capable of binding to Vβ17 binds a Vβ17 epitope.
A112. The process of any one of embodiments A104 to A111, wherein the binding domain capable of binding to BCMA binds a BCMA antigen.
A113. The process of any one of embodiments A104 to A111, wherein the binding domain capable of binding to BCMA binds a BCMA epitope.
A114. The process of any one of embodiments A104 to A113, wherein the binding domain capable of binding to CD28 binds a CD28 antigen.
A115. The process of any one of embodiments A104 to A113, wherein the binding domain capable of binding to CD28 binds a CD28 epitope.

In a second set of embodiments, provided are:
B1. A trispecific antibody comprising: (a) a first binding domain that binds to Vβ17, (b) a second binding domain that binds to cancer antigen, and (c) a third binding domain that binds to CD28;
   wherein optionally the cancer antigen is BCMA, or
   wherein optionally the cancer antigen is PSMA.
B2. The trispecific antibody of embodiment B1, wherein the first binding domain that binds to Vβ17 comprises:
   (1) (i) a VH comprising a VH CDR1, a VH CDR2, and a VH CDR3 having an amino acid sequence of a VH CDR1, VH CDR2, and VH CDR3, respectively, of SEQ ID NO:9; and (ii) a VL comprising a VL CDR1, a VL CDR2, and a VL CDR3 having an amino acid sequence of a VL CDR1, VL CDR2, and VL CDR3, respectively, of SEQ ID NO:10;
   (2) (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:22;
   (3) (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:23;
   (4) (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:24;
   (5) (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:22;
   (6) (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:23;
   (7) (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:24;
   (8) (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:22;
   (9) (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:23;
   (10) (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:24;
   (11) (i) a VH comprising a VH CDR1, a VH CDR2, and a VH CDR3 having an amino acid sequence of a VH CDR1, VH CDR2, and VH CDR3, respectively, of SEQ ID NO:46; and (ii) a VL comprising a VL CDR1, a VL CDR2, and a VL CDR3 having an amino acid sequence of a VL CDR1, VL CDR2, and VL CDR3, respectively, of SEQ ID NO:49;
   (12) (i) a VH comprising a VH CDR1, a VH CDR2, and a VH CDR3 having an amino acid sequence of a VH CDR1, VH CDR2, and VH CDR3, respectively, of SEQ ID NO:77; and (ii) a VL comprising a VL CDR1, a VL CDR2, and a VL CDR3 having an amino acid sequence of a VL CDR1, VL CDR2, and VL CDR3, respectively, of SEQ ID NO:78;
   (13) (i) a VH comprising a VH CDR1, a VH CDR2, and a VH CDR3 having an amino acid sequence of a VH CDR1, VH CDR2, and VH CDR3, respectively, of SEQ ID NO:79; and (ii) a VL comprising a VL CDR1, a VL CDR2, and a VL CDR3 having an amino acid sequence of a VL CDR1, VL CDR2, and VL CDR3, respectively, of SEQ ID NO:80. In some embodiments, the first binding domain comprises a VH having an amino acid sequence of SEQ ID NO:79;
   (14) (i) a VH comprising a VH CDR1, a VH CDR2, and a VH CDR3 having an amino acid sequence of a VH CDR1, VH CDR2, and VH CDR3, respectively, of SEQ ID NO:81; and (ii) a VL comprising a VL CDR1, a VL CDR2, and a VL CDR3 having an amino acid sequence of a VL CDR1, VL CDR2, and VL CDR3, respectively, of SEQ ID NO:82;
   (15) (i) a VH comprising a VH CDR1, a VH CDR2, and a VH CDR3 having an amino acid sequence of a VH CDR1, VH CDR2, and VH CDR3, respectively, of SEQ ID NO:83; and (ii) a VL comprising a VL CDR1, a VL CDR2, and a VL CDR3 having an amino acid sequence of a VL CDR1, VL CDR2, and VL CDR3, respectively, of SEQ ID NO:84;
   (16) (1) a VH comprising a VH CDR1, a VH CDR2, and a VH CDR3 having an amino acid sequence of a VH CDR1, VH CDR2, and VH CDR3, respectively, of SEQ ID NO:85; and (ii) a VL comprising a VL CDR1, a VL CDR2, and a VL CDR3 having an amino acid sequence of a VL CDR1, VL CDR2, and VL CDR3, respectively, of SEQ ID NO:86;
   (17) (i) a VH comprising a VH CDR1, a VH CDR2, and a VH CDR3 having an amino acid sequence of a VH CDR1, VH CDR2, and VH CDR3, respectively, of SEQ ID NO:87; and (ii) a VL comprising a VL CDR1, a VL CDR2, and a VL CDR3 having an amino acid sequence of a VL CDR1, VL CDR2, and VL CDR3, respectively, of SEQ ID NO:88;
   (18) (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:665; or
   (19) (i) a VH comprising a VH CDR1, a VH CDR2, and a VH CDR3 having an amino acid sequence of a VH CDR1, VH CDR2, and VH CDR3, respectively, of SEQ ID NO:1084; and (ii) a VL comprising a VL CDR1, a VL CDR2, and a VL CDR3 having an amino acid sequence of a VL CDR1, VL CDR2, and VL CDR3, respectively, of SEQ ID NO:1085.
B3. The trispecific antibody of embodiment B2, wherein
   i. the VH CDR1, VH CDR2, VH CDR3, VL CDR1, VL CDR2, and VL CDR3 amino acid sequences of the first binding domain are according to the Kabat numbering system;
   ii. the VH CDR1, VH CDR2, VH CDR3, VL CDR1, VL CDR2, and VL CDR3 amino acid sequences of the first binding domain are according to the Chothia numbering system;
   iii. the VH CDR1, VH CDR2, VH CDR3, VL CDR1, VL CDR2, and VL CDR3 amino acid sequences of the first binding domain are according to the AbM numbering system;
   iv. the VH CDR1, VH CDR2, VH CDR3, VL CDR1, VL CDR2, and VL CDR3 amino acid sequences of the first binding domain are according to the Contact numbering system;
   v. the VH CDR1, VH CDR2, VH CDR3, VL CDR1, VL CDR2, and VL CDR3 amino acid sequences of the first binding domain are according to the IMGT numbering system; or
   vi. the VH CDR1, VH CDR2, VH CDR3, VL CDR1, VL CDR2, and VL CDR3 amino acid sequences of the first binding domain are according to the Exemplary numbering system.
B4. The trispecific antibody of any one of embodiments B1 to B3, wherein the first binding domain binds to Vβ17 that is present on the surface of a T cell.
B5. The trispecific antibody of any one of embodiments B1 to B4, wherein the cancer antigen is present on the surface of a cell.
B6. The trispecific antibody of any one of embodiments B1 to B5, wherein the cancer antigen is BCMA.
B7. The trispecific antibody of embodiment B6, wherein the BCMA is present on the surface of a B cell.
B8. The trispecific antibody of embodiment B6 or B7, wherein the second binding domain that binds to BCMA comprises:
   (1) (i) a VH comprising a VH CDR1, a VH CDR2, and a VH CDR3 having an amino acid sequence of a VH CDR1, VH CDR2, and VH CDR3, respectively, of SEQ ID NO:95; and (ii) a VL comprising a VL CDR1, a VL CDR2, and a VL CDR3 having an amino acid sequence of a VL CDR1, VL CDR2, and VL CDR3, respectively, of SEQ ID NO:96; or
   (2) (i) a VH comprising a VH CDR1, a VH CDR2, and a VH CDR3 having an amino acid sequence of a VH CDR1, VH CDR2, and VH CDR3, respectively, of SEQ ID NO:1052; and (ii) a VL comprising a VL CDR1, a VL CDR2, and a VL CDR3 having an amino acid sequence of a VL CDR1, VL CDR2, and VL CDR3, respectively, of SEQ ID NO:1053.
B9. The trispecific antibody of embodiment B8, wherein
   i. the VH CDR1, VH CDR2, VH CDR3, VL CDR1, VL CDR2, and VL CDR3 amino acid sequences of the second binding domain are according to the Kabat numbering system;
   ii. the VH CDR1, VH CDR2, VH CDR3, VL CDR1, VL CDR2, and VL CDR3 amino acid sequences of the second binding domain are according to the Chothia numbering system;
   iii. the VH CDR1, VH CDR2, VH CDR3, VL CDR1, VL CDR2, and VL CDR3 amino acid sequences of the second binding domain are according to the AbM numbering system;
   iv. the VH CDR1, VH CDR2, VH CDR3, VL CDR1, VL CDR2, and VL CDR3 amino acid sequences of the second binding domain are according to the Contact numbering system;
   v. the VH CDR1, VH CDR2, VH CDR3, VL CDR1, VL CDR2, and VL CDR3 amino acid sequences of the second binding domain are according to the IMGT numbering system; or
   vi. the VH CDR1, VH CDR2, VH CDR3, VL CDR1, VL CDR2, and VL CDR3 amino acid sequences of the second binding domain are according to the Exemplary numbering system.
B10. The trispecific antibody of any one of embodiments B1 to B5, wherein the cancer antigen is PSMA.
B11. The trispecific antibody of embodiment B10, wherein the PSMA is present on the surface of a prostate cell.
B12. The trispecific antibody of embodiment B10 or B11, wherein the second binding domain that binds to PSMA comprises:
   (i) a VH comprising a VH CDR1, a VH CDR2, and a VH CDR3 having an amino acid sequence of a VH CDR1, VH CDR2, and VH CDR3, respectively, of SEQ ID NO:1046; and (ii) a VL comprising a VL CDR1, a VL CDR2, and a VL CDR3 having an amino acid sequence of a VL CDR1, VL CDR2, and VL CDR3, respectively, of SEQ ID NO:1047.
B13. The trispecific antibody of embodiment B8, wherein
   i. the VH CDR1, VH CDR2, VH CDR3, VL CDR1, VL CDR2, and VL CDR3 amino acid sequences of the second binding domain are according to the Kabat numbering system;
   ii. the VH CDR1, VH CDR2, VH CDR3, VL CDR1, VL CDR2, and VL CDR3 amino acid sequences of the second binding domain are according to the Chothia numbering system;
   iii. the VH CDR1, VH CDR2, VH CDR3, VL CDR1, VL CDR2, and VL CDR3 amino acid sequences of the second binding domain are according to the AbM numbering system;
   iv. the VH CDR1, VH CDR2, VH CDR3, VL CDR1, VL CDR2, and VL CDR3 amino acid sequences of the second binding domain are according to the Contact numbering system;
   v. the VH CDR1, VH CDR2, VH CDR3, VL CDR1, VL CDR2, and VL CDR3 amino acid sequences of the second binding domain are according to the IMGT numbering system; or
   vi. the VH CDR1, VH CDR2, VH CDR3, VL CDR1, VL CDR2, and VL CDR3 amino acid sequences of the second binding domain are according to the Exemplary numbering system.
B14. The trispecific antibody of any one of embodiments B1 to B13, wherein the third binding domain binds to CD28 that is present on the surface of a T cell.
B15. The trispecific antibody of any one of embodiments B1 to B14, wherein the third binding domain that binds to CD28 comprises:
   (1) (i) a VH comprising a VH CDR1, a VH CDR2, and a VH CDR3 having an amino acid sequence of a VH CDR1, VH CDR2, and VH CDR3, respectively, of SEQ ID NO:690; and (ii) a VL comprising a VL CDR1, a VL CDR2, and a VL CDR3 having an amino acid sequence of a VL CDR1, VL CDR2, and VL CDR3, respectively, of SEQ ID NO:696;
   (2) (i) a VH comprising a VH CDR1, a VH CDR2, and a VH CDR3 having an amino acid sequence of a VH CDR1, VH CDR2, and VH CDR3, respectively, of SEQ ID NO:691; and (ii) a VL comprising a VL CDR1, a VL CDR2, and a VL CDR3 having an amino acid sequence of a VL CDR1, VL CDR2, and VL CDR3, respectively, of SEQ ID NO:697;
   (3) (i) a VH comprising a VH CDR1, a VH CDR2, and a VH CDR3 having an amino acid sequence of a VH CDR1, VH CDR2, and VH CDR3, respectively, of SEQ ID NO:692; and (ii) a VL comprising a VL CDR1, a VL CDR2, and a VL CDR3 having an amino acid sequence of a VL CDR1, VL CDR2, and VL CDR3, respectively, of SEQ ID NO:698; or
   (4) (i) a VH comprising a VH CDR1, a VH CDR2, and a VH CDR3 having an amino acid sequence of a VH CDR1, VH CDR2, and VH CDR3, respectively, of SEQ ID NO:693; and (ii) a VL comprising a VL CDR1, a VL CDR2, and a VL CDR3 having an amino acid sequence of a VL CDR1, VL CDR2, and VL CDR3, respectively, of SEQ ID NO:699.
B16. The trispecific antibody of embodiment B15, wherein
   i. the VH CDR1, VH CDR2, VH CDR3, VL CDR1, VL CDR2, and VL CDR3 amino acid sequences of the third binding domain are according to the Kabat numbering system;
   ii. the VH CDR1, VH CDR2, VH CDR3, VL CDR1, VL CDR2, and VL CDR3 amino acid sequences of the third binding domain are according to the Chothia numbering system;
   iii. the VH CDR1, VH CDR2, VH CDR3, VL CDR1, VL CDR2, and VL CDR3 amino acid sequences of the third binding domain are according to the AbM numbering system;
   iv. the VH CDR1, VH CDR2, VH CDR3, VL CDR1, VL CDR2, and VL CDR3 amino acid sequences of the third binding domain are according to the Contact numbering system;
   v. the VH CDR1, VH CDR2, VH CDR3, VL CDR1, VL CDR2, and VL CDR3 amino acid sequences of the third binding domain are according to the IMGT numbering system; or
   vi. the VH CDR1, VH CDR2, VH CDR3, VL CDR1, VL CDR2, and VL CDR3 amino acid sequences of the third binding domain are according to the Exemplary numbering system.
B17. The trispecific antibody of any one of embodiments B1 to B16, wherein
   i. the first binding domain is humanized,
   ii. the second binding domain is humanized,
   iii. the third binding domain is humanized,
   iv. the first binding domain and second binding domain are humanized,
   v. the first binding domain and third binding domain are humanized,
   vi. the second binding domain and third binding domain are humanized, or
   vii. the first binding domain, the second binding domain and the third binding domain are humanized.
B18. The trispecific antibody of any one of embodiments B1 to B17, wherein the trispecific antibody is an IgG antibody.
B19. The trispecific antibody of embodiment B18, wherein the IgG antibody is an IgG1, IgG2, IgG3, or IgG4 antibody.
B20. The trispecific antibody of embodiment B18 or B19, wherein the antibody comprises a kappa light chain.
B21. The trispecific antibody of embodiment B18 or B19, wherein the antibody comprises a lambda light chain.
B22. The trispecific antibody of any one of embodiments B1 to B21, wherein the first binding domain binds a Vβ17 antigen.
B23. The trispecific antibody of any one of embodiments B1 to B21, wherein the first binding domain binds a Vβ17 epitope.
B24. The trispecific antibody of any one of embodiments B1 to B21, wherein the VH CDR1, VH CDR2, VH CDR3, VL CDR1, VL CDR2 and VL CDR3 form a binding site for an antigen of the Vβ17.
B25. The trispecific antibody of any one of embodiments B1 to B21, wherein the VH CDR1, VH CDR2, VH CDR3, VL CDR1, VL CDR2 and VL CDR3 form a binding site for an epitope of the Vβ17.
B26. The trispecific antibody of any one of embodiments B1 to B25, wherein the first binding domain specifically binds to Vβ17.
B27. The trispecific antibody of any one of embodiments B1 to B26, wherein the second binding domain binds an antigen of BCMA.
B28. The trispecific antibody of any one of embodiments B1 to B26, wherein the second binding domain binds an epitope of BCMA.
B29. The trispecific antibody of any one of embodiments B1 to B26, wherein the VH CDR1, VH CDR2, VH CDR3, VL CDR1, VL CDR2 and VL CDR3 form a binding site for an antigen of BCMA.
B30. The trispecific antibody of any one of embodiments B1 to B26, wherein the VH CDR1, VH CDR2, VH CDR3, VL CDR1, VL CDR2 and VL CDR3 form a binding site for an epitope of BCMA.
B31. The trispecific antibody of any one of embodiments B27 to B30, wherein the second binding domain specifically binds to BCMA.
B32. The trispecific antibody of any one of embodiments B1 to B26, wherein the second binding domain binds an antigen of PSMA.
B33. The trispecific antibody of any one of embodiments B1 to B26, wherein the second binding domain binds an epitope of PSMA.
B34. The trispecific antibody of any one of embodiments B1 to B26, wherein the VH CDR1, VH CDR2, VH CDR3, VL CDR1, VL CDR2 and VL CDR3 form a binding site for an antigen of PSMA.
B35. The trispecific antibody of any one of embodiments B1 to B26, wherein the VH CDR1, VH CDR2, VH CDR3, VL CDR1, VL CDR2 and VL CDR3 form a binding site for an epitope of PSMA.
B36. The trispecific antibody of any one of embodiments B32 to B35, wherein the second binding domain specifically binds to PSMA.
B37. The trispecific antibody of any one of embodiments B1 to B36, wherein the third binding domain binds an antigen of CD28.
B38. The trispecific antibody of any one of embodiments B1 to B36, wherein the third binding domain binds an epitope of CD28.
B39. The trispecific antibody of any one of embodiments B1 to B36, wherein the VH CDR1, VH CDR2, VH CDR3, VL CDR1, VL CDR2 and VL CDR3 form a binding site for an antigen of CD28.
B40. The trispecific antibody of any one of embodiments B1 to B36, wherein the VH CDR1, VH CDR2, VH CDR3, VL CDR1, VL CDR2 and VL CDR3 form a binding site for an epitope of CD28.
B41. The trispecific antibody of any one of embodiments B1 to B40, wherein the third binding domain specifically binds to CD28.
B42. The trispecific antibody of any one of embodiments B1 to B35, wherein the trispecific antibody is multivalent.
B43. A trispecific antibody comprising: a first means capable of binding Vβ17 on the surface of a T cell; a second means capable of binding a cancer antigen on the surface of a cancer cell; and a third means capable of binding CD28 on the surface of the T cell.
B44. The trispecific antibody of embodiment B43, wherein the cancer antigen is BCMA and the cancer cell is a B cell cancer cell.
B45. The trispecific antibody of embodiment B43, wherein the cancer antigen is PSMA and the cancer cell is a prostate cancer cell.
B46. A nucleic acid encoding the trispecific antibody of any one of embodiments B1 to B45.
B47. A vector comprising the nucleic acid of embodiment B46.
B48. A host cell comprising the vector of embodiment B47.
B49. A kit comprising the vector of embodiment B47 and packaging for the same.
B50. A kit comprising the trispecific antibody of any one of embodiments B1 to B45 and packaging for the same.
B51. A pharmaceutical composition comprising the trispecific antibody of any one of embodiments B1 to B45, and a pharmaceutically acceptable carrier.
B52. A method of producing the pharmaceutical composition of embodiment B51, comprising combining the trispecific antibody with a pharmaceutically acceptable carrier to obtain the pharmaceutical composition.
B53. A method of activating a T cell expressing Vβ17, comprising contacting the T cell with the trispecific antibody of any one of embodiments B1 to B45.
B54. A process for making an antibody that binds to more than one target molecule, the process comprising:
   a step for performing a function of obtaining a first binding domain capable of binding to Vβ17 on a T cell;
   a step for performing a function of obtaining a second binding domain capable of binding to cancer antigen on a cancer cell;
   a step for performing a function of obtaining a third binding domain capable of binding to CD28 on a T cell; and
   a step for performing a function of providing an antibody capable of binding to a Vβ17 antigen on the T cell, a cancer antigen on the cancer cell and the CD28 antigen on the T cell.
B55. The process of embodiment B54, wherein (i) the step for performing a function of obtaining a second binding domain that binds to the cancer antigen on the cancer cell is repeated n times, and further comprising n steps for performing a function of providing a first binding domain that binds to Vβ17 present on a T cell and n number of target molecules, wherein n is at least 2, or (ii) the step for performing a function of obtaining a third binding domain that binds to the CD28 on the T cell is repeated n times, and further comprising n steps for performing a function of providing a first binding domain that binds to Vβ17 present on a T cell and n number of target molecules, wherein n is at least 2.
B56. A method of directing a T cell expressing Vβ17 and CD28 to a B cell, the method comprising contacting the T cell with the trispecific antibody of any one of embodiments B1 to B45, wherein the contacting directs the T cell to the B cell.
B57. A method of inhibiting growth or proliferation of B cells expressing BCMA on the cell surface, the method comprising contacting the B cells with the trispecific antibody of any one of embodiments B1 to B45, wherein contacting the B cells with the pharmaceutical composition or the antibody or the trispecific antibody inhibits growth or proliferation of the B cells.
B58. The method of embodiment B57, wherein the B cells are in the presence of a T cell expressing Vβ17 while in contact with the trispecific antibody.
B59. The method of embodiment B57, wherein the B cells are in the presence of a T cell expressing CD28 while in contact with the trispecific antibody.
B60. A method of directing a T cell expressing Vβ17 to a cancer cell, the method comprising contacting the T cell with the trispecific antibody of any one of embodiments B1 to B45, wherein the contacting directs the T cell to the cancer cell.
B61. A method of inhibiting the growth or proliferation of a cancer cell, comprising contacting the trispecific antibody of any one of embodiments B1 to B45 with the cancer cell having the cancer antigen present on the surface of the cancer cell, wherein the contacting is in the presence of a T cell expressing the Vβ17, and wherein the contacting results in the inhibition of the growth or proliferation of the cancer cell.
B62. A method of eliminating a cancer cell in a subject, comprising contacting the trispecific antibody of any one of embodiments B1 to B45 with the cancer cell having the cancer antigen present on the surface of the cancer cell, wherein the contacting is in the presence of a T cell expressing the Vβ17, and wherein the contacting results in the elimination of the cancer cell.
B63. A method of treating a disease in a subject, comprising administering an effective amount of the trispecific antibody of any one of embodiments B1 to B45 to the subject, wherein the disease is caused all or in part by a cancer cell having the cancer antigen present on the surface of the cancer cell.
B64. The method of embodiment B62 or B63, wherein the subject is a human.
B65. The method of any one of embodiments B62 to B64, wherein the subject is a subject in need thereof.
B66. The trispecific antibody of any one of embodiments B1 to B45 or the method of any one of embodiments B56 to B65, wherein the cancer antigen is present on the surface of a cancer cell.
B67. The trispecific antibody or method of embodiment B66, wherein
   (i) the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer;
   (ii) cancer antigen is an angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PAP, PDGFRα, PSA, PSA3, PSMA, RANKL, SLAMF7, STEAPI, TARP, TROP2, VEGF, or VEGF-R antigen; and/or
   (iii) the cancer antigen is a CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, or BRCA1 antigen.
B68. The trispecific antibody or method embodiment B67, wherein
   (i) the adrenal cancer is an adrenocortical carcinoma (ACC), adrenal cortex cancer, pheochromocytoma, or neuroblastoma;
   (ii) the anal cancer is a squamous cell carcinoma, cloacogenic carcinoma, adenocarcinoma, basal cell carcinoma, or melanoma;
   (iii) the appendix cancer is a neuroendocrine tumor (NET), mucinous adenocarcinoma, goblet cell carcinoid, intestinal-type adenocarcinoma, or signet-ring cell adenocarcinoma;
   (iv) the bile duct cancer is an extrahepatic bile duct cancer, adenocarcinomas, hilar bile duct cancer, perihilar bile duct cancer, distal bile duct cancer, or intrahepatic bile duct cancer;
   (v) the bladder cancer is transitional cell carcinoma (TCC), papillary carcinoma, flat carcinoma, squamous cell carcinoma, adenocarcinoma, small-cell carcinoma, or sarcoma;
   (vi) the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer;
   (vii) the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma;
   (viii) the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer;
   (ix) the cervical cancer is a squamous cell carcinoma, or adenocarcinoma;
   (x) the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma;
   (xi) the esophageal cancer is an adenocarcinoma or squamous cell carcinoma;
   (xii) the gall bladder cancer is an adenocarcinoma, papillary adenocarcinoma, adenosquamous carcinoma, squamous cell carcinoma, small cell carcinoma, or sarcoma;
   (xiii) the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT);
   (xiv) the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer;
   (xv) the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL);
   (xvi) the intestinal cancer is a small intestine cancer, small bowel cancer, adenocarcinoma, sarcoma, gastrointestinal stromal tumors, carcinoid tumors, or lymphoma;
   (xvii) the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma;
   (xviii) the leukemia is an acute lymphocytic leukemia (ALL), acute myeloid leukemia (AMIL), chronic lymphocytic leukemia (CLL), chronic myeloid leukemia (CML), hairy cell leukemia (HCL), or a myelodysplastic syndrome (MDS);
   (xix) the liver cancer is a hepatocellular carcinoma (HCC), fibrolamellar HCC, cholangiocarcinoma, angiosarcoma, or liver metastasis;
   (xx) the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor;
   (xxi) the melanoma is a superficial spreading melanoma, nodular melanoma, acral-lentiginous melanoma, lentigo maligna melanoma, amelanotic melanoma, desmoplastic melanoma, ocular melanoma, or metastatic melanoma;
   (xxii) the mesothelioma is a pleural mesothelioma, peritoneal mesothelioma, pericardial mesothelioma, or testicular mesothelioma;
   (xxiii) the multiple myeloma is an active myeloma or smoldering myeloma;
   (xxiv) the neuroendocrine tumor, is a gastrointestinal neuroendocrine tumor, pancreatic neuroendocrine tumor, or lung neuroendocrine tumor;
   (xxv) the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma;
   (xxvi) the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer;
   (xxvii) the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst;
   (xxviii) the pancreatic cancer is a pancreatic exocrine gland cancer, pancreatic endocrine gland cancer, or pancreatic adenocarcinoma, islet cell tumor, or neuroendocrine tumor;
   (xxix) the prostate cancer is a prostate adenocarcinoma, prostate sarcoma, transitional cell carcinoma, small cell carcinoma, or neuroendocrine tumor;
   (xxx) the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer;
   (xxxi) the skin cancer is a basal cell carcinoma, squamous cell carcinoma, melanoma, Merkel cell carcinoma, Kaposi sarcoma (KS), actinic keratosis, skin lymphoma, or keratoacanthoma;
   (xxxii) the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma;
   (xxxiii) the spinal cancer is a spinal metastatic tumor;
   (xxxiv) the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid;
   (xxxv) the testicular cancer is a seminoma, non-seminoma, embryonal carcinoma, yolk sac carcinoma, choriocarcinoma, teratoma, gonadal stromal tumor, leydig cell tumor, or sertoli cell tumor;
   (xxxiv) the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer;
   (xxxv) the thyroid cancer is a papillary carcinoma, follicular carcinoma, Hürthle cell carcinoma, medullary thyroid carcinoma, or anaplastic carcinoma;
   (xxxvi) the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma;
   (xxxvii) the vaginal cancer is a squamous cell carcinoma, adenocarcinoma, melanoma, or sarcoma; or
   (xxxviii) the vulvar cancer is a squamous cell carcinoma or adenocarcinoma.
B69. The trispecific antibody or method of embodiment B66, wherein the cancer antigen is BCMA.
B70. The trispecific antibody or method of embodiment B69, wherein the cancer cell is a B cell.
B71. The trispecific antibody or method of embodiment B69 or B70, wherein the cancer is a lymphoma.
B72. The trispecific antibody or method of embodiment B69 or B70, wherein the cancer is a leukemia.
B73. The trispecific antibody or method of embodiment B66, wherein the cancer antigen is PSMA.
B74. The trispecific antibody or method of embodiment B69, wherein the cancer cell is a prostate cancer cell.
B75. The trispecific antibody or method of embodiment B69 or B70, wherein the cancer is a prostate cancer.

Representative amino acid sequences of trispecific antibodies provided herein are shown in the Tables provided in the Examples section and the sequence listing and are contemplated as certain embodiments. In addition, representative nucleic acid sequences encoding antibodies provided herein are shown in the Tables provided in the Examples section and the sequence listing and are contemplated as certain embodiments.

Also provided in the Examples herein are exemplary multi-specific (trispecific) antibodies that bind to Vβ17, CD28 and BCMA. These Examples are illustrative of additional exemplary trispecific antibodies that can effectively target a variety of cells and tissues in a subject. In some embodiments, provided herein is a trispecific antibody comprising: (a) a first binding domain that binds to a Vβ17 antigen, (b) a second binding domain that binds to BCMA, and (c) a third binding domain that binds to CD28.

Exemplary binding agents that bind to Vβ17, exemplary binding agents that bind to CD28, as well as exemplary binding agents that bind to BCMA are provided elsewhere herein.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### EXAMPLES

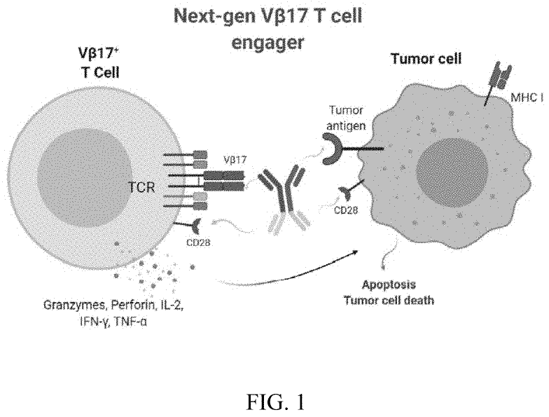

The following examples are based on the premise that T cells demonstrate potent anti-tumor functions. These cells express TCR-haplotype-Vβ17 and majority of these cells exhibit efficient cytotoxicity of tumor target cells. This ability is then harnessed using trispecific antibodies constructed such that one arm binds to the Vβ17 structure, the other arm binds to CD28 on the T cells and the third arm binds BCMA on B cells, often in B-cell-mediated tumor cells. Thus, the trispecific antibody bridges the effector and target cells together-resulting in tumor killing. This mechanism of action is described in the schematic outlined in FIG. 1.

### Example 1: Human Framework Adaptation of Anti-Vβ17 MAB E17.5F

The mouse IgG1 anti-human T cell receptor Vβ17 clone E17.5F was commercially sourced. Sample preparation and LC/MSMS analysis were performed at Protea Bioscience Inc. (Morgantown, W. Va.). The sample was reduced and alkylated, divided into seven aliquots, and proteolytically digested with Trypsin/LysC, Chymotrypsin, LysC, Pepsin, and AspN, Elastase, and Proteinase K enzymes. Resulting peptides were desalted using a ZIPTIP C18 Pipette Tips and separated on-line using reverse phase chromatography. Mass spectrometry was performed on Thermo Q-EXACTIVE spectrometer using HCD fragmentation. MS data sets were analyzed using PEAKS software by matching de novo sequence tags to an IMGT-based antibody sequences database. Gaps in the sequence were assigned using Contig sequence assembly of de novo identified peptides. All CDRs and hyper-mutations were confirmed by inspecting the MS/MS spectra

The sequences obtained are shown in Tables 1 and 2.

TABLE 1
CDR Sequences of TCR Vβ17 clone E17.5F.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| E17.5F | GYSITSGYFWN | 1 | YISYDGSNN | 2 | PSPGTGYAVDY | 3 |
| | | SEQ | | SEQ | | SEQ |
| | | ID | | ID | | ID |
| Antibody | LCDR1 | NO: | LCDR2 | NO: | LCDR3 | NO: |
| E17.5F | RSSQSLVHSNG | 4 | KVSNRFS | 5 | SQSTHVPFT | 6 |
| | NTYLH | | | | | |

TABLE 2
Heavy chain and light chain sequences of TCR Vb17 clone E17.5F.

| | | SEQ |
|---|---|---|
| mAb | | ID |
| ID | Heavy Chain Amino Acid Sequence | NO: |
| B17B01 | NVQLQESGPGLVKPSQSLSLTCSVAGYSITSGYFWNWIRQFPGNKLEWMGYIS | 7 |
| | YDGSNNYNPSLKNRISITRDTSKNQFFLKLNSVTTEDTATYYCASPSPGTGYAV | |
| | DYWGQGTSVTVSSAKTTPPSVYPLAPGSAAQTNSMVTLGCLVKGYFPEPVTV | |
| | TWNSGSLSSGVHTFPAVLQSDLYTLSSSVTVPSSTWPSQTVTCNVAHPASSTKV | |
| | DKKIVPRDCGCKPCICTVPEVSSVFIFPPKPKDVLTITLTPKVTCVVVDISKDDPE | |
| | VQFSWFVDDVEVHTAQTKPREEQINSTFRSVSELPIMHQDWLNGKEFKCRVNS | |
| | AAFPAPIEKTISKTYGRPKAPQVYTIPPPKEQMAKDKVSLTCMITNFFPEDITVE | |
| | WQWNGQPAENYKNTQPIMDTDGSYFVYSKLNVQKSNWEAGNTFTCSVLHEG | |
| | LHNHHTEKSLSHSPGK | |
| | | SEQ |
| | | ID |
| | Light Chain Amino Acid Sequence | NO: |
| B17B01 | NVVMTQTPLSLPVSLGDQASISCRSSQSLVHSNGNTYLHWYLQKPGQSPKFLIY | 8 |
| | KVSNRFSGVPDRFSGGGSGTEFTLKISRVEAEDLGVYFCSQSTHVPFTFGSGTK | |
| | LEIKRADAAPTVSIFPPSSEQLTSGGASVVCFLNNFYPKDINVKWKIDGSERQN | |
| | GVLNSWTDQDSKDSTYSMSSTLTLTKDEYERHNSYTCEATHKTSTSPIVKSFN | |
| | RNEC | |

Changes were made in the sequences for the preparation of bispecific antibodies (Table 3). The changes include the following: (1) a framework mutation Asn1 of the heavy chain was not conserved, so the sequence has been modified to have the DVQLW sequence; (2) another mutation identified in the Fc, K337Y, was deemed uncharacteristic, and, thus, a construct without this mutation was synthesized; and (3) a potential secondary glycosylation site on the heavy chain was observed, and, thus, two versions of this mAb with and without the N-linked site

TABLE 3
Heavy and Light Chain sequences for Vβ17 clone E17.5F
antibody variants

| | | SEQ |
|---|---|---|
| mAb | | ID |
| ID | Heavy Chain Amino Acid Sequence | NO: |
| B17B1 | NVQLQESGPGLVKPSQSLSLTCSVAGYSITSGYFWNWIRQFPGNKLEWMGYIS | 9 |
| | YDGSNNYNPSLKNRISITRDTSKNQFFLKLNSVTTEDTATYYCASPSPGTGYAV | |
| | DYWGQGTSVTVSSASTKGPSVFPLAPCSRSTSESTAALGCLVKDYFPEPVTVS | |
| | WNSGALTSGVHTFPAVLQSSGLYSLSSVVTVPSSSLGTKTYTCNVDHKPSNTK | |
| | VDKRVESKYGPPCPPCPAPEAAGGPSVFLFPPKPKDTLMISRTPEVTCVVVDVS | |
| | QEDPEVQFNWYVDGVEVHNAKTKPREEQFNSTYRVVSVLTVLHQDWLNGKE | |
| | YKCKVSNKGLPSSIEKTISKAKGQPREPQVYTLPPSQEEMTKNQVSLTCLVKGF | |
| | YPSDIAVEWESNGQPENNYKTTPPVLDSDGSFLLYSKLTVDKSRWQEGNVFSC | |
| | SVMHEALHNHYTQKSLSLSLGK | |
| B17B2 | DVQLKESGPGLVKPSQSLSVTCSVTGYSITSGYYWNWYRQFPGNKLEWMGYI | 11 |
| | SYDGSNNYNPSLKNRISITRDTSKNQILLKLTYVTTEDTATYYCTRPSPGTGYA | |
| | VDYWGQGTLVTVSSASTKGPSVFPLAPCSRSTSESTAALGCLVKDYFPEPVTVS | |
| | WNSGALTSGVHTFPAVLQSSGLYSLSSVVTVPSSSLGTKTYTCNVDHKPSNTK | |
| | VDKRVESKYGPPCPPCPAPEAAGGPSVFLFPPKPKDTLMISRTPEVTCVVVDVS | |
| | QEDPEVQFNWYVDGVEVHNAKTKPREEQFNSTYRVVSVLTVLHQDWLNGKE | |
| | YKCKVSNKGLPSSIEKTISKAKGQPREPQVYTLPPSQEEMTKNQVSLTCLVKGF | |
| | YPSDIAVEWESNGQPENNYKTTPPVLDSDGSFLLYSKLTVDKSRWQEGNVFSC | |
| | SVMHEALHNHYTQKSLSLSLGK | |
| | | SEQ |
| | | ID |
| | Light Chain Amino Acid Sequence | NO: |
| B17B1 | NVVMTQTPLSLPVSLGDQASISCRSSQSLVHSNGNTYLHWYLQKPGQSPKFLIY | 10 |
| | KVSNRFSGVPDRFSGGGSGTEFTLKISRVEAEDLGVYFCSQSTHVPFTFGSGTK | |
| | LEIKRADAAPTVSIFPPSSEQLTSGGASVVCFLNNFYPKDINVKWKIDGSERQN | |
| | GVLNSWTDQDSKDSTYSMSSTLTLTKDEYERHNSYTCEATHKTSTSPIVKSFN | |
| | RNEC | |
| B17B2 | DIVMTQSPDSLAVSLGERATINCRSSQSLVHSNGNTYLHWYQQKPGQPPKLLI | 12 |
| | YKVSNRFSGVPDRFSGSGSGTDFTLTISSLQAEDVAVYYCSQSTHVPFTFGQGT | |
| | KVEIKRADAAPTVSIFPPSSEQLTSGGASVVCFLNNFYPKDINVKWKIDGSERQ | |
| | NGVLNSWTDQDSKDSTYSMSSTLTLTKDEYERHNSYTCEATHKTSTSPIVKSF | |
| | NRNEC | |

The two antibodies (B17B1 and B17B2) were expressed in HEK293Expi cells. The supernatants were tested for Vβ17 binding (B17B1 and B17B2) and only B17B1 demonstrated binding. Thus, B17B1 was expressed having an IgG4 constant region with Fc substitutions.

The anti-human TCR Vβ17 mouse mAb B17B1 was humanized using the Human Framework Adaptation (HFA) method (Fransson J, et al. J. Mol. Biol. 2010; 398:214-231). To find the best combination of humanized heavy and light chains, several human V-region sequences were selected for testing (Table 4). Selection of human germlines was based solely on the overall sequence similarity to the mouse antibody in the framework (FR) region. Neither the CDR sequences, nor their length or canonical structures, were considered in this selection.

The CDR definition used in HFA is described in (Fransson J, et al. J. Mol. Biol. 2010; 398:214-231) and corresponds to the Martin's definition (Abhinandan K R and Martin A C. Mol. Immunol. 2008; 45:3832-3839). The CDRs (Table 1) were defined as described below (using the Chothia numbering scheme [Chothia C, and Lesk A. J. Mol. Biol. 1987; 196:901-917]):

| | |
|---|---|
| HCDR1 (SEQ ID NO: 1) | 26-35 |
| HCDR2 (SEQ ID NO: 2) | 50-58 |
| HCDR3 (SEQ ID NO: 3) | 95-102 |
| LCDR1 (SEQ ID NO: 4) | 24-34 |
| LCDR2 (SEQ ID NO: 5) | 50-56 |
| LCDR3 (SEQ ID NO: 6) | 89-97 |

The selected human germlines are provided in Table 4 (in the IMGT notation).

TABLE 4
VH and VL variants

| | | SEQ ID |
|---|---|---|
| | Sequence | NO: |
| Ab VH | | |
| B17H1 | NVQLQESGPGLVKPSQSLSLTCSVAGYSITSGYFWNWIRQFP | 25 |
| | GNKLEWMGYISYDGSNNYNPSLKNRISITRDTSKNQFFLKL | |
| | NSVTTEDTATYYCASPSPGTGYAVDYWGQGTSVTVSS | |
| B17H3 | EVQLLESGGGLVQPGGSLRLSCAASGYSITSGYFWNWVRQ | 19 |
| | APGKGLEWVSYISYDGSNNYADSVKGRFTISRDNSKNTLYL | |
| | QMNSLRAEDTAVYYCAKPSPGTGYAVDYWGQGTLVTVSS | |
| B17H4 | EVQLLESGGGLVQPGGSLRLSCAASGYSITSGYFWNWVRQ | 20 |
| | APGKGLEWVSYISYDGSNNYADSVKGRFTISRDNSKNTLYL | |
| | QMNSLRAEDTAVYYCASPSPGTGYAVDYWGQGTLVTVSS | |
| B17H5 | QVQLQESGPGLVKPSETLSLTCTVSGYSITSGYFWNWIRQPP | 21 |
| | GKGLEWIGYISYDGSNNYNPSLKSRVTISRDTSKNQFSLKLS | |
| | SVTAADTAVYYCASPSPGTGYAVDYWGQGTLVTVSS | |
| Ab VL | | |
| B17L1 | NVVMTQTPLSLPVSLGDQASISCRSSQSLVHSNGNTYLHWY | 26 |
| | LQKPGQSPKFLIYKVSNRFSPGVPDRFSGGGSGTEFTLKISRVE | |
| | AEDLGVYFCSQSTHVPFTFGSGTKLEIK | |
| B17L3 | DIQMTQSPSSLSASVGDRVTITCRSSQSLVHSNGNTYLHWY | 22 |
| | QQKPGKAPKLLIYKVSNRFSGVPSRFSGSGSGTDFTLTISSLQ | |
| | PEDFATYYCSQSTHVPFTFGQGTKLEIK | |
| B17L4 | DIQMTQSPSSLSASVGDRVTITCRSSQSLVHSNGNTYLHWY | 23 |
| | QQKPGKAPKFLIYKVSNRFSGVPSRFSGSGSGTDFTLTISSLQ | |
| | PEDFATYYCSQSTHVPFTFGQGTKLEIK | |
| B17L5 | DVVMTQSPLSLPVTLGQPASISCRSSQSLVHSNGNTYLHWF | 24 |
| | QQRPGQSPRFLIYKVSNRFSGVPDRFSGSGSGTDFTLKISRVE | |
| | AEDVGVYYCSQSTHVPFTFGQGTKLEIK | |

CDRs1-3 are underlined

“Back mutations” in several variants were introduced at FR positions that are known to be important for VL/VH pairing and CDR conformation. The selected human germlines are provided in Table 5 (in the IMGT notation), with the back mutations noted.

TABLE 5
The selected J-regions

| | | SEQ ID |
|---|---|---|
| J-region | Sequence | NO: |
| IGHJ1*01 HC | WGQGTLVTVSS | 42 |
| IGKJ2*01 LC | FGQGTKLEIK | 43 |

Amino acid sequences of all nine pairwise combinations of three heavy chains and three light chains were back-translated to DNA, and cDNA was prepared using gene synthesis techniques (U.S. Pat. Nos. 6,670,127; 6,521,427). Heavy chain (HC) variable regions were subcloned onto human IgG4 constant region using an in-house expression vector with the CMV promoter using standard molecular biology techniques. Light chain (LC) variable regions were subcloned onto a human Lambda (λ) constant regions using an in-house expression vector with the CMV promoter using standard molecular biology techniques. Resulting plasmids were transfected into HEK EXPI cells (LifeTechnologies; Carlsbad, Calif.) and mAbs were expressed. Purification was by standard methods using a Protein A column (HITRAP MABSELECT SURE column). After elution, the pools were dialyzed into D-PBS, pH 7.2.

TABLE 6
Heavy and Light chains of nine humanized Vβ17 antibodies

| | | | | | Concentration |
|---|---|---|---|---|---|
| mAh | Hc | SEQ ID NO: | Lc | SEQ ID NO: | (μg/mL) |
| B17B14 | B17H3 | 19 | B17L3 | 22 | 686.3 |
| B17B15 | B17H3 | 19 | B17L4 | 23 | 13.8 |
| B17B16 | B17H3 | 19 | B17L5 | 24 | 14.6 |
| B17B17 | B17H4 | 20 | B17L3 | 22 | 335.1 |
| B17B18 | B17H4 | 20 | B17L4 | 23 | 45.2 |
| B17B19 | B17H4 | 20 | B17L5 | 24 | 27.5 |
| B17B20 | B17H5 | 21 | B17L3 | 22 | 602.1 |
| B17B21 | B17H5 | 21 | B17L4 | 23 | 570.9 |
| B17B22 | B17H5 | 21 | B17L5 | 24 | 320.5 |

The humanized antibodies were screened for binding to a TCRVβ17 (SEQ ID NO:27)/Va10.2-Fc (SEQ ID NO:44) fusion protein by ELISA. Biotinylated TCRVβ17/Va10.2-Fc fusion protein was added to a streptavidin-coated ELISA plate. Unbound protein was washed away and mAb was added at a range of concentrations (0.01-10 μg/mL). Plates were washed and anti-kappa:HRP detection antibody was added. Plates were washed, chemiluminescent detection reagent was added, and the plates were read on a Perkin Elmer ENVISION plate reader for luminescence. B17B20 and B17B21 showed positive binding to the TCR-Vβ17 protein. B17B22 showed weak binding to this protein. These antibodies were then purified as described above for further studies. B17B21 demonstrated the best binding to recombinant TCR-Vβ17 protein and to M1-stimulated T-cells and was thus chosen as the molecule for further functional studies, specifically T-cell re-directed cancer cell killing as a bispecific antibody.

Thus, the variable region sequence of B17B21 (anti-Vβ17) and I3RB217 (anti-CD123 antibody) was used to generate a bispecific antibody to be tested for T-cell re-directed killing of acute myeloid leukemia (AML) cells.

### Example 2—Multispecific Antibodies that Bind Vβ17 and BCMA

### Example 2.1: Anti-Vβ17 Antibody Generation

Immunogen.

A recombinant human TCR Vβ17×Vα10.2 fused to a human Fc was used as an immunogen, and the sequence is listed in Table 7.

TABLE 7
Amino acid sequence of the immunogen.

| | | | SEQ |
|---|---|---|---|
| | | | ID |
| | Protein ID | “Knob” arm and “hole” arm amino acid sequence | NO: |
| TCR | Vβ17-TRBC2- | VDGGITQSPKYLFRKEGQNVTLSCEQNLNHDAMYWYRQDPGQGLR | 560 |
| Vβ17 × | hFc | LIYYSQIVNDFQKGDIAEGYSVSREKKESFPLTVTSAQKNPTAFY | |
| Vα10.2 | | LCASSSRSSYEQYFGPGTRLTVTEDLKNVFPPEVAVFEPSEAEIS | |
| fused to | | HTQKATLVCLATGFYPDHVELSWWVNGKEVHSGVSTDPQPLKEQP | |
| human Fc | | ALNDSRYSLSSRLRVSATFWQNPRNHFRCQVQFYGLSENDEWTQD | |
| | | RAKPVTQIVSAEAWGRADEPKSCDKTHTCPPCPAPELLGGPSVFL | |
| | | FPPKPKDTLMISRTPEVTCVVVDVSHEDPEVKFNWYVDGVEVHNA | |
| | | KTKPREEQYNSTYRVVSVLTVLHQDWLNGKEYKCKVSNKALPAPI | |
| | | EKTISKAKGQPREPQVYVLPPSREEMTKNQVSLLCLVKGFYPSDI | |
| | | AVEWESNGQPENNYLTWPPVLDSDGSFFLYSKLTVDKSRWQQGNV | |
| | | FSCSVMHEALHNHYTQKSLSLSPGK | |
| | Vα10.2-TRAC- | QLLEQSPQFLSIQEGENLTVYCNSSSVFSSLQWYRQEPGEGPVLL | 561 |
| | hFc | VTVVTGGEVKKLKRLTFQFGDARKDSSLHITAAQPGDTGLYLCAG | |
| | | AGSQGNLIFGKGTKLSVKPNIQNPDPAVYQLRDSKSSDKSVCLFT | |
| | | DFDSQTNVSQSKDSDVYITDKTVLDMRSMDFKSNSAVAWSNKSDF | |
| | | ACANAFNNSIIPEDTFFPSEPKSCDKTHTCPPCPAPELLGGPSVF | |
| | | LFPPKPKDTLMISRTPEVTCVVVDVSHEDPEVKFNWYVDGVEVHN | |
| | | AKTKPREEQYNSTYRVVSVLTVLHQDWLNGKEYKCKVSNKALPAP | |
| | | IEKTISKAKGQPREPQVYVYPPSREEMTKNQVSLTCLVKGFYPSD | |
| | | IAVEWESNGQPENNYKTTPPVLDSDGSFALVSKLTVDKSRWQQGN | |
| | | VFSCSVMHEALHNHYTQKSLSLSPGK | |

Protein Production of the Immunogen.

Expression plasmids encoding the immunogen (see Table 7) were transfected into CHO cell at a DNA ratio of 1:1. Total amount of DNA for a 750 mL expression scale was 750 ug. Final expression volume was 1 L after two feedings and enhancer additions. Using an AKTAprime plus instrument (GE Healthcare Life Sciences), supernatant (1 L) after 7 days was applied with a flow-rate of 5 mL/min to a MAB SELECT SURE (GE Life Sciences) with a column volume (CV) of 10 mL pre-equilibrated with Phosphate buffered saline (PBS), pH 6.8. Non-specific proteins binding to the column material was washed off with PBS supplemented with 500 mM NaCl, pH 6.8 (5 CV). The Fc containing the immunogen was eluted stepwise with 40 mM sodium acetate pH 5.0 (5 CV), pH 4.5 (5 CV), pH 4.0 (10 CV), pH 3.5 (5 CV), and pH 3.0 (5 CV). Majority of the target protein eluted at the pH 4.0 step. Fractions were pooled, and applied (5 mL) at a flow-rate of 0.2 mL/min on to a HILOAD 16/600 SUPERDEX (GE Healthcare) column pre-equilibrated with PBS (pH 6.8). Target protein was eluted, pooled, and analyzed by SDS-PAGE, analytic SEC, intact mass by MS. Purity estimated to 99.5%.

Immunization in Mouse and Screening of Vβ17 Binder.

Wild type mouse with 6 different MHC combinations was immunized using rapid immunization protocol. Eight mice were selected for cell fusion based on serum titer. Hybridoma supernatants were screened by LUMINEX using the immunogen and expanded Vβ17+ T cells. Hits were V-region recovered and formatted directly into bispecific antibodies.

### Example 2.2: Production and De Novo Sequencing of Anti-BCMA MAB

An anti-BCMA clone was obtained and sequenced. The three VH CDR and three VL CDR sequences of anti-BCMA (BCMB519) are shown in Table 8 (SEQ ID NOs:89-94, respectively); and the VH and VL sequences of the anti-BCMA antibody are shown in Table 9 (SEQ ID NOs:95 and 96, respectively).

TABLE 8
CDR sequences of anti-BCMA mAb.

| | | SEQ | | SEQ | | SEQ |
|---|---|---|---|---|---|---|
| | | ID | | ID | | ID |
| mAb ID | HCDR1 | NO: | HCDR2 | NO: | HCDR3 | NO: |
| BCMB519 | GFTFSSYA | 89 | ISGSGGST | 90 | AKDEGYSSGHYYGMDV | 91 |
| | | SEQ | | SEQ | | SEQ |
| | | ID | | ID | | ID |
| mAb ID | LCDR1 | NO: | LCDR2 | NO: | LCDR3 | NO: |
| BCMB519 | QSISSSF | 92 | GAS | 93 | QHYGSSPMYT | 94 |

TABLE 9
VH and VL sequences of anti-BCMA mAb.

| | | SEQ |
|---|---|---|
| | | ID |
| mAb ID | VH Amino Acid Sequence | NO: |
| BCMB519 | EVQLLESGGGLVQPGGSLRLSCAASGFTFSSYAMSWVRQAPGKGLE | 95 |
| | WVSAISGSGGSTYYADSVKGRFTISRDNSKNTLYLQMNSLRAEDTAV | |
| | YYCAKDEGYSSGHYYGMDVWGQGTTVTVSS | |
| | | SEQ |
| | | ID |
| mAb ID | VL Amino Acid Sequence | NO: |
| BCMB519 | EIVLTQSPGTLSLSPGERATLSCRASQSISSSFLTWYQQKPGQAPRLLIY | 96 |
| | GASSRATGIPDRFSGGGSGTDFTLTISRLEPEDFAVYYCQHYGSSPMYT | |
| | FGQGTKLEIK | |

### Example 2.3: Preparation of Anti-Vβ17/Anti-BCMA Bispecific Antibodies

All the bispecific antibodies were produced as full-length antibodies in the knob-into-hole format as human IgG1, as previously described (Atwell et al. J. Mol. Biol. 270: 26-35, 1997). Nucleic acid sequences encoding variable regions were subcloned into a custom mammalian expression vectors containing constant region of IgG1 Fc silent expression cassettes using standard PCR restriction enzyme based cloning techniques. The bispecific antibodies were expressed by transient transfection in Chinese hamster ovary cell line.

The sequences of the bispecific antibodies expressed in the CHO cells are shown in Table 10 below.

TABLE 10
Sequences of antibodies expressed in CHO cells

| | | | SEQ |
|---|---|---|---|
| Bispecific | | | ID |
| Antibody | Chain info | Amino Acid Sequence | NO: |
| Vb17 × | Heavy Chain 1 | MAWVWTLLFLMAAAQSIQAQVQLKESGPGLVAPSQSLSITCT | 562 |
| BCMA | Vb17_202B4D1 | VSGFSLTSYGVIIWVRQPPGKGLEWLGIIWAGGGTNYNSALMS | |
| (B17B622) | | RLSITKDNSKSQVSLKMNSLQTDDTAMYYCARGTFFNYDYFD | |
| | | YWGQGTTLTVSSASTKGPSVFPLAPSSKSTSGGTAALGCLVKD | |
| | | YFPEPVTVSWNSGALTSGVHTFPAVLQSSGLYSLSSVVTVPSSS | |
| | | LGTQTYICNVNHKPSNTKVDKKVEPKSCDKTHTCPPCPAPEAA | |
| | | GGPSVFLFPPKPKDTLMISRTPEVTCVVVSVSHEDPEVKFNWY | |
| | | VDGVEVHNAKTKPREEQYNSTYRVVSVLTVLHQDWLNGKEY | |
| | | KCKVSNKALPAPIEKTISKAKGQPREPQVYVYPPSREEMTKNQ | |
| | | VSLTCLVKGFYPSDIAVEWESNGQPENNYKTTPPVLDSDGSFA | |
| | | LVSKLTVDKSRWQQGNVFSCSVMHEALHNRFTQKSLSLSPG | |
| | Light Chain 1 | MAWVWTLLFLMAAAQSIQADIVMTQSQKFMSTSVGDRVSITC | 563 |
| | Vb17_202B4D1 | KASQDVGTDVAWYQQKPGQSPKLMIYWASTRHTGVPDRFTG | |
| | | SGSGTDFTLTISNVQSEDLADYFCQQYSRYPWTFGAGTKLELK | |
| | | RTVAAPSVFIFPPSDEQLKSGTASVVCLLNNFYPREAKVQWKV | |
| | | DNALQSGNSQESVTEQDSKDSTYSLSSTLTLSKADYEKHKVYA | |
| | | CEVTHQGLSSPVTKSFNRGEC | |
| | Heavy Chain 2 | MAWVWTLLFLMAAAQSIQAEIVLTQSPGTLSLSPGERATLSCR | 559 |
| | BCMB519 | ASQSISSSFLTWYQQKPGQAPRLLIYGASSRATGIPDRFSGGGS | |
| | | GTDFTLTISRLEPEDFAVYYCQHYGSSPMYTFGQGTKLEIKGGS | |
| | | EGKSSGSGSESKSTGGSEVQLLESGGGLVQPGGSLRLSCAASGF | |
| | | TFSSYAMSWVRQAPGKGLEWVSAISGSGGSTYYADSVKGRFTI | |
| | | SRDNSKNTLYLQMNSLRAEDTAVYYCAKDEGYSSGHYYGMD | |
| | | VWGQGTTVTVSSEPKSSDKTHTCPPCPAPEAAGGPSVFLFPPKP | |
| | | KDTLMISRTPEVTCVVVSVSHEDPEVKFNWYVDGVEVHNAKT | |
| | | KPREEQYNSTYRVVSVLTVLHQDWLNGKEYKCKVSNKALPAP | |
| | | IEKTISKAKGQPREPQVYVLPPSREEMTKNQVSLLCLVKGFYPS | |
| | | DIAVEWESNGQPENNYLTWPPVLDSDGSFFLYSKLTVDKSRW | |
| | | QQGNVFSCSVMHEALHNHYTQKSLSLSPG | |
| Vb17 × | Heavy Chain 1 | MAWVWTLLFLMAAAQSIQAQVQLKESGPVLVAPSQSLSITCT | 564 |
| BCMA | Vb17_210E10A1 | VSGFSLTSYGVHWVRQPPGKGLEWLGIIWAGGNTNSNSALMS | |
| (B17B624) | | RLSISKDNSKSQVFLKMNSLQTDDTAMYYCARGSFYSYLYFDY | |
| | | WGQGTTLTVSSASTKGPSVFPLAPSSKSTSGGTAALGCLVKDY | |
| | | FPEPVTVSWNSGALTSGVHTFPAVLQSSGLYSLSSVVTVPSSSL | |
| | | GTQTYICNVNHKPSNTKVDKKVEPKSCDKTHTCPPCPAPEAAG | |
| | | GPSVFLFPPKPKDTLMISRTPEVTCVVVSVSHEDPEVKFNWYV | |
| | | DGVEVHNAKTKPREEQYNSTYRVVSVLTVLHQDWLNGKEYK | |
| | | CKVSNKALPAPIEKTISKAKGQPREPQVYVYPPSREEMTKNQV | |
| | | SLTCLVKGFYPSDIAVEWESNGQPENNYKTTPPVLDSDGSFAL | |
| | | VSKLTVDKSRWQQGNVFSCSVMHEALHNRFTQKSLSLSPG | |
| | Light Chain 1 | MAWVWTLLFLMAAAQSIQADIVMTQSPSYLAASPGETITINCR | 565 |
| | Vb17_210E10A1 | ASKSISKYLAWYQEKPGKTNKLLIYSGSTLQSGIPSRFSGSGSGT | |
| | | DFTLTISSLEPEDFAMYYCQQHNDYPLTFGAGTKLELKRTVAA | |
| | | PSVFIFPPSDEQLKSGTASVVCLLNNFYPREAKVQWKVDNALQ | |
| | | SGNSQESVTEQDSKDSTYSLSSTLTLSKADYEKHKVYACEVTH | |
| | | QGLSSPVTKSFNRGEC | |
| | Heavy Chain 2 | MAWVWTLLFLMAAAQSIQAEIVLTQSPGTLSLSPGERATLSCR | 559 |
| | BCMB519 | ASQSISSSFLTWYQQKPGQAPRLLIYGASSRATGIPDRFSGGGS | |
| | | GTDFTLTISRLEPEDFAVYYCQHYGSSPMYTFGQGTKLEIKGGS | |
| | | EGKSSGSGSESKSTGGSEVQLLESGGGLVQPGGSLRLSCAASGF | |
| | | TFSSYAMSWVRQAPGKGLEWVSAISGSGGSTYYADSVKGRFTI | |
| | | SRDNSKNTLYLQMNSLRAEDTAVYYCAKDEGYSSGHYYGMD | |
| | | VWGQGTTVTVSSEPKSSDKTHTCPPCPAPEAAGGPSVFLFPPKP | |
| | | KDTLMISRTPEVTCVVVSVSHEDPEVKFNWYVDGVEVHNAKT | |
| | | KPREEQYNSTYRVVSVLTVLHQDWLNGKEYKCKVSNKALPAP | |
| | | IEKTISKAKGQPREPQVYVLPPSREEMTKNQVSLLCLVKGFYPS | |
| | | DIAVEWESNGQPENNYLTWPPVLDSDGSFFLYSKLTVDKSRW | |
| | | QQGNVFSCSVMHEALHNHYTQKSLSLSPG | |
| Null × | Heavy Chain 1 | MAWVWTLLFLMAAAQSIQAQITLKESGPTLVKPTQTLTLTCTF | 566 |
| BCMA | B21M | SGFSLSTSGMGVSWIRQPPGKALEWLAHIYWDDDKRYNPSLKS | |
| (B17B612) | | RLTITKDTSKNQVVLTMTNMDPVDTATYYCARLYGFTYGFAY | |
| | | WGQGTLVTVSSASTKGPSVFPLAPSSKSTSGGTAALGCLVKDY | |
| | | FPEPVTVSWNSGALTSGVHTFPAVLQSSGLYSLSSVVTVPSSSL | |
| | | GTQTYICNVNHKPSNTKVDKKVEPKSCDKTHTCPPCPAPEAAG | |
| | | GPSVFLFPPKPKDTLMISRTPEVTCVVVSVSHEDPEVKFNWYV | |
| | | DGVEVHNAKTKPREEQYNSTYRVVSVLTVLHQDWLNGKEYK | |
| | | CKVSNKALPAPIEKTISKAKGQPREPQVYVYPPSREEMTKNQV | |
| | | SLTCLVKGFYPSDIAVEWESNGQPENNYKTTPPVLDSDGSFAL | |
| | | VSKLTVDKSRWQQGNVFSCSVMHEALHNRFTQKSLSLSPG | |
| | Light Chain 1 | METHSQVFVYMLLWLSGVEGDIVMTQSPDSLAVSLGERATINC | 567 |
| | B21M | RASQSVDYNGISYMHWYQQKPGQPPKLLIYAASNPESGVPDRF | |
| | | SGSGSGTDFTLTISSLQAEDVAVYYCQQIIEDPWTFGQGTKVEI | |
| | | KRTVAAPSVFIFPPSDEQLKSGTASVVCLLNNFYPREAKVQWK | |
| | | VDNALQSGNSQESVTEQDSKDSTYSLSSTLTLSKADYEKHKVY | |
| | | ACEVTHQGLSSPVTKSFNRGEC | |
| | Heavy Chain 2 | MAWVWTLLFLMAAAQSIQAEIVLTQSPGTLSLSPGERATLSCR | 559 |
| | BCMB519 | ASQSISSSFLTWYQQKPGQAPRLLIYGASSRATGIPDRFSGGGS | |
| | | GTDFTLTISRLEPEDFAVYYCQHYGSSPMYTFGQGTKLEIKGGS | |
| | | EGKSSGSGSESKSTGGSEVQLLESGGGLVQPGGSLRLSCAASGF | |
| | | TFSSYAMSWVRQAPGKGLEWVSAISGSGGSTYYADSVKGRFTI | |
| | | SRDNSKNTLYLQMNSLRAEDTAVYYCAKDEGYSSGHYYGMD | |
| | | VWGQGTTVTVSSEPKSSDKTHTCPPCPAPEAAGGPSVFLFPPKP | |
| | | KDTLMISRTPEVTCVVVSVSHEDPEVKFNWYVDGVEVHNAKT | |
| | | KPREEQYNSTYRVVSVLTVLHQDWLNGKEYKCKVSNKALPAP | |
| | | IEKTISKAKGQPREPQVYVLPPSREEMTKNQVSLLCLVKGFYPS | |
| | | DIAVEWESNGQPENNYLTWPPVLDSDGSFFLYSKLTVDKSRW | |
| | | QQGNVFSCSVMHEALHNHYTQKSLSLSPG | |

ScFv Sequences

| | | | |
|---|---|---|---|
| B17B621 | Heavy Chain 1 | MAWVWTLLFLMAAAQSIQAQVQLQQSGPELVKPGASVKMSC | 568 |
| ZWA: | Va10.2- | KASGNTIPTYVMHWVKLKPGQGLEWIGYINPYNDGTKYNEKF | |
| Va10.2- | TRVAB1 | KGKATLTSDKSSSTAYMELSSLTSEDSAVYYCAKDGTYVEFAY | |
| TRVAB1- | | WGQGTLVTVSAASTKGPSVFPLAPSSKSTSGGTAALGCLVKDY | |
| Fab-RF | | FPEPVTVSWNSGALTSGVHTFPAVLQSSGLYSLSSVVTVPSSSL | |
| ZWB: | | GTQTYICNVNHKPSNTKVDKKVEPKSCDKTHTCPPCPAPEAAG | |
| BCMB519- | | GPSVFLFPPKPKDTLMISRTPEVTCVVVSVSHEDPEVKFNWYV | |
| scFv | | DGVEVHNAKTKPREEQYNSTYRVVSVLTVLHQDWLNGKEYK | |
| | | CKVSNKALPAPIEKTISKAKGQPREPQVYVYPPSREEMTKNQV | |
| | | SLTCLVKGFYPSDIAVEWESNGQPENNYKTTPPVLDSDGSFAL | |
| | | VSKLTVDKSRWQQGNVFSCSVMHEALHNRFTQKSLSLSPG | |
| | Light Chain 1 | MAWVWTLLFLMAAAQSIQADIVMTQSRKFMSTSVGDRVNITC | 569 |
| | Va10.2- | KASQNIRTAVAWFQQRPGQSPKLLFYLASNRHTGVPDRFTGSG | |
| | TRVAB1 | SGTDFTLTINDVQSEDLADYFCLQHWNYPYTFGSGTKLEMKRT | |
| | | VAAPSVFIFPPSDEQLKSGTASVVCLLNNFYPREAKVQWKVDN | |
| | | ALQSGNSQESVTEQDSKDSTYSLSSTLTLSKADYEKHKVYACE | |
| | | VTHQGLSSPVTKSFNRGEC | |
| | Heavy Chain 2 | MAWVWTLLFLMAAAQSIQAEIVLTQSPGTLSLSPGERATLSCR | 559 |
| | BCMB519 | ASQSISSSFLTWYQQKPGQAPRLLIYGASSRATGIPDRFSGGGS | |
| | | GTDFTLTISRLEPEDFAVYYCQHYGSSPMYTFGQGTKLEIKGGS | |
| | | EGKSSGSGSESKSTGGSEVQLLESGGGLVQPGGSLRLSCAASGF | |
| | | TFSSYAMSWVRQAPGKGLEWVSAISGSGGSTYYADSVKGRFTI | |
| | | SRDNSKNTLYLQMNSLRAEDTAVYYCAKDEGYSSGHYYGMD | |
| | | VWGQGTTVTVSSEPKSSDKTHTCPPCPAPEAAGGPSVFLFPPKP | |
| | | KDTLMISRTPEVTCVVVSVSHEDPEVKFNWYVDGVEVHNAKT | |
| | | KPREEQYNSTYRVVSVLTVLHQDWLNGKEYKCKVSNKALPAP | |
| | | IEKTISKAKGQPREPQVYVLPPSREEMTKNQVSLLCLVKGFYPS | |
| | | DIAVEWESNGQPENNYLTWPPVLDSDGSFFLYSKLTVDKSRW | |
| | | QQGNVFSCSVMHEALHNHYTQKSLSLSPG | |
| B17B627 | Heavy Chain 1 | MAWVWTLLFLMAAAQSIQAEVQLQQSGPELVKPGASVKMSC | 570 |
| ZWA: | Va10.2- | KASGYTFTRYVIHWMKQKPGQGLEWIGYVNPYNNGTKYTEKF | |
| Va10.2- | TRVAB2 | KGKATLTSDKSSSTAYMELNSLTSEDSAVYYCARPRLSGEDAM | |
| TRVAB2- | | EYWGQGTSVTVSSASTKGPSVFPLAPSSKSTSGGTAALGCLVK | |
| Fab-RF | | DYFPEPVTVSWNSGALTSGVHTFPAVLQSSGLYSLSSVVTVPSS | |
| ZWB: | | SLGTQTYICNVNHKPSNTKVDKKVEPKSCDKTHTCPPCPAPEA | |
| BCMB519- | | AGGPSVFLFPPKPKDTLMISRTPEVTCVVVSVSHEDPEVKFNW | |
| scFv | | YVDGVEVHNAKTKPREEQYNSTYRVVSVLTVLHQDWLNGKE | |
| | | YKCKVSNKALPAPIEKTISKAKGQPREPQVYVYPPSREEMTKN | |
| | | QVSLTCLVKGFYPSDIAVEWESNGQPENNYKTTPPVLDSDGSF | |
| | | ALVSKLTVDKSRWQQGNVFSCSVMHEALHNRFTQKSLSLSPG | |
| | Light Chain 1 | MAWVWTLLFLMAAAQSIQADIQMTQSPKSMSMSVGERVTLT | 571 |
| | Va10.2- | CKASENVVTYVSWYQQKPEQSPKLLIYGASNRYTGVPDRFTGS | |
| | TRVAB2 | GSATDFTLTISSVQAEDLAEYHCGQSHSFPYTFGSGTKLELKRT | |
| | | VAAPSVFIFPPSDEQLKSGTASVVCLLNNFYPREAKVQWKVDN | |
| | | ALQSGNSQESVTEQDSKDSTYSLSSTLTLSKADYEKHKVYACE | |
| | | VTHQGLSSPVTKSFNRGEC | |
| | Heavy Chain 2 | MAWVWTLLFLMAAAQSIQAEIVLTQSPGTLSLSPGERATLSCR | 559 |
| | BCMB519 | ASQSISSSFLTWYQQKPGQAPRLLIYGASSRATGIPDRFSGGGS | |
| | | GTDFTLTISRLEPEDFAVYYCQHYGSSPMYTFGQGTKLEIKGGS | |
| | | EGKSSGSGSESKSTGGSEVQLLESGGGLVQPGGSLRLSCAASGF | |
| | | TFSSYAMSWVRQAPGKGLEWVSAISGSGGSTYYADSVKGRFTI | |
| | | SRDNSKNTLYLQMNSLRAEDTAVYYCAKDEGYSSGHYYGMD | |
| | | VWGQGTTVTVSSEPKSSDKTHTCPPCPAPEAAGGPSVFLFPPKP | |
| | | KDTLMISRTPEVTCVVVSVSHEDPEVKFNWYVDGVEVHNAKT | |
| | | KPREEQYNSTYRVVSVLTVLHQDWLNGKEYKCKVSNKALPAP | |
| | | IEKTISKAKGQPREPQVYVLPPSREEMTKNQVSLLCLVKGFYPS | |
| | | DIAVEWESNGQPENNYLTWPPVLDSDGSFFLYSKLTVDKSRW | |
| | | QQGNVFSCSVMHEALHNHYTQKSLSLSPG | |

The antibodies were initially purified by Mab Select SuRe Protein A column (GE healthcare, Piscataway, N.J.) (Brown, Bottomley et al. 1998). The column was equilibrated with Phosphate Buffer Saline (PBS), pH 7.2 and loaded with fermentation supernatant at a flow rate of 2 mL/min. After loading, the column was washed with PBS (4 CV) followed by elution in 30 mM sodium acetate, pH 3.5. Fractions containing protein peaks as monitored by Absorbance at 280 nm in AKTA Explorer (GE healthcare) were pooled together and were neutralized to pH 5.0 by adding 100 of 3 M sodium acetate, pH 9.0. As a polishing step, the antibodies were purified on a preparative size exclusion chromatography (SEC) using a SUPERDEX 200 column (GE healthcare). The integrity of the sample was assessed by endotoxin measurement and SDS polyacrylamide gel electrophoresis under reducing and non-reducing conditions. The intact mass was confirmed by mass spectrometry.

TABLE 11
Anti-Vβ17 and Anti-BCMA Heavy and Light Chain Sequences

| | | | SEQ |
|---|---|---|---|
| | | | ID |
| mAb name | Chain info | Amino Acid Sequence | NO: |
| B17B663 | Heavy Chain | MAWVWTLLFLMAAAQSIQADVQLQESGPGLVAPSQSLS | 572 |
| | | ITCTVSGFSLSSYAISWVRQPPGKGLEWLGVIWAGGGTN | |
| | | YNSALKSRLSISKDNSKSQVFLKMKSLQTDDTARYYCAR | |
| | | NFFYDYDDGMDYWGQGTTVTVSSASTKGPSVFPLAPSS | |
| | | KSTSGGTAALGCLVKDYFPEPVTVSWNSGALTSGVHTFP | |
| | | AVLQSSGLYSLSSVVTVPSSSLGTQTYICNVNHKPSNTKV | |
| | | DKKVEPKSCDKTHTCPPCPAPELLGGPSVFLFPPKPKDTL | |
| | | MISRTPEVTCVVVDVSHEDPEVKFNWYVDGVEVHNAKT | |
| | | KPREEQYNSTYRVVSVLTVLHQDWLNGKEYKCKVSNK | |
| | | ALPAPIEKTISKAKGQPREPQVYTLPPSREEMTKNQVSLT | |
| | | CLVKGFYPSDIAVEWESNGQPENNYKTTPPVLDSDGSFF | |
| | | LYSKLTVDKSRWQQGNVFSCSVMHEALHNHYTQKSLSL | |
| | | SPGK | |
| | Light Chain | MARSALLILALLLLGLFSPGAWGNTQMNQTPLSLPVSLG | 573 |
| | | DQASISCRSSQSLVHSNGNTYLHWYLQKPGQSPKLLIYK | |
| | | VSNRFSGVPDRFSGSGSGTDFTLKINRVEAEDLGVYFCSQ | |
| | | STHVPLTFGAGTRLEIKRTVAAPSVFIFPPSDEQLKSGTAS | |
| | | VVCLLNNFYPREAKVQWKVDNALQSGNSQESVTEQDSK | |
| | | DSTYSLSSTLTLSKADYEKHKVYACEVTHQGLSSPVTKS | |
| | | FNRGEC | |
| B17B694 | Heavy Chain | MAWVWTLLFLMAAAQSIQAQIQLVQSGPELKKPGETVK | 574 |
| | | ISCKASGYTFTNYGMNWVKQAPGKGLKWMGWINTYTG | |
| | | EPTYADDFKGRFAFSLETSASTAYLQINNLKNEDMATYF | |
| | | CARPYFGDYAMDYWGQGTLVTVSAASTKGPSVFPLAPS | |
| | | SKSTSGGTAALGCLVKDYFPEPVTVSWNSGALTSGVHTF | |
| | | PAVLQSSGLYSLSSVVTVPSSSLGTQTYICNVNHKPSNTK | |
| | | VDKKVEPKSCDKTHTCPPCPAPELLGGPSVFLFPPKPKDT | |
| | | LMISRTPEVTCVVVDVSHEDPEVKFNWYVDGVEVHNAK | |
| | | TKPREEQYNSTYRVVSVLTVLHQDWLNGKEYKCKVSN | |
| | | KALPAPIEKTISKAKGQPREPQVYTLPPSREEMTKNQVSL | |
| | | TCLVKGFYPSDIAVEWESNGQPENNYKTTPPVLDSDGSF | |
| | | FLYSKLTVDKSRWQQGNVFSCSVMHEALHNHYTQKSLS | |
| | | LSPGK | |
| | Light Chain | MARSALLILALLLLGLFSPGAWGDVQMIQSPASLSVSVG | 575 |
| | | ETVTITCRASENIYSNLAWYQQKQGKSPQVLVYTATNLA | |
| | | DGVPSRFSGSGSGTQYSLKINSLQSEDFGSYYCQHFWGN | |
| | | PWTFGGGTKLEIKRTVAAPSVFIFPPSDEQLKSGTASVVC | |
| | | LLNNFYPREAKVQWKVDNALQSGNSQESVTEQDSKDST | |
| | | YSLSSTLTLSKADYEKHKVYACEVTHQGLSSPVTKSFNR | |
| | | GEC | |
| B17B698 | Heavy Chain | MAWVWTLLFLMAAAQSIQAQVQLQQSGPGLVAPSQSLS | 576 |
| | | ITCTVSGFSLTSYAISWIRQPPGKGLEWLGIIWAGGGTNY | |
| | | NSALKSRLSISKDNSKSQVFLKMNSLQIDDTARYYCARN | |
| | | PFYDYDEGLDYWGQGTTLTVSSASTKGPSVFPLAPSSKS | |
| | | TSGGTAALGCLVKDYFPEPVTVSWNSGALTSGVHTFPA | |
| | | VLQSSGLYSLSSVVTVPSSSLGTQTYICNVNHKPSNTKVD | |
| | | KKVEPKSCDKTHTCPPCPAPELLGGPSVFLFPPKPKDTLM | |
| | | ISRTPEVTCVVVDVSHEDPEVKFNWYVDGVEVHNAKTK | |
| | | PREEQYNSTYRVVSVLTVLHQDWLNGKEYKCKVSNKA | |
| | | LPAPIEKTISKAKGQPREPQVYTLPPSREEMTKNQVSLTC | |
| | | LVKGFYPSDIAVEWESNGQPENNYKTTPPVLDSDGSFFL | |
| | | YSKLTVDKSRWQQGNVFSCSVMHEALHNHYTQKSLSLS | |
| | | PGK | |
| | Light Chain | MARSALLILALLLLGLFSPGAWGDIVMTQSPLSLPVSLGD | 577 |
| | | QASISCRSSQSLVHSNGNTYLHWYLQKPGQSPKLLIYKV | |
| | | SNRFSGVPDRFSGSGSGTDFTLKISRVEAEDLGVYFCSQS | |
| | | THVPWTFGGGTKLEIKRTVAAPSVFIFPPSDEQLKSGTAS | |
| | | VVCLLNNFYPREAKVQWKVDNALQSGNSQESVTEQDSK | |
| | | DSTYSLSSTLTLSKADYEKHKVYACEVTHQGLSSPVTKS | |
| | | FNRGEC | |
| B17B733 | Heavy Chain | MAWVWTLLFLMAAAQSIQAQVQLKESGPVLVAPSQSLS | 578 |
| | | ITCTVSGFSLTSYGVHWIRQPPGKGLEWLGVIWAGGNTN | |
| | | YNSTLMSRLSISKDNSKSQVFLKMNSLQTDDTAMYYCA | |
| | | RGSFYDYLYFDYWGQGTTLTVSSASTKGPSVFPLAPSSK | |
| | | STSGGTAALGCLVKDYFPEPVTVSWNSGALTSGVHTFPA | |
| | | VLQSSGLYSLSSVVTVPSSSLGTQTYICNVNHKPSNTKVD | |
| | | KKVEPKSCDKTHTCPPCPAPELLGGPSVFLFPPKPKDTLM | |
| | | ISRTPEVTCVVVDVSHEDPEVKFNWYVDGVEVHNAKTK | |
| | | PREEQYNSTYRVVSVLTVLHQDWLNGKEYKCKVSNKA | |
| | | LPAPIEKTISKAKGQPREPQVYTLPPSREEMTKNQVSLTC | |
| | | LVKGFYPSDIAVEWESNGQPENNYKTTPPVLDSDGSFFL | |
| | | YSKLTVDKSRWQQGNVFSCSVMHEALHNHYTQKSLSLS | |
| | | PGK | |
| | Light Chain | MARSALLILALLLLGLFSPGAWGDIQMTQSPSYLAASPG | 579 |
| | | ETITINCRASKSISKYLAWYQEKPGKTNELLIYSGSTLQSG | |
| | | IPSRFSGSGSGTDFTLTISSLEPEDFAMYYCQQHNEYPLTF | |
| | | GGGTKLEIKRTVAAPSVFIFPPSDEQLKSGTASVVCLLNN | |
| | | FYPREAKVQWKVDNALQSGNSQESVTEQDSKDSTYSLS | |
| | | STLTLSKADYEKHKVYACEVTHQGLSSPVTKSFNRGEC | |

### Example 2.4: Evaluation of Binding and Cytotoxic Properties of the Anti-Vβ17/Anti-BCMA Bispecific Antibody Using H929 Cells and Human T Cells

The VB17 antibody (Vb17_202B4D1-Fab-RF, BCMB519-scFv (B17B622.001)) binds T cells and mediates T cell cytotoxicity against BCMA expressing H929 cells in vitro (data not shown). The VB17 antibody (Vb17_210E10A1-Fab-RF, BCMB519-scFv (B17B624.001)) binds T cells and mediates T cell cytotoxicity against BCMA expressing H929 cells in vitro (data not shown). The control antibody (B21M-Fab-RF×BCMB519-LH-scFv (B17B612.001)) does not bind T cells or mediate T cell cytotoxicity against BCMA expressing H929 cells in vitro (data not shown). EC₅₀ values were calculated as described in methods. Representative data shown in these figures are from a single experiment. For the binding assays, αβ-enriched T cells were used, and samples incubated for 1 hour at 37° C. prior to measurements. For the killing assays, human pan T cells (effectors) were co-cultured with H929 at 5:1 E:T ratios in the presence of various concentrations of the bispecific antibody for 72 hours at 37° C. Bispecific constructs were tested in 11-point titration curve with a 3-fold dilution series starting at 50 nM antibody concentration. Human pan T cells were used as effector cells. H929-WT tumor cell line was used as target cells. Dose response curves show anti-Vβ17/anti-BCMA bispecific mediated T cell cytotoxicity against BCMA expressing H929 cells in a dose dependent manner.

### Example 3: Preparation of Anti-Vβ17/Anti-BCMA/Anti-CD28 Trispecific Antibodies

### Example 3.1: Materials and Methods

Anti-CD28 antibody generation.

OMNIRATS were immunized twice weekly with recombinant human CD28 (R&D Systems, Inc., MN, USA; Catalog #: 342-CD-200; LOT #: XT321505A) for a total of 12 immunization boosts by following a Repetitive Immunizations Multiple Sites (RIMMS) protocol. Sera was collected and assessed for circulating IgG specific antibodies to CD28 and titers were determined via a solid phase Elisa with antigen being coated directly on the plate. Lymph nodes were harvested for B lymphocytes fusion. Hybridoma supernatants were screened by LUMINEX using the immunogen and expanded pan-T cells. Hits were V-region recovered and formatted human IgG1 antibody.

Cell Culture.

NCI-H929 myeloma cells were cultured in RPMI-1640 Medium (A1049101, Thermofisher) containing fetal bovine serum (10099-141, Gibco) to a final concentration of 20%. Cells were subcultured every 2-3 days by spinning the culture at 1500 rpm for 5 mins at 37° C. Culture supernatant was discarded, and cells were seeded back in fresh media at a density of 0.5-1×106/ml. Frozen PBMCs were obtained from Hemacare. PBMCs were also isolated from fresh blood from normal healthy volunteers from Clinigene after informed consent.

Binding Assay.

PBMCs (Donor lot #19054141, Hemacare) were thawed rapidly in a 37° C. water bath and subjected to CD3 T cell isolation using EasySep™ Human T Cell Isolation Kit (17951, Stemcell). Post Pan T cell isolation, 0.1×10⁶ cells were seeded per well of a 96 well V-bottom plate. Cells were stained with the fixable violet live/dead stain (L34955, Thermofisher) for 20 mins on ice in dark according to the manufacturer's protocol. Post staining cells were washed with FACS buffer (PBS+2% FBS) by spinning at 1500 rpm for 5 mins. Supernatant was discarded and cells were suspended in 100 μl the respective antibody dilutions with a starting concentration of 5 μg/ml and 3-fold serial dilutions. Antibody dilutions were prepared in FACS buffer. Cells were incubated with the antibody dilutions for 30 mins at 37° C. At the end of the incubation period, cells were washed twice with FACS buffer as above followed by staining with PE conjugated Goat polyclonal antibody to human IgG (ab98596, Abcam) at a 1:50 dilution in FACS buffer. Cells were incubated with the secondary antibody for 30 mins on ice. At the end of incubation period the cells were washed with FACS buffer as above. Cells were fixed by resuspending in 100 μl BD cytofix buffer (554655, BD Bioscience) and incubated for 20 mins on ice. Cells were pelleted and resuspended in FACS buffer for acquisition on the NOVOCYTE flow cytometer (ACEA Biosciences). For binding assays using the H929 cells, 0.1×10⁶ H929 cells were seeded per well of a 96 well V-bottom plate and stained as above. Samples were analyzed by gating on the live cells and percentage binding was obtained by subtracting the background fluorescence from the secondary only control.

Agonism Assay.

Antibody dilutions was prepared in PBS at a starting concentration of 1 μg/ml followed by 4-fold serial dilutions. Wells with no antibody addition were used as negative controls. 100 μl of the antibody dilutions were used to coat 96 well flat bottom cell culture plates with incubation at 37° C. for 2 hours. PBMCs (Donor lots #19054456, 19057652, Hemacare) were thawed and subjected to Pan T cells isolation using the EasySep™ Human T Cell Isolation Kit (17951, Stemcell). Isolated Pan T cells were counted and stained with cell trace violet dye (C34557, Thermofisher) as per the manufacturer's protocol. Antibody coated plates were washed with 200 μl media and 0.3×10⁶ CTV labeled Pan T cells were plated per well. Plates were incubated at 37° C. for 96 hours in a 5% CO2 incubator. At the end of the incubation period, the cells were spun down at 1500 rpm for 5 mins. The 150 μl of cell culture supernatant was collected and stored at −20° C. for cytokine profiling using LUMINEX. The cell pellet was subjected to APC-Cy7 live/dead stain (L10119, Thermofisher). Post live/dead staining the cells were washed with FACS buffer. The pellet was then resuspended in FACS buffer containing Fc block (564220, BD Biosciences) and incubated on ice for 10 mins following which the cells were stained with Brilliant Violet 785™ conjugated CD25 (302638, Biolegend) and PE/Cy7 conjugated CD71 (334112, Biolegend) antibodies and incubated on ice for 30 mins. At the end of incubation period the cells were washed with FACS buffer and the cells were fixed by resuspending in 100 μl BD cytofix buffer (554655, BD bioscience) and incubated for 20 mins on ice. Post fixation, the cells were washed, and the samples were resuspended in FACS buffer and acquired on the NOVOCYTE flow cytometer. Proliferation was monitored by CTV dye. Cells were gated on the live cell population, followed by gating on Vβ17+ T cells and Vβ17− T cells. Expression of CD25, CD71 and CTV dye was monitored on each cell population and plotted as % positive cells against log antibody concentration using a 4-parameter non-linear regression curve.

Effector Profiling.

PBMCs (Donors HPU-00284 from Clinigene and donor lot #19054141, 19054456, 20061101 from Hemacare) were thawed and subjected to EasySep™ Human T Cell Isolation Kit (17951, Stemcell). H929 cells were counted and plated at 10,000 cells per well in a 96 well U-bottom plate in 100 μl of media. Isolated Pan T cells were counted and stained with cell trace violet dye (C34557, Thermofisher) as per the manufacturer's protocol. Vβ17 T cell frequency was determined in the Pan T cells from each donor using the PE conjugated TCR Vβ17 antibody (IM2048, Beckman Coulter). CTV labeled Pan T cells were then added to the plated H929 cells such that an effector to target ratio of 1 Vβ17:1 H929 cell was obtained. 80 μl of the effector cell suspension in RPMI media+10% FBS was added per well. For example, the Vβ17 percentage for HPU-00284 was 4.3%, so 0.23×10⁶ cells were plated per well in 80 μl media. 10× concentration of the antibodies were prepared (10 μg/ml) followed by 4-fold serial dilutions in RPMI media+10% FBS. 20 μl of the serially diluted Ab was added to the 180 μl of the co-culture making the final concentration of the antibodies in coculture as 1×. The cell culture plates were incubated at 37° C. for 96 hrs. At the end of the incubation period, the cells were spun down at 1500 rpm for 5 mins. The 150 μl of cell culture supernatant was collected and stored at −20° C. for cytokine profiling by LUMINEX. Cells were stained with APC-Cy7 live/dead stain (L10119, Thermofisher) followed by staining with Fc block (564220, BD Biosciences). The cell pellet was taken for staining with Brilliant Violet 785™ anti-human CD25 (302638, Biolegend), PE/Cy7 anti-human CD71 (334112, Biolegend), BV 650 Anti human TIM3 (345028, Biolegend), Alexa Fluor® 488 anti-human LAG3 (369326, Biolegend), and Brilliant Violet 711 anti-human PD1 antibodies (cat #329928, Biolegend) as per the manufacturer's recommendation. Cells were washed post staining in FACS buffer and fixed with BD cytofix buffer (554655, BD Bioscience). Post fixation, samples were resuspended in FACS buffer and acquired on the NOVOCYTE flow cytometer. Cells were gated on the live cell population, followed by gating on Vβ17+ T cells and Vβ17− T cells. Expression of CD25, CD71, TIM3, LAG3, PD1 and CTV dye was monitored on each cell population and plotted as % positive cells against log antibody concentration using a 4-parameter non-linear regression curve using GRAPHPAD Prism version 8.1.1.

Luminex Analysis.

Supernatants from the effector profiling assay were slowly thawed and diluted 1:10 using RPMI media+10% FBS. Cytokine analysis was carried out using the MILLIPLEX MAP Human CD8+ T Cell Magnetic Bead Panel Immunology Multiplex Assay (HCD8MAG-15K, Millipore). Plates were read using the LUMINEX plate reader (Magpix).

In Vitro Cytotoxicity Assay.

PBMCs (Donor lots #18047563, 19056279 from Hemacare) were thawed and subjected to Pan T cell isolation using the EasySep™ Human T Cell Isolation Kit (17951, Stemcell). Another set was subjected to Vβ17 depletion using EasySep™ Human PE Positive Selection Kit (18551, Stemcells) followed by Pan T cell isolation. H929 cells were labeled with 0.5 μm of CTV dye (C34557, Thermofisher) as per the manufacturer's protocol, counted and plated at 10,000 cells per well in a 96 well U-bottom plate in 100 μl of RPMI media (ATCC modification)+20% FBS. Vβ17 T cell frequency was determined in the Pan T cells from each donor using the PE conjugated TCR Vβ17 antibody (IM2048, Beckman Coulter). CTV labeled Pan T cells were then added to the plated H929 cells such that an effector to target ratio of 1 Vβ17:1 H929 cell was obtained. 80 μl of the effector cell suspension in RPMI media+10% FBS was added per well. 10× concentration of the antibodies were prepared (10 μg/ml) followed by 4-fold serial dilutions in media. 20 μl of the serially diluted antibody was added to the 180 μl of the co-culture making the final concentration of the antibodies in coculture as 1×. The cell culture plates were incubated at 37° C. for 96 hrs. At the end of the incubation period, the cells were spun down at 1500 rpm for 5 mins. At the end of incubation period, the cells were spun down, 150 μl of cell culture supernatant removed and cells were resuspended in 50 μl of 7AAD (420404, Biolegend) diluted 1:50 in PBS and acquired on the NOVOCYTE flow cytometer. Target cells were identified as CTV positive cells and percentage of dead cells within the target cells was gated as 7AAD+ cells. Antibody specific percentage dead cells were calculated by subtracting the lysis observed in wells containing only Pan T cells and H929 cells. Percentage dead cells were plotted against log concentration of the antibody in a 4-parameter non-linear regression curve using GRAPHPAD Prism version 8.1.1.

De Novo Sequencing of Mouse Anti-Human CD28 Clone Obtained from Commercial Source:

A CD28 antibody was obtained from a commercial source and internally designated as clone C28B19. Antibody isotype was mouse IgG1, kappa. Sample preparation and LC/MSMS analysis were performed at Protea Bioscience Inc. (Morgantown, W. Va.). The sample was reduced and alkylated, divided into seven aliquots, and proteolytically digested with Trypsin/LysC, Chymotrypsin, LysC, Pepsin, and AspN, Elastase, and Proteinase K enzymes. Resulting peptides were desalted using a ZIPTIP C18 Pipette Tips and separated on-line using reverse phase chromatography. Mass spectrometry was performed on Thermo Q-EXACTIVE spectrometer using HCD fragmentation. MS data sets were analyzed using PEAKS software by matching de novo sequence tags to an IMGT-based antibody sequences database. Gaps in the sequence were assigned using Contig sequence assembly of de novo identified peptides. All CDRs and hyper-mutations were confirmed by inspecting the MS/MS spectra. Leu and Ile amino acid residues are practically indistinguishable by mass spectrometry. Leu/Ile in the CDR regions were identified by aligning the determined sequence to a V-region sequence database and confirmed by chymotrypsin enzyme specificity. The expected confidence in Leu/Ile identifications in the CDRs is 80%.

### Example 3.2: Anti-Vβ17/Anti-BCMA/Anti-CD28 Trispecific Antibody Production

The variable region sequence of anti-Vβ17, anti-CD28 and anti-BCMA antibodies were used to generate a trispecific human IgG1 antibody to be tested for T cell re-directed killing of H929 cells.

The trispecific antibodies were produced as Fab (CD28)×scFv (Vβ17)×scFv (BCMA) antibodies in the knob-into-hole format as human IgG1 with silent Fc. Nucleic acid sequences encoding variable regions were sub-cloned into a custom mammalian expression vectors containing constant region of human IgG1 expression cassettes using standard PCR restriction enzyme based standard cloning techniques, and sequences verified. The bispecific antibodies were expressed by transient transfection in Chinese hamster ovary cell line. The antibodies were initially purified by MABSELECT SURE Protein A column (GE Healthcare). The column was equilibrated with PBS pH 7.2 and loaded with fermentation supernatant at a flow rate of 2 mL/min. After loading, the column was washed with 4 column volumes of PBS followed by elution in 30 mM sodium acetate, pH 3.5. Fractions containing protein peaks as monitored by absorbance at 280 nm were pooled and neutralized to pH 5.0 by adding 1% 3 M sodium acetate pH 9.0. The bispecific mAbs were further purified on a preparative SUPERDEX 200 10/300 GL (GE healthcare) size exclusion chromatography (SEC) column equilibrated with PBS buffer. The integrity of sample was assessed by endotoxin measurement (<3.0 EU/mg), SDS-PAGE under reducing and non-reducing conditions, SEC, and intact mass by MS.

The design of the trispecific antibodies is shown in Table 12 below.

TABLE 12
Design of the trispecific antibodies

| | | Chain A | | Chain B | | |
|---|---|---|---|---|---|---|
| | | description | | description | | |
| | | N-term | | N-term | | |
| # | Name | (Fab) | C-term | (Fab) | C-term | Molecule Description |
| 1 | VB28B1 | C28B11 | B17B21 | C28B11 | BCMB519 | C28B11 × B17B21 × BCMB519 |
| 2 | VB28B2 | C28B19 | B17B21 | C28B19 | BCMB519 | C28B19 × B17B21 × BCMB519 |
| 3 | VB28B3 | C28B103 | B17B21 | C28B103 | BCMB519 | C28B103 × B17B21 × |
| | | | | | | BCMB519 |
| 4 | VB28B4 | C28B105 | B17B21 | C28B105 | BCMB519 | C28B105 × B17B21 × |
| | | | | | | BCMB519 |
| 5 | VB28B5 | B21M | B17B21 | B21M | BCMB519 | B21M × B17B21 × BCMB519 |
| 6 | VB28B6 | C28B11 | Null | C28B11 | BCMB519 | C28B11 × Null × BCMB519 |
| 7 | VB28B7 | C28B19 | Null | C28B19 | BCMB519 | C28B19 × Null × BCMB519 |
| 8 | VB28B8 | C28B103 | Null | C28B103 | BCMB519 | C28B103 × Null × BCMB519 |
| 9 | VB28B9 | C28B105 | Null | C28B105 | BCMB519 | C28B105 × Null × BCMB519 |
| 10 | VB28B10 | B21M | Null | B21M | BCMB519 | B21M × Null × BCMB519 |

### Example 3.3: Anti-Vβ17/Anti-BCMA/Anti-CD28 Trispecific Antibodies Show Potent Binding on Pan T Cells

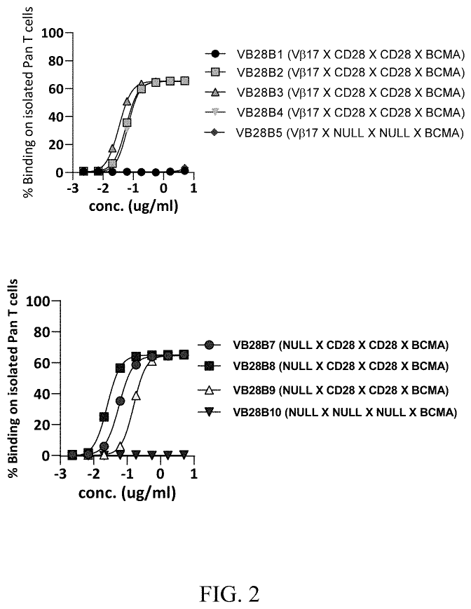

To test the engagement of CD28 on Vβ17 T cells, Pan T cells were isolated from human PBMCs and tested for binding with the Vβ17×CD28×BCMA antibodies containing various CD28 binders. Antibodies with C28B19, C28B103 and C28B105 clones showed robust binding to pan T cells in a dose dependent manner. See FIG. 2 and Table 13. EC50 values for binding were determined to be 0.06 μg/ml, 0.03 μg/ml and 0.06 μg/ml respectively for C28B19, C28B103 and C28B105. Antibody with C28B11 clone was observed to be a poor binder for CD28 and showed binding to only 5% of pan T cells because of the Vβ17 binding arm. Vβ17×BCMA antibody also showed binding to 5% of pan T cells only which was also the frequency of Vβ17 T cells in the tested donors. Binding to pan T cells was dependent on the CD28 arm of the trispecific antibody as the EC50 values for binding of trispecific antibodies lacking the Vβ17 arm was similar to the full trispecific antibody. No binding was observed in antibodies lacking the Vβ17 and CD28 binding arms.

TABLE 13
Cell binding to Pan T cells

| Name | hIgG1 AAS format | EC₅₀ (μg/ml) |
|---|---|---|
| VB28B1 | Vb17xCD28xCD28xBCMA | No binding |
| VB28B2 | Vb17xCD28xCD28xBCMA | 0.06 |
| VB28B3 | Vb17xCD28xCD28xBCMA | 0.03 |
| VB28B4 | Vb17xCD28xCD28xBCMA | 0.06 |
| VB28B5 | Vb17xnullxnullxBCMA | Binding only observed |
| | | to Vb17 + T cells |
| VB28B6 | NullxCD28xCD28xBCMA | NA |
| VB28B7 | NullxCD28xCD28xBCMA | 0.06 |
| VB28B8 | NullxCD28xCD28xBCMA | 0.02 |
| VB28B9 | NullxCD28xCD28xBCMA | 0.15 |
| VB28B10 | NullxNullxBCMA | No binding |

### Example 3.4: Anti-Vβ17/Anti-BCMA/Anti-CD28 Trispecific Antibodies Show Potent Binding on H929 Cells Using BCMA and CD28

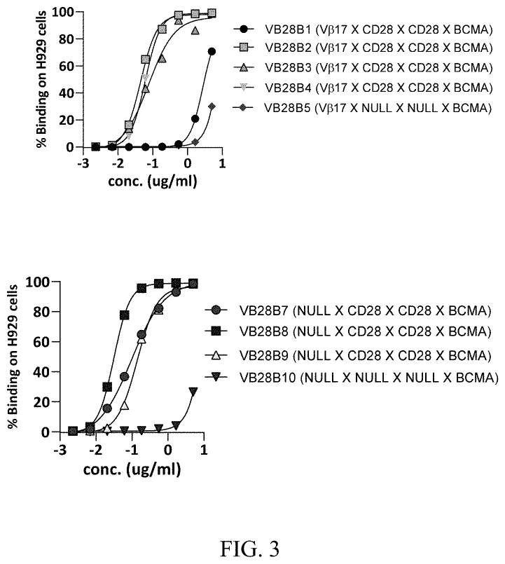

Abc. H929 cells were observed to express CD28 (data not shown). This is in line with multiple myeloma cells expressing CD28. Vβ17×CD28×BCMA trispecific antibodies showed potent binding to H929 cells in a CD28 and BCMA dependent manner. See FIG. 3 and Table 14. EC50 values for binding were determined to be 0.10 μg/ml, 0.03p g/ml and 0.14 μg/ml for trispecific antibodies with C28B19, C28B103 and C28B105 clones respectively. Binding of the trispecific antibodies was largely dependent upon CD28 arm as a potent decrease in binding was observed in absence of CD28 arm (EC50>5 μg/ml). VB28B1 antibody with C28B11 clone showed poorer binding on H929 cells also as compared to the other antibodies because of the weak affinity of the C28B 11 clone.

TABLE 14
Cell binding to BCMA expressing H929 cell line

| | | FC₅₀ |
|---|---|---|
| Name | hIgG1 AAS format | (μg/ml) |
| VB28B1 | Vb17xCD28xCD28xBCMA | >5 |
| VB28B2 | Vb17xCD28xCD28xBCMA | 0.04 |
| VB28B3 | Vb17xCD28xCD28xBCMA | 0.08 |
| VB28B4 | Vb17xCD28xCD28xBCMA | 0.06 |
| VB28B5 | Vb17xnullxnullxBCMA | >5 |
| VB28B6 | NullxCD28xCD28xBCMA | NA |
| VB28B7 | NullxCD28xCD28xBCMA | 0.10 |
| VB28B8 | NullxCD28xCD28xBCMA | 0.03 |
| VB28B9 | NullxCD28xCD28xBCMA | 0.14 |
| VB28B10 | NullxNullxBCMA | >5 |

### Example 3.5: Engagement of CD28 Potently Enhances the Activation of Vβ17 T Cells in Plate Bound Agonism Assay

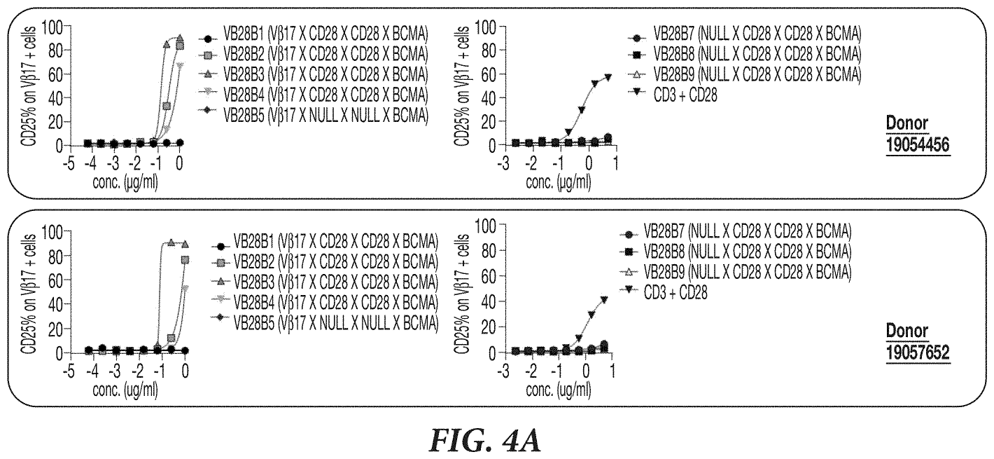

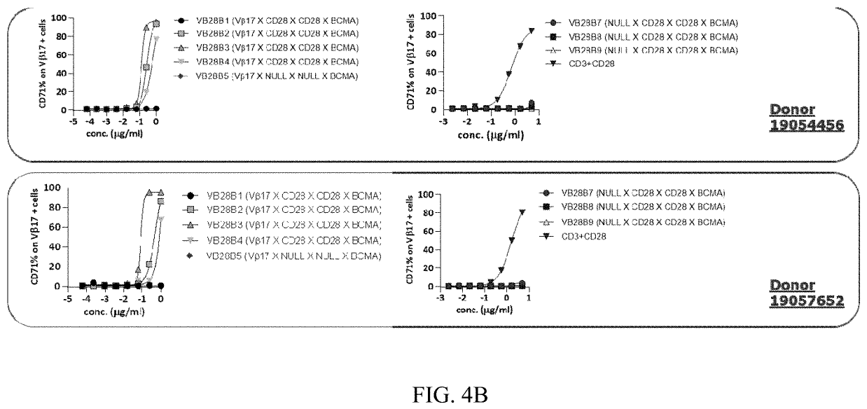

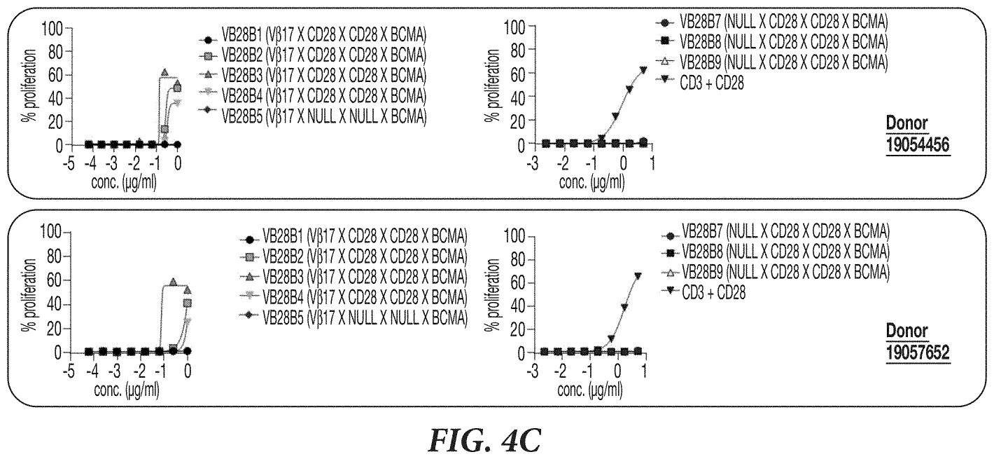

To specifically examine the effect of CD28 stimulation on Vβ17 T cells, pan T cells were cultured on plates coated with Vβ17×CD28×BCMA or null×CD28×BCMA antibodies for 96 hours. At the end of the culture period activation of Vβ17 T cells was checked using CD25 (FIG. 4A), CD71 (FIG. 4B) and proliferation (FIG. 4C). VB28B2, VB28B3 and VB28B4 antibodies showed a strong enhancement of Vβ17 T cell activation as indicated by the upregulation of CD25 and CD71 expression on Vβ17 T cells and an increase in the proliferation of Vβ17 T cells. VB28B1 antibody did not show activation of Vβ17 T cells which was in line with the poor binding observed with this antibody. Activation with VB28B3 (clone C28B103) was observed to be the strongest. No increase in activation was observed with CD28 engagement in the absence of Vβ17 arm. As expected stimulation with CD3 and CD28 combination resulted in the activation of Vβ17 T cells. Overall CD28 costimulation resulted in robust enhancement of Vβ17 T cell activation and this was independent of the CD28 expression on target cells.

### Example 3.6: Engagement of CD28 Potently Enhances the Activation of Vβ17 T Cells in the Presence of H929 Cells

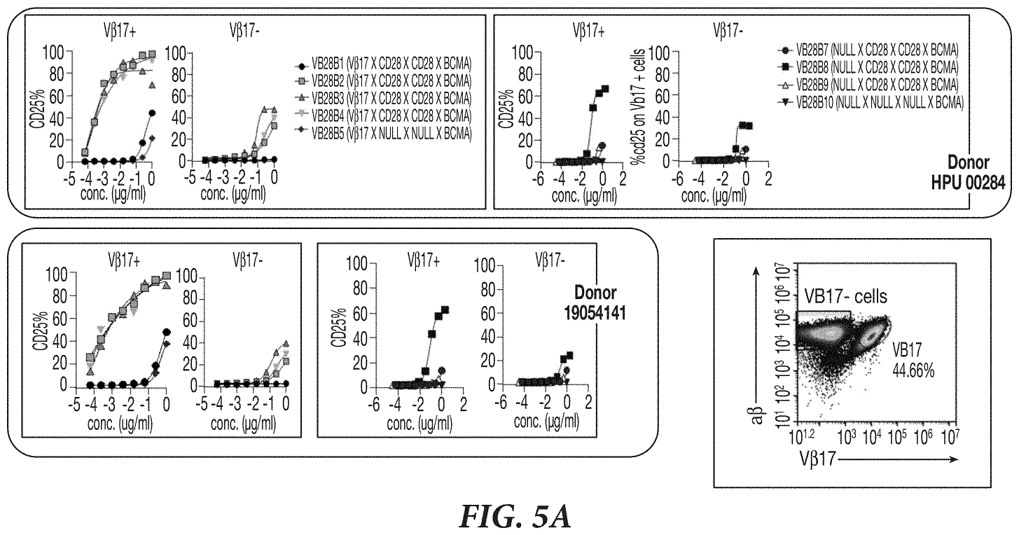

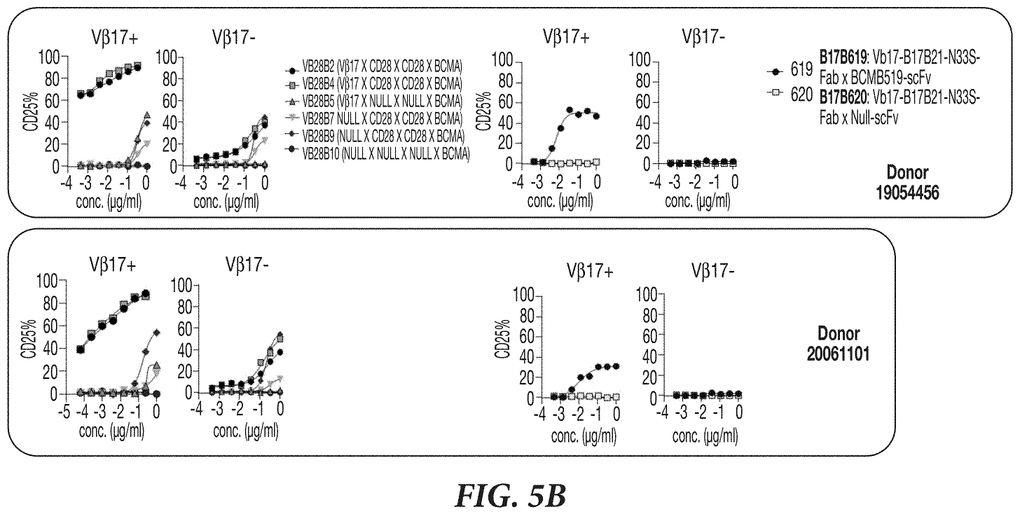

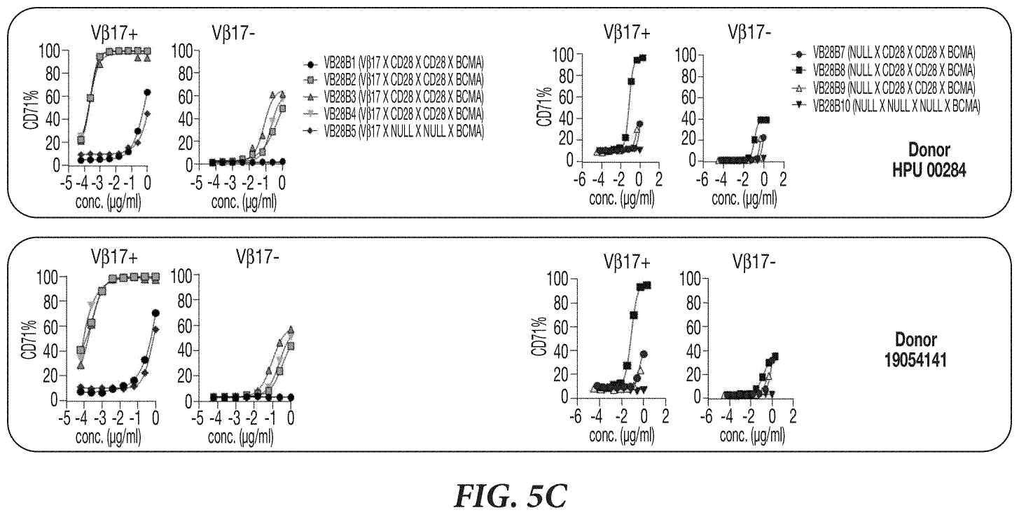

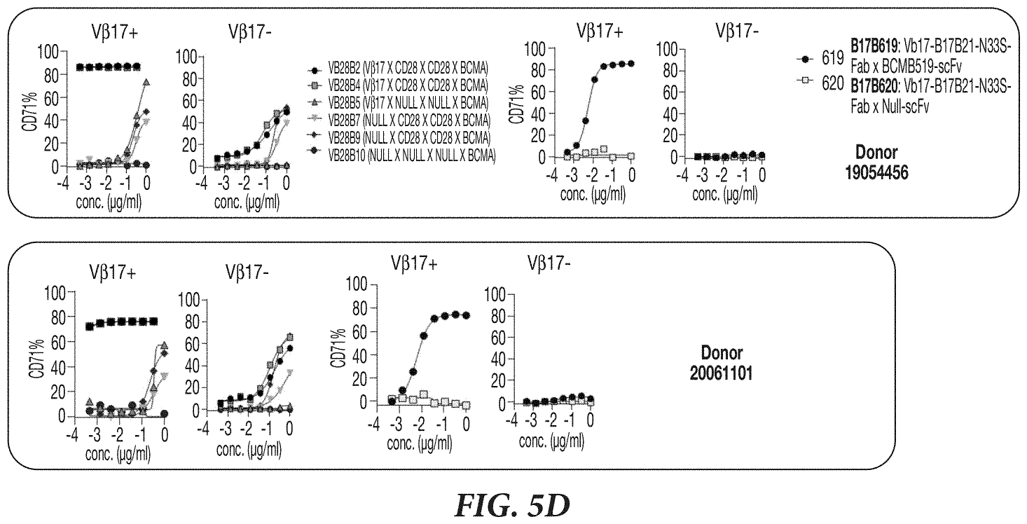

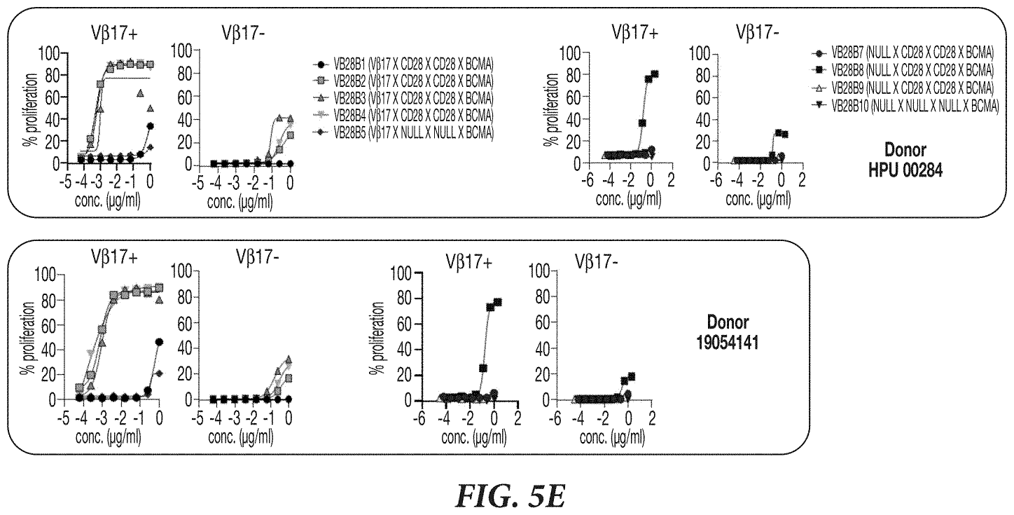

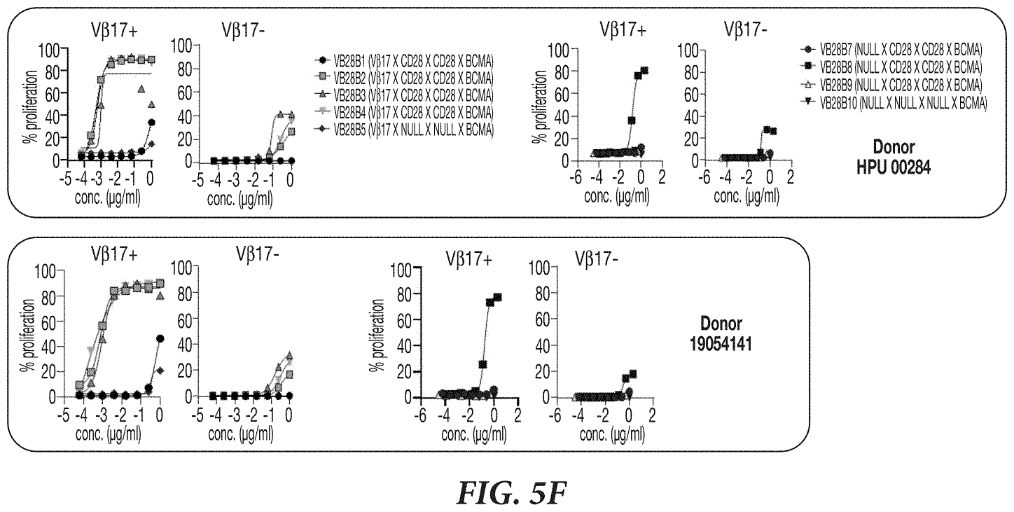

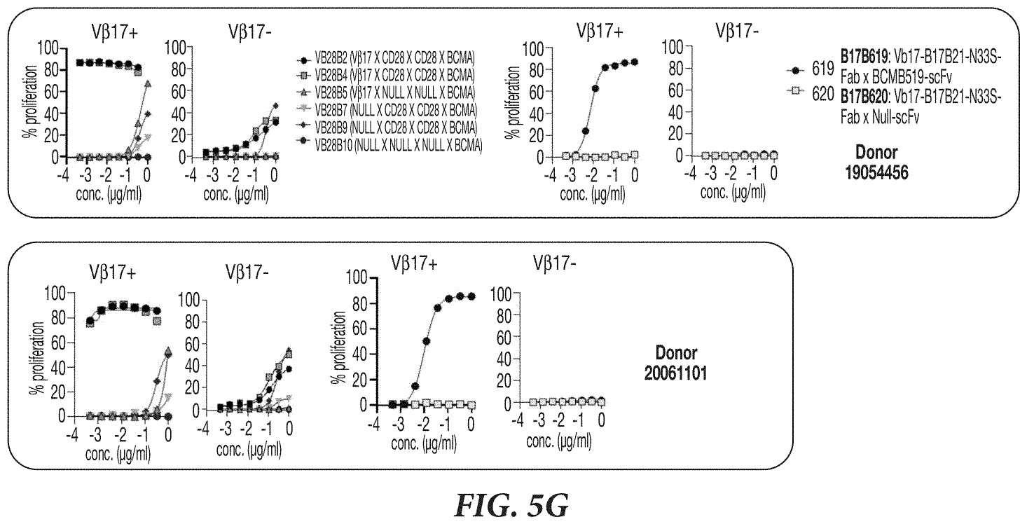

To investigate the effect of CD28 stimulation on Vβ17 T cells and the result of CD28 engagement on H929 cells, pan T cells were cultured with H929 cells at a 1:1 ET ratio of Vβ17 to H929 cells in the presence of the antibodies for 96 hours. Activation of Vβ17 T cells was observed in a dose dependent manner with the addition of Vβ17×BCMA antibody as indicated by the upregulation of CD25 (FIGS. 5A and 5B) and CD71 (FIGS. 5C and 5D) on Vβ17 T cells and an increase in the proliferation of Vβ17 T cells (FIGS. 5E, 5F and 5G). Two formats of Vβ17×BCMA antibodies were tested and activation with B17B619 antibody (Vβ17-Fab×BCMA-ScFv) was observed to stronger than the activation induced by VB28B5 antibody. Importantly the activation induced by Vβ17×CD28×BCMA antibodies was enhanced almost 100-fold as compared to the activation by Vβ17×BCMA antibody. This was true for VB28B2, VB28B3 and VB28B4 antibodies. VB28B1 antibody as before did not show any increase in the activation. Activation of Vβ17 negative T cells with the trispecific antibodies was also observed albeit at much lower levels than the Vβ17+ cells. Strong dose dependent activation of Vβ17+ cells was also observed with Null×CD28×BCMA antibody with the C28B103 binder indicating the agonistic activity of this CD28 clone. Interestingly activation of the Vβ17− cells with this clone of the null×CD28×BCMA antibody was lower than that of the Vβ17+ cells suggesting that Vβ17+ T cells may be inherently more activated than Vβ17− T cells. Among the other two CD28 binders, CD28B19 containing antibody VB28B2 and its VB17 null control VB28B7 was observed to be the best for inducing specific activation of Vβ17 T cells only. Vβ17×BCMA antibody as expected did not show any activation of Vβ17− T cells.

### Example 3.7: Engagement of CD28 does not Induce Exhaustion of Vβ17 T Cells

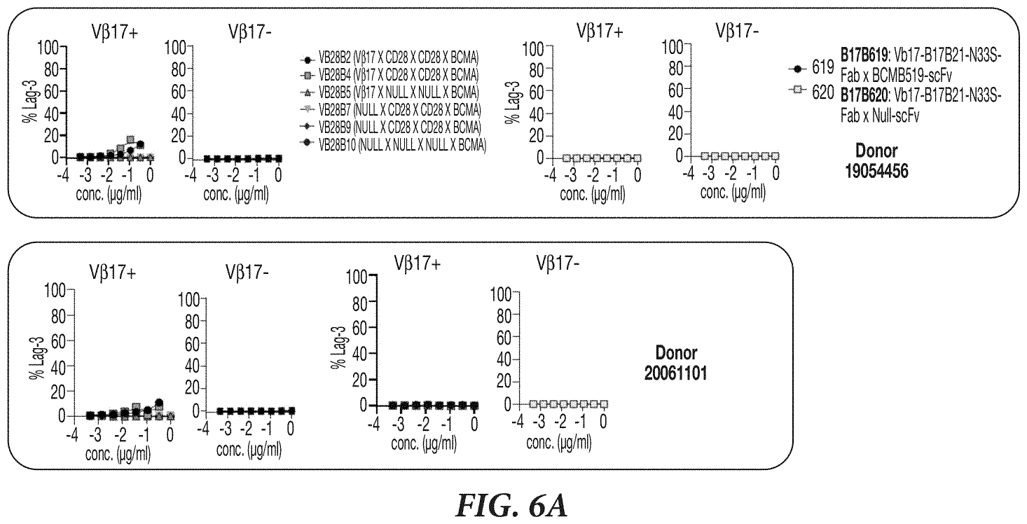

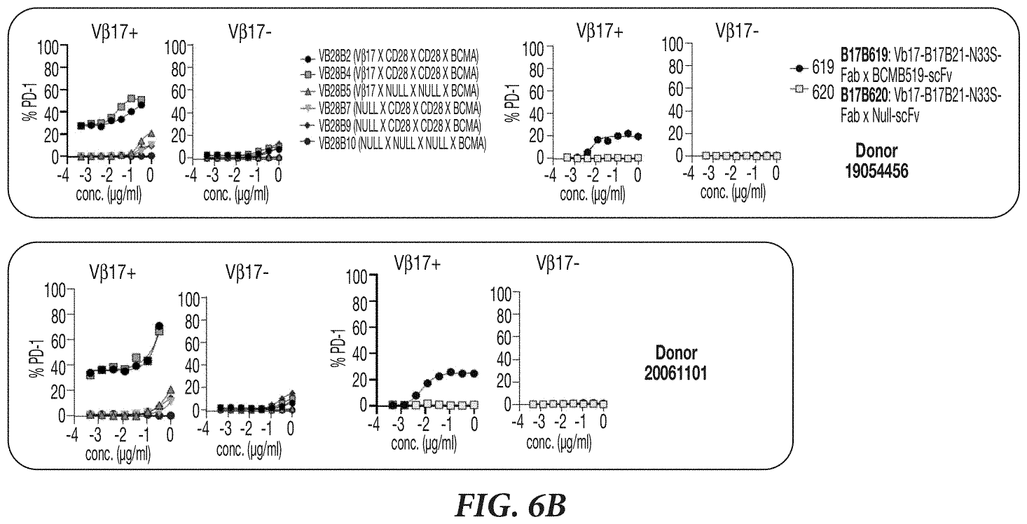

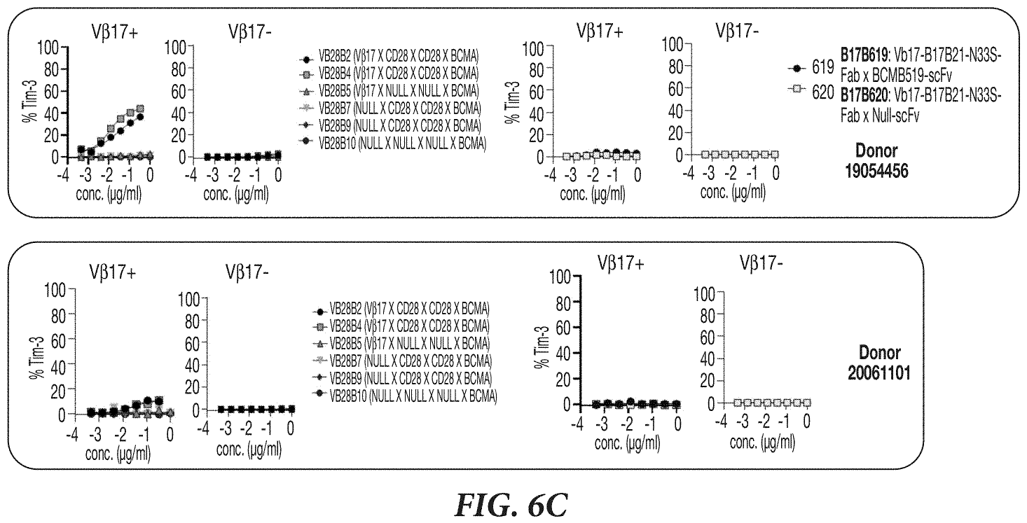

To test whether the increased activation induced by the engagement of CD28 on T cells would result in higher exhaustion of the Vβ17 T cells, pan T cells were cocultured with H929 cells in the presence of the Vβ17×CD28×BCMA trispecific antibodies or Vβ17×BCMA antibodies and their Null arm controls. To identify exhausted cells, TIM3, LAG3 and PD1 markers were used although PD1 upregulation is also a sign of T cell activation. PD1 was found to be upregulated on Vβ17+ T cells in the presence of both the Vβ17×BCMA antibodies and the Vβ17×CD28×BCMA antibodies (See FIG. 6B). As was observed with the activation markers, percentage of PD1+VB17 T cells was higher with Vβ17×CD28×BCMA antibodies as compared to Vβ17×BCMA antibody. LAG3 and TIM3 were observed to be induced only on a small fraction of the Vβ17 T cells and no upregulation was seen on the Vβ17− T cells (See FIGS. 6A and 6C). Overall, only 20% of the Vβ17 T cells were found to express TIM3 and LAG3.

### Example 3.8: Engagement of CD28 Potently Enhances the Cytotoxicity Induced by VB17 T Cells

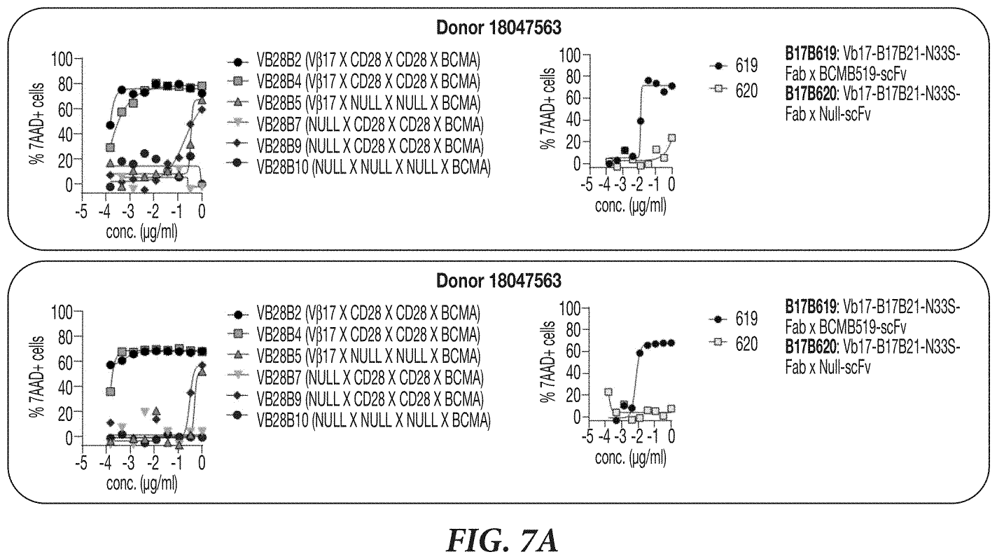

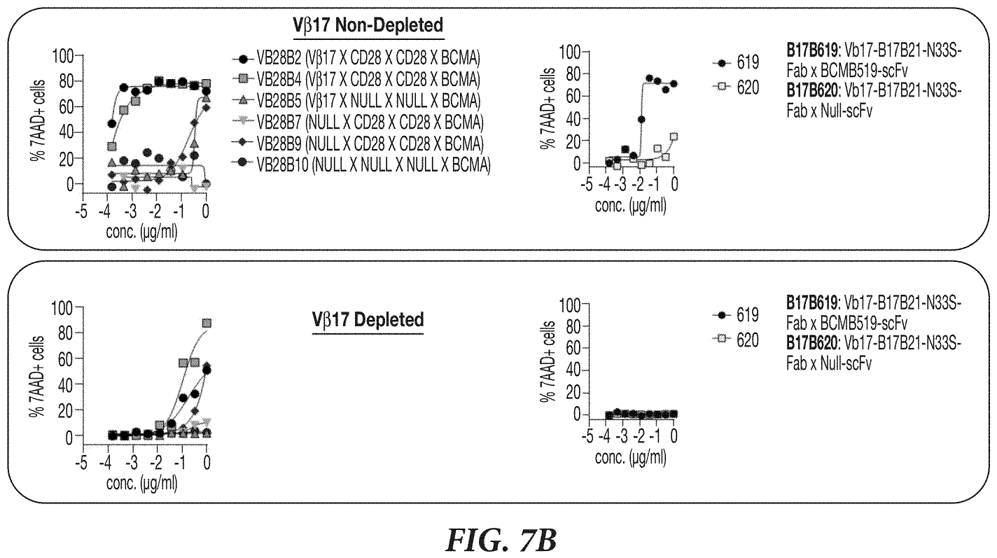

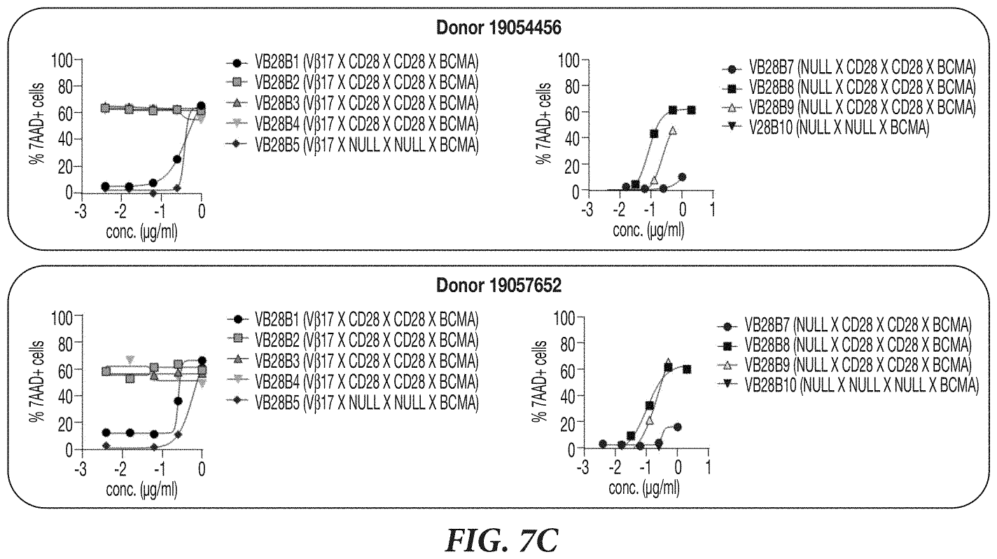

To examine if the increased activation of the Vβ17 T cells in the presence of the Vβ17×CD28×BCMA trispecific antibodies also resulted in an increase in the functional activity of the Vβ17 T cells, cytotoxicity assays using H929 cells were set up (See FIGS. 7A, 7B and 7C). Vβ17×BCMA antibodies induced H929 target cell death in a dose dependent manner with an EC₅₀ of ˜0.01 μg/ml. This cytotoxicity was very strongly enhanced by the Vβ17×CD28×BCMA antibodies by about 100-fold. VB28B7 antibody (null×CD28×BCMA) did not show any cytotoxicity thus showing the specificity of the increased cytotoxic response. VB28B8 and VB28B9 (null×CD28×BCMA) antibodies which had shown activation of the Vβ17 T cells in the absence of the Vβ17 arm also showed cytotoxicity against H929 cells albeit at lower levels than Vβ17×CD28×BCMA antibodies. To show the specificity of the cytotoxic response induced by the Vβ17×CD28×BCMA antibodies, cytotoxic activity of Vβ17 T cells depleted Pan T cells was examined. Depletion of the Vβ17 T cells resulted in almost complete abrogation of the cytotoxic activity of the Vβ17×CD28×BCMA antibodies. Activity of the Vβ17×BCMA antibody was completely lost with the depletion of the Vβ17 T cells.

### Example 3.9: Engagement of CD28 Potently Enhances the Cytokine Secretion

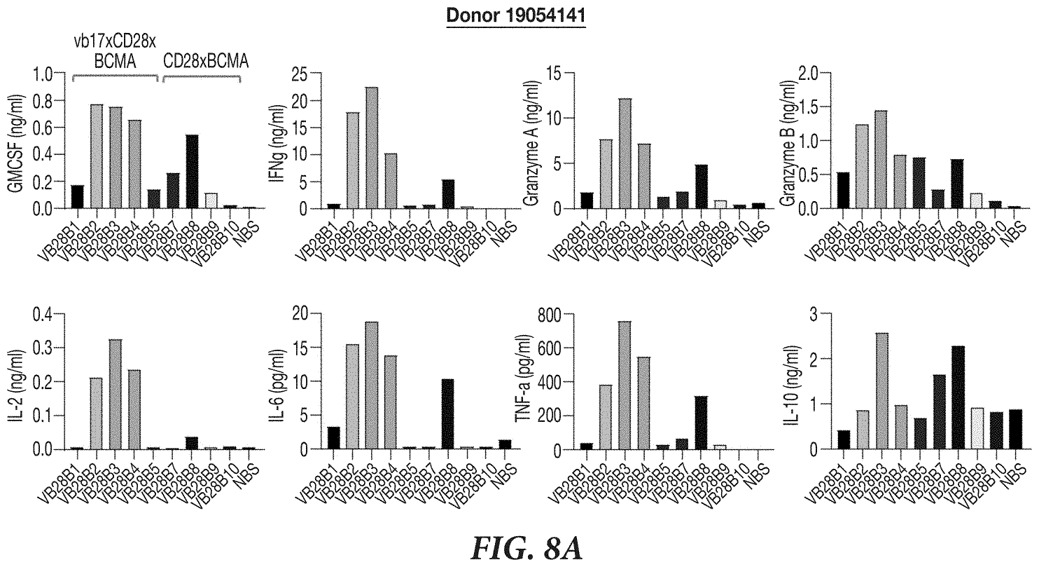

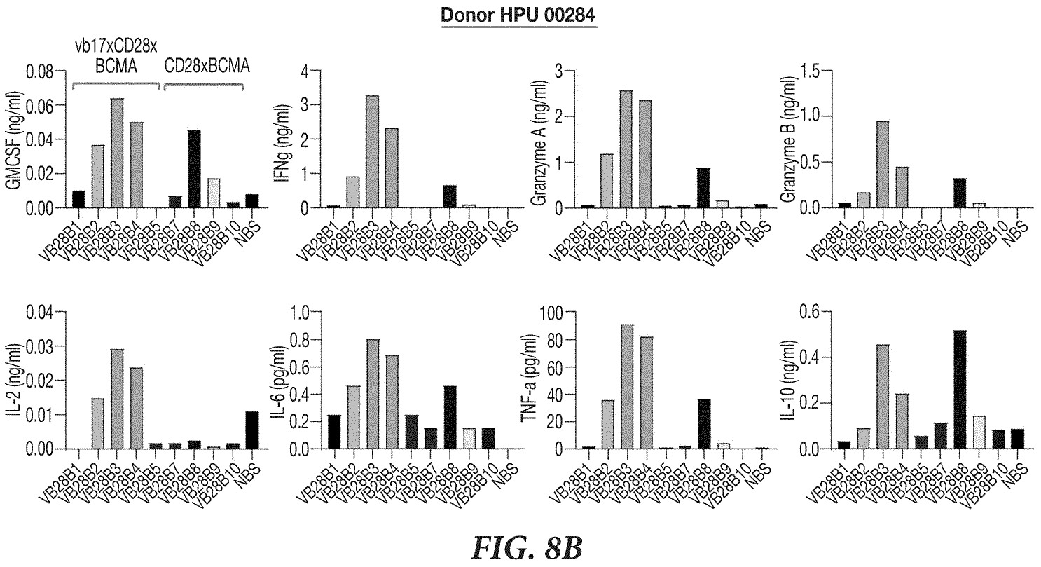

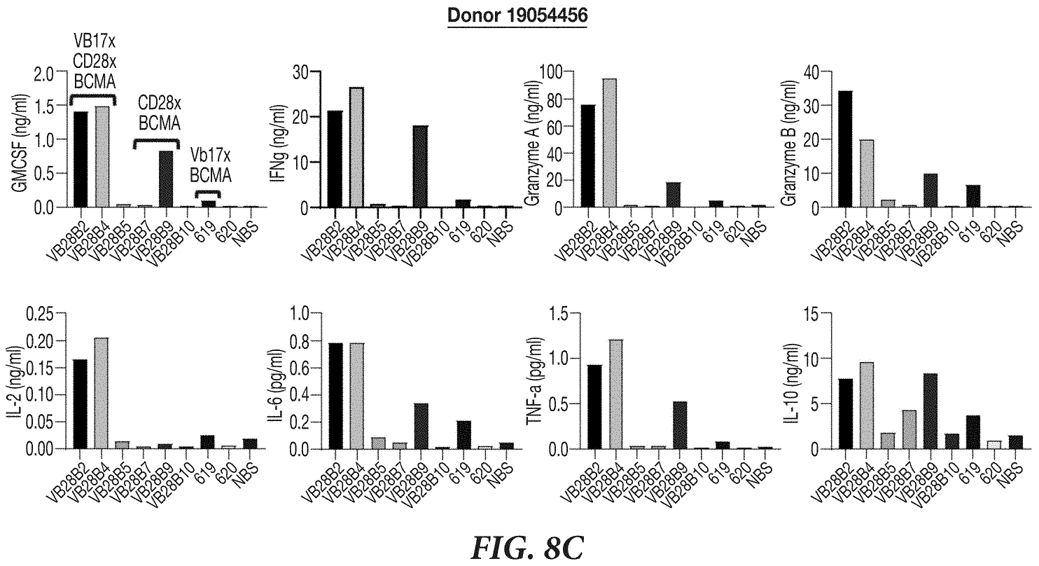

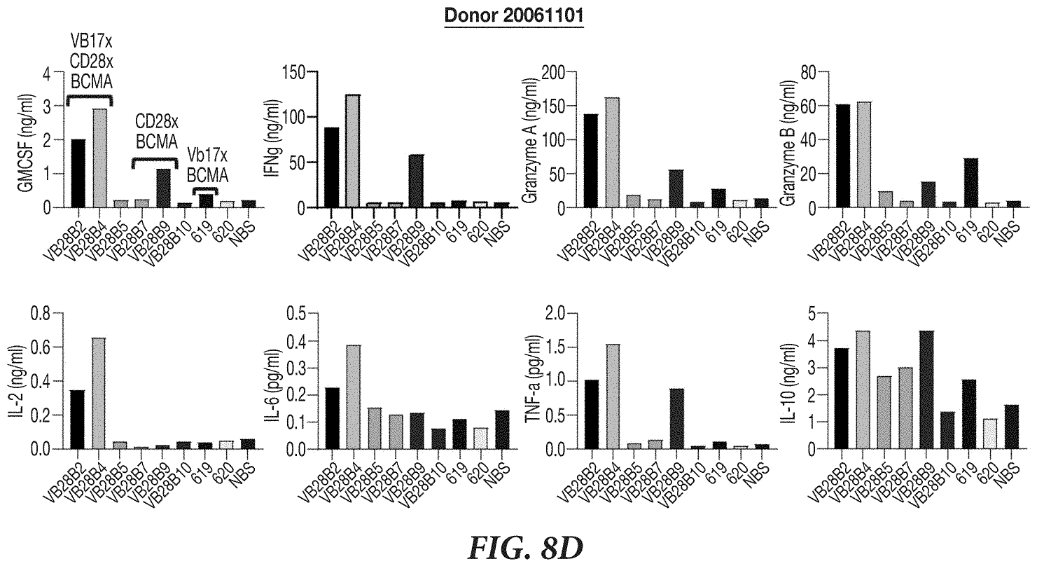

Consistent with the enhancement of T cell activation and cytotoxicity, Vβ17×CD28×BCMA trispecifics also showed superior cytokine release in comparison to Vβ17×BCMA antibodies and Null×CD28×BCMA antibodies (See FIGS. 8A, 8B, 8C and 8D). VB28B1 antibody as expected did not show an increased cytokine release since this antibody showed poor CD28 binding and no Vβ17 T cell activation or cytotoxicity against H929 target cells. VB28B8 and VB29B9 antibodies also showed potent cytokine release although the levels were lower than the Vβ17×CD28×BCMA antibodies. This was in line with the activation profile of Vβ17 T cells observed with these antibodies.

### Example 3.10: Expression of Costimulatory Ligands on BCMA Expressing H929 Cell Lines

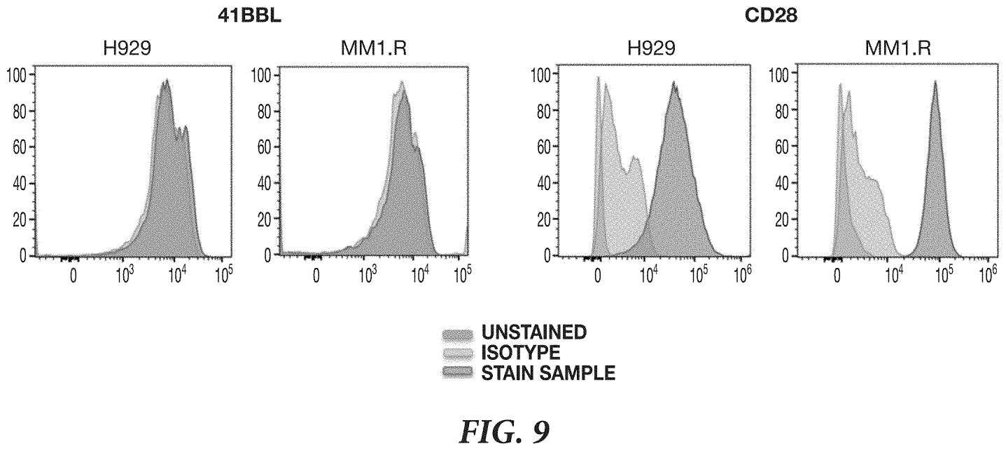

To check for expression of costimulatory ligands on multiple myeloma cell lines, multiple myeloma cell lines MM1.R and H929 were stained with anti-human CD28 (purified anti human CD28, cat #555725, BD Pharmingen) for 30 minutes followed by staining with goat anti-mouse IgG (cat #405307, Biolegend). For 41BBL expression, cells were stained with anti-human CD137 antibody (cat #311506) for 30 minutes on ice as per the manufacturer's protocol. All staining was done post Fc block. Following the staining, cells were acquired on the NOVOCYTE flow cytometer. Cells were gated on FSC/SSC, followed by live cell gating and CD28 expression and 41BB expression was plotted as histograms. As shown in FIG. 9, both tested multiple myeloma cell lines, MM1.R and H929 were found to express CD28 while no expression of 4IBBL was observed on either of the cell lines. This is in line with previous reports that have shown CD28 expression on primary myeloma plasma cells.

### Example 3.11: Evaluation of Anti-Vβ17/Anti-CD28/Anti-BCMA Trispecific Antibody

The variable region sequence of anti-Vβ17, anti-CD28 and anti-BCMA antibodies were used to generate a trispecific human IgG1 antibody to be tested for cytotoxicity, activation, cytokine release and proliferation. The trispecific antibodies were produced and the below described assays performed according to Example 3.1.

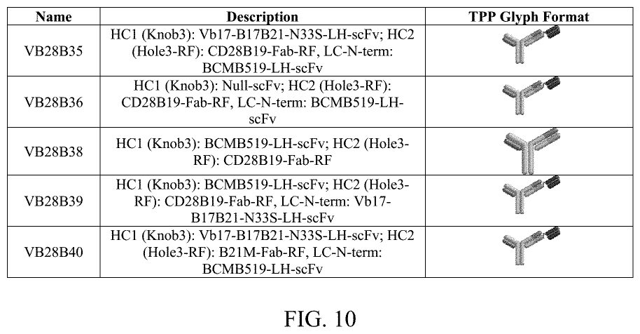

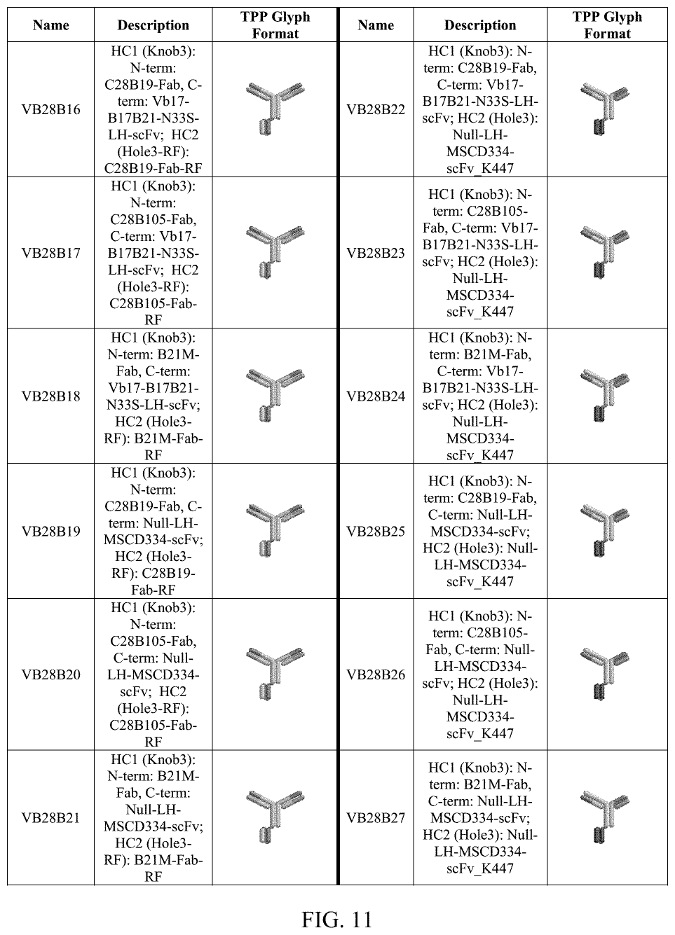

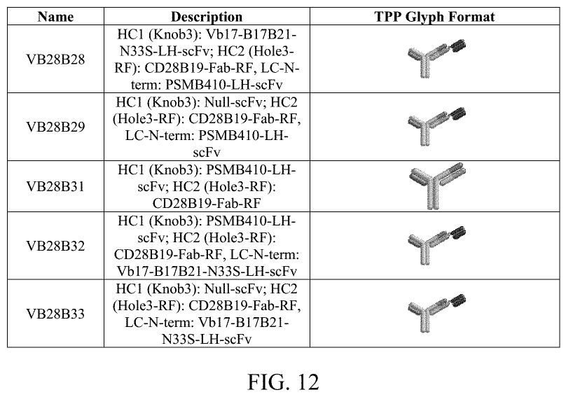

Exemplary anti-Vβ17/anti-CD28/anti-BCMA antibodies with their description are shown in FIG. 10. Exemplary control antibodies for anti-Vβ17/anti-CD28/anti-BCMA antibodies and their description are shown in FIG. 11.

### Evaluation of Anti-Vβ17/Anti-CD28/Anti-BCMA Antibodies

Cytotoxicity assays were performed according to Example 3.1 in CD28 KO H929 cells. CD28 KO has no effect on cytotoxicity or Vβ17 T cell activation induced by CD28 engagement for anti-Vβ17/anti-CD28/anti-BCMA antibodies as shown in Table 15. Activation profile of Vβ17+ T cells for anti-Vβ17/anti-CD28/anti-BCMA antibodies by expression of CD25 and CD71 was monitored in CD28 KO H929 cells and is shown in Table 16.

TABLE 15
EC₅₀ of anti-Vβ17/anti-CD28/anti-BMCA trispecific antibody.

| Antibody | Cytotoxicity EC₅₀ (μg/ml) |
|---|---|
| VB28B2-H929 WT | <0.000152 |
| VB28B7-H929 WT | >1 |
| VB28B2-H929 CD28 KO | <0.000152 |
| VB28B7-H929 CD28 KO | >1 |
| B17B619-H929 WT | 0.3201 |
| B17B620-H929 WT | ND |
| B17B619-H929 CD28 KO | 0.1328 |
| B17B620-H929 CD28 KO | ND |

TABLE 16
EC₅₀ of anti-Vβ17/anti-CD28/anti-BMCA trispecific antibody.

| Antibody | CD25 EC₅₀ (μg/ml) | CD71 EC₅₀ (μg/ml) |
|---|---|---|
| VB28B2-H929 WT | <0.000152 | <0.000152 |
| VB28B7-H929 WT | >1 | >1 |
| VB28B2-H929 CD28 KO | 0.0005493 | 0.0004558 |
| VB28B7-H929 CD28 KO | >1 | >1 |
| B17B619-H929 WT | 0.2734 | 0.2821 |
| B17B620-H929 WT | ND | ND |
| B17B619-H929 CD28 KO | 0.1502 | 0.1465 |
| B17B620-H929 CD28 KO | ND | ND |

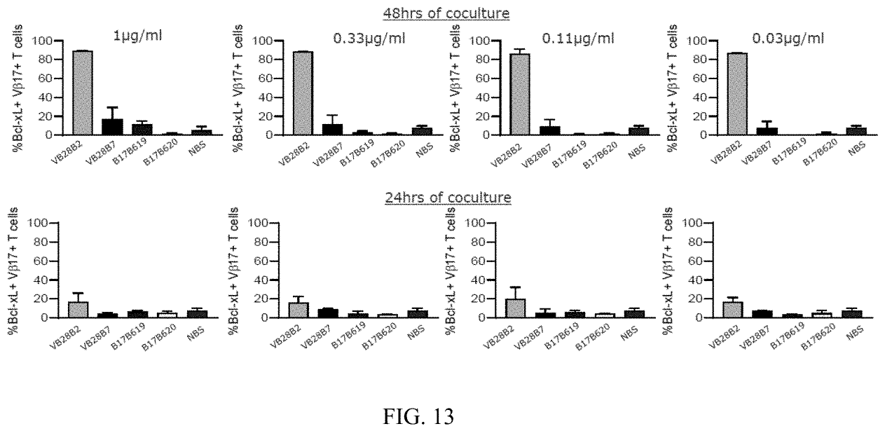

Next, Bcl-xL level was analyzed in a Pan T cell assay comprising a 1:1 ratio of Vβ17 T cells:H929 cells. Bcl-xL expression was determined as described below. As shown in FIG. 13, apoptotic protein Bcl-xL is induced in Vβ17 T cells with CD28 activation.

BCL-XL Expression.

PBMCs were thawed and subjected to Pan T cell isolation using the EasySep™ Human T Cell Isolation Kit (17951, Stemcell). H929 cells were counted and plated at 10,000 cells per well in a 96 well U-bottom plate in 100 μl of RPMI media (ATCC modification)+20% FBS. Vβ17 T cell frequency was determined in the Pan T cells from each donor using the PE conjugated TCR Vβ17 antibody (IM2048, Beckman Coulter). Pan T cells were then added to the plated H929 cells such that an effector to target ratio of 1 Vβ17:1 H929 cell was obtained. 80 μl of the effector cell suspension in RPMI media+10% FBS was added per well. 10× concentration of the antibodies were prepared (10 μg/ml) followed by 3 fold serial dilutions in media. 20 μl of the serially diluted antibody was added to the 180 μl of the co-culture making the final concentration of the antibodies in coculture as 1×. The cell culture plates were incubated at 37° C. for 24 and 48 hrs. At the end of the incubation period, the cells were spun down at 1500 rpm for 5 mins and were stained with APC-Cy7 live/dead stain (L10119, Thermofisher) followed by staining with Fc block (422302, Biolegend). The cell pellet was taken for staining with Brilliant Violet 785™ anti-human CD25 (302638, Biolegend), PE conjugated TCR Vβ17 antibody (IM2048, Beckman Coulter) and Brilliant Violet 510™ anti-human αβ (306734, Biolegend) as per the manufacturer's recommendation. Cells were washed post staining in FACS buffer and fixed with BD cytofix buffer (554655, BD Bioscience). Post fixation, samples were permeabilised by adding 90% methanol and was incubated for 10 min on ice. The cells were washed post incubation and then stained with Bcl-XL (54H6) Rabbit mAb APC conjugated (1:50 dilution) (12099S, Cell signalling). The cells were incubated for 45 min at 4° C. Post incubation, the cells were washed and then resuspended in FACS buffer and acquired on the NOVOCYTE flow cytometer. Cells were gated on the live cell population, followed by gating on Vβ17+ T cells. Expression of Bcl-XL was monitored at 24 and 48 hrs at different concentration and plotted using GRAPHPAD Prism version 8.1.1.

### Evaluation of Exemplary Control and Null Antibodies

Cytotoxicity assays were performed according to Example 3.1 in Vβ17+ T cells and H929 cells (1:1 ET ratio). As shown in Table 17, combining CD28 costimulation with Vβ17 TCR stimulation potently enhances cytotoxicity compared to Vβ17 TCR stimulation alone. Anti-Vβ17/anti-CD28/anti-CD28 antibody shows similar cytotoxicity to anti-Vβ17/anti-BCMA antibody when using the C28B19 binder as H929 cells also express CD28. No cytotoxicity was shown with CD28 bispecific, however, when CD28 was combined with BCMA, cytotoxicity was induced.

TABLE 17
EC₅₀ of anti-Vβ17/anti-CD28/anti-BMCA trispecific antibody.

| Antibody | Cytotoxicity EC₅₀ (μg/ml) |
|---|---|
| VB28B2 | <0.000152 |
| VB28B7 | >1 |
| VB28B16 | 0.01714 |
| VB28B19 | ND |
| VB28B4 | <0.000152 |
| VB28B9 | 0.08105 |
| VB28B17 | 0.005312 |
| VB28B20 | ND |
| B17B619 | 0.04087 |
| B17B620 | ND |

Table 18 shows the activation profile of Vβ17+ T cells with the C28B19 binder. Combining CD28 costimulation with Vβ17 TCR stimulation potently enhances the activation of Vβ17+ cells compared to Vβ17 TCR stimulation alone. Anti-Vβ17/anti-CD28/anti-CD28 antibody shows higher activation as compared to anti-Vβ17/anti-BCMA antibody. No activation was observed with anti-CD28/null bispecific, but activation was observed with anti-CB28/anti-BCMA antibody.

TABLE 18
Summary of activation profile of Vβ17 cells with the C28B19 binder.

| | | | | | proliferation EC₅₀ | |
|---|---|---|---|---|---|---|
| | CD25 EC₅₀ (μg/ml) | | CD71 EC₅₀ (μg/ml) | | (μg/ml) | |
| | Donor 1 | Donor 2 | Donor 1 | Donor 2 | Donor 1 | Donor 2 |
| Antibody | (20001461) | (20001464) | (20001461) | (20001464) | (20001461) | (20001464) |
| VB28B2 | <0.000152 | <0.000152 | <0.000152 | <0.000152 | 0.0007426 | <0.000152 |
| VB28B7 | >1 | >1 | >1 | >1 | >1 | >1 |
| VB28B16 | 0.009315 | 0.005331 | 0.008532 | 0.003268 | 0.01215 | 0.006202 |
| VB28B19 | >1 | >1 | >1 | >1 | ND | ND |
| B17B619 | 0.05358 | 0.01947 | 0.04002 | 0.01204 | 0.1203 | 0.02921 |
| B17B620 | >1 | >1 | >1 | >1 | >1 | >1 |

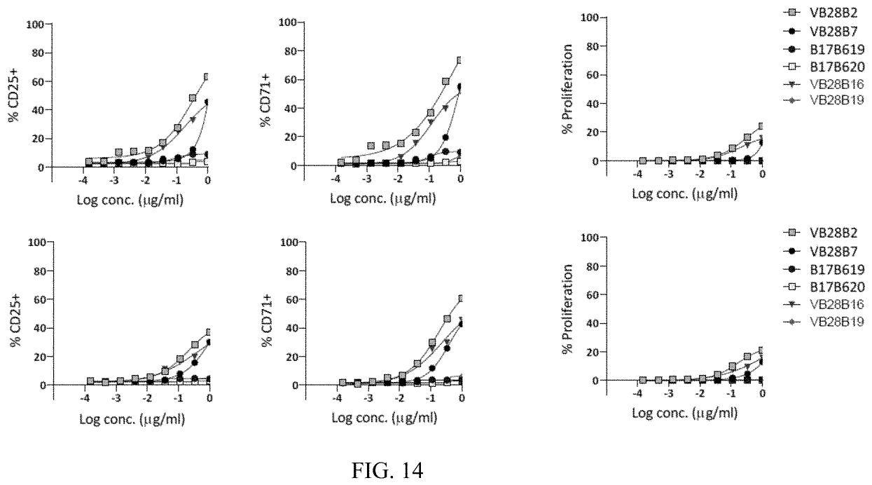

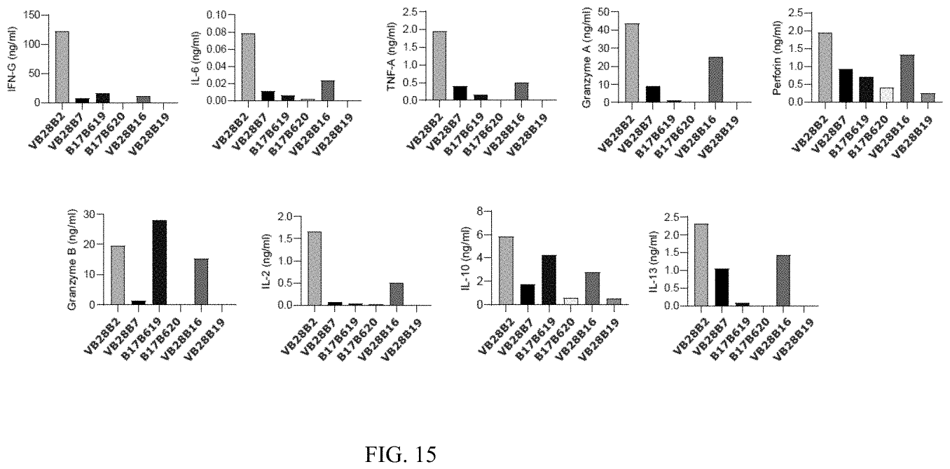

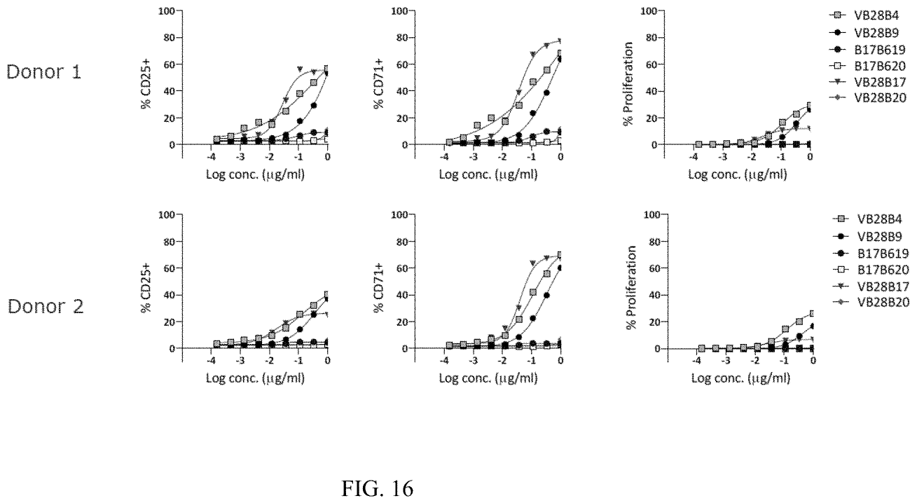

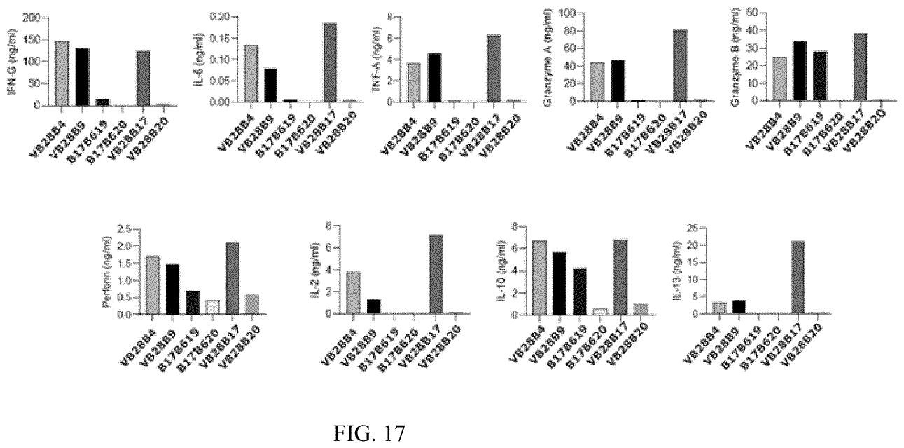

FIG. 14 shows the activation profile of Vβ17− T cells with anti-Vβ17/anti-CD28 control antibodies containing a C28B19 binder, while FIG. 16 shows the activation profile of Vβ17− T cells with C28B105 binder. FIG. 15 shows cytokine release from anti-Vβ17/anti-CD28 control antibodies containing a C28B19 binder, while FIG. 17 shows cytokine release with C28B105 binder. Cytokine secretion is enhanced with CD28 costimulation in both FIG. 15 and FIG. 17. Table 19 shows the activation profile of Vβ17+ T cells with the C28B105 binder.

TABLE 19
Summary of activation profile of Vβ17 cells with the C28B105 binder.

| | | | | | proliferation EC₅₀ | |
|---|---|---|---|---|---|---|
| | CD25 EC₅₀ (μg/ml) | | CD71 EC₅₀ (μg/ml) | | (μg/ml) | |
| | Donor 1 | Donor 2 | Donor 1 | Donor 2 | Donor 1 | Donor 2 |
| Antibody | (20001461) | (20001464) | (20001461) | (20001464) | (20001461) | (20001464) |
| VB28B4 | <0.000152 | <0.000152 | <0.000152 | <0.000152 | 0.0007701 | <0.000152 |
| VB28B9 | >1 | >1 | >1 | >1 | >1 | >1 |
| VB28B17 | 0.002414 | 0.001535 | 0.002262 | 0.001300 | 0.004898 | 0.001532 |
| VB28B20 | >1 | >1 | >1 | >1 | >1 | ND |
| B17B619 | 0.05358 | 0.01947 | 0.04002 | 0.01204 | 0.1203 | 0.02921 |
| B17B620 | >1 | >1 | >1 | >1 | >1 | >1 |

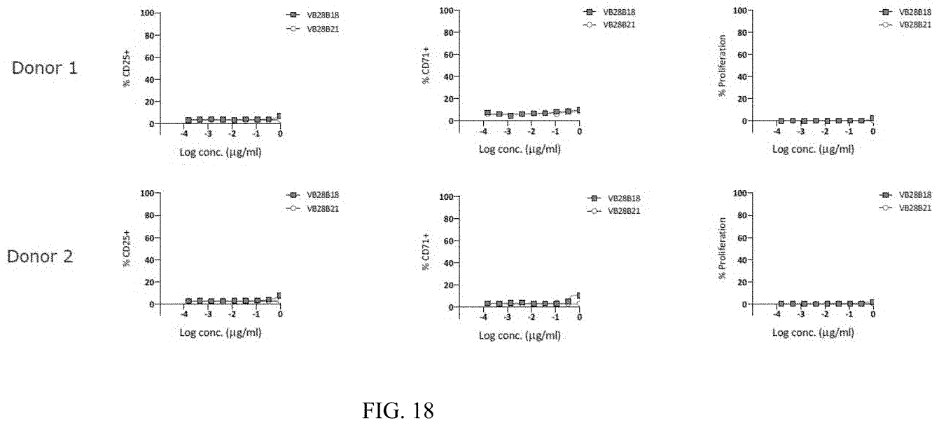

FIG. 18 shows the activation profile of Vβ17+ T cells with anti-Vβ17/null or null/null controls. There was no activation of Vβ17+ T cells with null arm controls.

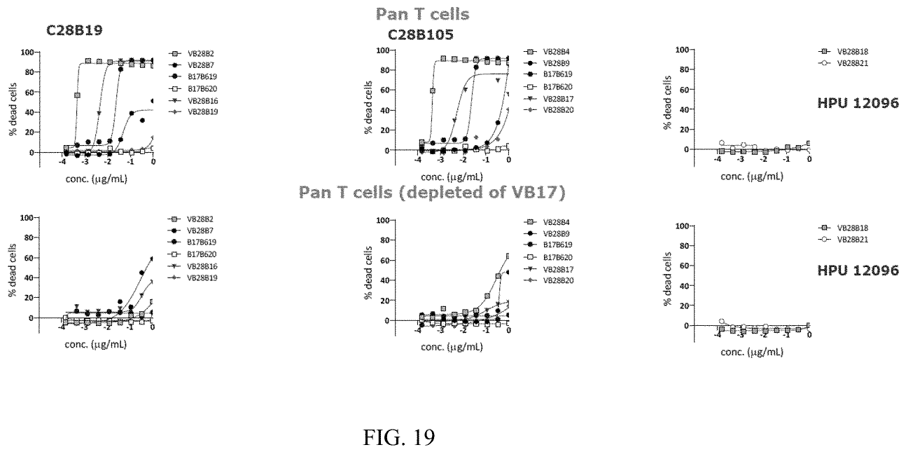

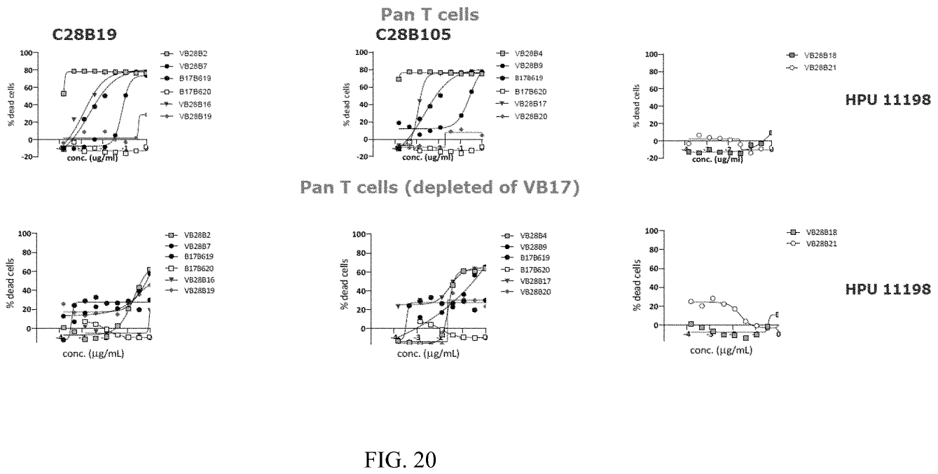

Next, the performance of anti-Vβ17/anti-CD28/anti-BCMA antibodies were compared to their respective controls. Anti-Vβ17/anti-CD28/anti-BCMA antibodies with C28B19 or C28B105 binders depleted Vβ17 T cells, which resulted in complete loss of cytotoxicity induced by anti-Vβ17/anti-BCMA antibody (See FIG. 19 and FIG. 20). There was also no observed cytotoxicity with anti-CD28/null bispecific antibody, only with anti-CD28/anti-BCMA antibody. Table 20 shows a summary of the Vβ17 T cell cytotoxicity observed.

TABLE 20
Summary of cytotoxicity observed in presence of Vβ17 T cells.

Cytotoxicity EC₅₀ (μg/ml)

| | Donor 1 | Donor 2 |
|---|---|---|
| Antibody | (HPU 12096) | (HPU 11198) |
| VB28B2 | <0.000152 | <0.000152 |
| VB28B7 | 0.04744 | 0.07568 |
| VB28B16 | 0.004458 | 0.001278 |
| VB28B19 | >1 | ND |
| VB28B4 | <0.000152 | <0.000152 |
| VB28B9 | >1 | >1 |
| VB28B17 | 0.005208 | 0.001171 |
| VB28B20 | >1 | ND |
| B17B619 | 0.02318 | 0.002288 |
| B17B620 | >1 | ND |

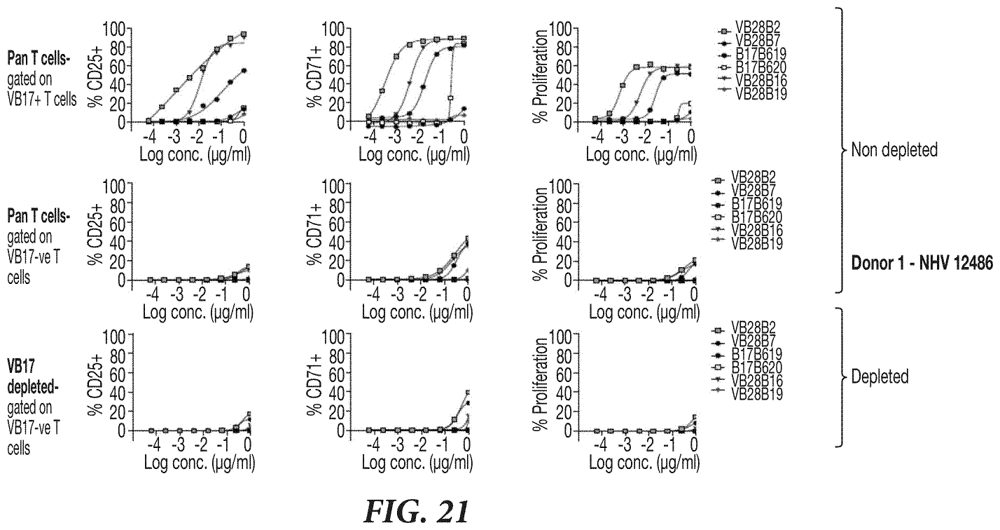

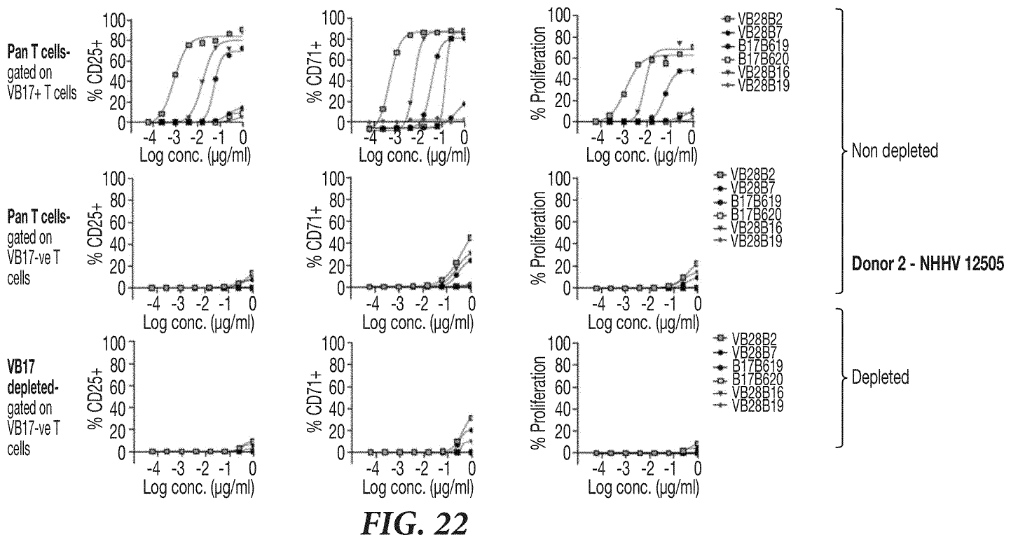

Next, cytotoxicity was measured in Pan T cells using anti-Vβ17/anti-CD28/anti-BCMA antibodies and their control antibodies. FIG. 21 shows the activation profile of Vβ17+ T cells and Vβ17− T cells, with anti-Vβ17/anti-CD28/anti-BCMA antibodies in Donor 1. Combining CD28 costimulation with Vβ17 TCR stimulation potently enhances the activation of Vβ17+ T cells compared to Vβ17 TCR stimulation alone. Anti-Vβ17/anti-CD28/anti-BCMA antibody shows higher activation compared to anti-Vβ17/anti-BCMA antibody, there was no activation with anti-CD28/null bispecific antibody, and activation was observed with anti-CD28/anti-BCMA antibody. FIG. 22 shows the activation profile of Vβ17+ T cells and Vβ17− T cells, with anti-Vβ17/anti-CD28/anti-BCMA antibodies in Donor 2. Combining CD28 costimulation with Vβ17 TCR stimulation potently enhances the activation of Vβ17+ T cells compared to Vβ17 TCR stimulation alone. Anti-Vβ17/anti-CD28/anti-BCMA antibody shows higher activation compared to anti-Vβ17/anti-BCMA antibody, there was no activation with anti-CD28/null bispecific antibody, and activation was observed with anti-CD28/anti-BCMA antibody. Table 21 provides a summary of the activation profile of Vβ17 cells with the C29B19 binder.

TABLE 21
summary of the activation profile of Vβ17 cells with the C29B19 binder.

| | CD25 EC₅₀ (μg/ml) | | CD71 EC₅₀ (μg/ml) | | proliferation EC₅₀ (μg/ml) | |
|---|---|---|---|---|---|---|
| | Donor 1 | Donor 2 | Donor 1 | Donor 2 | Donor 1 | Donor 2 |
| Antibody | (NHV12486) | (NHHV12505) | (NHV12486) | (NHHV12505) | (NHV12486) | (NHHV12505) |
| VB28B2 | 0.001219 | 0.0008328 | 0.0003172 | 0.0004703 | 0.0007165 | 0.001188 |
| VB28B7 | >1 | >1 | >1 | >1 | >1 | >1 |
| VB28B16 | 0.01181 | 0.01634 | 0.004179 | 0.005700 | 0.005637 | 0.007809 |
| VB28B19 | >1 | >1 | >1 | ND | >1 | >1 |
| B17B619 | >1 | 0.05333 | 0.01854 | 0.03407 | 0.02637 | 0.05754 |
| B17B620 | ND | ND | ND | ND | ND | ND |

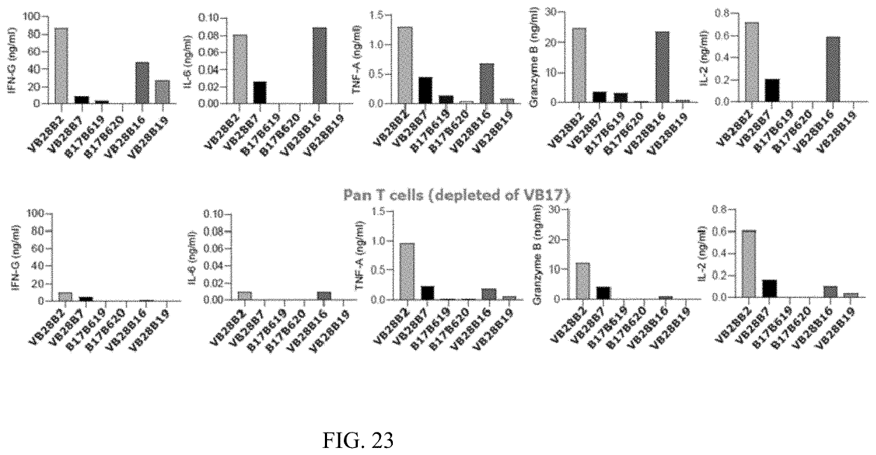

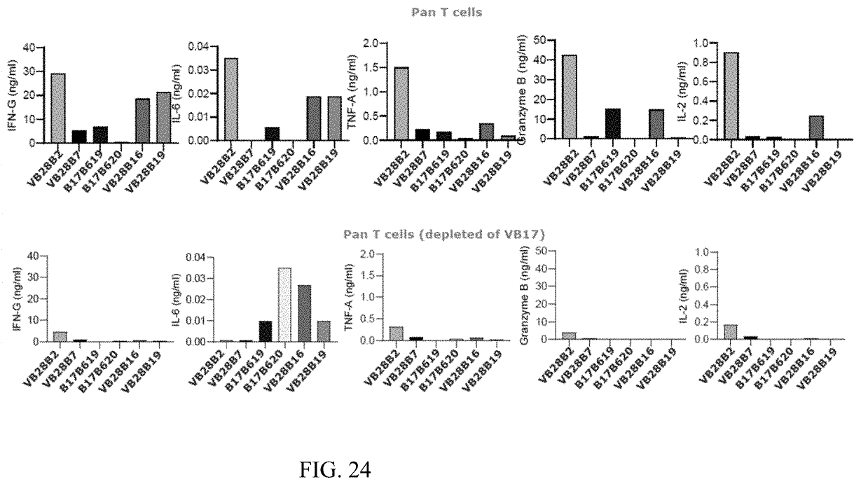

FIG. 23 and FIG. 24 show cytokine release in Pan T cells expressing or depleted of Vβ17 with a C28B19 binder in Donor 1 (FIG. 23) Donor 2 (FIG. 24). Cytokine secretion is enhanced with CD28 costimulation in a Vβ17 T cell dependent manner.

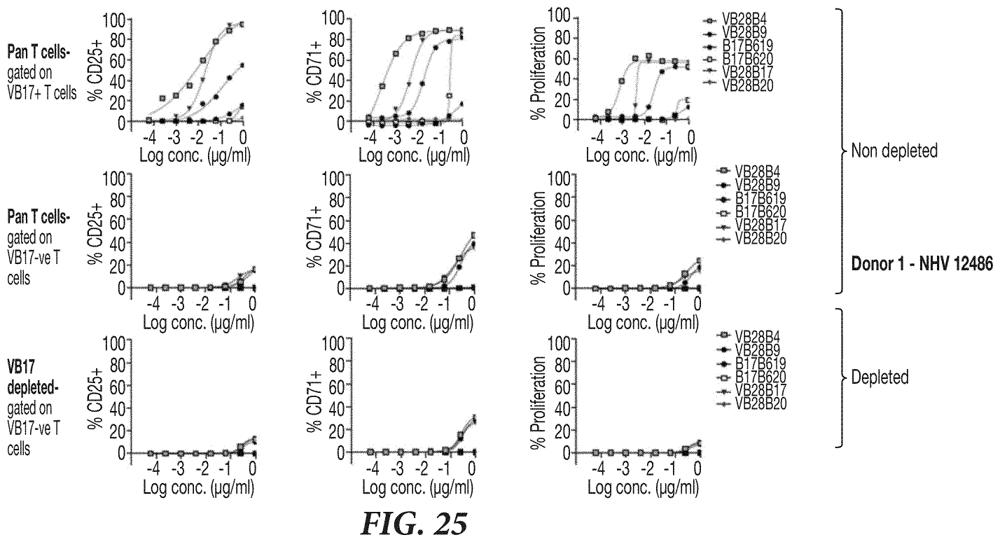

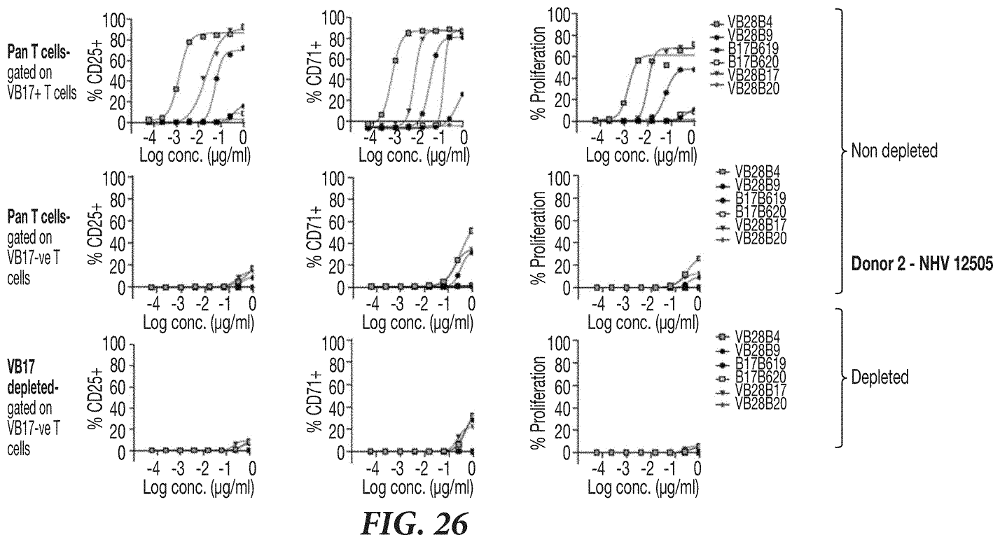

FIG. 25 and FIG. 26 show the activation profile of Vβ17+ T cells and Vβ17− T cells with anti-Vβ17/anti-CD28/anti-BCMA antibodies containing C28B105 in Donor 1 (FIG. 25) or Donor 2 (FIG. 26). Combining CD28 costimulation with Vβ17 TCR stimulation potently enhances the activation of Vβ17+ cells compared to Vβ17 TCR stimulation alone. Anti-Vβ17/anti-CD28/anti-BCMA antibody shows higher activation as compared to anti-Vβ17/anti-BCMA antibody. There was no activation observed with anti-CD28/null bispecific antibody but activation was observed with anti-CD28/anti-BCMA antibody. Table 22 further summarizes the activation profile of Vβ17 cells with the C28B105 binder.

TABLE 22
Summary of activation profile of Vβ17 cells with the C28B105 binder.

| | CD25 EC₅₀ (μg/ml) | | CD71 EC₅₀ (μg/ml) | | proliferation EC₅₀ (μg/ml) | |
|---|---|---|---|---|---|---|
| | Donor 1 | Donor 2 | Donor 1 | Donor 2 | Donor 1 | Donor 2 |
| Antibody | (NHV12486) | (NHHV12505) | (NHV12486) | (NHHV12505) | (NHV12486) | (NHHV12505) |
| VB28B4 | 0.009595 | 0.0001320 | 0.0002389 | 0.0006759 | 0.0007455 | 0.001514 |
| VB28B9 | >1 | >1 | >1 | >1 | >1 | >1 |
| VB28B17 | 0.02020 | 0.02107 | 0.004712 | 0.006995 | ND | 0.01027 |
| VB28B20 | >1 | >1 | ND | ND | ND | ND |
| B17B619 | 0.1181 | 0.05333 | 0.01854 | 0.03407 | 0.02637 | 0.05754 |
| B17B620 | ND | ND | ND | ND | ND | ND |

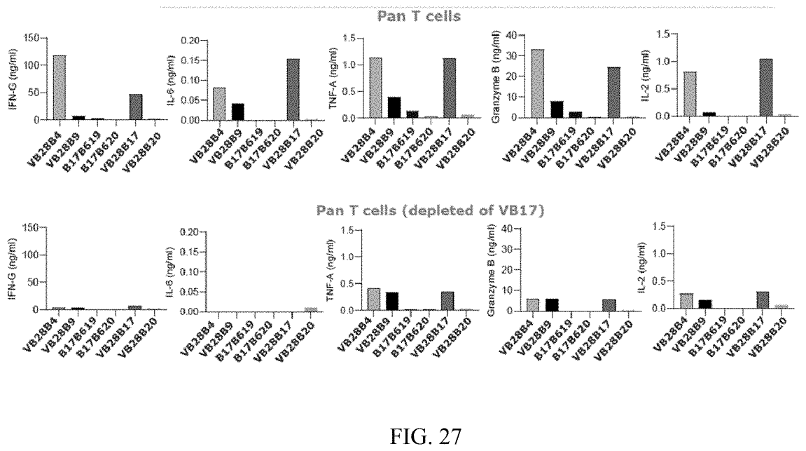

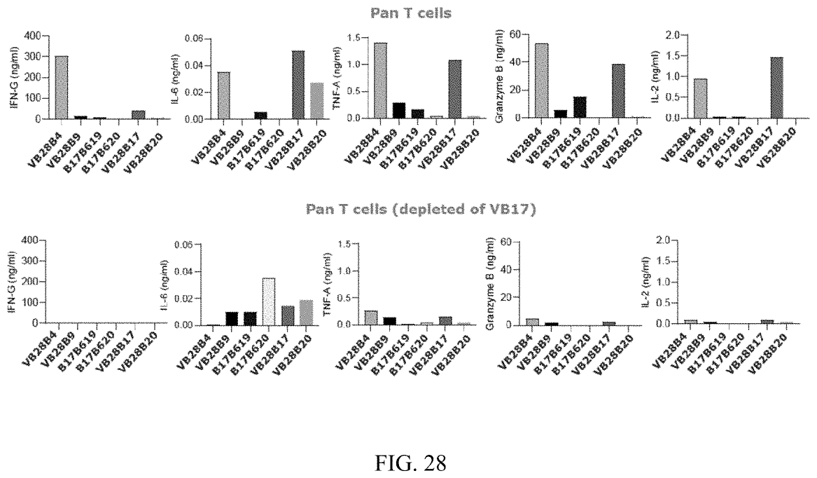

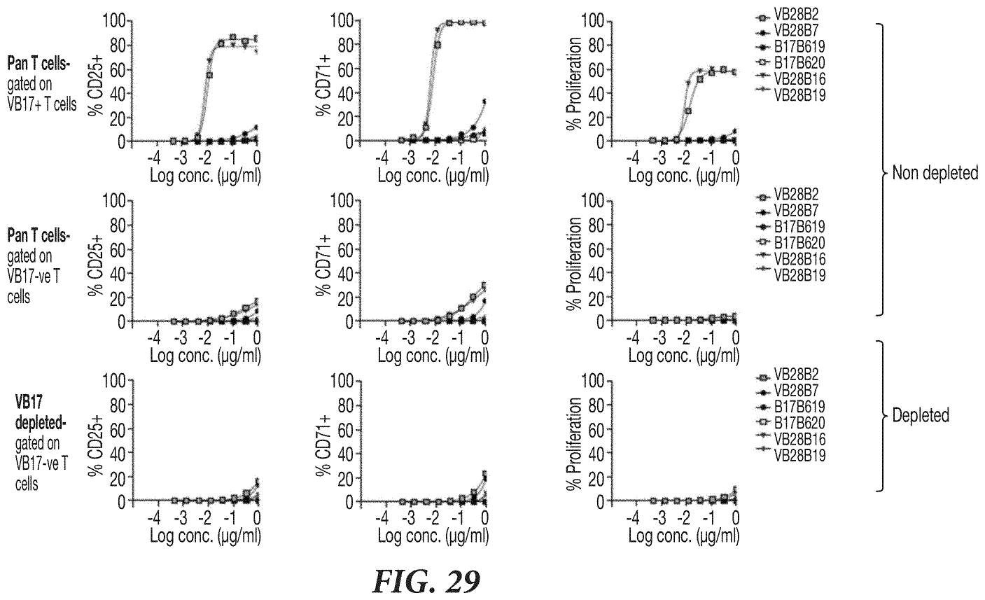

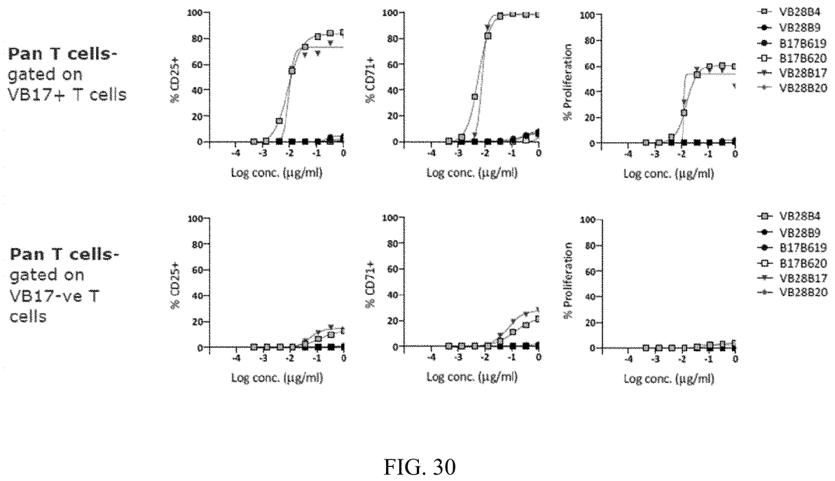

FIG. 27 and FIG. 28 show cytokine release in Pan T cells with or depleted of Vβ17 in Donor 1 (FIG. 29) or Donor 2 (FIG. 30).

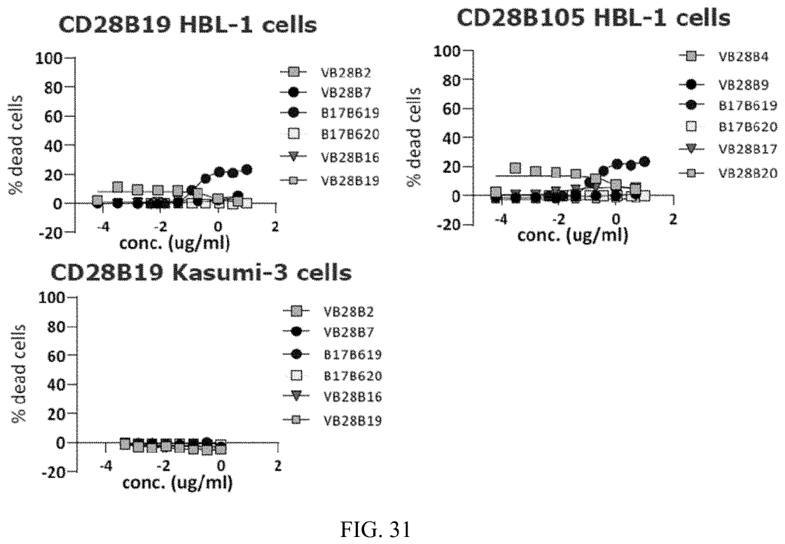

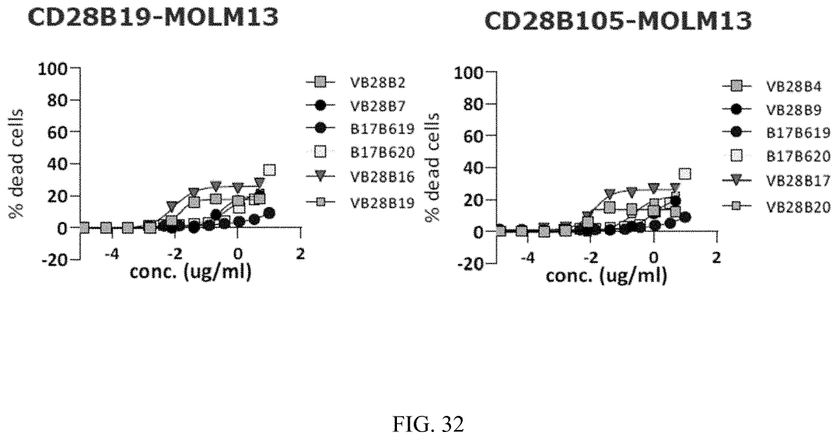

FIG. 31 shows activation profile of Vβ17+ and Vβ17− T cells with anti-Vβ17/anti-CD28/anti-BCMA antibodies containing C28B19 in the absence of target cells, whereas FIG. 32 shows the activation profile of Vβ17+ and Vβ17− T cells with anti-Vβ17/anti-CD28/anti-BCMA antibodies containing C28B105. In FIG. 31, combining CD28 costimulation with Vβ17 TCR stimulation enhanced the activation of Vβ17+ cells compared to Vβ17 TCR stimulation alone or in the absence of target cells. Anti-Vβ17/anti-CD28/anti-CD28 antibodies show similar activation as compared to anti-Vβ17/anti-CD28/anti-BCMA antibodies, activation is observed with anti-CD28/anti-BCMA antibody, but there is no activation with anti-CD28/null or anti-CD28/anti-BCMA bispecific antibodies. In FIG. 32, combining CD28 costimulation with Vβ17 TCR stimulation enhanced the activation of Vβ17+ cells compared to Vβ17 TCR stimulation alone. Anti-Vβ17/anti-CD28/anti-CD28 antibodies show higher activation as compared to anti-Vβ17/anti-BCMA antibodies, activation is observed with anti-CD28/anti-BCMA antibody, but there is no activation with anti-CD28/null bispecific antibodies. Table 23 further summarizes Vβ17 T cell activation in absence of target cells.

TABLE 23
Summary of Vβ17 T cell activation in absence of target cells.

| | | | proliferation |
|---|---|---|---|
| Antibody | CD25 EC₅₀ (μg/ml) | CD71 EC₅₀ (μg/ml) | EC₅₀ (μg/ml) |
| VB28B2 | 0.01035 | 0.008070 | 0.01406 |
| VB28B7 | >1 | >1 | >1 |
| VB28B16 | 0.008087 | 0.006496 | 0.008845 |
| VB28B19 | ND | >1 | ND |
| VB28B4 | 0.008640 | 0.005567 | 0.01513 |
| VB28B9 | ND | >1 | >1 |
| VB28B17 | 0.009397 | 0.007823 | ND |
| VB28B20 | ND | >1 | >1 |
| B17B619 | ND | >1 | ND |
| B17B620 | ND | ND | ND |

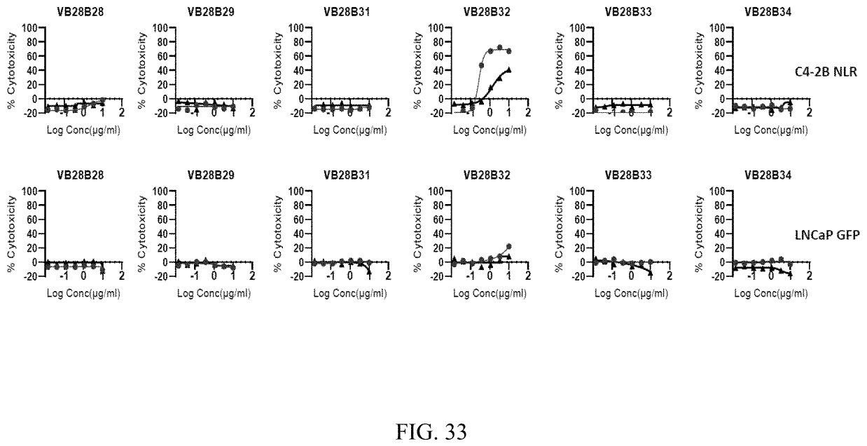

FIG. 33 and FIG. 34 show that anti-Vβ17/anti-CD28/anti-BCMA antibodies do not induce cytotoxicity against TAA cell lines.

### Example 4: Multispecific Antibodies that Bind Vβ17, CD28 and PSMA

The variable region sequence of anti-Vβ17, anti-CD28 and anti-PSMA antibodies were used to generate a trispecific human IgG1 antibody to be tested for binding and cytotoxicity. The trispecific antibodies were produced, and the below described assays performed, according to Example 3.1. Exemplary anti-Vβ17/anti-CD28/anti-PSMA antibodies are shown in FIG. 14.

Cytotoxicity Assay (INCUCYTE).

One day prior to the assay setup, C42B-NLR and LnCAP-GFP were plated 7000/well and kept overnight at 37° C. in a 5% CO2 incubator. On the day of the assay, PBMCs were thawed and subjected to Pan T cell isolation using the EasySep™ Human T Cell Isolation Kit (17951, Stemcell). Vβ17 T cell frequency was determined in the Pan T cells from each donor using the PE conjugated TCR Vβ17 antibody (IM2048, Beckman Coulter). CTV labeled Pan T cells were then added to the plated C42B-NLR and LnCAP-GFP cells such that an effector to target ratio of 1 Vβ17:1 target cell was obtained. 10× concentration of the antibodies were prepared followed by serial dilutions in media. 20 μl of the serially diluted antibody was added to the 180 μl of the co-culture making the final concentration of the antibodies in coculture as 1×. The cell culture plates were incubated at 37° C. Incucyte. Appropriate settings were selected with scans were scheduled after every 3 hrs. Post 72 Hrs and 96 Hrs of incubation, analysis was performed using INCUCYTE software. From the analysis, Fluorescence intensity from each well was obtained. Fluorescence intensity recorded is directly proportional to the live cell population in the well. Antibody specific percentage dead cells were calculated from the live population observed in wells as per the below mentioned formula. Percentage dead cells were plotted against log concentration of the antibody in a 4-parameter non-linear regression curve using GRAPHPAD Prism version 8.1.1. Percent live cells is calculated by evaluating intensity in the well with the antibody/intensity in the well with no antibody×100. The percent of lysis (dead cells) is calculated as 100−% live cells.

Pan T cell, LnCaP cell, and C4-2B cell binding using anti-Vβ17/anti-CD28/anti-PSMA antibodies is shown in Table 24 and FIG. 33.

TABLE 24
Summary of Binding to Pan T cells and Target cells.

| | Pan T cell Binding EC₅₀ | | LnCaP cell | C4-2B cell |
|---|---|---|---|---|
| | (μg/ml) | | Binding | Binding |
| Antibody | Donor 1 | Donor 2 | EC₅₀ (μg/ml) | EC₅₀ (μg/ml) |
| VB28B28 | >10 | >10 | 0.1596 | 1.095 |
| VB28B29 | >10 | >10 | 0.1349 | 0.9797 |
| VB28B31 | 1.292 | 0.9500 | 0.1498 | 1.2222 |
| VB28B32 | >10 | >10 | 0.2331 | 1.722 |
| VB28B33 | >10 | >10 | ND | ND |
| VB28B34 | >10 | >10 | 0.1455 | 1.030 |

Dose-dependent cytotoxicity was observed with only anti-Vβ17/anti-CD28/anti-PSMA antibodies on C4-2B cells FIG. 33. No cytotoxicity was observed with anti-Vβ17/null/anti-PSMA antibody or for antibodies with PMSA on the CD28 light chain N-terminus.

Table 25 further demonstrates a summary of anti-Vβ17/anti-CD28/anti-PSMA antibodies binding to Pan T cells and H929 Target Cells.

TABLE 25
Binding data for anti-Vβ17/anti-CD28/anti-PSMA antibodies

| | | | H929 cell |
|---|---|---|---|
| | Pan T cell Binding EC₅₀ (μg/ml) | | Binding |
| Antibody | Donor 1 | Donor 2 | EC₅₀ (μg/ml) |
| VB28B33 | >10 | >10 | 1.404 |
| VB28B35 | >10 | >10 | <0.0046 |
| VB28B36 | ND | ND | <0.0046 |
| VB28B38 | 1.267 | 1.076 | <0.0046 |
| VB28B39 | >10 | >10 | 0.01673 |
| VB28B40 | 1.124 | 1.213 | 0.1206 |
| B17B619 | 0.1589 | 0.1244 | 0.1223 |
| B17B620 | >10 | >10 | >10 |

Table 26 demonstrates a summary of anti-Vβ17/anti-CD28/anti-PSMA antibody cytotoxicity using H929 Target Cells.

TABLE 26
Cytotoxicity data for anti-Vβ17/anti-CD28/anti-PSMA antibodies

H929 cell Binding EC₅₀ (μg/ml)

| Antibody | Donor 1 | Donor 2 |
|---|---|---|
| VB28B33 | 0.6249 | 0.3780 |
| VB28B35 | <0.0046 | <0.0046 |
| VB28B36 | ND | ND |
| VB28B38 | ND | ND |
| VB28B39 | <0.0046 | <0.0046 |
| VB28B40 | ND | ND |
| B17B619 | 0.01692 | 0.01282 |
| B17B620 | ND | ND |

### Example 5: Exemplary Antibodies and Sequences

Additional details of exemplary antibodies used in certain Examples herein are provided in Table 27 and Table 28.

TABLE 27
Exemplary Vβ17, CD28, BCMA and PSMA Antibody Clones.

| | | VH | VL | VH CDR1* | VH CDR2 | VH CDR3 | VL CDR1 | VL CDR2 | VL CDR3 |
|---|---|---|---|---|---|---|---|---|---|
| | Target | SEQ ID | SEQ ID | SEQ ID | SEQ ID | SEQ ID | SEQ ID | SEQ ID | SEQ ID |
| E17.5F | Vβ17 | 25 | 26 | 1 | 2 | 3 | 4 | 5 | 6 |
| B17B1 | Vβ17 | 25 | 26 | 1 | 2 | 3 | 4 | 5 | 6 |
| B17H1 | Vβ17 | 25 | 26 | 1 | 2 | 3 | 4 | 5 | 6 |
| B17H3 | Vβ17 | 19 | 22 | 1 | 2 | 3 | 4 | 5 | 6 |
| B17H4 | Vβ17 | 20 | 23 | 1 | 2 | 3 | 4 | 5 | 6 |
| B17H5 | Vβ17 | 21 | 24 | 1 | 2 | 3 | 4 | 5 | 6 |
| B17B14 | Vβ17 | 19 | 22 | 1 | 2 | 3 | 4 | 5 | 6 |
| B17B15 | Vβ17 | 19 | 23 | 1 | 2 | 3 | 4 | 5 | 6 |
| B17B16 | Vβ17 | 19 | 24 | 1 | 2 | 3 | 4 | 5 | 6 |
| B17B17 | Vβ17 | 20 | 22 | 1 | 2 | 3 | 4 | 5 | 6 |
| B17B18 | Vβ17 | 20 | 23 | 1 | 2 | 3 | 4 | 5 | 6 |
| B17B19 | Vβ17 | 20 | 24 | 1 | 2 | 3 | 4 | 5 | 6 |
| B17B20 | Vβ17 | 21 | 22 | 1 | 2 | 3 | 4 | 5 | 6 |
| B17B21 | Vβ17 | 21 | 23 | 1 | 2 | 3 | 4 | 5 | 6 |
| B17B22 | Vβ17 | 21 | 24 | 1 | 2 | 3 | 4 | 5 | 6 |
| B17B2 | Vβ17 | 46 | 47 | 45 | 2 | 3 | 4 | 5 | 6 |
| Vb17_202B4D1 | Vβ17 | 77 | 78 | 48 | 49 | 50 | 51 | 52 | 53 |
| Vb17_210E10A1 | Vβ17 | 79 | 80 | 48 | 54 | 55 | 56 | 57 | 58 |
| B17B663 | Vβ17 | 81 | 82 | 59 | 49 | 60 | 61 | 62 | 63 |
| B17B694 | Vβ17 | 83 | 84 | 64 | 65 | 66 | 67 | 68 | 69 |
| B17B698 | Vβ17 | 85 | 86 | 70 | 49 | 71 | 61 | 72 | 73 |
| B17B733 | Vβ17 | 87 | 88 | 48 | 74 | 75 | 56 | 57 | 76 |
| Vb17_N33S | Vβ17 | 21 | 665 | 1 | 2 | 3 | 664 | 5 | 6 |
| C28B11 | CD28 | 690 | 696 | 721 | 727 | 733 | 812 | 818 | 824 |
| C28B19 | CD28 | 691 | 697 | 722 | 728 | 734 | 813 | 819 | 825 |
| C28B103 | CD28 | 692 | 698 | 723 | 729 | 735 | 814 | 820 | 826 |
| C28B105 | CD28 | 693 | 699 | 724 | 730 | 736 | 815 | 821 | 827 |
| BCMB519 | BCMA | 92 | 96 | 89 | 90 | 91 | 92 | 93 | 94 |
| PSMB410 | PSMA | 1046 | 1047 | 1019 | 1020 | 1021 | 1034 | 1035 | 1036 |

*VH CDR1-3 and VL CDR1-3 sequences shown by single definition only. Other CDR definitions known in the art may also be utilized (e.g., Exemplary, Kabat, Chothia, AbM, IMGT, and/or Contact).

TABLE 28
Exemplary Multispecific Antibodies.

| | | | | HC 1 | | LC 1 | | HC 2 | | LC 1 | Variable |
|---|---|---|---|---|---|---|---|---|---|---|---|
| | | | HC 1 | SEQ | LC 1 | SEQ | HC 2 | SEQ | LC 2 | SEQ | Region |
| Name | Description | Isotype | Isotype | ID | Isotype | ID | Isotype | ID | Isotype | ID | Name |
| B17B619 | HC1 (ZWA): | IgG1, | IgG1 | 942 | Kappa | 965 | IgG1 | 978 | NA | NA | BCMB519, |
| | Vb17-B17B21- | Kappa, | | | | | | | | | Vb17- |
| | N33S-Fab-RF; | IgG1 | | | | | | | | | B17B21-LC- |
| | HC2 (ZWB): | | | | | | | | | | N33S |
| | BCMB519-LH-scFv | | | | | | | | | | |
| B17B620 | HC1 (ZWA): | IgG1, | IgG1 | 943 | Kappa | 966 | IgG1 | 1049 | NA | NA | Vb17- |
| | Vb17-B17B21- | Kappa, | | | | | | | | | B17B21-LC- |
| | N33S-Fab-RF; | IgG1 | | | | | | | | | N33S, |
| | HC2 (ZWB): | | | | | | | | | | Null-LH- |
| | Null-LH- | | | | | | | | | | MSCD334 |
| | MSCD334-scFv | | | | | | | | | | |
| VB28B2 | HC1 (KIHA): N- | IgG1, | IgG1 | — | Kappa | — | IgG1 | — | Kappa | — | BCMB519, |
| | term: C28B19- | Kappa, | | | | | | | | | Vb17- |
| | Fab, C-term: | IgG1, | | | | | | | | | B17B21-LC- |
| | Vb17-B17B21- | Kappa | | | | | | | | | N33S, |
| | N33S-LH-scFv; | | | | | | | | | | C28B19; |
| | HC2 (KIHB): N- | | | | | | | | | | C28F72 |
| | term: C28B19- | | | | | | | | | | |
| | Fab-RF, C-term: | | | | | | | | | | |
| | BCMB519-LH-scFv | | | | | | | | | | |
| VB28B4 | HC1 (KIHA): N- | IgG1, | IgG1 | — | Kappa | — | IgG1 | — | Kappa | — | BCMB519, |
| | term: C28B105 | Kappa, | | | | | | | | | Vb17- |
| | (C28Y3/4_093_B10)- | IgG1, | | | | | | | | | B17B21-LC- |
| | Fab, C-term: | Kappa | | | | | | | | | N33S, |
| | Vb17-B17B21- | | | | | | | | | | C28F102 |
| | N33S-LH-scFv; | | | | | | | | | | |
| | HC2 (KIHB): N- | | | | | | | | | | |
| | term: C28B105 | | | | | | | | | | |
| | (C28Y3/4_093_B10)- | | | | | | | | | | |
| | Fab-RF, C- | | | | | | | | | | |
| | term: BCMB519- | | | | | | | | | | |
| | LH-scFv | | | | | | | | | | |
| VB28B7 | HC1 (KIHA): N- | IgG1, | IgG1 | — | Kappa | — | IgG1 | — | Kappa | — | BCMB519, |
| | term: C28B19- | Kappa, | | | | | | | | | C28B19; |
| | Fab, C-term: | IgG1, | | | | | | | | | C28F72, |
| | Null-LH- | Kappa | | | | | | | | | Null-LH- |
| | MSCD334-scFv; | | | | | | | | | | MSCD334 |
| | HC2 (KIHB): N- | | | | | | | | | | |
| | term: C28B19- | | | | | | | | | | |
| | Fab-RF, C-term: | | | | | | | | | | |
| | BCMB519-LH-scFv | | | | | | | | | | |
| VB28B9 | HC1 (KIHA): N- | IgG1, | IgG1 | — | Kappa | — | IgG1 | — | Kappa | — | BCMB519, |
| | term: C28B105 | Kappa, | | | | | | | | | C28F102, |
| | (C28Y3/4_093_B10)- | IgG1, | | | | | | | | | Null-LH- |
| | Fab, C-term: | Kappa | | | | | | | | | MSCD334 |
| | Null-LH- | | | | | | | | | | |
| | MSCD334-scFv; | | | | | | | | | | |
| | HC2 (KIHB): N- | | | | | | | | | | |
| | term: C28B105 | | | | | | | | | | |
| | (C28Y3/4_093_B10)- | | | | | | | | | | |
| | Fab-RF, C- | | | | | | | | | | |
| | term: BCMB519- | | | | | | | | | | |
| | LH-scFv | | | | | | | | | | |
| VB28B16 | HC1 (Knob3): N- | IgG1, | IgG1 | 944 | Kappa | 967 | IgG1 | 980 | Kappa | 1001 | Vb17- |
| | term: C28B19- | Kappa, | | | | | | | | | B17B21-LC- |
| | Fab, C-term: | IgG1, | | | | | | | | | N33S, |
| | Vb17-B17B21- | Kappa | | | | | | | | | C28B19; |
| | N33S-LH-scFv; | | | | | | | | | | C28F72 |
| | HC2 (Hole3-RF): | | | | | | | | | | |
| | C28B19-Fab-RF | | | | | | | | | | |
| VB28B17 | HC1 (Knob3): N- | IgG1, | IgG1 | 945 | Kappa | 968 | IgG1 | 981 | Kappa | 1002 | Vb17- |
| | term: C28B105- | Kappa, | | | | | | | | | B17B21-LC- |
| | Fab, C-term: | IgG1, | | | | | | | | | N33S, |
| | Vb17-B17B21- | Kappa | | | | | | | | | C28F102 |
| | N33S-LH-scFv; | | | | | | | | | | |
| | HC2 (Hole3-RF): | | | | | | | | | | |
| | C28B105-Fab-RF | | | | | | | | | | |
| VB28B18 | HC1 (Knob3): N- | IgG1, | IgG1 | 946 | Kappa | 969 | IgG1 | 982 | Kappa | 1003 | B21M; |
| | term: B21M-Fab, | Kappa, | | | | | | | | | 12782L_19, |
| | C-term: Vb17- | IgG1, | | | | | | | | | Vb17- |
| | B17B21-N33S-LH- | Kappa | | | | | | | | | B17B21-LC- |
| | scFv; HC2 | | | | | | | | | | N33S |
| | (Hole3-RF): | | | | | | | | | | |
| | B21M-Fab-RF | | | | | | | | | | |
| VB28B19 | HC1 (Knob3): N- | IgG1, | IgG1 | 947 | Kappa | 970 | IgG1 | 983 | Kappa | 1004 | C28B19; |
| | term: C28B19- | Kappa, | | | | | | | | | C28F72, |
| | Fab, C-term: | IgG1, | | | | | | | | | Null-LH- |
| | Null-LH- | Kappa | | | | | | | | | MSCD334 |
| | MSCD334-scFv; | | | | | | | | | | |
| | HC2 (Hole3-RF): | | | | | | | | | | |
| | C28B19-Fab-RF | | | | | | | | | | |
| VB28B20 | HC1 (Knob3): N- | IgG1, | IgG1 | 948 | Kappa | 971 | IgG1 | 984 | Kappa | 1005 | C28F102, |
| | term: C28B105- | Kappa, | | | | | | | | | Null-LH- |
| | Fab, C-term: | IgG1, | | | | | | | | | MSCD334 |
| | Null-LH- | Kappa | | | | | | | | | |
| | MSCD334-scFv; | | | | | | | | | | |
| | HC2 (Hole3-RF): | | | | | | | | | | |
| | C28B105-Fab-RF | | | | | | | | | | |
| VB28B21 | HC1 (Knob3): N- | IgG1, | IgG1 | 949 | Kappa | 972 | IgG1 | 985 | Kappa | 1006 | B21M; |
| | term: B21M-Fab, | Kappa, | | | | | | | | | 12782L_19, |
| | C-term: Null- | IgG1, | | | | | | | | | Null-LH- |
| | LH-MSCD334- | Kappa | | | | | | | | | MSCD334 |
| | scFv; HC2 | | | | | | | | | | |
| | (Hole3-RF): | | | | | | | | | | |
| | B21M-Fab-RF | | | | | | | | | | |
| VB28B22 | HC1 (Knob3): N- | IgG1, | IgG1 | 950 | Kappa | 973 | IgG1 | 986 | NA | NA | Vb17- |
| | term: C28B19- | Kappa, | | | | | | | | | B17B21-LC- |
| | Fab, C-term: | IgG1 | | | | | | | | | N33S, |
| | Vb17-B17B21- | | | | | | | | | | C28B19; |
| | N33S-LH-scFv; | | | | | | | | | | C28F72, |
| | HC2 (Hole3): | | | | | | | | | | Null-LH- |
| | Null-LH- | | | | | | | | | | MSCD334 |
| | MSCD334- | | | | | | | | | | |
| | scFv_K447 | | | | | | | | | | |
| VB28B23 | HC1 (Knob3): N- | IgG1, | IgG1 | 951 | Kappa | 974 | IgG1 | 987 | NA | NA | Vb17- |
| | term: C28B105- | Kappa, | | | | | | | | | B17B21-LC- |
| | Fab, C-term: | IgG1 | | | | | | | | | N33S, |
| | Vb17-B17B21- | | | | | | | | | | C28F102, |
| | N33S-LH-scFv; | | | | | | | | | | Null-LH- |
| | HC2 (Hole3): | | | | | | | | | | MSCD334 |
| | Null-LH- | | | | | | | | | | |
| | MSCD334- | | | | | | | | | | |
| | scFv_K447 | | | | | | | | | | |
| VB28B24 | HC1 (Knob3): N- | IgG1, | IgG1 | 952 | Kappa | 975 | IgG1 | 988 | NA | NA | B21M; |
| | term: B21M-Fab, | Kappa, | | | | | | | | | 12782L_19, |
| | C-term: Vb17- | IgG1 | | | | | | | | | Vb17- |
| | B17B21-N33S-LH- | | | | | | | | | | B17B21-LC- |
| | scFv; HC2 | | | | | | | | | | N33S, |
| | (Hole3): Null- | | | | | | | | | | Null-LH- |
| | LH-MSCD334- | | | | | | | | | | MSCD334 |
| | scFv_K447 | | | | | | | | | | |
| VB28B25 | HC1 (Knob3): N- | IgG1, | IgG1 | 953 | Kappa | 976 | IgG1 | 989 | NA | NA | C28B19; |
| | term: C28B19- | Kappa, | | | | | | | | | C28F72, |
| | Fab, C-term: | IgG1 | | | | | | | | | Null-LH- |
| | Null-LH- | | | | | | | | | | MSCD334 |
| | MSCD334-scFv; | | | | | | | | | | |
| | HC2 (Hole3): | | | | | | | | | | |
| | Null-LH- | | | | | | | | | | |
| | MSCD334- | | | | | | | | | | |
| | scFv_K447 | | | | | | | | | | |
| VB28B26 | HC1 (Knob3): N- | IgG1, | IgG1 | 954 | Kappa | 977 | IgG1 | 990 | NA | NA | C28F102, |
| | term: C28B105- | Kappa, | | | | | | | | | Null-LH- |
| | Fab, C-term: | IgG1 | | | | | | | | | MSCD334 |
| | Null-LH- | | | | | | | | | | |
| | MSCD334-scFv; | | | | | | | | | | |
| | HC2 (Hole3): | | | | | | | | | | |
| | Null-LH- | | | | | | | | | | |
| | MSCD334- | | | | | | | | | | |
| | scFv_K447 | | | | | | | | | | |
| VB28B27 | HC1 (Knob3): N- | IgG1, | IgG1 | 955 | Kappa | 978 | IgG1 | 991 | NA | NA | B21M; |
| | term: B21M-Fab, | Kappa, | | | | | | | | | 12782L_19, |
| | C-term: Null- | IgG1 | | | | | | | | | Null-LH- |
| | LH-MSCD334- | | | | | | | | | | MSCD334 |
| | scFv; HC2 | | | | | | | | | | |
| | (Hole3): Null- | | | | | | | | | | |
| | LH-MSCD334- | | | | | | | | | | |
| | scFv_K447 | | | | | | | | | | |
| VB28B28 | HC1 (Knob3): | IgG1, | IgG1 | 1048 | NA | NA | IgG1 | 1050 | Kappa | 1051 | PSMB410_PS |
| | Vb17-B17B21- | IgG1, | | | | | | | | | 3F63, |
| | N33S-LH-scFv; | Kappa | | | | | | | | | Vb17- |
| | HC2 (Hole3-RF): | | | | | | | | | | B17B21-LC- |
| | CD28B19-Fab-RF, | | | | | | | | | | N33S |
| | LC-N-term: | | | | | | | | | | |
| | PSMB410-LH-scFv | | | | | | | | | | |
| VB28B29 | HC1 (Knob3): | IgG1, | IgG1 | 956 | NA | NA | IgG1 | 992 | Kappa | 1007 | PSMB410_PS |
| | Null-scFv; HC2 | IgG1, | | | | | | | | | 3F63, |
| | (Hole3-RF): | Kappa | | | | | | | | | Null-LH- |
| | CD28B19-Fab-RF, | | | | | | | | | | MSCD334 |
| | LC-N-term: | | | | | | | | | | |
| | PSMB410-LH-scFv | | | | | | | | | | |
| VB28B31 | HC1 (Knob3): | IgG1, | IgG1 | 957 | NA | NA | IgG1 | 993 | Kappa | 1008 | PSMB410_PS |
| | PSMB410-LH- | IgG1, | | | | | | | | | 3F63 |
| | scFv; HC2 | Kappa | | | | | | | | | |
| | (Hole3-RF): | | | | | | | | | | |
| | CD28B19-Fab-RF | | | | | | | | | | |
| VB28B32 | HC1 (Knob3): | IgG1, | IgG1 | 958 | NA | NA | IgG1 | 994 | Kappa | 1009 | PSMB410_PS |
| | PSMB410-LH- | IgG1, | | | | | | | | | 3F63, |
| | scFv; HC2 | Kappa | | | | | | | | | Vb17- |
| | (Hole3-RF): | | | | | | | | | | B17B21-LC- |
| | CD28B19-Fab-RF, | | | | | | | | | | N33S, |
| | LC-N-term: | | | | | | | | | | C28B19; |
| | Vb17-B17B21- | | | | | | | | | | C28F72 |
| | N33S-LH-scFv | | | | | | | | | | |
| VB28B33 | HC1 (Knob3): | IgG1, | IgG1 | 959 | NA | NA | IgG1 | 995 | Kappa | 1010 | Vb17- |
| | Null-scFv; HC2 | IgG1, | | | | | | | | | B17B21-LC- |
| | (Hole3-RF): | Kappa | | | | | | | | | N33S, |
| | CD28B19-Fab-RF, | | | | | | | | | | C28B19; |
| | LC-N-term: | | | | | | | | | | C28F72, |
| | Vb17-B17B21- | | | | | | | | | | Null-LH- |
| | N33S-LH-scFv | | | | | | | | | | MSCD334 |
| VB28B35 | HC1 (Knob3): | IgG1, | IgG1 | 960 | NA | NA | IgG1 | 996 | Kappa | 1011 | BCMB519, |
| | Vb17-B17B21- | IgG1, | | | | | | | | | Vb17- |
| | N33S-LH-scFv; | Kappa | | | | | | | | | B17B21-LC- |
| | HC2 (Hole3-RF): | | | | | | | | | | N33S |
| | CD28B19-Fab-RF, | | | | | | | | | | |
| | LC-N-term: | | | | | | | | | | |
| | BCMB519-LH-scFv | | | | | | | | | | |
| VB28B36 | HC1 (Knob3): | IgG1, | IgG1 | 961 | NA | NA | IgG1 | 997 | Kappa | 1012 | BCMB519, |
| | Null-scFv; HC2 | IgG1, | | | | | | | | | Null-LH- |
| | (Hole3-RF): | Kappa | | | | | | | | | MSCD334 |
| | CD28B19-Fab-RF, | | | | | | | | | | |
| | LC-N-term: | | | | | | | | | | |
| | BCMB519-LH-scFv | | | | | | | | | | |
| VB28B38 | HC1 (Knob3): | IgG1, | IgG1 | 962 | NA | NA | IgG1 | 998 | Kappa | 1013 | BCMB519 |
| | BCMB519-LH- | IgG1, | | | | | | | | | |
| | scFv; HC2 | Kappa | | | | | | | | | |
| | (Hole3-RF): | | | | | | | | | | |
| | CD28B19-Fab-RF | | | | | | | | | | |
| VB28B39 | HC1 (Knob3): | IgG1, | IgG1 | 963 | NA | NA | IgG1 | 999 | Kappa | 1014 | BCMB519, |
| | BCMB519-LH- | IgG1, | | | | | | | | | Vb17- |
| | scFv; HC2 | Kappa | | | | | | | | | B17B21-LC- |
| | (Hole3-RF): | | | | | | | | | | N33S, |
| | CD28B19-Fab-RF, | | | | | | | | | | C28B19; |
| | LC-N-term: | | | | | | | | | | C28F72 |
| | Vb17-B17B21- | | | | | | | | | | |
| | N33S-LH-scFv | | | | | | | | | | |
| VB28B40 | HC1 (Knob3): | IgG1, | IgG1 | 964 | NA | 965 | IgG1 | 1000 | Kappa | 1015 | BCMB519, |
| | Vb17-B17B21- | IgG1, | | | | | | | | | B21M; |
| | N33S-LH-scFv; | Kappa | | | | | | | | | 12782L_19, |
| | HC2 (Hole3-RF): | | | | | | | | | | Vb17- |
| | B21M-Fab-RF, | | | | | | | | | | B17B21-LC- |
| | LC-N-term: | | | | | | | | | | N33S |
| | BCMB519-LH-scFv | | | | | | | | | | |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A trispecific antibody comprising: (a) a first binding domain that binds to Vβ17, (b) a second binding domain that binds to cancer antigen, and (c) a third binding domain that binds to CD28;
wherein optionally the cancer antigen is BCMA, or
wherein optionally the cancer antigen is PSMA.

2. The trispecific antibody of claim 1, wherein:
(a) the first binding domain that binds to Vβ17 comprises:
(1) (i) a VH comprising a VH CDR1, a VH CDR2, and a VH CDR3 having an amino acid sequence of a VH CDR1, VH CDR2, and VH CDR3, respectively, of SEQ ID NO:9; and (ii) a VL comprising a VL CDR1, a VL CDR2, and a VL CDR3 having an amino acid sequence of a VL CDR1, VL CDR2, and VL CDR3, respectively, of SEQ ID NO:10;
(2) (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:22;
(3) (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:23;
(4) (i) a VH comprising a VH CDR1, a VH CDR2, and a VH CDR3 having an amino acid sequence of a VH CDR1, VH CDR2, and VH CDR3, respectively, of SEQ ID NO:19; and (ii) a VL comprising a VL CDR1, a VL CDR2, and a VL CDR3 having an amino acid sequence of a VL CDR1, VL CDR2, and VL CDR3, respectively, of SEQ ID NO:24;
(5) (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:22;
(6) (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:23;
(7) (i) a VH comprising a VH CDR1, a VH CDR2, and a VH CDR3 having an amino acid sequence of a VH CDR1, VH CDR2, and VH CDR3, respectively, of SEQ ID NO:20; and (ii) a VL comprising a VL CDR1, a VL CDR2, and a VL CDR3 having an amino acid sequence of a VL CDR1, VL CDR2, and VL CDR3, respectively, of SEQ ID NO:24;
(8) (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:22;
(9) (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:23;
(10) (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:24;
(11) (i) a VH comprising a VH CDR1, a VH CDR2, and a VH CDR3 having an amino acid sequence of a VH CDR1, VH CDR2, and VH CDR3, respectively, of SEQ ID NO:46; and (ii) a VL comprising a VL CDR1, a VL CDR2, and a VL CDR3 having an amino acid sequence of a VL CDR1, VL CDR2, and VL CDR3, respectively, of SEQ ID NO:49;
(12) (i) a VH comprising a VH CDR1, a VH CDR2, and a VH CDR3 having an amino acid sequence of a VH CDR1, VH CDR2, and VH CDR3, respectively, of SEQ ID NO:77; and (ii) a VL comprising a VL CDR1, a VL CDR2, and a VL CDR3 having an amino acid sequence of a VL CDR1, VL CDR2, and VL CDR3, respectively, of SEQ ID NO:78;
(13) (i) a VH comprising a VH CDR1, a VH CDR2, and a VH CDR3 having an amino acid sequence of a VH CDR1, VH CDR2, and VH CDR3, respectively, of SEQ ID NO:79; and (ii) a VL comprising a VL CDR1, a VL CDR2, and a VL CDR3 having an amino acid sequence of a VL CDR1, VL CDR2, and VL CDR3, respectively, of SEQ ID NO:80;
(14) (i) a VH comprising a VH CDR1, a VH CDR2, and a VH CDR3 having an amino acid sequence of a VH CDR1, VH CDR2, and VH CDR3, respectively, of SEQ ID NO:81; and (ii) a VL comprising a VL CDR1, a VL CDR2, and a VL CDR3 having an amino acid sequence of a VL CDR1, VL CDR2, and VL CDR3, respectively, of SEQ ID NO:82;
(15) (i) a VH comprising a VH CDR1, a VH CDR2, and a VH CDR3 having an amino acid sequence of a VH CDR1, VH CDR2, and VH CDR3, respectively, of SEQ ID NO:83; and (ii) a VL comprising a VL CDR1, a VL CDR2, and a VL CDR3 having an amino acid sequence of a VL CDR1, VL CDR2, and VL CDR3, respectively, of SEQ ID NO:84;
(16) (i) a VH comprising a VH CDR1, a VH CDR2, and a VH CDR3 having an amino acid sequence of a VH CDR1, VH CDR2, and VH CDR3, respectively, of SEQ ID NO:85; and (ii) a VL comprising a VL CDR1, a VL CDR2, and a VL CDR3 having an amino acid sequence of a VL CDR1, VL CDR2, and VL CDR3, respectively, of SEQ ID NO:86;
(17) (i) a VH comprising a VH CDR1, a VH CDR2, and a VH CDR3 having an amino acid sequence of a VH CDR1, VH CDR2, and VH CDR3, respectively, of SEQ ID NO:87; and (ii) a VL comprising a VL CDR1, a VL CDR2, and a VL CDR3 having an amino acid sequence of a VL CDR1, VL CDR2, and VL CDR3, respectively, of SEQ ID NO:88;
(18) (i) a VH comprising a VH CDR1, a VH CDR2, and a VH CDR3 having an amino acid sequence of a VH CDR1, VH CDR2, and VH CDR3, respectively, of SEQ ID NO:21; and (ii) a VL comprising a VL CDR1, a VL CDR2, and a VL CDR3 having an amino acid sequence of a VL CDR1, VL CDR2, and VL CDR3, respectively, of SEQ ID NO:665; or
(19) (i) a VH comprising a VH CDR1, a VH CDR2, and a VH CDR3 having an amino acid sequence of a VH CDR1, VH CDR2, and VH CDR3, respectively, of SEQ ID NO:1084; and (ii) a VL comprising a VL CDR1, a VL CDR2, and a VL CDR3 having an amino acid sequence of a VL CDR1, VL CDR2, and VL CDR3, respectively, of SEQ ID NO:1085;
(b) the cancer antigen is BCMA; optionally wherein the second binding domain that binds to BCMA comprises:
(1) (i) a VH comprising a VH CDR1, a VH CDR2, and a VH CDR3 having an amino acid sequence of a VH CDR1, VH CDR2, and VH CDR3, respectively, of SEQ ID NO:95; and (ii) a VL comprising a VL CDR1, a VL CDR2, and a VL CDR3 having an amino acid sequence of a VL CDR1, VL CDR2, and VL CDR3, respectively, of SEQ ID NO:96;
(2) (i) a VH comprising a VH CDR1, a VH CDR2, and a VH CDR3 having an amino acid sequence of a VH CDR1, VH CDR2, and VH CDR3, respectively, of SEQ ID NO:1052; and (ii) a VL comprising a VL CDR1, a VL CDR2, and a VL CDR3 having an amino acid sequence of a VL CDR1, VL CDR2, and VL CDR3, respectively, of SEQ ID NO:1053; or
(c) the cancer antigen is PSMA; optionally wherein the second binding domain that binds to PSMA comprises:
(1) a VH comprising a VH CDR1, a VH CDR2, and a VH CDR3 having an amino acid sequence of a VH CDR1, VH CDR2, and VH CDR3, respectively, of SEQ ID NO:1046; and (ii) a VL comprising a VL CDR1, a VL CDR2, and a VL CDR3 having an amino acid sequence of a VL CDR1, VL CDR2, and VL CDR3, respectively, of SEQ ID NO:1047; or
(d) the third binding domain that binds to CD28 comprises:
(1) (i) a VH comprising a VH CDR1, a VH CDR2, and a VH CDR3 having an amino acid sequence of a VH CDR1, VH CDR2, and VH CDR3, respectively, of SEQ ID NO:690; and (ii) a VL comprising a VL CDR1, a VL CDR2, and a VL CDR3 having an amino acid sequence of a VL CDR1, VL CDR2, and VL CDR3, respectively, of SEQ ID NO:696;
(2) (i) a VH comprising a VH CDR1, a VH CDR2, and a VH CDR3 having an amino acid sequence of a VH CDR1, VH CDR2, and VH CDR3, respectively, of SEQ ID NO:691; and (ii) a VL comprising a VL CDR1, a VL CDR2, and a VL CDR3 having an amino acid sequence of a VL CDR1, VL CDR2, and VL CDR3, respectively, of SEQ ID NO:697;
(3) (i) a VH comprising a VH CDR1, a VH CDR2, and a VH CDR3 having an amino acid sequence of a VH CDR1, VH CDR2, and VH CDR3, respectively, of SEQ ID NO:692; and (ii) a VL comprising a VL CDR1, a VL CDR2, and a VL CDR3 having an amino acid sequence of a VL CDR1, VL CDR2, and VL CDR3, respectively, of SEQ ID NO:698; or
(4) (i) a VH comprising a VH CDR1, a VH CDR2, and a VH CDR3 having an amino acid sequence of a VH CDR1, VH CDR2, and VH CDR3, respectively, of SEQ ID NO:693; and (ii) a VL comprising a VL CDR1, a VL CDR2, and a VL CDR3 having an amino acid sequence of a VL CDR1, VL CDR2, and VL CDR3, respectively, of SEQ ID NO:699.

3. The trispecific antibody of claim 2, wherein
i. the VH CDR1, VH CDR2, VH CDR3, VL CDR1, VL CDR2, and VL CDR3 amino acid sequences of the first, second, or third binding domain are according to the Kabat numbering system;
ii. the VH CDR1, VH CDR2, VH CDR3, VL CDR1, VL CDR2, and VL CDR3 amino acid sequences of the first, second, or third binding domain are according to the Chothia numbering system;
iii. the VH CDR1, VH CDR2, VH CDR3, VL CDR1, VL CDR2, and VL CDR3 amino acid sequences of the first, second, or third binding domain are according to the AbM numbering system;
iv. the VH CDR1, VH CDR2, VH CDR3, VL CDR1, VL CDR2, and VL CDR3 amino acid sequences of the first, second, or third binding domain are according to the Contact numbering system;
v. the VH CDR1, VH CDR2, VH CDR3, VL CDR1, VL CDR2, and VL CDR3 amino acid sequences of the first, second, or third binding domain are according to the IMGT numbering system; or
vi. the VH CDR1, VH CDR2, VH CDR3, VL CDR1, VL CDR2, and VL CDR3 amino acid sequences of the first, second, or third binding domain are according to the Exemplary numbering system.

4. The trispecific antibody of claim 1, wherein the first binding domain:
(1) binds to Vβ17 that is present on the surface of a T cell;
(2) binds to a Vβ17 antigen;
(3) to a Vβ17 epitope; or
(4) specifically binds to a Vβ17 antigen.

5. The trispecific antibody of claim 1, wherein the cancer antigen is:
(1) present on the surface of a cell;
(2) BCMA, wherein optionally, the BCMA is present on the surface of a B cell;
or
(3) PSMA, wherein optionally, the PSMA is present on the surface of a prostate cell.

6.-13. (canceled)

14. The trispecific antibody of claim 1, wherein the third binding domain:
(a) binds to CD28 that is present on the surface of a T cell;
(b) binds an antigen of CD28;
(2) binds an epitope of CD28; or
(3) specifically binds to CD28.

15. (canceled)

16. (canceled)

17. The trispecific antibody of claim 1, wherein
i. the first binding domain is humanized,
ii. the second binding domain is humanized,
iii. the third binding domain is humanized,
iv. the first binding domain and second binding domain are humanized,
v. the first binding domain and third binding domain are humanized,
vi. the second binding domain and third binding domain are humanized, or
vii. the first binding domain, the second binding domain and the third binding domain are humanized.

18. The trispecific antibody of claim 1, wherein the trispecific antibody:
(1) is an IgG antibody, wherein optionally, the IgG antibody is an IgG1, IgG2, IgG3, or IgG4;
(2) comprises a kappa light chain;
(3) comprises a lambda light chain;
(4) is multivalent.

19.-42. (canceled)

43. A trispecific antibody comprising: a first means capable of binding Vβ17 on the surface of a T cell; a second means capable of binding a cancer antigen on the surface of a cancer cell; and a third means capable of binding CD28 on the surface of the T cell;
wherein optionally, the cancer antigen is BCMA and the cancer cell is a B cell cancer cell, or
wherein optionally, the cancer antigen is PSMA and the cancer cell is a prostate cancer cell.

44. (canceled)

45. (canceled)

46. A nucleic acid encoding the trispecific antibody of claim 1.

47. A vector comprising the nucleic acid of claim 46.

48. A host cell comprising the vector of claim 47.

49. A kit comprising the vector of claim 47 and packaging for the same.

50. A kit comprising the trispecific antibody of claim 1 and packaging for the same.

51. A pharmaceutical composition comprising the trispecific antibody of claim 1, and a pharmaceutically acceptable carrier.

52. A method of producing the pharmaceutical composition of claim 51, comprising combining the trispecific antibody with a pharmaceutically acceptable carrier to obtain the pharmaceutical composition.

53. A method of:
(1) activating a T cell expressing Vβ17, comprising contacting the T cell with the trispecific antibody of claim 1;
(2) directing a T cell expressing Vβ17 and CD28 to a B cell, the method comprising contacting the T cell with the trispecific antibody of claim 1, wherein the contacting directs the T cell to the B cell;
(3) inhibiting growth or proliferation of B cells expressing BCMA on the cell surface, the method comprising contacting the B cells with the trispecific antibody of claim 1, wherein contacting the B cells with the pharmaceutical composition or the antibody or the trispecific antibody inhibits growth or proliferation of the B cells;
wherein:
(a) the B cells are in the presence of a T cell expressing Vβ17 while in contact with the trispecific antibody; or
(b) the B cells are in the presence of a T cell expressing CD28 while in contact with the trispecific antibody;
(4) directing a T cell expressing Vβ17 to a cancer cell, the method comprising contacting the T cell with the trispecific antibody of claim 1, wherein the contacting directs the T cell to the cancer cell;
(5) inhibiting the growth or proliferation of a cancer cell, comprising contacting the trispecific antibody of claim 1, with the cancer cell having the cancer antigen present on the surface of the cancer cell, wherein the contacting is in the presence of a T cell expressing the Vβ17, and wherein the contacting results in the inhibition of the growth or proliferation of the cancer cell;
(6) eliminating a cancer cell in a subject, comprising contacting the trispecific antibody of claim 1 with the cancer cell having the cancer antigen present on the surface of the cancer cell, wherein the contacting is in the presence of a T cell expressing the Vβ17, and wherein the contacting results in the elimination of the cancer cell; wherein optionally, the subject is a human or wherein optionally, the subject is a subject in need thereof; or
(7) treating a disease in a subject, comprising administering an effective amount of the trispecific antibody of claim 1 to the subject, wherein the disease is caused all or in part by a cancer cell having the cancer antigen present on the surface of the cancer cell; wherein optionally, the subject is a human or wherein optionally, the subject is a subject in need thereof.

54. A process for making an antibody that binds to more than one target molecule, the process comprising:
a step for performing a function of obtaining a first binding domain capable of binding to Vβ17 on a T cell; a step for performing a function of obtaining a second binding domain capable of binding to cancer antigen on a cancer cell; a step for performing a function of obtaining a third binding domain capable of binding to CD28 on a T cell; and a step for performing a function of providing an antibody capable of binding to a Vβ17 antigen on the T cell, a cancer antigen on the cancer cell and the CD28 antigen on the T cell;
wherein (i) the step for performing a function of obtaining a second binding domain that binds to the cancer antigen on the cancer cell is repeated n times, and further comprising n steps for performing a function of providing a first binding domain that binds to Vβ17 present on a T cell and n number of target molecules, wherein n is at least 2, or (ii) the step for performing a function of obtaining a third binding domain that binds to the CD28 on the T cell is repeated n times, and further comprising n steps for performing a function of providing a first binding domain that binds to Vβ17 present on a T cell and n number of target molecules, wherein n is at least 2.

55. (canceled)

56. A method of directing a T cell expressing Vβ17 and CD28 to a B cell, the method comprising contacting the T cell with the trispecific antibody of claim 1, wherein the contacting directs the T cell to the B cell.

57.-65. (canceled)

66. The method of claim 56, wherein the cancer antigen is present on the surface of a cancer cell.

67. The method of claim 66, wherein
(i) the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer; wherein
(i) the adrenal cancer is an adrenocortical carcinoma (ACC), adrenal cortex cancer, pheochromocytoma, or neuroblastoma;
(ii) the anal cancer is a squamous cell carcinoma, cloacogenic carcinoma, adenocarcinoma, basal cell carcinoma, or melanoma;
(iii) the appendix cancer is a neuroendocrine tumor (NET), mucinous adenocarcinoma, goblet cell carcinoid, intestinal-type adenocarcinoma, or signet-ring cell adenocarcinoma;
(iv) the bile duct cancer is an extrahepatic bile duct cancer, adenocarcinomas, hilar bile duct cancer, perihilar bile duct cancer, distal bile duct cancer, or intrahepatic bile duct cancer;
(v) the bladder cancer is transitional cell carcinoma (TCC), papillary carcinoma, flat carcinoma, squamous cell carcinoma, adenocarcinoma, small-cell carcinoma, or sarcoma;
(vi) the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer;
(vii) the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma;
(viii) the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer;
(ix) the cervical cancer is a squamous cell carcinoma, or adenocarcinoma;
(x) the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma;
(xi) the esophageal cancer is an adenocarcinoma or squamous cell carcinoma;
(xii) the gall bladder cancer is an adenocarcinoma, papillary adenocarcinoma, adenosquamous carcinoma, squamous cell carcinoma, small cell carcinoma, or sarcoma;
(xiii) the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT);
(xiv) the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer;
(xv) the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL);
(xvi) the intestinal cancer is a small intestine cancer, small bowel cancer, adenocarcinoma, sarcoma, gastrointestinal stromal tumors, carcinoid tumors, or lymphoma;
(xvii) the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma;
(xviii) the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS);
(xix) the liver cancer is a hepatocellular carcinoma (HCC), fibrolamellar HCC, cholangiocarcinoma, angiosarcoma, or liver metastasis;
(xx) the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor;
(xxi) the melanoma is a superficial spreading melanoma, nodular melanoma, acral-lentiginous melanoma, lentigo maligna melanoma, amelanotic melanoma, desmoplastic melanoma, ocular melanoma, or metastatic melanoma;
(xxii) the mesothelioma is a pleural mesothelioma, peritoneal mesothelioma, pericardial mesothelioma, or testicular mesothelioma;
(xxiii) the multiple myeloma is an active myeloma or smoldering myeloma;
(xxiv) the neuroendocrine tumor, is a gastrointestinal neuroendocrine tumor, pancreatic neuroendocrine tumor, or lung neuroendocrine tumor;
(xxv) the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma;
(xxvi) the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer;
(xxvii) the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst;
(xxviii) the pancreatic cancer is a pancreatic exocrine gland cancer, pancreatic endocrine gland cancer, or pancreatic adenocarcinoma, islet cell tumor, or neuroendocrine tumor;
(xxix) the prostate cancer is a prostate adenocarcinoma, prostate sarcoma, transitional cell carcinoma, small cell carcinoma, or neuroendocrine tumor;
(xxx) the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer;
(xxxi) the skin cancer is a basal cell carcinoma, squamous cell carcinoma, melanoma, Merkel cell carcinoma, Kaposi sarcoma (KS), actinic keratosis, skin lymphoma, or keratoacanthoma;
(xxxii) the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma;
(xxxiii) the spinal cancer is a spinal metastatic tumor;
(xxxiv) the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid;
(xxxv) the testicular cancer is a seminoma, non-seminoma, embryonal carcinoma, yolk sac carcinoma, choriocarcinoma, teratoma, gonadal stromal tumor, leydig cell tumor, or sertoli cell tumor;
(xxxiv) the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer;
(xxxv) the thyroid cancer is a papillary carcinoma, follicular carcinoma, Hürthle cell carcinoma, medullary thyroid carcinoma, or anaplastic carcinoma;
(xxxvi) the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma;
(xxxvii) the vaginal cancer is a squamous cell carcinoma, adenocarcinoma, melanoma, or sarcoma; or
(xxxviii) the vulvar cancer is a squamous cell carcinoma or adenocarcinoma;
(ii) cancer antigen is an angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PAP, PDGFRα, PSA, PSA3, PSMA, RANKL, SLAMF7, STEAPI, TARP, TROP2, VEGF, or VEGF-R antigen; and/or
(iii) the cancer antigen is a CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, or BRCA1 antigen.

68. (canceled)

69. The method of claim 66, wherein the cancer antigen is BCMA or PSMA; wherein optionally the cancer is a lymphoma, leukemia or prostate cancer.

70. The method of claim 69, wherein the cancer cell is a B cell or a prostate cancer cell; wherein optionally the cancer is a lymphoma, leukemia or prostate cancer.

71.-75. (canceled)
